SEQUENCE LISTING (1 sequence)

SEQ ID NO: 1 - protein, 15 aa, synthetic construct
EQEDEPEGDYFEWLE

(19) United States
(12) Patent Application Publication
(10) Pub. No.: US 2024/0287086 A1
(43) Pub. Date: Aug. 29, 2024

(54) TRICYCLIC HETEROCYCLES AS FGFR INHIBITORS

(71) Applicant: Incyte Corporation, Wilmington, DE (US)

(72) Inventors: Matthew S. McCammant, Newark, DE (US); Evan Styduhar, Media, PA (US); Oleg Vechorkin, Wilmington, DE (US); Robert Swyka, Wilmington, DE (US); Wenqing Yao, Chadds Ford, PA (US)

(21) Appl. No.: 18/545,487

(22) Filed: Dec. 19, 2023

Related U.S. Application Data
(63) Continuation of application No. 17/110,740, filed on Dec. 3, 2020, now Pat. No. 11,897,891.
(60) Provisional application No. 62/943,540, filed on Dec. 4, 2019.

(51) Int. Cl. C07D 487/14; A61P 35/00; C07D 471/14

(52) U.S. Cl. CPC C07D 487/14; A61P 35/00; C07D 471/14

(57) ABSTRACT

The present disclosure relates to tricyclic heterocycles, and pharmaceutical compositions of the same, that are inhibitors of the FGFR enzyme and are useful in the treatment of FGFR-associated diseases such as cancer.

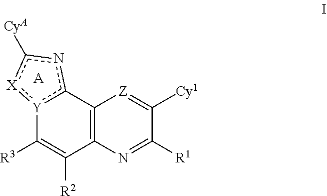

### FIELD

The present disclosure relates to tricyclic heterocycles, and pharmaceutical compositions of the same, that are inhibitors of the enzyme FGFR and are useful in the treatment of FGFR-associated diseases such as cancer.

### SEQUENCE LISTING

This application contains a Sequence Listing that has been submitted electronically as an XML file named 20443-0658002_SL_ST26.xml. The XML file, created on Dec. 19, 2023, is 1,922 bytes in size. The material in the XML file is hereby incorporated by reference in its entirety.

### BACKGROUND

The Fibroblast Growth Factor Receptors (FGFR) are receptor tyrosine kinases that bind to fibroblast growth factor (FGF) ligands. There are four FGFR proteins (FGFR1-4) that are capable of binding ligands and are involved in the regulation of many physiological processes including tissue development, angiogenesis, wound healing, and metabolic regulation. Upon ligand binding, the receptors undergo dimerization and phosphorylation leading to stimulation of the protein kinase activity and recruitment of many intracellular docking proteins. These interactions facilitate the activation of an array of intracellular signaling pathways including Ras-MAPK, AKT-PI3K, and phospholipase C that are important for cellular growth, proliferation and survival (Reviewed in Eswarakumar et al. Cytokine & Growth Factor Reviews, 2005, 16, 139-149). Aberrant activation of this pathway either through overexpression of FGF ligands or FGFR or activating mutations in the FGFRs can lead to tumor development, progression, and resistance to conventional cancer therapies. In human cancer, genetic alterations including gene amplification, chromosomal translocations and somatic mutations that lead to ligand-independent receptor activation have been described (Reviewed in Knights and Cook, Pharmacology & Therapeutics, 2010, 125, 105-117; Turner and Grose, Nature Reviews Cancer, 2010, 10, 116-129). Large scale DNA sequencing of thousands of tumor samples has revealed that FGFR genes are altered in many cancers (Helsten et al. Clin Cancer Res. 2016, 22, 259-267). Some of these activating mutations are identical to germline mutations that lead to skeletal dysplasia syndromes (Gallo et al. Cytokine & Growth Factor Reviews 2015, 26, 425-449). Mechanisms that lead to aberrant ligand-dependent signaling in human disease include overexpression of FGFs and changes in FGFR splicing that lead to receptors with more promiscuous ligand binding abilities. Therefore, development of inhibitors targeting FGFR may be useful in the clinical treatment of diseases that have elevated FGF or FGFR activity.

The cancer types in which FGF/FGFRs are implicated include, but are not limited to: carcinomas (e.g., bladder, breast, colorectal, endometrial, gastric, head and neck, kidney, lung, ovarian, prostate); hematopoietic malignancies (e.g., multiple myeloma, acute myelogenous leukemia, and myeloproliferative neoplasms); and other neoplasms (e.g., glioblastoma and sarcomas). In addition to a role in oncogenic neoplasms, FGFR activation has also been implicated in skeletal and chondrocyte disorders including, but not limited to, achrondroplasia and craniosynostosis syndromes.

There is a continuing need for the development of new drugs for the treatment of cancer, and the FGFR inhibitors described herein help address this need.

### SUMMARY

The present disclosure is directed to compounds having Formula (I):

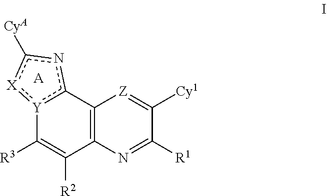

or pharmaceutically acceptable salts thereof, wherein constituent variables are defined herein.

The present disclosure is further directed to pharmaceutical compositions comprising a compound of Formula (I), or a pharmaceutically acceptable salt thereof, and at least one pharmaceutically acceptable carrier.

The present disclosure is further directed to methods of inhibiting an FGFR enzyme (e.g., an FGFR3 enzyme) comprising contacting the enzyme with a compound of Formula (I), or a pharmaceutically acceptable salt thereof.

The present disclosure is further directed to a method of treating a disease associated with abnormal activity or expression of an FGFR enzyme (e.g., an FGFR3 enzyme), comprising administering a compound of Formula (I), or a pharmaceutically acceptable salt thereof, to a patient in need thereof.

The present disclosure is further directed to compounds of Formula (I) for use in treating a disease associated with abnormal activity or expression of an FGFR enzyme (e.g., an FGFR3 enzyme).

The present disclosure is further directed to a method for treating a disorder mediated by an FGFR enzyme (e.g., an FGFR3 enzyme), or a mutant thereof, in a patient in need thereof, comprising the step of administering to said patient a compound of Formula (I), or pharmaceutically acceptable composition thereof.

The present disclosure is further directed to a method for treating a disorder mediated by an FGFR enzyme (e.g., an FGFR3 enzyme), or a mutant thereof, in a patient in need thereof, comprising the step of administering to the patient a compound of Formula (I), or a pharmaceutically acceptable salt thereof, or a composition comprising a compound of Formula (I), or a pharmaceutically acceptable salt thereof, in combination with another therapy or therapeutic agent as described herein.

The present disclosure is further directed to the use of compounds of Formula (I) in the preparation of a medicament for use in therapy.

### DETAILED DESCRIPTION

### Compounds

In one aspect, the present disclosure provides compounds of Formula (I):

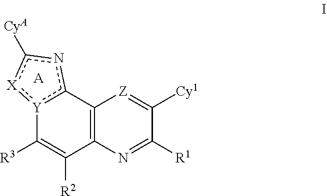

or a pharmaceutically acceptable salt thereof, wherein:
ring A is aromatic;
Y is N or C;
X is CR⁴ when Y is N, or
X is NR⁵ when Y is C;
[symbol] represents a single bond or a double bond;
Z is N or CR⁶;
Cy¹ is selected from C₆₋₁₀ aryl and 5-10 membered heteroaryl; wherein each 5-10 membered heteroaryl has at least one ring-forming carbon atom and 1, 2, 3, or 4 ring-forming heteroatoms independently selected from N, O, and S; wherein the N and S are optionally oxidized; wherein a ring-forming carbon atom of 5-10 membered heteroaryl is optionally substituted by oxo to form a carbonyl group; and wherein the C₆₋₁₀ aryl and 5-10 membered heteroaryl are each optionally substituted with 1, 2, 3 or 4 substituents independently selected from R¹⁰;
Cy^{A} is selected from C₃₋₁₀ cycloalkyl, 4-10 membered heterocycloalkyl, C₆₋₁₀aryl and 5-10 membered heteroaryl; wherein the 4-10 membered heterocycloalkyl and 5-10 membered heteroaryl each has at least one ring-forming carbon atom and 1, 2, 3, or 4 ring-forming heteroatoms independently selected from N, O, and S; wherein the N and S are optionally oxidized; wherein a ring-forming carbon atom of 5-10 membered heteroaryl and 4-10 membered heterocycloalkyl is optionally substituted by oxo to form a carbonyl group; and wherein the C₃₋₁₀ cycloalkyl, 4-10 membered heterocycloalkyl, C₆₋₁₀ aryl and 5-10 membered heteroaryl are each optionally substituted with 1, 2, 3 or 4 substituents independently selected from R²⁰;
R¹, R², R³, and R⁶ are each independently selected from H, D, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, halo, CN, OR^{a9}, SR^{a9}, C(O)R^{b9}, C(O)NR^{c9}R^{d9}, C(O)OR^{a9}, NR^{c9}R^{d9}, NR^{c9}C(O)R^{b9}, NR^{c9}C(O)OR^{a9}, NR^{c9}C(O)NR^{c9}R^{d9}, NR^{c9}S(O)R^{b9}, NR^{c9}S(O)₂R^{b9}, NR^{c9}S(O)₂NR^{c9}R^{d9}, S(O)R^{b9}, S(O)NR^{c9}R^{d9}, S(O)₂R^{b9}, and S(O)₂NR^{c9}R^{d9}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, and 5-10 membered heteroaryl, are each optionally substituted with 1, 2, 3, or 4 substituents independently selected from R³⁰;
R⁴ is selected from H, D, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, halo, CN, OR^{a7}, SR^{a7}, C(O)R^{b7}, C(O)NR^{c7}R^{d7}, C(O)OR^{a7}, NR^{c7}R^{d7}, NR^{c7}C(O)R^{b7}, NR^{c7}C(O)OR^{a7}, NR^{c7}C(O)NR^{c7}R^{d7} NR^{c7}S(O)R^{b7} NR^{c7}S(O)₂R^{b7} NR^{c7}S(O)₂NR^{c7}R^{d7} S(O)R^{b7}, S(O)NR^{c7}R^{d7}, S(O)₂R^{b7}, and S(O)₂NR^{c7}R^{d7}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, and 5-10 membered heteroaryl, are each optionally substituted with 1, 2, 3, or 4 substituents independently selected from R⁴⁰;
R⁵ is selected from H, D, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, and 5-10 membered heteroaryl, are each optionally substituted with 1, 2, 3, or 4 substituents independently selected from R⁵⁰;
each R¹⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NOR^{a1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R¹¹;
each R¹¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a3}, SR^{a3}, C(O)R^{b3}, C(O)NR^{c3}R^{d3}, C(O)OR^{a3}, NR^{c3}R^{d3} NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R¹²;
each R¹² is independently selected from C₁₋₆ alkyl, C₂₋₆ alkenyl, C₂₋₆ alkynyl, C₁₋₆ haloalkyl, C₃₋₆ cycloalkyl, C₆₋₁₀ aryl, 5-6 membered heteroaryl, 4-7 membered heterocycloalkyl, halo, D, CN, OR^{a5}, SR^{a5}, C(O)R^{b5}, C(O)NR^{c5}R^{d5}, C(O)OR^{a5}, NR^{c5}R^{d5} NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5}; wherein said C₁₋₆ alkyl, C₂₋₆ alkenyl, C₂₋₆ alkynyl, C₃₋₆ cycloalkyl, C₆₋₁₀ aryl, 5-6 membered heteroaryl and 4-7 membered heterocycloalkyl, are each optionally substituted with 1, 2, 3, or 4 substituents independently selected from R^{g};
each R²⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NOR^{a2})R^{b2}, C(═NR^{e2})NR^{c2}R^{d2}, NR^{c2}C(═NR^{c2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R²¹;
each R²¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a4}, SR^{a4}, C(O)R^{b4}, C(O)NR^{c4}R^{d4}, C(O)OR^{a4}, NR^{c4}R^{d4} NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R²²;
each R²² is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a6}, SR^{a6}, C(O)R^{b6}, C(O)NR^{c6}R^{d6}, C(O)OR^{a6}, NR^{c6}R^{d6}, NR^{c6}C(O)R^{b6}, NR^{c6}C(O)OR^{a6}, NR^{c6}C(O)NR^{c6}R^{d6} NR^{c6}S(O)R^{b6}, NR^{c6}S(O)₂R^{b6}, NR^{c6}S(O)₂NR^{c6}R^{d6}, S(O)R^{b6}, S(O)NR^{c6}R^{d6}, S(O)₂R^{b6}, and S(O)₂NR^{c6}R^{d6}; wherein said C₁₋₆ alkyl, C₂₋₆ alkenyl, C₂₋₆ alkynyl, C₃₋₆ cycloalkyl, phenyl, 5-6 membered heteroaryl and 4-7 membered heterocycloalkyl, are each optionally substituted with 1, 2, 3, or 4 substituents independently selected from R^{g};
each R³⁰ is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a10}, SR^{a10}, C(O)R^{b10}, C(O)NR^{c10}R^{d10}, C(O)OR^{a10}, NR^{c10}R^{d10}, NR^{c10}C(O)R^{b10}, NR^{c10}C(O)OR^{a10}, NR^{c10}C(O)NR^{c10}R^{d10}, NR^{c10}S(O)R^{b10}, NR^{c10}S(O)₂R^{b10}, NR^{c10}S(O)₂NR^{c10}R^{d10}, S(O)R^{b10}, S(O)NR^{c10}R^{d10}, S(O)₂R^{b10}, and S(O)₂NR^{c10}R^{d10}; wherein said C₁₋₆ alkyl, C₂₋₆ alkenyl, C₂₋₆ alkynyl, C₃₋₆ cycloalkyl, phenyl, 5-6 membered heteroaryl and 4-7 membered heterocycloalkyl, are each optionally substituted with 1, 2, 3, or 4 substituents independently selected from R^{g};
each R⁴⁰ is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a8}, SR^{a8}, C(O)R^{b8}, C(O)NR^{c8}R^{d8}, C(O)OR^{a8}, NR^{c8}R^{d8}, NR^{c8}C(O)R^{b8}, NR^{c8}C(O)OR^{a8}, NR^{c8}C(O)NR^{c8}R^{d8}, NR^{c8}S(O)R^{b8}, NR^{c8}S(O)₂R^{b8}, NR^{c8}S(O)₂NR^{c8}R^{d8}, S(O)R^{b8}, S(O)NR^{c8}R^{d8}, S(O)₂R^{b8}, and S(O)₂NR^{c8}R^{d8}; wherein said C₁₋₆ alkyl, C₂₋₆ alkenyl, C₂₋₆ alkynyl, C₃₋₆ cycloalkyl, phenyl, 5-6 membered heteroaryl and 4-7 membered heterocycloalkyl, are each optionally substituted with 1, 2, 3, or 4 substituents independently selected from R^{g};
each R⁵⁰ is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a11}, SR^{a11}, C(O)R^{b11}, C(O)NR^{c11}R^{b11}, C(O)OR^{a11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)R^{b11}, NR^{c11}S(O)₂R^{b11}, NR^{c11}S(O)₂NR^{c11}R^{d11}, S(O)R^{b11}, S(O)NR^{c11}R^{d11}, S(O)₂R^{b11}, and S(O)₂NR^{c11}R^{d11}; wherein said C₁₋₆ alkyl, C₂₋₆ alkenyl, C₂₋₆ alkynyl, C₃₋₆ cycloalkyl, phenyl, 5-6 membered heteroaryl and 4-7 membered heterocycloalkyl, are each optionally substituted with 1, 2, 3, or 4 substituents independently selected from R^{g};
each R^{a1}, R^{c1} and R^{d1} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R¹¹;
or any R^{c1} and R^{d1} attached to the same N atom, together with the N atom to which they are attached, form a 4-, 5-, 6- or 7-membered heterocycloalkyl group optionally substituted with 1, 2, 3, or 4 substituents independently selected from R¹¹;
each R^{b1} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R¹¹;
each R^{e1} is independently selected from H, CN, C₁₋₆ alkyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{a2}, R^{c2} and R^{d2}, is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl are each optionally substituted with 1, 2, 3, or 4 substituents independently selected from R²¹;
or any R^{c2} and R^{d2} attached to the same N atom, together with the N atom to which they are attached, form a 4-, 5-, 6- or 7-membered heterocycloalkyl group optionally substituted with 1, 2 or 3 substituents independently selected from R²¹;
each R^{b2} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl are each optionally substituted with 1, 2, 3, or 4 substituents independently selected from R²¹;
each R^{e2} is independently selected from H, CN, C₁₋₆ alkyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{a3}, R^{c3} and R^{d3}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R¹²;
or any R^{b3} and R^{d3} attached to the same N atom, together with the N atom to which they are attached, form a 4-, 5-, 6- or 7-membered heterocycloalkyl group optionally substituted with 1, 2 or 3 substituents independently selected from R¹²;
each R^{b3} is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl, are each optionally substituted with 1, 2, 3, or 4 substituents independently selected from R¹²;
each R^{a4}, R^{c4} and R^{d4}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R²²;
or any R^{c4} and R^{d4} attached to the same N atom, together with the N atom to which they are attached, form a 4-, 5-, 6- or 7-membered heterocycloalkyl group optionally substituted with 1, 2 or 3 substituents independently selected from R²²;
each R^{b4} is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R²²;
each R^{a5}, R^{c5} and R^{d5}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
or any R^{c5} and R^{d5} attached to the same N atom, together with the N atom to which they are attached, form a 4-, 5-, 6- or 7-membered heterocycloalkyl group optionally substituted with 1, 2, 3, or 4 substituents independently selected from R^{g};
each R^{b5} is independently selected from C₁₋₆ alkyl, C₂₋₆ alkenyl, C₂₋₆ alkynyl and C₁₋₆ haloalkyl; wherein said C₁₋₆ alkyl C₂₋₆ alkenyl and C₂₋₆ alkynyl are each optionally substituted with 1, 2, 3, or 4 substituents independently selected from R^{g};
each R^{a6}, R^{c6} and R^{d6}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{b6} is independently selected from C₁₋₆ alkyl, C₂₋₆ alkenyl, C₂₋₆ alkynyl, and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{a7}, R^{c7}, and R^{d7} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R⁴⁰;
or any R^{c7} and R^{d7} attached to the same N atom, together with the N atom to which they are attached, form a 4-, 5-, 6- or 7-membered heterocycloalkyl group optionally substituted with 1, 2 or 3 substituents independently selected from R⁴⁰;
each R^{b7} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R⁴⁰;
each R^{a8}, R^{c8} and R^{d8}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{b8} is independently selected from C₁₋₆ alkyl, C₂₋₆ alkenyl, C₂₋₆ alkynyl, and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{a9}, R^{c9}, and R^{d9} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³⁰;
or any R^{c9} and R^{d9} attached to the same N atom, together with the N atom to which they are attached, form a 4-, 5-, 6- or 7-membered heterocycloalkyl group optionally substituted with 1, 2 or 3 substituents independently selected from R³⁰;
each R^{b9} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³⁰;
each Ra¹⁰, R^{c10} and R^{d10}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{b10} is independently selected from C₁₋₆ alkyl, C₂₋₆ alkenyl, C₂₋₆ alkynyl, and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{a11}, R^{c11} and R^{d11}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{b11} is independently selected from C₁₋₆ alkyl, C₂₋₆ alkenyl, C₂₋₆ alkynyl, and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g}; and
each R⁹ is independently selected from D, OH, NO₂, CN, halo, C₁₋₆ alkyl, C₂₋₆ alkenyl, C₂₋₆ alkynyl, C₁₋₆ haloalkyl, C₃₋₆ cycloalkyl, C₃₋₆ cycloalkyl-C₁₋₂ alkylene, C₁₋₆ alkoxy, C₁₋₆ haloalkoxy, C₁₋₃ alkoxy-C₁₋₃ alkyl, C₁₋₃ alkoxy-C₁₋₃ alkoxy, HO—C₁₋₃ alkoxy, HO—C₁₋₃ alkyl, cyano-C₁₋₃ alkyl, H₂N—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkoxycarbonylamino, C₁₋₆ alkylcarbonyloxy, aminocarbonyloxy, C₁₋₆ alkylaminocarbonyloxy, di(C₁₋₆ alkyl)aminocarbonyloxy, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In another aspect, the present disclosure provides compounds of Formula (I):

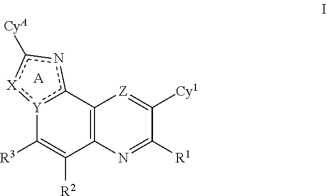

or a pharmaceutically acceptable salt thereof, wherein:
ring A is aromatic;
Y is N or C;
X is CR⁴ when Y is N, or
X is NR⁵ when Y is C;
[symbol] represents a single bond or a double bond;
Z is N or CR⁶;
Cy¹ is selected from C₆₋₁₀ aryl and 5-10 membered heteroaryl; wherein each 5-10 membered heteroaryl has at least one ring-forming carbon atom and 1, 2, 3, or 4 ring-forming heteroatoms independently selected from N, O, and S; wherein the N and S are optionally oxidized; wherein a ring-forming carbon atom of 5-10 membered heteroaryl is optionally substituted by oxo to form a carbonyl group; and wherein the C₆₋₁₀ aryl and 5-10 membered heteroaryl are each optionally substituted with 1, 2, 3 or 4 substituents independently selected from R¹⁰;
Cy^{A} is selected from C₃₋₁₀ cycloalkyl, 4-10 membered heterocycloalkyl, C₆₋₁₀ aryl and 5-10 membered heteroaryl; wherein the 4-10 membered heterocycloalkyl and 5-10 membered heteroaryl each has at least one ring-forming carbon atom and 1, 2, 3, or 4 ring-forming heteroatoms independently selected from N, O, and S; wherein the N and S are optionally oxidized; wherein a ring-forming carbon atom of 5-10 membered heteroaryl and 4-10 membered heterocycloalkyl is optionally substituted by oxo to form a carbonyl group; and wherein the C₃₋₁₀ cycloalkyl, 4-10 membered heterocycloalkyl, C₆₋₁₀ aryl and 5-10 membered heteroaryl are each optionally substituted with 1, 2, 3 or 4 substituents independently selected from R²⁰;
R¹, R², R³, and R⁶ are each independently selected from H, D, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, halo, CN, OR^{a9}, SR^{a9}, C(O)R^{b9}, C(O)NR^{c9}R^{d9}, C(O)OR^{a9}, NR^{c9}R^{d9} NR^{c9}C(O)R^{b9}, NR^{c9}C(O)OR^{a9}, NR^{c9}S(O)R^{b9}, NR^{c9}S(O)₂R^{b9}, NR^{c9}S(O)₂NR^{c9}R^{d9}, S(O)R^{b9}, S(O)NR^{c9}R^{d9}, S(O)₂R^{b9}, and S(O)₂NR^{c9}R^{d9}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, and 5-10 membered heteroaryl, are each optionally substituted with 1, 2, 3, or 4 substituents independently selected from R³⁰;
R⁴ is selected from H, D, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, halo, CN, OR^{a7}, SR^{a7}, C(O)R^{b7}, C(O)NR^{c7}R^{d7}, C(O)OR^{a7}, NR^{c7}R^{d7}, NR^{c7}C(O)R^{b7}, NR^{c7}C(O)OR^{a7}, NR^{c7}S(O)R^{b7}, NR^{c7}S(O)₂R^{b7}, NR^{c7}S(O)₂NR^{c7}R^{d7}, S(O)R^{b7}, S(O)NR^{c7}R^{d7}, S(O)₂R^{b7}, and S(O)₂NR^{c7}R^{d7}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, and 5-10 membered heteroaryl, are each optionally substituted with 1, 2, 3, or 4 substituents independently selected from R⁴⁰;
R⁵ is selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, and 5-10 membered heteroaryl, are each optionally substituted with 1, 2, 3, or 4 substituents independently selected from R⁵⁰;
each R¹⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NOR^{a1})R^{b1}, C(═NR^{c1})NR^{c1}R^{d1} NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1} S(O)₂R^{b1}, and S(O)₂NR^{c1}R^{d1}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R¹¹;
each R¹¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a3}, SR^{a3}, C(O)R^{b3}, C(O)NR^{c3}R^{d3}, C(O)OR^{a3}, NR^{c3}R^{d3}, NR^{c3}C(O)R^{b3}, NR^{c3}C(O)OR^{a3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R¹²;
each R¹² is independently selected from C₁₋₆ alkyl, C₂₋₆ alkenyl, C₂₋₆ alkynyl, C₁₋₆ haloalkyl, C₃₋₆ cycloalkyl, C₆₋₁₀ aryl, 5-6 membered heteroaryl, 4-7 membered heterocycloalkyl, halo, D, CN, OR^{a5}, SR^{a5}, C(O)R^{b5}, C(O)NR^{c5}R^{d5}, C(O)OR^{a5}, NR^{c5}R^{d5} NR^{c5}C(O)R^{b5}, NR^{c5}C(O)OR^{a5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5} S(O)₂R^{b5}, and S(O)₂NR^{c5}R^{d5}; wherein said C₁₋₆ alkyl, C₂₋₆ alkenyl, C₂₋₆ alkynyl, C₃₋₆ cycloalkyl, C₆₋₁₀ aryl, 5-6 membered heteroaryl and 4-7 membered heterocycloalkyl, are each optionally substituted with 1, 2, 3, or 4 substituents independently selected from R^{g};
each R²⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2} NR^{c2}C(O)R^{b2}, NR^{c2}C(O)OR^{a2}, NR^{c2}C(O)NR^{c2}R^{d2}, C(═NR^{e2})R^{b2}, C(═NOR^{a2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R²¹;
each R²¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a4}, SR^{a4}, C(O)R^{b4}, C(O)NR^{c4}R^{d4}, C(O)OR^{a4}, NR^{c4}R^{d4}, NR^{c4}C(O)R^{b4}, NR^{c4}C(O)OR^{a4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R²²;
each R²² is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a6}, SR^{a6}, C(O)R^{b6}, C(O)NR^{c6}R^{d6}, C(O)OR^{a6}, NR^{c6}R^{d6}, NR^{c6}C(O)R^{b6}, NR^{c6}C(O)OR^{a6}, NR^{c6}S(O)R^{b6}, NR^{c6}S(O)₂R^{b6}, NR^{c6}S(O)₂NR^{c6}R^{d6}, S(O)R^{b6}, S(O)NR^{c6}R^{d6}, S(O)₂R^{b6}, and S(O)₂NR^{c6}R^{d6}; wherein said C₁₋₆ alkyl, C₂₋₆ alkenyl, C₂₋₆ alkynyl, C₃₋₆ cycloalkyl, phenyl, 5-6 membered heteroaryl and 4-7 membered heterocycloalkyl, are each optionally substituted with 1, 2, 3, or 4 substituents independently selected from R^{g};
each R³⁰ is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a10}, SR^{a10}, C(O)R^{b10}, C(O)NR^{c10}R^{d10}, C(O)OR^{a10}, NR^{c10}R^{d10}, NR^{c10}C(O)R^{b10}, NR^{c10}C(O)OR^{a10}, NR^{c10}S(O)R^{b10}, NR^{c10}S(O)₂R^{b10}, NR^{c10}S(O)₂NR^{c10}R^{d10}, S(O)R^{b10}, S(O)NR^{c10}R^{d10}, S(O)₂R^{b10}, and S(O)₂NR^{c10}R^{d10}; wherein said C₁₋₆ alkyl, C₂₋₆ alkenyl, C₂₋₆ alkynyl, C₃₋₆ cycloalkyl, phenyl, 5-6 membered heteroaryl and 4-7 membered heterocycloalkyl, are each optionally substituted with 1, 2, 3, or 4 substituents independently selected from R^{g};
each R⁴⁰ is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a8}, SR^{a8}, C(O)R^{b8}, C(O)NR^{c8}R^{d8}, C(O)OR^{a8}, NR^{c8}R^{d8}, NR^{c8}C(O)R^{b8}, NR^{c8}C(O)OR^{a8}, NR^{c8}S(O)R^{b8}, NR^{c8}S(O)₂R^{b8}, NR^{c8}S(O)₂NR^{c8}R^{d8}, S(O)R^{b8}, S(O)NR^{c8}R^{d8}, S(O)₂R^{b8}, and S(O)₂NR^{c8}R^{d8}; wherein said C₁₋₆ alkyl, C₂₋₆ alkenyl, C₂₋₆ alkynyl, C₃₋₆ cycloalkyl, phenyl, 5-6 membered heteroaryl and 4-7 membered heterocycloalkyl, are each optionally substituted with 1, 2, 3, or 4 substituents independently selected from R^{g};
each R⁵⁰ is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a11}, SR^{a11}, C(O)R^{b11}, C(O)NR^{c11}R^{d11}, C(O)OR^{a11}, NR^{c11}R^{d11}, NR^{c11}C(O)R^{b11}, NR^{c11}C(O)OR^{a11}, NR^{c11}S(O)R^{b11}, NR^{c11}S(O)₂R^{b11}, NR^{c11}S(O)₂NR^{c11}R^{d11}, S(O)R^{b11}, S(O)NR^{c11}R^{d11}, S(O)₂R^{b11}, and S(O)₂NR^{c11}R^{d11}; wherein said C₁₋₆ alkyl, C₂₋₆ alkenyl, C₂₋₆ alkynyl, C₃₋₆ cycloalkyl, phenyl, 5-6 membered heteroaryl and 4-7 membered heterocycloalkyl, are each optionally substituted with 1, 2, 3, or 4 substituents independently selected from R^{g};
each R^{a1}, R^{c1} and R^{d1} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R¹¹;
or any R^{c1} and R^{d1} attached to the same N atom, together with the N atom to which they are attached, form a 4-, 5-, 6- or 7-membered heterocycloalkyl group optionally substituted with 1, 2, 3, or 4 substituents independently selected from R¹¹;
each R¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R¹¹;
each R^{e1} is independently selected from H, CN, C₁₋₆ alkyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{a2}, R^{c2} and R^{d2} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl are each optionally substituted with 1, 2, 3, or 4 substituents independently selected from R²¹;
or any R^{c2} and R^{d2} attached to the same N atom, together with the N atom to which they are attached, form a 4-, 5-, 6- or 7-membered heterocycloalkyl group optionally substituted with 1, 2 or 3 substituents independently selected from R²¹;
each R^{b2} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl are each optionally substituted with 1, 2, 3, or 4 substituents independently selected from R²¹;
each R^{e2} is independently selected from H, CN, C₁₋₆ alkyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{a3}, R^{c3} and R^{d3} is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R¹²;
or any R^{c3} and R^{d3} attached to the same N atom, together with the N atom to which they are attached, form a 4-, 5-, 6- or 7-membered heterocycloalkyl group optionally substituted with 1, 2 or 3 substituents independently selected from R¹²;
each R^{b3} is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl, are each optionally substituted with 1, 2, 3, or 4 substituents independently selected from R¹²;
each R^{a4}, R^{c4} and R^{d4} is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R²²;
or any R^{c4} and R^{d4} attached to the same N atom, together with the N atom to which they are attached, form a 4-, 5-, 6- or 7-membered heterocycloalkyl group optionally substituted with 1, 2 or 3 substituents independently selected from R²²;
each R^{b4} is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl, C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R²²;
each R^{a5}, R^{c5} and R^{d5} is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
or any R^{c5} and R^{d5} attached to the same N atom, together with the N atom to which they are attached, form a 4-, 5-, 6- or 7-membered heterocycloalkyl group optionally substituted with 1, 2, 3, or 4 substituents independently selected from R^{g};
each R^{b5} is independently selected from C₁₋₆ alkyl, C₂₋₆ alkenyl, C₂₋₆ alkynyl and C₁₋₆ haloalkyl; wherein said C₁₋₆ alkyl C₂₋₆ alkenyl and C₂₋₆ alkynyl are each optionally substituted with 1, 2, 3, or 4 substituents independently selected from R^{g};
each R^{a6}, R^{c6} and R^{d6} is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{b6} is independently selected from C₁₋₆ alkyl, C₂₋₆ alkenyl, C₂₋₆ alkynyl, and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{a7}, R^{c7}, and R^{d7} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R⁴⁰;
or any R^{c7} and R^{d7} attached to the same N atom, together with the N atom to which they are attached, form a 4-, 5-, 6- or 7-membered heterocycloalkyl group optionally substituted with 1, 2 or 3 substituents independently selected from R⁴⁰;
each R^{b7} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R⁴⁰;
each R^{a8}, R^{c8} and R^{d8} is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{b8} is independently selected from C₁₋₆ alkyl, C₂₋₆ alkenyl, C₂₋₆ alkynyl, and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{a9}, R^{c9}, and R^{d9} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³⁰;
or any R^{c9} and R^{d9} attached to the same N atom, together with the N atom to which they are attached, form a 4-, 5-, 6- or 7-membered heterocycloalkyl group optionally substituted with 1, 2 or 3 substituents independently selected from R³⁰;
each R^{b9} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³⁰;
each R^{a10}, R^{c10} and R^{d10} is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{b10} is independently selected from C₁₋₆ alkyl, C₂₋₆ alkenyl, C₂₋₆ alkynyl, and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{a11}, R^{c11} and R^{d11} is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{b11} is independently selected from C₁₋₆ alkyl, C₂₋₆ alkenyl, C₂₋₆ alkynyl, and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g}; and
each R^{g} is independently selected from D, OH, NO₂, CN, halo, C₁₋₆ alkyl, C₂₋₆ alkenyl, C₂₋₆ alkynyl, C₁₋₆ haloalkyl, C₃₋₆ cycloalkyl, C₃₋₆ cycloalkyl-C₁₋₂ alkylene, C₁₋₆ alkoxy, C₁₋₆ haloalkoxy, C₁₋₃ alkoxy-C₁₋₃ alkyl, C₁₋₃ alkoxy-C₁₋₃ alkoxy, HO—C₁₋₃ alkoxy, HO—C₁₋₃ alkyl, cyano-C₁₋₃ alkyl, H₂N—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkoxycarbonylamino, C₁₋₆ alkylcarbonyloxy, aminocarbonyloxy, C₁₋₆ alkylaminocarbonyloxy, di(C₁₋₆ alkyl)aminocarbonyloxy, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In one embodiment, the present disclosure provides compounds of Formula (I) or a pharmaceutically acceptable salt thereof, wherein:
ring A is aromatic;
Y is N or C;
X is CR⁴ when Y is N, or
X is NR⁵ when Y is C;
[symbol] represents a single bond or a double bond;
Z is N or CR⁶;
Cy¹ is selected from 5-10 membered heteroaryl; wherein each 5-10 membered heteroaryl has at least one ring-forming carbon atom and 1, 2, 3, or 4 ring-forming heteroatoms independently selected from N, O, and S; wherein the N and S are optionally oxidized; wherein a ring-forming carbon atom of 5-10 membered heteroaryl is optionally substituted by oxo to form a carbonyl group; and wherein the 5-10 membered heteroaryl are each optionally substituted with 1, 2, 3 or 4 substituents independently selected from R¹⁰;
Cy^{A} is selected from C₆₋₁₀ aryl and 5-10 membered heteroaryl; wherein the 5-10 membered heteroaryl each has at least one ring-forming carbon atom and 1, 2, 3, or 4 ring-forming heteroatoms independently selected from N, O, and S; wherein the N and S are optionally oxidized; wherein a ring-forming carbon atom of 5-10 membered heteroaryl is optionally substituted by oxo to form a carbonyl group; and wherein the C₆₋₁₀ aryl and 5-10 membered heteroaryl are each optionally substituted with 1, 2, 3 or 4 substituents independently selected from R²⁰;
R¹, R², R³, and R⁶ are each independently selected from H, D, C₁₋₆ alkyl, C₁₋₆ haloalkyl, halo, CN, OR^{a9}, and NR^{c9}R^{d9};
R⁴ is selected from H, D, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, halo, CN, OR^{a7}, and NR^{c7}R^{d7}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, and 5-10 membered heteroaryl, are each optionally substituted with 1, 2, 3, or 4 substituents independently selected from R⁴⁰;
R⁵ is selected from C₁₋₆ alkyl, C₁₋₆ haloalkyl, C₃₋₁₀ cycloalkyl, and C₆₋₁₀ aryl; wherein said C₁₋₆ alkyl, C₃₋₁₀ cycloalkyl, and C₆₋₁₀ aryl, are each optionally substituted with 1, 2, 3, or 4 substituents independently selected from R⁵⁰;
each R¹⁰ is independently selected from C₁₋₆ alkyl, C₁₋₆ haloalkyl, C₃₋₁₀ cycloalkyl, 4-10 membered heterocycloalkyl, halo, D, CN, OR^{a1}, and NR^{c1}R^{d1}; wherein said C₁₋₆ alkyl, C₃₋₁₀ cycloalkyl, and 4-10 membered heterocycloalkyl, are each optionally substituted with 1, 2, 3, or 4 substituents independently selected from R¹¹;
each R¹¹ is independently selected from C₁₋₆ alkyl, C₁₋₆ haloalkyl, C₃₋₁₀ cycloalkyl, halo, D, CN, OR^{a3}, C(O)R^{b3}, C(O)NR^{c3}R^{d3}, C(O)OR^{a3}, NR^{c3}R^{d3}, and NR^{c3}C(O)R^{b3}; wherein said C₁₋₆ alkyl, and C₃₋₁₀ cycloalkyl, are each optionally substituted with 1, 2, 3, or 4 substituents independently selected from R¹²;
each R¹² is independently selected from C₁₋₆ alkyl, halo, D, CN, OR^{a5} and NR^{c5}R^{d5};
each R²⁰ is independently selected from C₁₋₆ alkyl, C₁₋₆ haloalkyl, C₃₋₁₀ cycloalkyl, halo, D, CN, OR^{a2}, C(O)R^{b2}, C(O)NR^{c2}R^{d2}, C(O)OR^{a2}, S(O)NR^{c2}R^{d2}, and S(O)₂R^{b2}; wherein said C₁₋₆ alkyl, and C₃₋₁₀ cycloalkyl are each optionally substituted with 1, 2, 3, or 4 substituents independently selected from R²¹;
each R²¹ is independently selected from C₁₋₆ alkyl, C₁₋₆ haloalkyl, halo, D, CN, OR^{a4}, and NR^{c4}R^{d4};
each R⁴⁰ is independently selected from C₁₋₆ alkyl, C₁₋₆ haloalkyl, halo, D, CN, OR^{a8}, and NR^{c8}R^{d8};
each R⁵⁰ is independently selected from C₁₋₆ alkyl, C₁₋₆ haloalkyl, halo, D, CN, OR^{a11}, and NR^{c11}R^{d11};
each R^{a1}, R^{c1} and R^{d1} is independently selected from H, C₁₋₆ alkyl, C₁₋₆ haloalkyl, C₃₋₁₀ cycloalkyl, and 4-10 membered heterocycloalkyl; wherein said C₁₋₆ alkyl, C₃₋₁₀ cycloalkyl, and 4-10 membered heterocycloalkyl, are each optionally substituted with 1, 2, 3, or 4 substituents independently selected from R¹¹;
or any R^{c1} and R^{d1} attached to the same N atom, together with the N atom to which they are attached, form a 4-, 5-, or 6-membered heterocycloalkyl group optionally substituted with 1, 2, 3, or 4 substituents independently selected from R¹¹;
each R^{a2}, R^{c2} and R^{d2} is independently selected from H, C₁₋₆ alkyl, C₁₋₆ haloalkyl, and C₃₋₁₀ cycloalkyl; wherein said C₁₋₆ alkyl and C₃₋₁₀ cycloalkyl, are each optionally substituted with 1, 2, 3, or 4 substituents independently selected from R²¹;
each R^{b2} is independently selected from C₁₋₆ alkyl, C₁₋₆ haloalkyl, and C₃₋₁₀ cycloalkyl; wherein said C₁₋₆ alkyl and C₃₋₁₀ cycloalkyl, are each optionally substituted with 1, 2, 3, or 4 substituents independently selected from R²¹;
each R^{a3}, R^{c3} and R^{d3} is independently selected from H, C₁₋₆ alkyl, C₁₋₆ haloalkyl, and C₃₋₆ cycloalkyl; wherein said C₁₋₆ alkyl and C₃₋₆ cycloalkyl, are each optionally substituted with 1, 2, 3, or 4 substituents independently selected from R¹²;
each R^{b3} is independently selected from C₁₋₆ alkyl, C₁₋₆ haloalkyl, and C₃₋₆ cycloalkyl; wherein said C₁₋₆ alkyl and C₃₋₆ cycloalkyl, are each optionally substituted with 1, 2, 3, or 4 substituents independently selected from R¹²;
each R^{a4}, R^{c4} and R^{d4} is independently selected from H, C₁₋₆ alkyl, and C₁₋₆ haloalkyl;
each R^{a5}, R^{c5} and R^{d5} is independently selected from H, C₁₋₆ alkyl, and C₁₋₆ haloalkyl;
each R^{a7}, R^{c7}, and R^{d7} is independently selected from H, C₁₋₆ alkyl, and C₁₋₆ haloalkyl;
each R^{a8}, R^{c8} and R^{d8} is independently selected from H, C₁₋₆ alkyl, and C₁₋₆ haloalkyl;
each R^{a9}, R^{c9}, and R^{d9} is independently selected from H, C₁₋₆ alkyl, and C₁₋₆ haloalkyl; and
each R^{a11}, R^{c11}, and R^{d11} is independently selected from H, C₁₋₆ alkyl, and C₁₋₆ haloalkyl.

In one embodiment, the present disclosure provides compounds of Formula (I) or a pharmaceutically acceptable salt thereof, wherein:
ring A is aromatic;
Y is N or C;
X is CR⁴ when Y is N, or
X is NR⁵ when Y is C;
[symbol] represents a single bond or a double bond;
Z is N or CH;
Cy¹ is selected from 5-6 membered heteroaryl; wherein each 5-6 membered heteroaryl has at least one ring-forming carbon atom and 1, 2, 3, or 4 ring-forming heteroatoms independently selected from N, O, and S; and wherein the 5-6 membered heteroaryl are each optionally substituted with 1, 2, 3 or 4 substituents independently selected from R¹⁰;
Cy^{A} is selected from phenyl and 5-6 membered heteroaryl; wherein the 5-6 membered heteroaryl each has at least one ring-forming carbon atom and 1, 2, 3, or 4 ring-forming heteroatoms independently selected from N, O, and S; and wherein the phenyl and 5-6 membered heteroaryl are each optionally substituted with 1, 2, 3 or 4 substituents independently selected from R²⁰;
R¹, R², R³ are each H;
R⁴ is selected from H, halo, CN, C₁₋₆ alkyl, C₂₋₆ alkenyl, C₃₋₇ cycloalkyl, phenyl, and 5-6 membered heteroaryl, wherein said C₁₋₆ alkyl, C₂₋₆ alkenyl, C₃₋₇ cycloalkyl, phenyl, and 5-6 membered heteroaryl are each optionally substituted with 1 or 2 substituents selected from R⁴⁰;
R⁵ is C₁₋₆ alkyl;
each R¹⁰ is independently selected from C₁₋₆ alkyl, 4-6 membered heterocycloalkyl, and C₃₋₇ cycloalkyl, each of which is optionally substituted with 1 or 2 substituents independently selected from R¹¹;
each R¹¹ is independently selected from C₁₋₆ alkyl, C₃₋₇ cycloalkyl, CN, OR^{a3}, C(O)R^{b3}, C(O)NR^{c3}R^{d3}, C(O)OR^{a3}, and S(O)₂R^{b3}; wherein said C₁₋₆ alkyl, and C₃₋₇ cycloalkyl are each optionally substituted with 1 or 2 substituents independently selected from R¹²;
each R¹² is independently selected from C₁₋₆ alkyl, halo, D, CN, OR^{a5} and NR^{c5}R^{d5};
each R²⁰ is independently selected from halo, C₁₋₆ alkyl, and OR^{a2}
each R²¹ is independently selected from C₁₋₆ alkyl, C₁₋₆ haloalkyl, halo, D, CN, OR^{a4}, and NR^{c4}R^{d4}.
each R⁴⁰ is independently selected from C₁₋₆ alkyl, C₁₋₆ haloalkyl, halo, D, CN, OR^{a8}, and NR^{c8}R^{d8}.
each R^{a2} is selected from H, C₁₋₆ alkyl, C₁₋₆ haloalkyl, and C₃₋₁₀ cycloalkyl; wherein said C₁₋₆ alkyl and C₃₋₁₀ cycloalkyl, are each optionally substituted with 1, 2, 3, or 4 substituents independently selected from R²¹;
each R^{a3}, R^{c3} and R^{d3} is independently selected from H, C₁₋₆ alkyl, C₁₋₆ haloalkyl, and C₃₋₆ cycloalkyl; wherein said C₁₋₆ alkyl and C₃₋₆ cycloalkyl, are each optionally substituted with 1, 2, 3, or 4 substituents independently selected from R¹²;
each R^{b3} is independently selected from C₁₋₆ alkyl, C₁₋₆ haloalkyl, and C₃₋₆ cycloalkyl; wherein said C₁₋₆ alkyl and C₃₋₆ cycloalkyl, are each optionally substituted with 1, 2, 3, or 4 substituents independently selected from R¹²;
each R^{a4}, R^{c4} and R^{d4} is independently selected from H, C₁₋₆ alkyl, and C₁₋₆ haloalkyl;
each R^{a5}, R^{c5} and R^{d5} is independently selected from H, C₁₋₆ alkyl, and C₁₋₆ haloalkyl; and
each R^{a8}, R^{c8} and R^{d8} is independently selected from H, C₁₋₆ alkyl, and C₁₋₆ haloalkyl.

In one embodiment, the present disclosure provides compounds of Formula (I) or a pharmaceutically acceptable salt thereof, wherein:
ring A is aromatic;
Y is N or C;
X is CR⁴ when Y is N, or
X is NR⁵ when Y is C;
[symbol] represents a single bond or a double bond;
Z is N or CH;
Cy¹ is selected from phenyl and 5-6 membered heteroaryl; wherein each 5-6 membered heteroaryl has at least one ring-forming carbon atom and 1, 2, 3, or 4 ring-forming heteroatoms independently selected from N, O, and S; and wherein the phenyl and 5-6 membered heteroaryl are each optionally substituted with 1, 2, 3 or 4 substituents independently selected from R¹⁰;
Cy^{A} is selected from phenyl, 5-6 membered heteroaryl, 4-10 membered heterocycloalkyl, and C₃₋₇ cycloalkyl; wherein the 5-6 membered heteroaryl each has at least one ring-forming carbon atom and 1, 2, 3, or 4 ring-forming heteroatoms independently selected from N, O, and S; and wherein the phenyl, 5-6 membered heteroaryl, 4-10 membered heterocycloalkyl, and C₃₋₇ cycloalkyl are each optionally substituted with 1, 2, 3 or 4 substituents independently selected from R²⁰;
R¹ is H, OR^{a9}, or NR^{c9}R^{d9};
R² and R³ are each H;
R⁴ is selected from H, halo, CN, C₁₋₆ alkyl, C₂₋₆ alkenyl, C₃₋₇ cycloalkyl, phenyl, and 5-6 membered heteroaryl, wherein said C₁₋₆ alkyl, C₂₋₆ alkenyl, C₃₋₇ cycloalkyl, phenyl, and 5-6 membered heteroaryl are each optionally substituted with 1 or 2 substituents selected from R⁴⁰;
R⁵ is C₁₋₆ alkyl;
each R¹⁰ is independently selected from C₁₋₆ alkyl, C₁₋₆ haloalkyl, phenyl, 4-6 membered heterocycloalkyl, and C₃₋₇ cycloalkyl, each of which is optionally substituted with 1 or 2 substituents independently selected from R¹¹;
each R¹¹ is independently selected from C₁₋₆ alkyl, CN, C(O)R^{b3}, C(O)OR^{a3}, NR^{c3}C(O)R^{b3}, OR^{a3}, C(O)NR^{c3}R^{d3}, NR^{c3}R^{d3}, NR^{c3}C(O)NR^{c3}R^{d3}, S(O)₂NR^{c3}R^{d3}, S(O)₂R^{b3}, phenyl, and 4-6 membered heterocycloalkyl; wherein said C₁₋₆ alkyl, phenyl, and 4-6 membered heterocycloalkyl are each optionally substituted with 1 or 2 substituents independently selected from R¹²;
each R¹² is independently selected from C₁₋₆ alkyl, halo, D, CN, OR^{a5}, NR^{c5}R^{d5} and 5-6 membered heteroaryl;
each R²⁰ is independently selected from halo, CN, C₁₋₆ alkyl, C₁₋₆ haloalkyl, and NR^{c2}C(O)R^{b2} wherein the C₁₋₆ alkyl is optionally substituted with OR^{a4};
each R²¹ is independently selected from C₁₋₆ alkyl, C₁₋₆ haloalkyl, halo, D, CN, OR^{a4}, and NR^{c4}R^{d4}.
each R⁴⁰ is independently selected from C₁₋₆ alkyl, C₁₋₆ haloalkyl, halo, D, CN, OR^{a8}, and NR^{c8}R^{d8};
each R^{a2} is selected from H, C₁₋₆ alkyl, C₁₋₆ haloalkyl, and C₃₋₁₀ cycloalkyl; wherein said C₁₋₆ alkyl and C₃₋₁₀ cycloalkyl, are each optionally substituted with 1, 2, 3, or 4 substituents independently selected from R²¹;
each R^{a3}, R^{c3} and R^{d3} is independently selected from H, C₁₋₆ alkyl, C₁₋₆ haloalkyl, and C₃₋₆ cycloalkyl; wherein said C₁₋₆ alkyl and C₃₋₆ cycloalkyl, are each optionally substituted with 1, 2, 3, or 4 substituents independently selected from R¹²;
each R^{b3} is independently selected from C₁₋₆ alkyl, C₁₋₆ haloalkyl, C₃₋₆ cycloalkyl, 5-6 membered heteroaryl, and 4-6 membered heterocycloalkyl; each optionally substituted with 1, 2, 3, or 4 substituents independently selected from R¹²;
each R^{a4}, R^{c4} and R^{d4} is independently selected from H, C₁₋₆ alkyl, and C₁₋₆ haloalkyl;
each R^{a5}, R^{c5} and R^{d5} is independently selected from H, C₁₋₆ alkyl, and C₁₋₆ haloalkyl; and
each R^{a8}, R^{c8} and R^{d8} is independently selected from H, C₁₋₆ alkyl, and C₁₋₆ haloalkyl.

In one aspect, the present disclosure provides compounds of Formula (IA) or (IB):

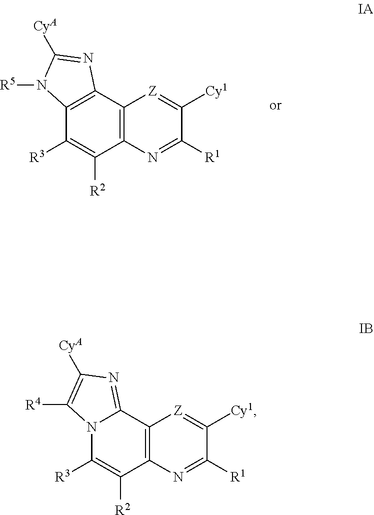

or a pharmaceutically acceptable salt thereof.

In one aspect, the present disclosure provides compounds of Formula (IA):

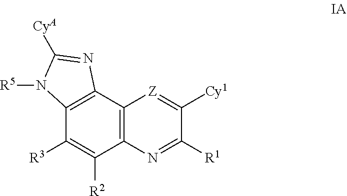

or a pharmaceutically acceptable salt thereof.

In one aspect, the present disclosure provides compounds of Formula (IB):

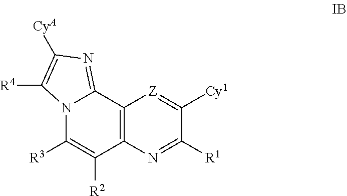

or a pharmaceutically acceptable salt thereof.

In one aspect, the present disclosure provides compounds of Formula (IA) or a pharmaceutically acceptable salt thereof, wherein:
Z is N;
Cy¹ is selected from C₆₋₁₀ aryl and 5-10 membered heteroaryl; wherein each 5-10 membered heteroaryl has at least one ring-forming carbon atom and 1, 2, 3, or 4 ring-forming heteroatoms independently selected from N, O, and S; wherein the N and S are optionally oxidized; wherein a ring-forming carbon atom of 5-10 membered heteroaryl is optionally substituted by oxo to form a carbonyl group; and wherein the C₆₋₁₀ aryl and 5-10 membered heteroaryl are each optionally substituted with 1, 2, 3 or 4 substituents independently selected from R¹⁰;
Cy^{A} is selected from C₃₋₁₀ cycloalkyl, 4-10 membered heterocycloalkyl, C₆₋₁₀ aryl and 5-10 membered heteroaryl; wherein the 4-10 membered heterocycloalkyl and 5-10 membered heteroaryl each has at least one ring-forming carbon atom and 1, 2, 3, or 4 ring-forming heteroatoms independently selected from N, O, and S; wherein the N and S are optionally oxidized; wherein a ring-forming carbon atom of 5-10 membered heteroaryl and 4-10 membered heterocycloalkyl is optionally substituted by oxo to form a carbonyl group; and wherein the C₃₋₁₀ cycloalkyl, 4-10 membered heterocycloalkyl, C₆₋₁₀ aryl and 5-10 membered heteroaryl are each optionally substituted with 1, 2, 3 or 4 substituents independently selected from R²⁰;
R¹, R², R³, and R⁶ are each independently selected from H, D, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, halo, CN, OR^{a9}, SR^{a9}, C(O)R^{b9}, C(O)NR^{c9}R^{d9}, C(O)OR^{a9}, NR^{c9}R^{d9}, NR^{c9}C(O)R^{b9}, NR^{c9}C(O)OR^{a9}, NR^{c9}C(O)NR^{c9}R^{d9}, NR^{c9}S(O)R^{b9}, NR^{c9}S(O)₂R^{b9}, NR^{c9}S(O)₂NR^{c9}R^{d9}, S(O)R^{b9}, S(O)NR^{c9}R^{d9}, S(O)₂R^{b9}, and S(O)₂NR^{c9}R^{d9}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, and 5-10 membered heteroaryl, are each optionally substituted with 1, 2, 3, or 4 substituents independently selected from R³⁰;
R⁵ is selected from H, D, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, and 5-10 membered heteroaryl, are each optionally substituted with 1, 2, 3, or 4 substituents independently selected from R⁵⁰;
each R¹⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NOR^{a1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R¹¹;
each R¹¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a3}, SR^{a3}, C(O)R^{b3}, C(O)NR^{c3}R^{d3}, C(O)OR^{a3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R¹²;
each R¹² is independently selected from C₁₋₆ alkyl, C₂₋₆ alkenyl, C₂₋₆ alkynyl, C₁₋₆ haloalkyl, C₃₋₆ cycloalkyl, C₆₋₁₀ aryl, 5-6 membered heteroaryl, 4-7 membered heterocycloalkyl, halo, D, CN, OR^{a5}, SR^{a5}, C(O)R^{b5}, C(O)NR^{c5}R^{d5}, C(O)OR^{a5}, NR^{c5}R^{d5} NR^{c5}C(O)R^{b5}, NR^{c5}C(O)OR^{a5}, NR^{c5}C(O)NR^{c5}R^{d5}, NR^{c5}S(O)R^{b5}, NR⁵S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5}; wherein said C₁₋₆ alkyl, C₂₋₆ alkenyl, C₂₋₆ alkynyl, C₃₋₆ cycloalkyl, C₆₋₁₀ aryl, 5-6 membered heteroaryl and 4-7 membered heterocycloalkyl, are each optionally substituted with 1, 2, 3, or 4 substituents independently selected from R^{g};
each R²⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NOR^{a2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R²¹;
each R²¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a4}, SR^{a4}, C(O)R^{b4}, C(O)NR^{c4}R^{d4}, C(O)OR^{a4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4} S(O)R^{b4}, S(O)NR^{c4}R^{d4}, S(O)₂R^{b4}, and S(O)₂NR^{c4}R^{d4}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R²²;
each R²² is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a6}, SR^{a6}, C(O)R^{b6}, C(O)NR^{c6}R^{d6}, C(O)OR^{a6}, NR^{c6}R^{d6}, NR^{c6}C(O)R^{b6}, NR^{c6}C(O)OR^{a6}, NR^{c6}C(O)NR^{c6}R^{d6}, NR^{c6}S(O)R^{b6}, NR^{c6}S(O)₂R^{b6}, NR^{c6}S(O)₂NR^{c6}R^{d6}, S(O)R^{b6}, S(O)NR^{c6}R^{d6}, S(O)₂R^{b6}, and S(O)₂NR^{c6}R^{d6}; wherein said C₁₋₆ alkyl, C₂₋₆ alkenyl, C₂₋₆ alkynyl, C₃₋₆ cycloalkyl, phenyl, 5-6 membered heteroaryl and 4-7 membered heterocycloalkyl, are each optionally substituted with 1, 2, 3, or 4 substituents independently selected from R^{g};
each R³⁰ is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a10}, SR^{a10}, C(O)R^{b10}, C(O)NR^{c10}R^{d10}, C(O)OR^{a10}, NR^{c10}R^{d10}, NR^{c10}C(O)R^{b10}, NR^{c10}C(O)OR^{a10}, NR^{c10}C(O)NR^{c10}R^{d10}, NR^{c10}S(O)R^{b10}, NR^{c10}S(O)₂R^{b10}, NR^{c10}S(O)₂NR^{c10}R^{d10}, S(O)R^{b10}, S(O)NR^{c10}R^{d10}, S(O)₂R^{b10}, and S(O)₂NR^{c10}R^{d10}; wherein said C₁₋₆ alkyl, C₂₋₆ alkenyl, C₂₋₆ alkynyl, C₃₋₆ cycloalkyl, phenyl, 5-6 membered heteroaryl and 4-7 membered heterocycloalkyl, are each optionally substituted with 1, 2, 3, or 4 substituents independently selected from R^{g};
each R⁵⁰ is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a11}, SR^{a11}, C(O)R^{b11}, C(O)NR^{c11}R^{d11}, C(O)OR^{a11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)R^{b11}, NR^{c11}S(O)₂R^{b11}, NR^{c11}S(O)₂NR^{c11}R^{d11}, S(O)R^{b11}, S(O)NR^{c11}R^{d11}, S(O)₂R^{b11}, and S(O)₂NR^{c11}R^{d11}; wherein said C₁₋₆ alkyl, C₂₋₆ alkenyl, C₂₋₆ alkynyl, C₃₋₆ cycloalkyl, phenyl, 5-6 membered heteroaryl and 4-7 membered heterocycloalkyl, are each optionally substituted with 1, 2, 3, or 4 substituents independently selected from R^{g};
each R^{a1}, R^{c1} and R^{d1} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R¹¹;
or any R^{c1} and R^{d1} attached to the same N atom, together with the N atom to which they are attached, form a 4-, 5-, 6- or 7-membered heterocycloalkyl group optionally substituted with 1, 2, 3, or 4 substituents independently selected from R¹¹;
each R^{b1} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R¹¹;
each R^{e1} is independently selected from H, CN, C₁₋₆ alkyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{a2}, R^{c2} and R^{d2}, is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl are each optionally substituted with 1, 2, 3, or 4 substituents independently selected from R²¹;
or any R^{c2} and R^{d2} attached to the same N atom, together with the N atom to which they are attached, form a 4-, 5-, 6- or 7-membered heterocycloalkyl group optionally substituted with 1, 2 or 3 substituents independently selected from R²¹;
each R^{b2} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl are each optionally substituted with 1, 2, 3, or 4 substituents independently selected from R²¹;
each R^{e2} is independently selected from H, CN, C₁₋₆ alkyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{a3}, R^{c3} and R^{d3}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R¹²;
or any R^{c3} and R^{d3} attached to the same N atom, together with the N atom to which they are attached, form a 4-, 5-, 6- or 7-membered heterocycloalkyl group optionally substituted with 1, 2 or 3 substituents independently selected from R¹²;
each R^{b3} is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl, are each optionally substituted with 1, 2, 3, or 4 substituents independently selected from R¹²;
each R^{a4}, R^{c4} and R^{d4}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R²²;
or any R^{c4} and R^{d4} attached to the same N atom, together with the N atom to which they are attached, form a 4-, 5-, 6- or 7-membered heterocycloalkyl group optionally substituted with 1, 2 or 3 substituents independently selected from R²²;
each R^{b4} is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R²²;
each R^{a5}, R^{c5} and R^{d5}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
or any R^{c5} and R^{d5} attached to the same N atom, together with the N atom to which they are attached, form a 4-, 5-, 6- or 7-membered heterocycloalkyl group optionally substituted with 1, 2, 3, or 4 substituents independently selected from R^{g};
each R^{b5} is independently selected from C₁₋₆ alkyl, C₂₋₆ alkenyl, C₂₋₆ alkynyl and C₁₋₆ haloalkyl; wherein said C₁₋₆ alkyl C₂₋₆ alkenyl and C₂₋₆ alkynyl are each optionally substituted with 1, 2, 3, or 4 substituents independently selected from R^{g};
each R^{a6}, R^{c6} and R^{d6}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{b6} is independently selected from C₁₋₆ alkyl, C₂₋₆ alkenyl, C₂₋₆ alkynyl, and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{a9}, R^{c9}, and R^{d9} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³⁰;
or any R^{c9} and R^{d9} attached to the same N atom, together with the N atom to which they are attached, form a 4-, 5-, 6- or 7-membered heterocycloalkyl group optionally substituted with 1, 2 or 3 substituents independently selected from R³⁰;
each R^{b9} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³⁰;
each R^{a10}, R^{c10} and R^{d10}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{b10} is independently selected from C₁₋₆ alkyl, C₂₋₆ alkenyl, C₂₋₆ alkynyl, and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{a11}, R^{c11} and R^{d11}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{b11} is independently selected from C₁₋₆ alkyl, C₂₋₆ alkenyl, C₂₋₆ alkynyl, and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g}; and
each R⁹ is independently selected from D, OH, NO₂, CN, halo, C₁₋₆ alkyl, C₂₋₆ alkenyl, C₂₋₆ alkynyl, C₁₋₆ haloalkyl, C₃₋₆ cycloalkyl, C₃₋₆ cycloalkyl-C₁₋₂ alkylene, C₁₋₆ alkoxy, C₁₋₆ haloalkoxy, C₁₋₃ alkoxy-C₁₋₃ alkyl, C₁₋₃ alkoxy-C₁₋₃ alkoxy, HO—C₁₋₃ alkoxy, HO—C₁₋₃ alkyl, cyano-C₁₋₃ alkyl, H₂N—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkoxycarbonylamino, C₁₋₆ alkylcarbonyloxy, aminocarbonyloxy, C₁₋₆ alkylaminocarbonyloxy, di(C₁₋₆ alkyl)aminocarbonyloxy, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In one aspect, the present disclosure provides compounds of Formula (IA), or a pharmaceutically acceptable salt thereof, wherein:
Z is N;
Cy¹ is selected from 5-10 membered heteroaryl; wherein each 5-10 membered heteroaryl has at least one ring-forming carbon atom and 1, 2, 3, or 4 ring-forming heteroatoms independently selected from N, O, and S; wherein a ring-forming carbon atom of 5-10 membered heteroaryl is optionally substituted by oxo to form a carbonyl group; and wherein the 5-10 membered heteroaryl are each optionally substituted with 1 or 2 substituents independently selected from R¹⁰;
Cy^{A} is selected from 4-10 membered heterocycloalkyl, C₆₋₁₀ aryl and 5-10 membered heteroaryl; wherein the 4-10 membered heterocycloalkyl and 5-10 membered heteroaryl each has at least one ring-forming carbon atom and 1, 2, 3, or 4 ring-forming heteroatoms independently selected from N, O, and S; wherein a ring-forming carbon atom of 5-10 membered heteroaryl and 4-10 membered heterocycloalkyl is optionally substituted by oxo to form a carbonyl group; and wherein the 4-10 membered heterocycloalkyl, C₆₋₁₀ aryl and 5-10 membered heteroaryl are each optionally substituted with 1, 2, or 3 substituents independently selected from R²⁰;
R¹, R², and R³ are each independently selected from H, D, C₁₋₆ alkyl, C₁₋₆ haloalkyl halo, and CN;
R⁵ is selected from H, D, C₁₋₆ alkyl, and C₁₋₆ haloalkyl;
each R¹⁰ is independently selected from C₁₋₆ alkyl, C₁₋₆ haloalkyl, C₃₋₁₀ cycloalkyl, 4-10 membered heterocycloalkyl, halo, D, CN, OR^{a1}, and NR^{c1}R^{d1}; wherein said C₁₋₆ alkyl, C₃₋₁₀ cycloalkyl, and 4-10 membered heterocycloalkyl, are each optionally substituted with 1, 2, or 3 substituents independently selected from R¹¹;
each R¹¹ is independently selected from C₁₋₆ alkyl, C₁₋₆ haloalkyl, halo, D, CN, OR³, C(O)R^{b3}, C(O)NR^{c3}R^{d3}, and NR^{c3}R^{d3};
each R²⁰ is independently selected from C₁₋₆ alkyl, C₁₋₆ haloalkyl, halo, D, CN, OR^{a2}, and NR^{c2}R^{d2}.
each R^{a1}, R^{c1} and R^{d1} is independently selected from H, C₁₋₆ alkyl, C₁₋₆ haloalkyl, C₃₋₁₀ cycloalkyl, and 4-10 membered heterocycloalkyl; wherein said C₁₋₆ alkyl, C₃₋₁₀ cycloalkyl, and 4-10 membered heterocycloalkyl, are each optionally substituted with 1 or 2 substituents independently selected from R¹¹;
or any R^{c1} and R^{d1} attached to the same N atom, together with the N atom to which they are attached, form a 4-, 5-, or 6-membered heterocycloalkyl group optionally substituted with 1 or 2 substituents independently selected from R¹¹;
each R^{a2}, R^{c2} and R^{d2}, is independently selected from H, C₁₋₆ alkyl, and C₁₋₆ haloalkyl;
each R^{a3}, R^{c3} and R^{d3}, is independently selected from H, C₁₋₆ alkyl, and C₁₋₆ haloalkyl; and
each R^{b3} is independently selected from C₁₋₆ alkyl, and C₁₋₆ haloalkyl.

In one aspect, the present disclosure provides compounds of Formula (IA) or a pharmaceutically acceptable salt thereof, wherein:
Z is N;
Cy¹ is 5-10 membered heteroaryl; wherein each 5-10 membered heteroaryl has at least one ring-forming carbon atom and 1, 2, 3, or 4 ring-forming heteroatoms independently selected from N, O, and S; wherein the N and S are optionally oxidized; wherein a ring-forming carbon atom of 5-10 membered heteroaryl is optionally substituted by oxo to form a carbonyl group; and wherein the C₆₋₁₀ aryl and 5-10 membered heteroaryl are each optionally substituted with 1, 2, 3 or 4 substituents independently selected from R¹⁰;
Cy^{A} is selected from C₃₋₁₀ cycloalkyl, 4-10 membered heterocycloalkyl, C₆₋₁₀ aryl and 5-10 membered heteroaryl; wherein the 4-10 membered heterocycloalkyl and 5-10 membered heteroaryl each has at least one ring-forming carbon atom and 1, 2, 3, or 4 ring-forming heteroatoms independently selected from N, O, and S; wherein the N and S are optionally oxidized; wherein a ring-forming carbon atom of 5-10 membered heteroaryl and 4-10 membered heterocycloalkyl is optionally substituted by oxo to form a carbonyl group; and wherein the C₃₋₁₀ cycloalkyl, 4-10 membered heterocycloalkyl, C₆₋₁₀ aryl and 5-10 membered heteroaryl are each optionally substituted with 1, 2, 3 or 4 substituents independently selected from R²⁰;
R¹, R², and R³ are each independently selected from H, D, C₁₋₆ alkyl, C₁₋₆ haloalkyl, halo, CN, OR^{a9}, and NR^{c9}R^{d9};
R⁵ is selected from C₁₋₆ alkyl, C₁₋₆ haloalkyl, C₃₋₁₀ cycloalkyl, and C₆₋₁₀ aryl; wherein said C₁₋₆ alkyl, C₃₋₁₀ cycloalkyl, and C₆₋₁₀ aryl, are each optionally substituted with 1, 2, 3, or 4 substituents independently selected from R⁵⁰;
each R¹⁰ is independently selected from C₁₋₆ alkyl, C₁₋₆ haloalkyl, C₃₋₁₀ cycloalkyl, 4-10 membered heterocycloalkyl, halo, D, CN, OR^{a1}, and NR^{c1}R^{d1}; wherein said C₁₋₆ alkyl, C₃₋₁₀ cycloalkyl, and 4-10 membered heterocycloalkyl, are each optionally substituted with 1, 2, 3, or 4 substituents independently selected from R¹¹;
each R¹¹ is independently selected from C₁₋₆ alkyl, C₁₋₆ haloalkyl, C₃₋₁₀ cycloalkyl, halo, D, CN, OR^{a3}, C(O)R^{b3}, C(O)NR^{c3}R^{d3}, C(O)OR^{a3}, NR^{c3}R^{d3}, and S(O)₂R^{b3}; wherein said C₁₋₆ alkyl, and C₃₋₁₀ cycloalkyl, are each optionally substituted with 1, 2, 3, or 4 substituents independently selected from R¹²;
each R¹² is independently selected from C₁₋₆ alkyl, halo, D, CN, OR^{a5} and NR^{c5}R^{d5};
each R²⁰ is independently selected from C₁₋₆ alkyl, C₁₋₆ haloalkyl, C₃₋₁₀ cycloalkyl, halo, D, CN, OR^{a2}, C(O)R^{b2}, C(O)NR^{c2}R^{d2}, C(O)OR^{a2}, S(O)NR^{c2}R^{d2}, and S(O)₂R^{b2}; wherein said C₁₋₆ alkyl, and C₃₋₁₀ cycloalkyl are each optionally substituted with 1, 2, 3, or 4 substituents independently selected from R²¹;
each R²¹ is independently selected from C₁₋₆ alkyl, C₁₋₆ haloalkyl, halo, D, CN, OR^{a4}, and NR^{c4}R^{d4};
each R⁵⁰ is independently selected from C₁₋₆ alkyl, C₁₋₆ haloalkyl, halo, D, CN, OR^{a11}, and NR^{c11}R^{d11}.
each R^{a1}, R^{c1} and R^{d1} is independently selected from H, C₁₋₆ alkyl, C₁₋₆ haloalkyl, C₃₋₁₀ cycloalkyl, and 4-10 membered heterocycloalkyl; wherein said C₁₋₆ alkyl, C₃₋₁₀ cycloalkyl, and 4-10 membered heterocycloalkyl, are each optionally substituted with 1, 2, 3, or 4 substituents independently selected from R¹¹;
or any R^{cc} and R^{d1} attached to the same N atom, together with the N atom to which they are attached, form a 4-, 5-, or 6-membered heterocycloalkyl group optionally substituted with 1, 2, 3, or 4 substituents independently selected from R¹¹;
each R^{a2}, R^{c2} and R^{d2} is independently selected from H, C₁₋₆ alkyl, C₁₋₆ haloalkyl, and C₃₋₁₀ cycloalkyl; wherein said C₁₋₆ alkyl and C₃₋₁₀ cycloalkyl, are each optionally substituted with 1, 2, 3, or 4 substituents independently selected from R²¹;
each R^{b2} is independently selected from C₁₋₆ alkyl, C₁₋₆ haloalkyl, and C₃₋₁₀ cycloalkyl; wherein said C₁₋₆ alkyl and C₃₋₁₀ cycloalkyl, are each optionally substituted with 1, 2, 3, or 4 substituents independently selected from R²¹;
each R^{a3}, R^{c3} and R^{d3} is independently selected from H, C₁₋₆ alkyl, C₁₋₆ haloalkyl, and C₃₋₆ cycloalkyl; wherein said C₁₋₆ alkyl and C₃₋₆ cycloalkyl, are each optionally substituted with 1, 2, 3, or 4 substituents independently selected from R¹²;
each R^{b3} is independently selected from C₁₋₆ alkyl, C₁₋₆ haloalkyl, and C₃₋₆ cycloalkyl; wherein said C₁₋₆ alkyl and C₃₋₆ cycloalkyl, are each optionally substituted with 1, 2, 3, or 4 substituents independently selected from R¹²;
each R^{a4}, R^{c4} and R^{d4} is independently selected from H, C₁₋₆ alkyl, and C₁₋₆ haloalkyl;
each R^{a5}, R^{c5} and R^{d5} is independently selected from H, C₁₋₆ alkyl, and C₁₋₆ haloalkyl;
each R^{a9}, R^{c9}, and R^{d9} is independently selected from H, C₁₋₆ alkyl, and C₁₋₆ haloalkyl; and
each R^{a11}, R^{c11} and R^{d11} is independently selected from H, C₁₋₆ alkyl, and C₁₋₆ haloalkyl.

In one aspect, the present disclosure provides compounds of Formula (IB) or a pharmaceutically acceptable salt thereof, wherein:
Z is N or CR⁶;
Cy¹ is selected from C₆₋₁₀ aryl and 5-10 membered heteroaryl; wherein each 5-10 membered heteroaryl has at least one ring-forming carbon atom and 1, 2, 3, or 4 ring-forming heteroatoms independently selected from N, O, and S; wherein the N and S are optionally oxidized; wherein a ring-forming carbon atom of 5-10 membered heteroaryl is optionally substituted by oxo to form a carbonyl group; and wherein the C₆₋₁₀ aryl and 5-10 membered heteroaryl are each optionally substituted with 1, 2, 3 or 4 substituents independently selected from R¹⁰;
Cy^{A} is selected from C₃₋₁₀ cycloalkyl, 4-10 membered heterocycloalkyl, C₆₋₁₀ aryl and 5-10 membered heteroaryl; wherein the 4-10 membered heterocycloalkyl and 5-10 membered heteroaryl each has at least one ring-forming carbon atom and 1, 2, 3, or 4 ring-forming heteroatoms independently selected from N, O, and S; wherein the N and S are optionally oxidized; wherein a ring-forming carbon atom of 5-10 membered heteroaryl and 4-10 membered heterocycloalkyl is optionally substituted by oxo to form a carbonyl group; and wherein the C₃₋₁₀ cycloalkyl, 4-10 membered heterocycloalkyl, C₆₋₁₀ aryl and 5-10 membered heteroaryl are each optionally substituted with 1, 2, 3 or 4 substituents independently selected from R²⁰;
R¹, R², R³, and R⁶ are each independently selected from H, D, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, halo, CN, OR^{a9}, SR^{a9}, C(O)R^{b9}, C(O)NR^{c9}R^{d9}, C(O)OR^{a9}, NR^{c9}R^{d9}, NR^{c9}C(O)R^{b9}, NR^{c9}C(O)OR^{a9}, NR^{c9}C(O)NR^{c9}R^{d9}, NR^{c9}S(O)R^{b9}, NR^{c9}S(O)₂R^{b9}, NR^{c9}S(O)₂NR^{c9}R^{d9}, S(O)R^{b9}, S(O)NR^{c9}R^{d9}, S(O)₂R^{b9}, and S(O)₂NR^{c9}R^{d9}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, and 5-10 membered heteroaryl, are each optionally substituted with 1, 2, 3, or 4 substituents independently selected from R³⁰;
R⁴ is selected from H, D, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, halo, CN, OR^{a7}, SR^{a7}, C(O)R^{b7}, C(O)NR^{c7}R^{d7}, C(O)OR^{a7}, NR^{c7}R^{d7}, NR^{c7}C(O)R^{b7}, NR^{c7}C(O)OR^{a7}, NR^{c7}C(O)NR^{c7}R^{d7}, NR^{c7}S(O)R^{b7}, NR^{c7}S(O)₂R^{b7}, NR^{c7}S(O)₂NR^{c7}R^{d7}, S(O)R^{b7}, S(O)NR^{c7}R^{d7}, S(O)₂R^{b7}, and S(O)₂NR^{c7}R^{d7}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, and 5-10 membered heteroaryl, are each optionally substituted with 1, 2, 3, or 4 substituents independently selected from R⁴⁰;
each R¹⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NOR^{a1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R¹¹;
each R¹¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a3}, SR^{a3}, C(O)R^{b3}, C(O)NR^{c3}R^{d3}, C(O)OR^{a3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R¹²;
each R¹² is independently selected from C₁₋₆ alkyl, C₂₋₆ alkenyl, C₂₋₆ alkynyl, C₁₋₆ haloalkyl, C₃₋₆ cycloalkyl, C₆₋₁₀ aryl, 5-6 membered heteroaryl, 4-7 membered heterocycloalkyl, halo, D, CN, OR^{a5}, SR^{a5}, C(O)R^{b5}, C(O)NR^{c5}R^{d5}, C(O)OR^{a5}, NR^{c5}R^{d5} NR^{c5}C(O)R^{b5}, NR^{c5}C(O)OR^{a5}, NR^{c5}C(O)NR^{c5}R^{d5}, NR^{c5}S(O)R^{b5}, NR⁵S(O)₂R^{b5}, NR⁵S(O)₂NR^{c5}R^{d5}, S(O)R^{b5}, S(O)NR^{c5}R^{d5}, S(O)₂R^{b5}, and S(O)₂NR^{c5}R^{d5}; wherein said C₁₋₆ alkyl, C₂₋₆ alkenyl, C₂₋₆ alkynyl, C₃₋₆ cycloalkyl, C₆₋₁₀ aryl, 5-6 membered heteroaryl and 4-7 membered heterocycloalkyl, are each optionally substituted with 1, 2, 3, or 4 substituents independently selected from R^{g}; each R²⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2} NR^{c2}C(O)NR^{c2}R^{d2}, C(═NR^{e2})R^{b2}, C(═NOR^{a2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R²¹;
each R²¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a4}, SR^{a4}, C(O)R^{b4}, C(O)NR^{c4}R^{d4}, C(O)OR^{a4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R²²;
each R²² is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR a, SR^{a6}, C(O)R^{b6}, C(O)NR^{c6}R^{d6}, C(O)OR^{a6}, NR^{c6}R^{d6} NR^{c6}C(O)R^{b6}, NR^{c6}C(O)OR^{a6}, NR^{c6}C(O)NR^{c6}R^{d6}, NR^{c6}S(O)R^{b6}, NR^{c6}S(O)₂R^{b6}, NR^{c6}S(O)₂NR^{c6}R^{d6}, S(O)R^{b6}, S(O)NR^{c6}R^{d6}, S(O)₂R^{b6}, and S(O)₂NR^{c6}R^{d6}; wherein said C₁₋₆ alkyl, C₂₋₆ alkenyl, C₂₋₆ alkynyl, C₃₋₆ cycloalkyl, phenyl, 5-6 membered heteroaryl and 4-7 membered heterocycloalkyl, are each optionally substituted with 1, 2, 3, or 4 substituents independently selected from R^{g};
each R³⁰ is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a10}, SR^{a10}, C(O)R^{b10}, C(O)NR^{c10}R^{d10}, C(O)OR^{a10}, NR^{c10}R^{d10}, NR^{c10}C(O)R^{b10}, NR^{c10}C(O)OR^{a10}, NR^{c10}C(O)NR^{c10}R^{d10}, NR^{c10} S(O)R^{b10}, NR^{c10} S(O)₂R^{b10}, NR^{C10}S(O)₂NR^{c10}R^{d10}, S(O)R^{b10}, S(O)NR^{c10}R^{d10}, S(O)₂R^{b10}, and S(O)₂NR^{c10}R^{d10}; wherein said C₁₋₆ alkyl, C₂₋₆ alkenyl, C₂₋₆ alkynyl, C₃₋₆ cycloalkyl, phenyl, 5-6 membered heteroaryl and 4-7 membered heterocycloalkyl, are each optionally substituted with 1, 2, 3, or 4 substituents independently selected from R^{g};
each R⁴⁰ is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a8}, SR^{a8}, C(O)R^{b8}, C(O)NR^{c8}R^{d8}, C(O)OR^{a8}, NR^{c8}R^{d8}, NR^{c8}C(O)R^{b8}, NR^{c8}C(O)OR^{a8}, NR^{c8}C(O)NR^{c8}R^{d8}, NR^{c8} S(O)R^{b8}, NR^{c8}S(O)₂R^{b8} NR^{c8}S(O)₂NR^{c8}R^{d8}, S(O)R^{b8}, S(O)NR^{c8}R^{d8}, S(O)₂R^{b8}, and S(O)₂NR^{c8}R^{d8}; wherein said C₁₋₆ alkyl, C₂₋₆ alkenyl, C₂₋₆ alkynyl, C₃₋₆ cycloalkyl, phenyl, 5-6 membered heteroaryl and 4-7 membered heterocycloalkyl, are each optionally substituted with 1, 2, 3, or 4 substituents independently selected from R^{g};
each R^{a1}, R^{c1} and R^{d1} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R¹¹;
or any R^{c1} and R^{d1} attached to the same N atom, together with the N atom to which they are attached, form a 4-, 5-, 6- or 7-membered heterocycloalkyl group optionally substituted with 1, 2, 3, or 4 substituents independently selected from R¹¹;
each R^{b1} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R¹¹;
each R^{e1} is independently selected from H, CN, C₁₋₆ alkyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{a2}, R^{c2} and R^{d2}, is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl are each optionally substituted with 1, 2, 3, or 4 substituents independently selected from R²¹;
or any R^{c2} and R^{d2} attached to the same N atom, together with the N atom to which they are attached, form a 4-, 5-, 6- or 7-membered heterocycloalkyl group optionally substituted with 1, 2 or 3 substituents independently selected from R²¹;
each R^{b2} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl are each optionally substituted with 1, 2, 3, or 4 substituents independently selected from R²¹;
each R^{e2} is independently selected from H, CN, C₁₋₆ alkyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{a3}, R^{c3} and R^{d3}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R¹²;
or any R^{c3} and R^{d3} attached to the same N atom, together with the N atom to which they are attached, form a 4-, 5-, 6- or 7-membered heterocycloalkyl group optionally substituted with 1, 2 or 3 substituents independently selected from R¹²;
each R^{b3} is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl, are each optionally substituted with 1, 2, 3, or 4 substituents independently selected from R¹²;
each R^{a4}, R^{c4} and R^{d4}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R²²;
or any R^{c4} and R^{d4} attached to the same N atom, together with the N atom to which they are attached, form a 4-, 5-, 6- or 7-membered heterocycloalkyl group optionally substituted with 1, 2 or 3 substituents independently selected from R²²;
each R^{b4} is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R²²;
each R^{a5}, R^{c5} and R^{d5}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
or any R^{c5} and R^{d5} attached to the same N atom, together with the N atom to which they are attached, form a 4-, 5-, 6- or 7-membered heterocycloalkyl group optionally substituted with 1, 2, 3, or 4 substituents independently selected from R^{g};
each R^{b5} is independently selected from C₁₋₆ alkyl, C₂₋₆ alkenyl, C₂₋₆ alkynyl and C₁₋₆ haloalkyl; wherein said C₁₋₆ alkyl C₂₋₆ alkenyl and C₂₋₆ alkynyl are each optionally substituted with 1, 2, 3, or 4 substituents independently selected from R^{g};
each R^{a6}, R^{c6} and R^{d6}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g}; each R^{b6} is independently selected from C₁₋₆ alkyl, C₂₋₆ alkenyl, C₂₋₆ alkynyl, and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{a7}, R^{c7}, and R^{d7} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R⁴⁰;
or any R^{c7} and R^{d7} attached to the same N atom, together with the N atom to which they are attached, form a 4-, 5-, 6- or 7-membered heterocycloalkyl group optionally substituted with 1, 2 or 3 substituents independently selected from R⁴⁰;
each R^{b7} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R⁴⁰;
each R^{a8}, R^{c8} and R^{d8}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{b8} is independently selected from C₁₋₆ alkyl, C₂₋₆ alkenyl, C₂₋₆ alkynyl, and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{a9}, R^{c9}, and R^{d9} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³⁰;
or any R^{c9} and R^{d9} attached to the same N atom, together with the N atom to which they are attached, form a 4-, 5-, 6- or 7-membered heterocycloalkyl group optionally substituted with 1, 2 or 3 substituents independently selected from R³⁰;
each R^{b9} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³⁰;
each R^{a10}, R^{c10} and R^{d10}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{b10} is independently selected from C₁₋₆ alkyl, C₂₋₆ alkenyl, C₂₋₆ alkynyl, and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g}; and
each R^{g} is independently selected from D, OH, NO₂, CN, halo, C₁₋₆ alkyl, C₂₋₆ alkenyl, C₂₋₆ alkynyl, C₁₋₆ haloalkyl, C₃₋₆ cycloalkyl, C₃₋₆ cycloalkyl-C₁₋₂ alkylene, C₁₋₆ alkoxy, C₁₋₆ haloalkoxy, C₁₋₃ alkoxy-C₁₋₃ alkyl, C₁₋₃ alkoxy-C₁₋₃ alkoxy, HO—C₁₋₃ alkoxy, HO—C₁₋₃ alkyl, cyano-C₁₋₃ alkyl, H₂N—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkoxycarbonylamino, C₁₋₆ alkylcarbonyloxy, aminocarbonyloxy, C₁₋₆ alkylaminocarbonyloxy, di(C₁₋₆ alkyl)aminocarbonyloxy, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In one aspect, the present disclosure provides compounds of Formula (IB) or a pharmaceutically acceptable salt thereof, wherein:
Z is N or CR⁶;
Cy¹ is selected from 5-10 membered heteroaryl; wherein each 5-10 membered heteroaryl has at least one ring-forming carbon atom and 1, 2, 3, or 4 ring-forming heteroatoms independently selected from N, O, and S; wherein the N and S are optionally oxidized; wherein a ring-forming carbon atom of 5-10 membered heteroaryl is optionally substituted by oxo to form a carbonyl group; and wherein the 5-10 membered heteroaryl are each optionally substituted with 1, 2, 3 or 4 substituents independently selected from R¹⁰;
Cy^{A} is selected from C₆₋₁₀ aryl and 5-10 membered heteroaryl; wherein the 5-10 membered heteroaryl each has at least one ring-forming carbon atom and 1, 2, 3, or 4 ring-forming heteroatoms independently selected from N, O, and S; wherein the N and S are optionally oxidized; wherein a ring-forming carbon atom of 5-10 membered heteroaryl is optionally substituted by oxo to form a carbonyl group; and wherein the C₆₋₁₀ aryl and 5-10 membered heteroaryl are each optionally substituted with 1, 2, 3 or 4 substituents independently selected from R²⁰;
R¹, R², R³, and R⁶ are each independently selected from H, D, C₁₋₆ alkyl, C₁₋₆ haloalkyl, halo, CN, OR^{a}s, and NR^{c9}R^{d9};
R⁴ is selected from H, D, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, halo, CN, OR^{a7}, and NR^{c7}R^{d7}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, and 5-10 membered heteroaryl, are each optionally substituted with 1, 2, 3, or 4 substituents independently selected from R⁴⁰;
each R¹⁰ is independently selected from C₁₋₆ alkyl, C₁₋₆ haloalkyl, C₃₋₁₀ cycloalkyl, 4-10 membered heterocycloalkyl, halo, D, CN, OR^{a1}, and NR^{c1}R^{d1}; wherein said C₁₋₆ alkyl, C₃₋₁₀ cycloalkyl, and 4-10 membered heterocycloalkyl, are each optionally substituted with 1, 2, 3, or 4 substituents independently selected from R¹¹;
each R¹¹ is independently selected from C₁₋₆ alkyl, C₁₋₆ haloalkyl, C₃₋₁₀ cycloalkyl, halo, D, CN, OR^{a3}, C(O)R^{b3}, C(O)NR^{c3}R^{d3}, C(O)OR^{a3}, NR^{c3}R^{d3}, and NR^{c3}C(O)R^{b3}; wherein said C₁₋₆ alkyl, and C₃₋₁₀ cycloalkyl, are each optionally substituted with 1, 2, 3, or 4 substituents independently selected from R¹²;
each R¹² is independently selected from C₁₋₆ alkyl, halo, D, CN, OR^{a5} and NR^{c5}R^{d5};
each R²⁰ is independently selected from C₁₋₆ alkyl, C₁₋₆ haloalkyl, C₃₋₁₀ cycloalkyl, halo, D, CN, OR^{a2}, C(O)R^{b2}, C(O)NR^{c2}R^{d2}, C(O)OR^{a2}, S(O)NR^{c2}R^{d2}, and S(O)₂R^{b2}; wherein said C₁₋₆ alkyl, and C₃₋₁₀ cycloalkyl are each optionally substituted with 1, 2, 3, or 4 substituents independently selected from R²¹;
each R²¹ is independently selected from C₁₋₆ alkyl, C₁₋₆ haloalkyl, halo, D, CN, OR^{a4}, and NR^{c4}R^{d4}.
each R⁴⁰ is independently selected from C₁₋₆ alkyl, C₁₋₆ haloalkyl, halo, D, CN, OR^{a8}, and NR^{c8}R^{d8};
each R^{a1}, R^{c1} and R^{d1} is independently selected from H, C₁₋₆ alkyl, C₁₋₆ haloalkyl, C₃₋₁₀ cycloalkyl, and 4-10 membered heterocycloalkyl; wherein said C₁₋₆ alkyl, C₃₋₁₀ cycloalkyl, and 4-10 membered heterocycloalkyl, are each optionally substituted with 1, 2, 3, or 4 substituents independently selected from R¹¹;
or any R^{c1} and R^{d1} attached to the same N atom, together with the N atom to which they are attached, form a 4-, 5-, or 6-membered heterocycloalkyl group optionally substituted with 1, 2, 3, or 4 substituents independently selected from R¹¹;
each R^{a2}, R^{c2} and R^{d2} is independently selected from H, C₁₋₆ alkyl, C₁₋₆ haloalkyl, and C₃₋₁₀ cycloalkyl; wherein said C₁₋₆ alkyl and C₃₋₁₀ cycloalkyl, are each optionally substituted with 1, 2, 3, or 4 substituents independently selected from R²¹;
each R^{b2} is independently selected from C₁₋₆ alkyl, C₁₋₆ haloalkyl, and C₃₋₁₀ cycloalkyl; wherein said C₁₋₆ alkyl and C₃₋₁₀ cycloalkyl, are each optionally substituted with 1, 2, 3, or 4 substituents independently selected from R²¹;
each R^{a3}, R^{c3} and R^{d3} is independently selected from H, C₁₋₆ alkyl, C₁₋₆ haloalkyl, and C₃₋₆ cycloalkyl; wherein said C₁₋₆ alkyl and C₃₋₆ cycloalkyl, are each optionally substituted with 1, 2, 3, or 4 substituents independently selected from R¹²;
each R^{b3} is independently selected from C₁₋₆ alkyl, C₁₋₆ haloalkyl, and C₃₋₆ cycloalkyl; wherein said C₁₋₆ alkyl and C₃₋₆ cycloalkyl, are each optionally substituted with 1, 2, 3, or 4 substituents independently selected from R¹²;
each R^{a4}, R^{c4} and R^{d4} is independently selected from H, C₁₋₆ alkyl, and C₁₋₆ haloalkyl;
each R^{a5}, R^{c5} and R^{d5} is independently selected from H, C₁₋₆ alkyl, and C₁₋₆ haloalkyl;
each R^{a7}, R^{c7}, and R^{d7} is independently selected from H, C₁₋₆ alkyl, and C₁₋₆ haloalkyl;
each R^{a8}, R^{c8} and R^{d8} is independently selected from H, C₁₋₆ alkyl, and C₁₋₆ haloalkyl; and
each R^{a9}, R^{c9}, and R^{d9} is independently selected from H, C₁₋₆ alkyl, and C₁₋₆ haloalkyl.

In one aspect, the present disclosure provides compounds of Formula (IB) or a pharmaceutically acceptable salt thereof, wherein:
Z is N or CR⁶;
Cy¹ is selected from C₆₋₁₀ aryl and 5-10 membered heteroaryl; wherein each 5-10 membered heteroaryl has at least one ring-forming carbon atom and 1, 2, 3, or 4 ring-forming heteroatoms independently selected from N, O, and S; wherein a ring-forming carbon atom of 5-10 membered heteroaryl is optionally substituted by oxo to form a carbonyl group; and wherein the C₆₋₁₀ aryl and 5-10 membered heteroaryl are each optionally substituted with 1 or 2 substituents independently selected from R¹⁰;
Cy^{A} is selected from C₃₋₁₀ cycloalkyl, and C₆₋₁₀ aryl; wherein the C₃₋₁₀ cycloalkyl, and C₆. 10 aryl are each optionally substituted with 1, 2, or 3 substituents independently selected from R²⁰;
R¹ is independently selected from H, D, C₁₋₆ alkyl, C₁₋₆ haloalkyl halo, CN, OR^{a9}, and NR^{c9}R^{d9}.
R² and R³ are each independently selected from H, D, and C₁₋₆ alkyl;
R⁶ are each independently selected from H, D, C₁₋₆ alkyl, C₁₋₆ haloalkyl halo, CN, OR^{a9}, and NR^{c9}R^{d9};
R⁴ is selected from H, D, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, halo, CN, OR^{a7} and NR^{c7}R^{d7}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, and 5-10 membered heteroaryl, are each optionally substituted with 1 or 2 substituents independently selected from R⁴⁰;
each R¹⁰ is independently selected from C₁₋₆ alkyl, C₁₋₆ haloalkyl, C₃₋₁₀ cycloalkyl, 4-10 membered heterocycloalkyl, C₆₋₁₀ aryl, and 5-10 membered heteroaryl, halo, D, CN, OR^{a1}, and NR^{c1}R^{d1}; wherein said C₁₋₆ alkyl, C₃₋₁₀ cycloalkyl, 4-10 membered heterocycloalkyl, C₆₋₁₀ aryl, and 5-10 membered heteroaryl, are each optionally substituted with 1, 2 or 3 substituents independently selected from R¹¹;
each R¹¹ is independently selected from C₁₋₆ alkyl, C₁₋₆ haloalkyl, 4-10 membered heterocycloalkyl, C₆₋₁₀ aryl, halo, D, CN, OR^{a3}, C(O)R^{b3}, C(O)NR^{c3}R^{d3}, C(O)OR^{a3}, NR^{c3}R^{d3} NR^{c3}C(O)R^{b3} NR^{c3}C(O)NR^{c3}R^{d3}, S(O)₂R^{b3}, and S(O)₂NR^{c3}R^{d3}; wherein said C₁₋₆ alkyl and C₆₋₁₀ aryl, are each optionally substituted with 1 or 2 substituents independently selected from R¹²;
each R¹² is independently selected from C₁₋₆ alkyl, C₁₋₆ haloalkyl, 5-6 membered heteroaryl, halo, D, CN, OR^{a5}, C(O)R⁵, C(O)NR^{c5}R^{d5}, C(O)OR^{a5}, and NR^{c5}R^{d5};
each R²⁰ is independently selected from C₁₋₆ alkyl, C₁₋₆ haloalkyl, halo, D, CN, OR^{a2}, C(O)R^{b2}, C(O)NR^{c2}R^{d2}, C(O)OR^{a2}, NR^{c2}R^{d2}, and NR^{c2}C(O)R^{b2}; wherein said C₁₋₆ alkyl, is optionally substituted with 1 or 2 substituents independently selected from R²¹;
each R²¹ is independently selected from C₁₋₆ alkyl, C₁₋₆ haloalkyl, halo, D, CN, OR^{a4}, and NR^{c4}R^{d4}.
each R⁴⁰ is independently selected from C₁₋₆ alkyl, C₁₋₆ haloalkyl, halo, D, CN, OR^{a8}, and NR^{c8}R^{d8}.
each R^{a1}, R^{c1} and R^{d1} is independently selected from H, C₁₋₆ alkyl, C₁₋₆ haloalkyl, C₃₋₁₀ cycloalkyl, and 4-10 membered heterocycloalkyl; wherein said C₁₋₆ alkyl, C₃₋₁₀ cycloalkyl, and 4-10 membered heterocycloalkyl, are each optionally substituted with 1 or 2 substituents independently selected from R¹¹;
or any R^{c1} and R^{d1} attached to the same N atom, together with the N atom to which they are attached, form a 4-, 5-, or 6-membered heterocycloalkyl group optionally substituted with 1 or 2 substituents independently selected from R¹¹;
each R^{a2}, R^{c2} and R^{d2}, is independently selected from H, C₁₋₆ alkyl, and C₁₋₆ haloalkyl; wherein said C₁₋₆ alkyl, is optionally substituted with 1 or 2 substituents independently selected from R²¹;
each R^{b2} is independently selected from C₁₋₆ alkyl, C₂₋₆ alkenyl, C₂₋₆ alkynyl, and C₁₋₆ haloalkyl; wherein said C₁₋₆ alkyl, C₂₋₆ alkenyl, and C₂₋₆ alkynyl, are each optionally substituted with 1 or 2 substituents independently selected from R²¹;
each R^{a3}, R^{c3} and R^{d3}, is independently selected from H, C₁₋₆ alkyl, and C₁₋₆ haloalkyl; wherein said C₁₋₆ alkyl is optionally substituted with 1 or 2 substituents independently selected from R¹²;
or any R^{c3} and R^{d3} attached to the same N atom, together with the N atom to which they are attached, form a 4-, 5-, 6-membered heterocycloalkyl group optionally substituted with 1 or 2 substituents independently selected from R¹²;
each R^{b3} is independently selected from C₁₋₆ alkyl, C₁₋₆ haloalkyl, C₃₋₆ cycloalkyl, phenyl, 5-6 membered heteroaryl and 4-7 membered heterocycloalkyl; wherein said C₁₋₆ alkyl, C₃₋₆ cycloalkyl, phenyl, 5-6 membered heteroaryl and 4-7 membered heterocycloalkyl, are each optionally substituted with 1 or 2 substituents independently selected from R¹²;
each R^{a4}, R^{c4} and R^{d4}, is independently selected from H, C₁₋₆ alkyl, and C₁₋₆ haloalkyl;
each R^{a5}, R^{c5} and R^{d5}, is independently selected from H, C₁₋₆ alkyl, and C₁₋₆ haloalkyl;
each R^{b5} is independently selected from C₁₋₆ alkyl, and C₁₋₆ haloalkyl;
each R^{a7}, R^{c7}, and R^{d7} is independently selected from H, C₁₋₆ alkyl, and C₁₋₆ haloalkyl; wherein said C₁₋₆ alkyl, is optionally substituted with 1 or 2 substituents independently selected from R⁴⁰;
each R^{a8}, R^{c8} and R^{d8}, is independently selected from H, C₁₋₆ alkyl, and C₁₋₆ haloalkyl; and
each R^{a9}, R^{c9}, and R^{d9} is independently selected from H, C₁₋₆ alkyl, C₁₋₆ haloalkyl, C₃₋₁₀ cycloalkyl, and 4-10 membered heterocycloalkyl;
or any R^{c9} and R^{d9} attached to the same N atom, together with the N atom to which they are attached, form a 4-, 5- or 6-membered heterocycloalkyl group.

In some embodiments, X is CR⁴ and Y is N.

In some embodiments, X is NR⁵ and Y is C.

In some embodiments, Z is N.

In some embodiments, Z is CR⁶.

In some embodiments, Cy¹ is 5-10 membered heteroaryl; wherein each 5-10 membered heteroaryl has at least one ring-forming carbon atom and 1, 2, 3, or 4 ring-forming heteroatoms independently selected from N, O, and S; wherein the N and S are optionally oxidized; wherein a ring-forming carbon atom of 5-10 membered heteroaryl is optionally substituted by oxo to form a carbonyl group; and wherein the 5-10 membered heteroaryl is optionally substituted with 1, 2, 3 or 4 substituents independently selected from R¹⁰.

In some embodiments, Cy¹ is 5-6 membered heteroaryl; wherein each 5-6 membered heteroaryl has at least one ring-forming carbon atom and 1, 2, 3, or 4 ring-forming heteroatoms independently selected from N, O, and S; wherein the N and S are optionally oxidized; wherein a ring-forming carbon atom of 5-6 membered heteroaryl is optionally substituted by oxo to form a carbonyl group; and wherein the 5-6 membered heteroaryl is optionally substituted with 1, 2, 3 or 4 substituents independently selected from R¹⁰.

In some embodiments, Cy¹ is 5-10 membered heteroaryl optionally substituted with 1, 2, 3 or 4 substituents independently selected from R¹⁰. In some embodiments, Cy¹ is 5-6 membered heteroaryl optionally substituted with 1, 2, 3 or 4 substituents independently selected from R¹⁰.

In some embodiments, Cy¹ is 5-10 membered heteroaryl optionally substituted with 1 or 2 substituents independently selected from R¹⁰. In some embodiments, Cy¹ is 5-6 membered heteroaryl optionally substituted with 1 or 2 substituents independently selected from R¹⁰. In some embodiments, Cy¹ is 5 membered heteroaryl optionally substituted with 1 or 2 substituents independently selected from R¹⁰. In some embodiments, Cy¹ is 6 membered heteroaryl optionally substituted with 1 or 2 substituents independently selected from R¹⁰.

In some embodiments, Cy¹ is 5-10 membered heteroaryl or phenyl, each optionally substituted with 1, 2, 3 or 4 substituents independently selected from R¹⁰. In some embodiments, Cy¹ is 5-10 membered heteroaryl or phenyl, each optionally substituted with 1 or 2 substituents independently selected from R¹⁰.

In some embodiments, Cy¹ is 5-6 membered heteroaryl or phenyl, each optionally substituted with 1, 2, 3 or 4 substituents independently selected from R¹⁰. In some embodiments, Cy¹ is 5-6 membered heteroaryl or phenyl, each optionally substituted with 1 or 2 substituents independently selected from R¹⁰.

In some embodiments, Cy¹ is phenyl optionally substituted with 1, 2, 3 or 4 substituents independently selected from R¹⁰. In some embodiments, Cy¹ is phenyl optionally substituted with 1 or 2 substituents independently selected from R¹⁰.

In some embodiments, Cy¹ is pyrazolyl or pyridinyl, each optionally substituted with 1 or 2 substituents independently selected from R¹⁰. In some embodiments, Cy¹ is pyrazolyl optionally substituted with 1 or 2 substituents independently selected from R¹⁰. In some embodiments, Cy¹ is pyridinyl optionally substituted with 1 or 2 substituents independently selected from R¹⁰

In some embodiments, Cy¹ is pyrazolyl, pyridinyl, or phenyl, each optionally substituted with 1 or 2 substituents independently selected from R¹⁰.

In some embodiments, Cy¹ is selected from 1-methyl-1H-pyrazol-4-yl, 1,3-dimethyl-1H-pyrazol-4-yl, 1-(1-acetylpyrrolidin-3-yl)-1H-pyrazol-4-yl, 1-(4-carboxycyclohexyl)-1H-pyrazol-4-yl, 1-(4-acetamidocyclohexyl)-1H-pyrazol-4-yl, 1-(3-hydroxycyclobutyl)-1H-pyrazol-4-yl, and 6-(4-acetylpiperazin-1-yl)pyridin-3-yl.

In some embodiments, Cy¹ is selected from 1-methyl-1H-pyrazol-4-yl, 1,3-dimethyl-1H-pyrazol-4-yl, 1-(1-acetylpyrrolidin-3-yl)-1H-pyrazol-4-yl, 1-(4-carboxycyclohexyl)-1H-pyrazol-4-yl, 1-(4-acetamidocyclohexyl)-1H-pyrazol-4-yl, 1-(3-hydroxycyclobutyl)-1H-pyrazol-4-yl, 6-(4-acetylpiperazin-1-yl)pyridin-3-yl, 1-(1-(2-hydroxyacetyl)pyrrolidin-3-yl)-1H-pyrazol-4-yl, 1-(1-(1-aminocyclopropane-1-carbonyl)azetidin-3-yl)-1H-pyrazol-4-yl, 1-(1-(2-(1H-imidazol-1-yl)acetyl)azetidin-3-yl)-1H-pyrazol-4-yl, 1-(1-(oxazole-4-carbonyl)azetidin-3-yl)-1H-pyrazol-4-yl, 1-(1-(4-aminotetrahydro-2H-pyran-4-carbonyl)azetidin-3-yl)-1H-pyrazol-4-yl, 1-(1-(2-cyanoacetyl)azetidin-3-yl)-1H-pyrazol-4-yl, 1-(4-carboxy-3-fluorobenzyl)-1H-pyrazol-4-yl, 1-(4-(dimethylcarbamoyl)phenyl)-1H-pyrazol-4-yl, 1-((1r,4r)-4-(2-methoxyacetamido)cyclohexyl)-1H-pyrazol-4-yl, 1-((1r,4r)-4-(3,3-dimethylureido)cyclohexyl)-1H-pyrazol-4-yl, 1-(2-acetyl-2-azaspiro[3.5]nonan-7-yl)-1H-pyrazol-4-yl, 1-(tetrahydro-2H-pyran-4-yl)-1H-pyrazol-4-yl, 1-((1r,4r)-4-hydroxycyclohexyl)-1H-pyrazol-4-yl, 1-(4-carboxy-4-methylcyclohexyl)-1H-pyrazol-4-yl, 1-(1-(dimethylcarbamoyl)pyrrolidin-3-yl)-1H-pyrazol-4-yl, 1-(1-(N,N-dimethylsulfamoyl)pyrrolidin-3-yl)-1H-pyrazol-4-yl, 1-(3-(2-hydroxyacetyl)-3-azabicyclo[3.1.0]hexan-1-yl)-1H-pyrazol-4-yl, 1-(1-(morpholine-4-carbonyl)pyrrolidin-3-yl)-1H-pyrazol-4-yl, 1-(3-(2-hydroxyacetyl)-3-azabicyclo[4.1.0]heptan-6-yl)-1H-pyrazol-4-yl, 1-(3-acetyl-3-azabicyclo[4.1.0]heptan-1-yl)-1H-pyrazol-4-yl, 6-(1-(2-hydroxyacetyl)azetidin-3-yl)pyridin-3-yl, 5-(1-acetylazetidin-3-yl)pyridin-3-yl, 4-(1-acetylazetidin-3-yl)phenyl), 1-cyclopropyl-1H-pyrazol-4-yl, 1-(tetrahydrofuran-3-yl)-1H-pyrazol-4-yl, 1-(oxetan-3-yl)-1H-pyrazol-4-yl, 1-(2-(dimethylamino)ethyl)-1H-pyrazol-4-yl, 1-isopropyl-1H-pyrazol-4-yl, 1-(difluoromethyl)-1H-pyrazol-4-yl, 1-(2-cyanoethyl)-1H-pyrazol-4-yl, 1-(2-hydroxy-2-methylpropyl)-1H-pyrazol-4-yl, 1-(cyanomethyl)-1H-pyrazol-4-yl, 1-(1-methylpiperidin-4-yl)-1H-pyrazol-4-yl, 1-(1-hydroxy-2-methylpropan-2-yl)-1H-pyrazol-4-yl, 1-(2-(methylsulfonyl)ethyl)-1H-pyrazol-4-yl, 1-(2,2,2-trifluoroethyl)-1H-pyrazol-4-yl, 1-(2-morpholinoethyl)-1H-pyrazol-4-yl, and 1-(2-hydroxyethyl)-1H-pyrazol-4-yl.

In some embodiments, Cy¹ is selected from 1-methyl-1H-pyrazol-4-yl, 1,3-dimethyl-1H-pyrazol-4-yl, 1-(1-acetylpyrrolidin-3-yl)-1H-pyrazol-4-yl, 1-(4-carboxycyclohexyl)-1H-pyrazol-4-yl, 1-(4-acetamidocyclohexyl)-1H-pyrazol-4-yl, 1-(3-hydroxycyclobutyl)-1H-pyrazol-4-yl, 6-(4-acetylpiperazin-1-yl)pyridin-3-yl, 1-(1-(2-hydroxyacetyl)pyrrolidin-3-yl)-1H-pyrazol-4-yl, 1-(1-(1-aminocyclopropane-1-carbonyl)azetidin-3-yl)-1H-pyrazol-4-yl, 1-(1-(2-(1H-imidazol-1-yl)acetyl)azetidin-3-yl)-1H-pyrazol-4-yl, 1-(1-(oxazole-4-carbonyl)azetidin-3-yl)-1H-pyrazol-4-yl, 1-(1-(4-aminotetrahydro-2H-pyran-4-carbonyl)azetidin-3-yl)-1H-pyrazol-4-yl, 1-(1-(2-cyanoacetyl)azetidin-3-yl)-1H-pyrazol-4-yl, 1-(4-carboxy-3-fluorobenzyl)-1H-pyrazol-4-yl, 1-(4-(dimethylcarbamoyl)phenyl)-1H-pyrazol-4-yl, 1-((1r,4r)-4-(2-methoxyacetamido)cyclohexyl)-1H-pyrazol-4-yl, 1-((1r,4r)-4-(3,3-dimethylureido)cyclohexyl)-1H-pyrazol-4-yl, 1-(2-acetyl-2-azaspiro[3.5]nonan-7-yl)-1H-pyrazol-4-yl, 1-(tetrahydro-2H-pyran-4-yl)-1H-pyrazol-4-yl, 1-((1r,4r)-4-hydroxycyclohexyl)-1H-pyrazol-4-yl, 1-(4-carboxy-4-methylcyclohexyl)-1H-pyrazol-4-yl, 1-(1-(dimethylcarbamoyl)pyrrolidin-3-yl)-1H-pyrazol-4-yl, 1-(1-(N,N-dimethylsulfamoyl)pyrrolidin-3-yl)-1H-pyrazol-4-yl, 1-(3-(2-hydroxyacetyl)-3-azabicyclo[3.1.0]hexan-1-yl)-1H-pyrazol-4-yl, 1-(1-(morpholine-4-carbonyl)pyrrolidin-3-yl)-1H-pyrazol-4-yl, 1-(3-(2-hydroxyacetyl)-3-azabicyclo[4.1.0]heptan-6-yl)-1H-pyrazol-4-yl, 1-(3-acetyl-3-azabicyclo[4.1.0]heptan-1-yl)-1H-pyrazol-4-yl, 6-(1-(2-hydroxyacetyl)azetidin-3-yl)pyridin-3-yl, 5-(1-acetylazetidin-3-yl)pyridin-3-yl, 1-cyclopropyl-1H-pyrazol-4-yl, 1-(tetrahydrofuran-3-yl)-1H-pyrazol-4-yl, 1-(oxetan-3-yl)-1H-pyrazol-4-yl, 1-(2-(dimethylamino)ethyl)-1H-pyrazol-4-yl, 1-isopropyl-1H-pyrazol-4-yl, 1-(difluoromethyl)-1H-pyrazol-4-yl, 1-(2-cyanoethyl)-1H-pyrazol-4-yl, 1-(2-hydroxy-2-methylpropyl)-1H-pyrazol-4-yl, 1-(cyanomethyl)-1H-pyrazol-4-yl, 1-(1-methylpiperidin-4-yl)-1H-pyrazol-4-yl, 1-(1-hydroxy-2-methylpropan-2-yl)-1H-pyrazol-4-yl, 1-(2-(methylsulfonyl)ethyl)-1H-pyrazol-4-yl, 1-(2,2,2-trifluoroethyl)-1H-pyrazol-4-yl, 1-(2-morpholinoethyl)-1H-pyrazol-4-yl, and 1-(2-hydroxyethyl)-1H-pyrazol-4-yl.

In some embodiments, each R¹⁰ is independently selected from C₁₋₆ alkyl, C₁₋₆ haloalkyl, C₃₋₁₀ cycloalkyl, 4-10 membered heterocycloalkyl, halo, D, CN, OR, and NR^{c1}R^{d1}; wherein said C₁₋₆ alkyl, C₃₋₁₀ cycloalkyl, and 4-10 membered heterocycloalkyl, are each optionally substituted with 1, 2, 3, or 4 substituents independently selected from R¹¹.

In some embodiments, each R¹⁰ is independently selected from C₁₋₆ alkyl, C₁₋₆ haloalkyl, C₆₋₁₀ aryl, C₃₋₁₀ cycloalkyl, 4-10 membered heterocycloalkyl, halo, D, CN, OR^{a1}, and NR^{c1}R^{d1}; wherein said C₁₋₆ alkyl, C₆₋₁₀ aryl, C₃₋₁₀ cycloalkyl, and 4-10 membered heterocycloalkyl, are each optionally substituted with 1, 2, 3, or 4 substituents independently selected from R¹¹.

In some embodiments, each R¹⁰ is independently selected from C₁₋₆ alkyl, C₁₋₆ haloalkyl, C₃₋₇ cycloalkyl, 4-6 membered heterocycloalkyl, halo, D, CN, OR^{a1}, and NR^{c1}R^{d1}; wherein said C₁₋₆ alkyl, C₃₋₇ cycloalkyl, and 4-6 membered heterocycloalkyl are each optionally substituted with 1 or 2 substituents independently selected from R¹¹.

In some embodiments, each R¹⁰ is independently selected from C₁₋₆ alkyl, C₁₋₆ haloalkyl, 10 C₆₋₁₀ aryl, C₃₋₇ cycloalkyl, 4-6 membered heterocycloalkyl, halo, D, CN, OR^{a1}, and NR^{c1}R^{d1}; wherein said C₁₋₆ alkyl, C₆₋₁₀ aryl, C₃₋₇ cycloalkyl, and 4-6 membered heterocycloalkyl are each optionally substituted with 1 or 2 substituents independently selected from R¹¹.

In some embodiments, each R¹⁰ is independently selected from C₁₋₆ alkyl, 4-10 membered heterocycloalkyl, and C₃₋₁₀ cycloalkyl, each of which is optionally substituted with 1 or 2 substituents independently selected from R¹¹.

In some embodiments, each R¹⁰ is independently selected from C₁₋₆ alkyl, 4-10 membered heterocycloalkyl, C₆₋₁₀ aryl, and C₃₋₁₀ cycloalkyl, each of which is optionally substituted with 1 or 2 substituents independently selected from R¹¹.

In some embodiments, each R¹⁰ is independently selected from C₁₋₆ alkyl, 4-6 membered heterocycloalkyl, and C₃₋₇ cycloalkyl, each of which is optionally substituted with 1 or 2 substituents independently selected from R¹¹. In some embodiments, each R¹⁰ is independently selected from C₁₋₆ alkyl optionally substituted with 1 or 2 substituents independently selected from R¹¹. In some embodiments, each R¹⁰ is independently selected from 4-6 membered heterocycloalkyl optionally substituted with 1 or 2 substituents independently selected from R¹¹. In some embodiments, each R¹⁰ is independently selected from C₃₋₇ cycloalkyl optionally substituted with 1 or 2 substituents independently selected from R¹¹.

In some embodiments, each R¹⁰ is independently selected from C₁₋₆ alkyl, 4-6 membered heterocycloalkyl, C₆₋₁₀ aryl, and C₃₋₇ cycloalkyl, each of which is optionally substituted with 1 or 2 substituents independently selected from R¹¹. In some embodiments, each R¹⁰ is independently selected from C₁₋₆ alkyl, 4-6 membered heterocycloalkyl, phenyl, and C₃₋₇ cycloalkyl, each of which is optionally substituted with 1 or 2 substituents independently selected from R¹¹. In some embodiments, each R¹⁰ is independently selected from methyl, pyrrolidinyl, cyclohexyl, cyclobutyl, and piperazinyl, each of which is optionally substituted with 1 or 2 substituents independently selected from R¹¹. In some embodiments, each R¹⁰ is independently selected from methyl, pyrrolidinyl, cyclohexyl, and cyclobutyl, each of which is optionally substituted with 1 or 2 substituents independently selected from R¹¹. In some embodiments, each R¹⁰ is independently selected from methyl and piperazinyl, each of which is optionally substituted with 1 or 2 substituents independently selected from R¹¹.

In some embodiments, each R¹⁰ is independently selected from methyl, ethyl, isopropyl, 2-methylpropyl, difluoromethyl, cyclopropyl, cyclobutyl, cyclohexyl, phenyl, piperzinyl, azetidinyl, pyrrolidinyl, azaspiro[3.5]nonanyl, tetrahydro-2H-pyranyl, azabicyclo[3.1.0]hexanyl, azabicyclo[4.1.0]heptanyl, tetrahydrofuranyl, oxetanyl, and piperidinyl, each of which is optionally substituted with 1 or 2 substituents independently selected from R¹¹.

In some embodiments, each R¹⁰ is independently selected from methyl, ethyl, isopropyl, 2-methylpropyl, and difluoromethyl, wherein each methyl and ethyl is optionally substituted with 1 or 2 substituents independently selected from R¹¹. In some embodiments, each R¹⁰ is independently selected from methyl, ethyl, isopropyl, 2-methylpropyl, and difluoromethyl.

In some embodiments, each R¹⁰ is independently selected from cyclopropyl, cyclobutyl, cyclohexyl, phenyl, piperzinyl, azetidinyl, pyrrolidinyl, azaspiro[3.5]nonanyl, tetrahydro-2H-pyranyl, azabicyclo[3.1.0]hexanyl, azabicyclo[4.1.0]heptanyl, tetrahydrofuranyl, oxetanyl, and piperidinyl, each of which is optionally substituted with 1 or 2 substituents independently selected from R¹¹.

In some embodiments, each R¹¹ is independently selected from C₁₋₆ alkyl, C₁₋₆ haloalkyl, C₆₋₁₀ aryl, halo, D, CN, OR^{a}, C(O)R^{b3}, C(O)NR^{c3}R^{d3}, C(O)OR^{a3}, NR^{c3}R^{d3} NR^{c3}C(O)R^{b3}, S(O)₂R^{b3}, and S(O)₂NR^{c3}R^{d3}; wherein said C₁₋₆ alkyl and C₆₋₁₀ aryl are each optionally substituted with 1 or 2 substituents independently selected from R¹².

In some embodiments, each R¹¹ is independently selected from C₁₋₆ alkyl, C₃₋₇ cycloalkyl, CN, OR^{a3}, C(O)R^{b3}, C(O)NR^{c3}R^{d3}, C(O)OR³, and S(O)₂R^{b3}; wherein said C₁₋₆ alkyl, and C₃₋₇ cycloalkyl are each optionally substituted with 1 or 2 substituents independently selected from R¹².

In some embodiments, each R¹¹ is independently selected from C₁₋₆ alkyl, C₁₋₆ haloalkyl, C₆₋₁₀ aryl, halo, D, CN, OR^{a3}, C(O)R^{b3}, C(O)NR^{c3}R^{d3}, C(O)OR^{a3}, NR^{c3}R^{d3}, NR^{c3}C(O)R^{b3}, S(O)₂R^{b3}, and S(O)₂NR^{c3}R^{d3}; wherein said C₁₋₆ alkyl and C₆₋₁₀ aryl, are each optionally substituted with 1 or 2 substituents independently selected from R¹²

In some embodiments, each R¹¹ is independently selected from C₁₋₆ alkyl, C₁₋₆ haloalkyl, C₃₋₇ cycloalkyl, halo, D, CN, OR^{a3}, C(O)R^{b3}, C(O)NR^{c3}R^{d3}, C(O)OR^{a3}, NR^{c3}R^{d3} and S(O)₂Rb.

In some embodiments, each R¹¹ is independently selected from C(O)R^{b3}, C(O)OR^{a3}, NR^{c3}C(O)R^{b3}, and OR^{a3}. In some embodiments, each R¹¹ is independently selected from C(O)CH₃, C(O)OH, NHC(O)CH₃, and OH.
each R¹¹ is independently selected from C₁₋₆ alkyl, CN, C(O)R^{b3}, C(O)OR^{a3}, NR^{c3}C(O)R^{b3}, OR^{a3}, C(O)NR^{c3}R^{d3}, NR^{c3}R^{d3}, NR^{c3}C(O)NR^{c3}R^{d3}, S(O)₂NR^{c3}R^{d3}, S(O)₂R^{b3}, phenyl, and 4-6 membered heterocycloalkyl, wherein said C₁₋₆ alkyl, phenyl, and 4-6 membered heterocycloalkyl are each optionally substituted with 1 or 2 substituents independently selected from R¹².

In some embodiments, each R¹¹ is independently selected from C(O)R^{b3}, C(O)OR^{a3}, NR^{c3}C(O)R^{b3}, OR^{a3}, C(O)NR^{c3}R^{d3}, S(O)₂NR^{c3}R^{d3}, NR^{c3}R^{d3}, CN, and S(O)₂R^{b3}.

In some embodiments, each R¹¹ is independently selected from C(O)R^{b3}, C(O)OR^{a3}, and NR^{c3}C(O)R^{b3}. In some embodiments, each R¹¹ is independently selected from C(O)CH₃, C(O)OH, and NHC(O)CH₃.

In some embodiments, each R¹¹ is independently selected from methyl, CN, OH, C(O)CH₃, C(O)OH, NHC(O)CH₃, C(O)CH₂OH, C(O)N(CH₃)₂, NHC(O)CH₂OCH₃, NHC(O)N(CH₃)₂, S(O)₂N(CH₃)₂, N(CH₃)₂, S(O)₂CH₃, 2-cyanoacetyl, morpholine-4-carbonyl, morpholinyl, 1-aminocyclopropane-1-carbonyl, (1H-imidazol-1-yl)acetyl, oxazole-4-carbonyl, 4-aminotetrahydro-2H-pyran-4-carbonyl, and 4-carboxy-3-fluorophenyl.

In some embodiments, R¹¹ is OR^{a3}. In some embodiments, R¹¹ is OH.

In some embodiments, each R¹² is independently selected from C₁₋₆ alkyl, halo, D, CN, OR^{a5} and NR^{c5}R^{d5}.

In some embodiments, each R¹² is independently selected from C₁₋₆ alkyl, C₁₋₆ haloalkyl, 5-6 membered heteroaryl, halo, D, CN, OR^{a5}, C(O)R^{b5}, C(O)NR^{c5}R^{d5}, C(O)OR^{a5}, and NR^{c5}R^{d5}.

In some embodiments, Cy^{A} is selected from C₃₋₇ cycloalkyl, 4-6 membered heterocycloalkyl, phenyl and 5-6 membered heteroaryl; wherein the 4-6 membered heterocycloalkyl and 5-6 membered heteroaryl each has at least one ring-forming carbon atom and 1, 2, 3, or 4 ring-forming heteroatoms independently selected from N, O, and S; wherein the N and S are optionally oxidized; wherein a ring-forming carbon atom of 5-6 membered heteroaryl and 4-6 membered heterocycloalkyl is optionally substituted by oxo to form a carbonyl group; and wherein the C₃₋₇ cycloalkyl, 4-6 membered heterocycloalkyl, phenyl and 5-6 membered heteroaryl are each optionally substituted with 1, 2, 3 or 4 substituents independently selected from R²⁰.

In some embodiments, Cy^{A} is selected from C₃₋₇ cycloalkyl, 4-6 membered heterocycloalkyl, phenyl and 5-6 membered heteroaryl; wherein the 4-6 membered heterocycloalkyl and 5-6 membered heteroaryl each has at least one ring-forming carbon atom and 1, 2, 3, or 4 ring-forming heteroatoms independently selected from N, O, and S; wherein the N and S are optionally oxidized; wherein a ring-forming carbon atom of 5-6 membered heteroaryl and 4-6 membered heterocycloalkyl is optionally substituted by oxo to form a carbonyl group; and wherein the C₃₋₇ cycloalkyl, 4-6 membered heterocycloalkyl, phenyl and 5-6 membered heteroaryl are each optionally substituted with 1 or 2 substituents independently selected from R²⁰.

In some embodiments, Cy^{A} is selected from C₆₋₁₀ aryl and 5-10 membered heteroaryl; wherein the 5-10 membered heteroaryl each has at least one ring-forming carbon atom and 1, 2, 3, or 4 ring-forming heteroatoms independently selected from N, O, and S; wherein the N and S are optionally oxidized; wherein a ring-forming carbon atom of 5-10 membered heteroaryl is optionally substituted by oxo to form a carbonyl group; and wherein the C₆₋₁₀ aryl and 5-10 membered heteroaryl are each optionally substituted with 1, 2, 3 or 4 substituents independently selected from R²⁰.

In some embodiments, Cy^{A} is selected from phenyl and 5-6 membered heteroaryl; wherein the 5-6 membered heteroaryl each has at least one ring-forming carbon atom and 1, 2, 3, or 4 ring-forming heteroatoms independently selected from N, O, and S; wherein the N and S are optionally oxidized; wherein a ring-forming carbon atom of 5-6 membered heteroaryl is optionally substituted by oxo to form a carbonyl group; and wherein the phenyl and 5-6 membered heteroaryl are each optionally substituted with 1, 2, 3 or 4 substituents independently selected from R²⁰.

In some embodiments, Cy^{A} is selected from phenyl and 5-6 membered heteroaryl, wherein the phenyl and 5-6 membered heteroaryl are each optionally substituted with 1, 2, 3 or 4 substituents independently selected from R²⁰. In some embodiments, Cy^{A} is selected from phenyl and 5-6 membered heteroaryl, wherein the phenyl and 5-6 membered heteroaryl are each optionally substituted with 1 or 2 substituents independently selected from R²⁰.

In some embodiments, Cy^{A} is phenyl or furanyl, each optionally substituted with 1, 2, or 3 substituents independently selected from R²⁰. In some embodiments, Cy^{A} is phenyl or furanyl, each optionally substituted with 1 or 2 substituents independently selected from R²⁰.

In some embodiments, Cy^{A} is phenyl, furanyl, piperidinyl, 2-oxo-3,8-diazabicyclo[3.2.1]octanyl, or cyclopropyl, each optionally substituted with 1, 2, or 3 substituents independently selected from R²⁰. In some embodiments, Cy^{A} is phenyl, furanyl, piperidinyl, 2-oxo-3,8-diazabicyclo[3.2.1]octanyl, or cyclopropyl, each optionally substituted with 1 or 2 substituents independently selected from R²⁰.

In some embodiments, Cy^{A} is phenyl optionally substituted with 1, 2, or 3 substituents independently selected from R²⁰. In some embodiments, Cy^{A} is phenyl substituted with 1 or 2 substituents independently selected from R²⁰. In some embodiments, Cy^{A} is phenyl substituted with 1 or 2 substituents independently selected from R²⁰ at the ortho and/or para positions of the phenyl. For example, Cy^{A} is phenyl substituted with 2 substituents independently selected from R²⁰ at the ortho and para positions of the phenyl.

In some embodiments, Cy^{A} is phenyl substituted with 1 or 2 substituents independently selected from R²⁰ at the ortho positions of the phenyl. For example, Cy^{A} is phenyl substituted with 1 substituent independently selected from R²⁰ at the ortho position of the phenyl. For example, Cy^{A} is phenyl substituted with 2 substituents independently selected from R²⁰ at the ortho positions of the phenyl.

In some embodiments, Cy^{A} is selected from 2-chlorophenyl, 2,6-difluorophenyl, 2-chloro-6-fluorophenyl, 2,6-dichlorophenyl, 2-fluoro-6-methylphenyl, 2-chloro-4-methoxyphenyl, and 3-methylfuran-2-yl.

In some embodiments, Cy^{A} is selected from 2-chlorophenyl, 2,6-difluorophenyl, 2-chloro-6-fluorophenyl, 2,6-dichlorophenyl, 2-fluoro-6-methylphenyl, 2-chloro-4-methoxyphenyl, 3-methylfuran-2-yl, 1-methylpiperidin-4-yl, (1S,5R)-2-oxo-3,8-diazabicyclo[3.2.1]octan-8-yl, 2-chloro-6-(trifluoromethyl)phenyl, 2,6-dimethylphenyl, 2-cyano-6-fluorophenyl, 2-fluoro-6-methoxyphenyl, 2,3-dimethylphenyl, 4-(hydroxymethyl)-2-methylphenyl, 3-acrylamido-2,6-dichlorophenyl, 3-acrylamidophenyl, 2-acrylamidophenyl, and cyclopropyl.

In some embodiments, each R²⁰ is independently selected from C₁₋₆ alkyl, C₁₋₆ haloalkyl, C₃₋₁₀ cycloalkyl, halo, D, CN, OR^{a2}, C(O)R^{b2}, C(O)NR^{c2}R^{d2}, C(O)OR^{a2}, S(O)NR^{c2}R^{d2}, and S(O)₂R^{b2}; wherein said C₁₋₆ alkyl and C₃₋₁₀ cycloalkyl are each optionally substituted with 1, 2, 3, or 4 substituents independently selected from R²¹.

In some embodiments, each R²⁰ is independently selected from C₁₋₆ alkyl, C₁₋₆ haloalkyl, C₃₋₁₀ cycloalkyl, halo, D, CN, OR^{a2}, C(O)R^{b2}, C(O)NR^{c2}R^{d2}, C(O)OR^{a2}, NR^{c2}C(O)R^{b2}, S(O)NR^{c2}R^{d2}, and S(O)₂R^{b2}; wherein said C₁₋₆ alkyl and C₃₋₁₀ cycloalkyl are each optionally substituted with 1, 2, 3, or 4 substituents independently selected from R²¹.

In some embodiments, each R²⁰ is independently selected from halo, CN, C₁₋₆ alkyl, C₁₋₆ haloalkyl, and NR^{c2}C(O)R^{b2}, wherein the C₁₋₆ alkyl is optionally substituted with OR^{a4}
In some embodiments, each R²⁰ is independently selected from halo, C₁₋₆ alkyl, and OR^{a2}.

In some embodiments, each R²⁰ is independently selected from chloro, fluoro, methyl, and methoxy.

In some embodiments, each R²⁰ is independently selected from CN, chloro, fluoro, methyl, methoxy, trifluoromethyl, hydroxymethyl, and acrylamido.

In some embodiments, each R²¹ is independently selected from C₁₋₆ alkyl, C₁₋₆ haloalkyl, halo, D, CN, OR^{a4}, and NR^{c4}R^{d4}.

In some embodiments, R¹, R², R³, and R⁶ are each independently selected from H, D, C₁₋₆ alkyl, C₁₋₆ haloalkyl, halo, CN, OR^{a9}, and NR^{c9}R^{d9}.

In some embodiments, R¹, R², and R³ are each independently selected from H, D, C₁₋₆ alkyl, C₁₋₆ haloalkyl, halo, CN, OR^{a9}, and NR^{c9}R^{d9}.

In some embodiments, R¹ is H. In some embodiments, R¹ is selected from H, D, C₁₋₆ alkyl, C₁₋₆ haloalkyl, halo, CN, OR^{a9}, and NR^{c9}R^{d9}.

In some embodiments, R¹ is H, OR^{a9}, or NR^{c9}R^{d9}. In some embodiments, R¹ is H, OH, or N(CH₃)₂.

In some embodiments, R² is H. In some embodiments, R² is selected from H, D, C₁₋₆ alkyl, C₁₋₆ haloalkyl, halo, CN, OR^{a9}, and NR^{c9}R^{d9}.

In some embodiments, R³ is H. In some embodiments, R³ is selected from H, D, C₁₋₆ alkyl, C₁₋₆ haloalkyl, halo, CN, OR^{a9}, and NR^{c9}R^{d9}.

In some embodiments, R⁴ is selected from H, D, C₁₋₆ alkyl, C₂₋₆ alkenyl, C₂₋₆ alkynyl, C₁. 6 haloalkyl, C₃₋₁₀ cycloalkyl, 4-10 membered heterocycloalkyl, C₆₋₁₀ aryl, 5-10 membered heteroaryl, halo, CN, OR^{a7}, and NR^{c7}R^{d7}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, and 5-10 membered heteroaryl, are each optionally substituted with 1, 2, 3, or 4 substituents independently selected from R⁴⁰.

In some embodiments, R⁴ is selected from H, halo, CN, C₁₋₆ alkyl, C₂₋₆ alkenyl, C₃₋₁₀ cycloalkyl, C₆₋₁₀ aryl, and 5-10 membered heteroaryl, wherein said C₁₋₆ alkyl, C₂₋₆ alkenyl, C₃₋₁₀ cycloalkyl, C₆₋₁₀ aryl, and 5-10 membered heteroaryl are each optionally substituted with 1 or 2 substituents selected from R⁴⁰.

In some embodiments, R⁴ is selected from H, halo, CN, C₁₋₆ alkyl, C₂₋₆ alkenyl, C₃₋₇ cycloalkyl, phenyl, and 5-6 membered heteroaryl, wherein said C₁₋₆ alkyl, C₂₋₆ alkenyl, C₃₋₇ cycloalkyl, phenyl, and 5-6 membered heteroaryl are each optionally substituted with 1 or 2 substituents selected from R⁴⁰.

In some embodiments, R⁴ is independently selected from H, methyl, ethyl, chloro, CN, cyclopropyl, cyclopentenyl, propenyl, phenyl, pyrazolyl, and furanyl; wherein said methyl, ethyl, cyclopropyl, cyclopentenyl, propenyl, phenyl, pyrazolyl, and furanyl are each optionally substituted with 1 or 2 substituents selected from R⁴⁰.

In some embodiments, R⁴ is selected from H, methyl, ethyl, chloro, CN, cyclopent-1-en-1-yl, prop-1-en-1-yl, 3,5-dimethoxyphenyl, 1-methyl-1H-pyrazol-4-yl, furan-2-yl, and cyclopropyl.

In some embodiments, R⁴ is selected from H, methyl, ethyl, isopropyl, hydroxymethyl, 1-hydroxyethyl, chloro, bromo, CN, cyclopent-1-en-1-yl, prop-1-en-1-yl, 3,5-dimethoxyphenyl, 1-methyl-1H-pyrazol-4-yl, furan-2-yl, and cyclopropyl.

In some embodiments, each R⁴⁰ is independently selected from C₁₋₆ alkyl, C₁₋₆ haloalkyl, halo, D, CN, OR^{a}s, and NR^{c8}R^{d8}.

In some embodiments, each R⁴⁰ is independently selected from C₁₋₆ alkyl and OR^{a8}.

In some embodiments, R⁵ is selected from C₁₋₆ alkyl, C₁₋₆ haloalkyl, C₃₋₁₀ cycloalkyl, and C₆₋₁₀ aryl; wherein said C₁₋₆ alkyl, C₃₋₁₀ cycloalkyl, and C₆₋₁₀ aryl are each optionally substituted with 1, 2, 3, or 4 substituents independently selected from R⁵⁰.

In some embodiments, R⁵ is C₁₋₆ alkyl.

In some embodiments, R⁵ is methyl.

In some embodiments, each R⁵⁰ is independently selected from C₁₋₆ alkyl, C₁₋₆ haloalkyl, halo, D, CN, OR^{a11}, and NR^{c11}R^{d11}.

In some embodiments, R⁶ is H.

In some embodiments, R⁶ is H or OR^{a9}. In some embodiments, R⁶ is H or (tetrahydrofuran-3-yl)oxy.

In some embodiments, each R^{a1}, R^{c1} and R^{d1} is independently selected from H, C₁₋₆ alkyl, C₁₋₆ haloalkyl, C₃₋₁₀ cycloalkyl, and 4-10 membered heterocycloalkyl; wherein said C₁₋₆ alkyl, C₃₋₁₀ cycloalkyl, and 4-10 membered heterocycloalkyl, are each optionally substituted with 1, 2, 3, or 4 substituents independently selected from R¹¹.

In some embodiments, each R^{a1}, R^{c1} and R^{d1} is independently selected from H, C₁₋₆ alkyl, and C₁₋₆ haloalkyl.

In some embodiments, any R^{c1} and R^{d1} attached to the same N atom, together with the N atom to which they are attached, form a 4-, 5-, or 6-membered heterocycloalkyl group optionally substituted with 1, 2, 3, or 4 substituents independently selected from R¹¹.

In some embodiments, each R^{b1} is independently selected from C₁₋₆ alkyl and C₁₋₆ haloalkyl.

In some embodiments, each R^{c1} is independently selected from H and C₁₋₆ alkyl.

In some embodiments, each R^{a2}, R^{c2} and R^{d2} is independently selected from H, C₁₋₆ alkyl, C₁₋₆ haloalkyl, and C₃₋₁₀ cycloalkyl; wherein said C₁₋₆ alkyl and C₃₋₁₀ cycloalkyl, are each optionally substituted with 1, 2, 3, or 4 substituents independently selected from R²¹.

In some embodiments, each R^{a2}, R^{c2} and R^{d2} is independently selected from H, C₁₋₆ alkyl, and C₁₋₆ haloalkyl.

In some embodiments, each R^{b2} is independently selected from C₁₋₆ alkyl, C₁₋₆ haloalkyl, and C₃₋₁₀ cycloalkyl; wherein said C₁₋₆ alkyl and C₃₋₁₀ cycloalkyl, are each optionally substituted with 1, 2, 3, or 4 substituents independently selected from R²¹.

In some embodiments, each R^{b2} is independently selected from C₁₋₆ alkyl, C₂₋₆ alkenyl, C₂₋₆ alkynyl, and C₁₋₆ haloalkyl; wherein said C₁₋₆ alkyl, C₂₋₆ alkenyl, and C₂₋₆ alkynyl, are each optionally substituted with 1 or 2 substituents independently selected from R²¹. In some embodiments, each R^{b2} is C₂₋₆ alkenyl,

In some embodiments, each R^{b2} is independently selected from C₁₋₆ alkyl and C₁₋₆ haloalkyl.

In some embodiments, each R^{e2} is independently selected from H and C₁₋₆ alkyl.

In some embodiments, each R^{a3}, R^{c3} and R^{d3} is independently selected from H, C₁₋₆ alkyl, C₁₋₆ haloalkyl, and C₃₋₆ cycloalkyl; wherein said C₁₋₆ alkyl and C₃₋₆ cycloalkyl, are each optionally substituted with 1, 2, 3, or 4 substituents independently selected from R¹².

In some embodiments, each R^{a3}, R^{c3} and R^{d3} is independently selected from H, C₁₋₆ alkyl, and C₁₋₆ haloalkyl.

In some embodiments, any R^{c3} and R^{d3} attached to the same N atom, together with the N atom to which they are attached, form a 4-, 5-, 6-membered heterocycloalkyl group optionally substituted with 1 or 2 substituents independently selected from R¹². In some embodiments, any R^{c3} and R^{d3} attached to the same N atom, together with the N atom to which they are attached, form a 6-membered heterocycloalkyl group.

In some embodiments, each R^{b3} is independently selected from C₁₋₆ alkyl, C₁₋₆ haloalkyl, and C₃₋₆ cycloalkyl; wherein said C₁₋₆ alkyl and C₃₋₆ cycloalkyl, are each optionally substituted with 1, 2, 3, or 4 substituents independently selected from R¹².

In some embodiments, each R^{b3} is independently selected from C₁₋₆ alkyl and C₁₋₆ haloalkyl.

In some embodiments, each R^{b3} is independently selected from C₁₋₆ alkyl, C₁₋₆ haloalkyl, C₃₋₆ cycloalkyl, phenyl, 5-6 membered heteroaryl and 4-7 membered heterocycloalkyl; wherein said C₁₋₆ alkyl, C₃₋₆ cycloalkyl, phenyl, 5-6 membered heteroaryl and 4-7 membered heterocycloalkyl are each optionally substituted with 1 or 2 substituents independently selected from R¹². In some embodiments, each R^{b3} is independently selected from C₁₋₆ alkyl, C₁₋₆ haloalkyl, C₃₋₆ cycloalkyl, phenyl, 5-6 membered heteroaryl and 4-7 membered heterocycloalkyl.

In some embodiments, each R^{a4}, R^{c4} and R^{d4} is independently selected from H, C₁₋₆ alkyl, and C₁₋₆ haloalkyl.

In some embodiments, each R^{b4} is independently selected from C₁₋₆ alkyl and C₁₋₆ haloalkyl.

In some embodiments, each R^{a5}, R^{c5} and R^{d5} is independently selected from H, C₁₋₆ alkyl, and C₁₋₆ haloalkyl.

In some embodiments, each R^{b5} is independently selected from C₁₋₆ alkyl and C₁₋₆ haloalkyl.

In some embodiments, each R^{a6}, R^{c6} and R^{d6} is independently selected from H, C₁₋₆ alkyl, and C₁₋₆ haloalkyl.

In some embodiments, each R^{b6} is independently selected from C₁₋₆ alkyl and C₁₋₆ haloalkyl.

In some embodiments, each R^{a7}, R^{c7} and R^{d7} is independently selected from H, C₁₋₆ alkyl, and C₁₋₆ haloalkyl.

In some embodiments, each R^{b7} is independently selected from C₁₋₆ alkyl and C₁₋₆ haloalkyl.

In some embodiments, each R^{a8}, R^{c8} and R^{d8} is independently selected from H, C₁₋₆ alkyl, and C₁₋₆ haloalkyl.

In some embodiments, each R^{b8} is independently selected from C₁₋₆ alkyl and C₁₋₆ haloalkyl.

In some embodiments, each R^{a9}, R^{c9}, and R^{d9} is independently selected from H, C₁₋₆ alkyl, and C₁₋₆ haloalkyl.

In some embodiments, each R^{a9}, R^{c9}, and R^{d9} is independently selected from H, C₁₋₆ alkyl, C₁₋₆ haloalkyl, C₃₋₁₀ cycloalkyl, and 4-10 membered heterocycloalkyl. In some embodiments, each R^{a9}, R^{c9}, and R^{d9} is independently selected from H, C₁₋₆ alkyl, and 4-10 membered heterocycloalkyl.

In some embodiments, each R^{b9} is independently selected from C₁₋₆ alkyl and C₁₋₆ haloalkyl.

In some embodiments, each R^{a10}, R^{c10} and R^{d10} is independently selected from H, C₁₋₆ alkyl, and C₁₋₆ haloalkyl.

In some embodiments, each R^{b10} is independently selected from C₁₋₆ alkyl and C₁₋₆ haloalkyl.

In some embodiments, each R^{a11}R^{c11} and R^{d11} is independently selected from H, C₁₋₆ alkyl, and C₁₋₆ haloalkyl.

In some embodiments, each R^{b11} is independently selected from C₁₋₆ alkyl and C₁₋₆ haloalkyl.

Provided herein is a compound of Formula IIa:

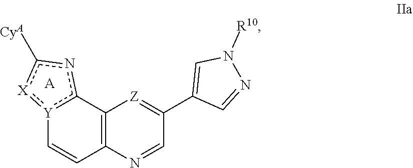

or a pharmaceutically acceptable salt thereof; wherein Cy^{A}, X, Y, Z, and R¹⁰ are as defined herein, and wherein ring A is aromatic.

Provided herein is a compound of Formula IIb:

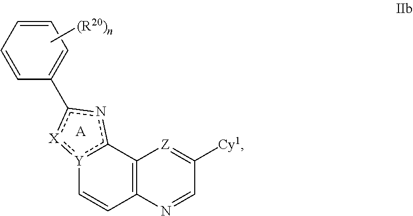

or a pharmaceutically acceptable salt thereof, wherein Cy¹, X, Y, Z, and each R²⁰ are as defined herein, wherein ring A is aromatic, and wherein n is 0, 1, 2, or 3. In some embodiments, n is 1 or 2.

Provided herein is a compound of Formula IIc:

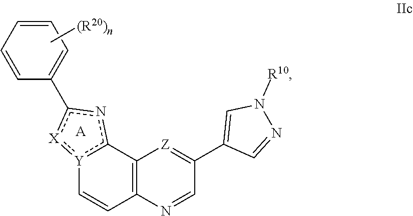

or a pharmaceutically acceptable salt thereof, wherein X, Y, Z, R¹⁰, and each R²⁰ are as defined herein, wherein ring A is aromatic, and wherein n is 0, 1, 2, or 3. In some embodiments, n is 1 or 2.

Provided herein is a compound of Formula IIIa:

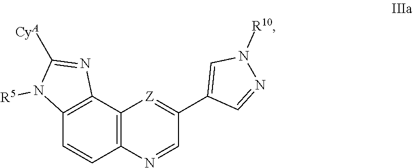

or a pharmaceutically acceptable salt thereof; wherein Cy^{A}, Z, R⁵, and R¹⁰ are as defined herein.

Provided herein is a compound of Formula IIIb:

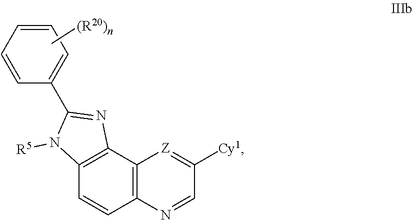

or a pharmaceutically acceptable salt thereof, wherein Cy¹, Z, R⁵, and each R²⁰ are as defined herein, and wherein n is 0, 1, 2, or 3. In some embodiments, n is 1 or 2.

Provided herein is a compound of Formula IIIc:

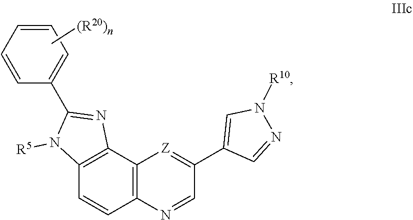

or a pharmaceutically acceptable salt thereof, wherein Z, R⁵, R¹⁰, and each R²⁰ are as defined herein, and wherein n is 0, 1, 2, or 3. In some embodiments, n is 1 or 2.

Provided herein is a compound of Formula IVa:

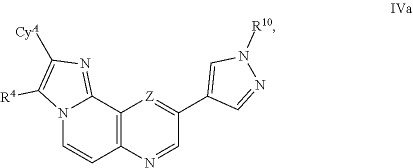

or a pharmaceutically acceptable salt thereof; wherein Cy^{A}, Z, R⁴, and R¹⁰ are as defined herein.

Provided herein is a compound of Formula IVb:

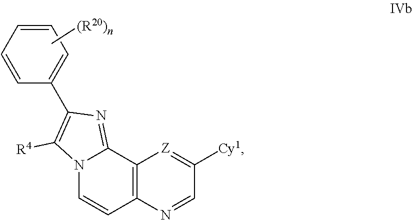

or a pharmaceutically acceptable salt thereof, wherein Cy¹, Z, R⁴, and each R²⁰ are as defined herein, and wherein n is 0, 1, 2, or 3. In some embodiments, n is 1 or 2.

Provided herein is a compound of Formula IVc:

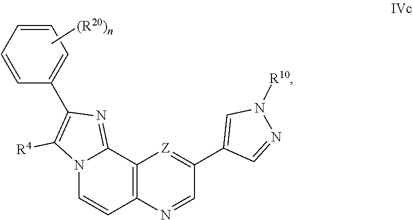

or a pharmaceutically acceptable salt thereof, wherein Z, R⁴, R¹⁰, and each R²⁰ are as defined herein, and wherein n is 0, 1, 2, or 3. In some embodiments, n is 1 or 2.

In some embodiments, provided herein is a compound selected from:
2-(2-Chlorophenyl)-3-methyl-9-(1-methyl-1H-pyrazol-4-yl)imidazo[2,1-f][1,6]naphthyridine;
2-(2-Chlorophenyl)-3-(cyclopent-1-en-1-yl)-9-(1-methyl-1H-pyrazol-4-yl)imidazo[2,1-f][1,6]naphthyridine;
2-(2-Chlorophenyl)-9-(1-methyl-1H-pyrazol-4-yl)-3-(prop-1-en-1-yl)imidazo[2,1-f][1,6]naphthyridine;
2-(2-Chlorophenyl)-3-(3,5-dimethoxyphenyl)-9-(1-methyl-1H-pyrazol-4-yl)imidazo[2,1-f][1,6]naphthyridine;
2-(2-Chlorophenyl)-3,9-bis(1-methyl-1H-pyrazol-4-yl)imidazo[2,1-f][1,6]naphthyridine;
2-(2-Chlorophenyl)-3-(furan-2-yl)-9-(1-methyl-1H-pyrazol-4-yl)imidazo[2,1-f][1,6]naphthyridine;
2-(2-Chlorophenyl)-9-(1-methyl-1H-pyrazol-4-yl)imidazo[2,1-f][1,6]naphthyridine-3-carbonitrile;
2-(2-Chlorophenyl)-3-cyclopropyl-9-(1-methyl-1H-pyrazol-4-yl)imidazo[2,1-f][1,6]naphthyridine;
2-(2-Chlorophenyl)-3-ethyl-9-(1-methyl-1H-pyrazol-4-yl)imidazo[2,1-f][1,6]naphthyridine;
3-Chloro-2-(2-chlorophenyl)-9-(1-methyl-1H-pyrazol-4-yl)imidazo[2,1-f][1,6]naphthyridine;
2-(2,6-Difluorophenyl)-9-(1-methyl-1H-pyrazol-4-yl)imidazo[2,1-f][1,6]naphthyridine;
2-(2-Chloro-6-fluorophenyl)-9-(1,3-dimethyl-1H-pyrazol-4-yl)imidazo[2,1-f][1,6]naphthyridine;
9-(2-Chlorophenyl)-8-cyclopropyl-2-(1-methyl-1H-pyrazol-4-yl)imidazo[1′,2′:1,2]pyrido[3,4-b]pyrazine;
9-(2-Chloro-6-fluorophenyl)-2-(1-methyl-1H-pyrazol-4-yl)imidazo[1′,2′:1,2]pyrido[3,4-b]pyrazine;
1-(3-(4-(2-(2,6-Dichlorophenyl)imidazo[2,1-f][1,6]naphthyridin-9-yl)-1H-pyrazol-1-yl)pyrrolidin-1-yl)ethan-1-one;
(1R,4R)-4-(4-(2-(2-Chlorophenyl)-3-cyclopropylimidazo[2,1-f][1,6]naphthyridin-9-yl)-1H-pyrazol-1-yl)cyclohexane-1-carboxylic acid;
N-((1R,4R)-4-(4-(2-(2-Chlorophenyl)-3-cyclopropylimidazo[2,1-f][1,6]naphthyridin-9-yl)-1H-pyrazol-1-yl)cyclohexyl)acetamide;
2-(2-Fluoro-6-methylphenyl)-3-methyl-8-(1-methyl-1H-pyrazol-4-yl)-3H-imidazo[4,5-f]quinoxaline;
2-(2-Chloro-4-methoxyphenyl)-3-methyl-8-(1-methyl-1H-pyrazol-4-yl)-3H-imidazo[4,5-f]quinoxaline;
3-Methyl-8-(1-methyl-1H-pyrazol-4-yl)-2-(3-methylfuran-2-yl)-3H-imidazo[4,5-f]quinoxaline;
3-(4-(2-(2-Chloro-6-fluorophenyl)-3-methyl-3H-imidazo[4,5-f]quinoxalin-8-yl)-1H-pyrazol-1-yl)cyclobutan-1-ol; and
1-(4-(5-(2-(2-Chloro-6-fluorophenyl)-3-methyl-3H-imidazo[4,5-f]quinoxalin-8-yl)pyridin-2-yl)piperazin-1-yl)ethan-1-one,
   or a pharmaceutically acceptable of any of the aforementioned.

In some embodiments, provided herein is a compound selected from:
3-Methyl-8-(1-methyl-1H-pyrazol-4-yl)-2-(1-methylpiperidin-4-yl)-3H-imidazo[4,5-f]quinoxaline;
(1S,5R)-8-(3-Methyl-8-(1-methyl-1H-pyrazol-4-yl)-3H-imidazo[4,5-f]quinoxalin-2-yl)-3,8-diazabicyclo[3.2.1]octan-2-one;
(S)-1-(3-(4-(2-(2-Chloro-6-(trifluoromethyl)phenyl)-3-methylimidazo[2,1-f][1,6]naphthyridin-9-yl)-1H-pyrazol-1-yl)pyrrolidin-1-yl)-2-hydroxyethan-1-one;
(S)-1-(3-(4-(2-(2,6-Dimethylphenyl)-3-methylimidazo[2,1-f][1,6]naphthyridin-9-yl)-1H-pyrazol-1-yl)pyrrolidin-1-yl)-2-hydroxyethan-1-one;
(S)-3-Fluoro-2-(9-(1-(1-(2-hydroxyacetyl)pyrrolidin-3-yl)-1H-pyrazol-4-yl)-3-methylimidazo[2,1-f][1,6]naphthyridin-2-yl)benzonitrile;
(S)-1-(3-(4-(2-(2-Fluoro-6-methoxyphenyl)-3-methylimidazo[2,1-f][1,6]naphthyridin-9-yl)-1H-pyrazol-1-yl)pyrrolidin-1-yl)-2-hydroxyethan-1-one;
2-(2,3-Dimethylphenyl)-3-methyl-9-(1-methyl-1H-pyrazol-4-yl)imidazo[2,1-f][1,6]naphthyridine;
(3-Methyl-4-(3-methyl-9-(1-methyl-1H-pyrazol-4-yl)imidazo[2,1-f][1,6]naphthyridin-2-yl)phenyl)methanol;
2-(2,6-Dichlorophenyl)-3-methyl-9-(1-methyl-1H-pyrazol-4-yl)imidazo[2,1-f][1,6]naphthyridin-8-ol;
(S)-2-(2,6-Dichlorophenyl)-3-methyl-9-(1-methyl-1H-pyrazol-4-yl)-10-((tetrahydrofuran-3-yl)oxy)imidazo[2,1-f][1,6]naphthyridine;
2-(2,6-Dichlorophenyl)-N,N,3-trimethyl-9-(1-methyl-1H-pyrazol-4-yl)imidazo[2,1-f][1,6]naphthyridin-8-amine;
N-(2,4-Dichloro-3-(3-methyl-9-(1-methyl-1H-pyrazol-4-yl)imidazo[2,1-f][1,6]naphthyridin-2-yl)phenyl)acrylamide;
N-(3-(3-Methyl-9-(1-methyl-1H-pyrazol-4-yl)imidazo[2,1-f][1,6]naphthyridin-2-yl)phenyl)acrylamide;
N-(2-(3-Methyl-9-(1-methyl-1H-pyrazol-4-yl)imidazo[2,1-f][1,6]naphthyridin-2-yl)phenyl)acrylamide;
(1-Aminocyclopropyl)(3-(4-(2-(2,6-dichlorophenyl)-3-methylimidazo[2,1-f][1,6]naphthyridin-9-yl)-1H-pyrazol-1-yl)azetidin-1-yl)methanone;
1-(3-(4-(2-(2,6-Dichlorophenyl)-3-methylimidazo[2,1-f][1,6]naphthyridin-9-yl)-1H-pyrazol-1-yl)azetidin-1-yl)-2-(1H-imidazol-1-yl)ethan-1-one;
(3-(4-(2-(2,6-Dichlorophenyl)-3-methylimidazo[2,1-f][1,6]naphthyridin-9-yl)-1H-pyrazol-1-yl)azetidin-1-yl)(oxazol-4-yl)methanone;
(4-Aminotetrahydro-2H-pyran-4-yl)(3-(4-(2-(2,6-dichlorophenyl)-3-methylimidazo[2,1-f][1,6]naphthyridin-9-yl)-1H-pyrazol-1-yl)azetidin-1-yl)methanone;
3-(3-(4-(2-(2,6-Dichlorophenyl)-3-methylimidazo[2,1-f][1,6]naphthyridin-9-yl)-1H-pyrazol-1-yl)azetidin-1-yl)-3-oxopropanenitrile;
4-((4-(2-(2,6-Dichlorophenyl)imidazo[2,1-f][1,6]naphthyridin-9-yl)-1H-pyrazol-1-yl)methyl)-2-fluorobenzoic acid;
4-(4-(2-(2,6-Dichlorophenyl)imidazo[2,1-f][1,6]naphthyridin-9-yl)-1H-pyrazol-1-yl)-N,N-dimethylbenzamide;
N-((1r,4r)-4-(4-(2-(2,6-Dichlorophenyl)imidazo[2,1-f][1,6]naphthyridin-9-yl)-1H-pyrazol-1-yl)cyclohexyl)-2-methoxyacetamide;
3-((1r,4r)-4-(4-(2-(2,6-Dichlorophenyl)imidazo[2,1-f][1,6]naphthyridin-9-yl)-1H-pyrazol-1-yl)cyclohexyl)-1,1-dimethylurea;
1-(7-(4-(2-(2,6-Dichlorophenyl)imidazo[2,1-f][1,6]naphthyridin-9-yl)-1H-pyrazol-1-yl)-2-azaspiro[3.5]nonan-2-yl)ethan-1-one;
2-(2,6-Dichlorophenyl)-3-methyl-9-(1-(tetrahydro-2H-pyran-4-yl)-1H-pyrazol-4-yl)imidazo[2,1-f][1,6]naphthyridine;
(1r,4r)-4-(4-(2-(2,6-Dichlorophenyl)-3-methylimidazo[2,1-f][1,6]naphthyridin-9-yl)-1H-pyrazol-1-yl)cyclohexan-1-ol;
4-(4-(2-(2,6-Dichlorophenyl)-3-methylimidazo[2,1-f][1,6]naphthyridin-9-yl)-1H-pyrazol-1-yl)-1-methylcyclohexane-1-carboxylic acid;
3-(4-(2-(2,6-Dichlorophenyl)imidazo[2,1-f][1,6]naphthyridin-9-yl)-1H-pyrazol-1-yl)-N,N-dimethylpyrrolidine-1-carboxamide;
3-(4-(2-(2,6-Dichlorophenyl)imidazo[2,1-f][1,6]naphthyridin-9-yl)-1H-pyrazol-1-yl)-N,N-dimethylpyrrolidine-1-sulfonamide;
(S)-1-(3-(4-(2-(2,6-Dichlorophenyl)-3-methylimidazo[2,1-f][1,6]naphthyridin-9-yl)-1H-pyrazol-1-yl)pyrrolidin-1-yl)ethan-1-one;
(S)-1-(3-(4-(2-(2,6-Dichlorophenyl)-3-methylimidazo[2,1-f][1,6]naphthyridin-9-yl)-1H-pyrazol-1-yl)pyrrolidin-1-yl)-2-hydroxyethan-1-one;
1-(1-(4-(2-(2,6-Dichlorophenyl)-3-methylimidazo[2,1-f][1,6]naphthyridin-9-yl)-1H-pyrazol-1-yl)-3-azabicyclo[3.1.0]hexan-3-yl)-2-hydroxyethan-1-one;
(S)-1-(3-(4-(2-(2,6-Dichlorophenyl)-3-(hydroxymethyl)imidazo[2,1-f][1,6]naphthyridin-9-yl)-1H-pyrazol-1-yl)pyrrolidin-1-yl)-2-hydroxyethan-1-one;
(S)-1-(3-(4-(2-(2,6-Dichlorophenyl)-3-(hydroxymethyl)imidazo[2,1-f][1,6]naphthyridin-9-yl)-1H-pyrazol-1-yl)pyrrolidin-1-yl)ethan-1-one;
(S)-(3-(4-(2-(2,6-Dichlorophenyl)-3-(hydroxymethyl)imidazo[2,1-f][1,6]naphthyridin-9-yl)-1H-pyrazol-1-yl)pyrrolidin-1-yl)(morpholino)methanone;
1-((3S)-3-(4-(2-(2,6-dichlorophenyl)-3-(1-hydroxyethyl)imidazo[2,1-f][1,6]naphthyridin-9-yl)-1H-pyrazol-1-yl)pyrrolidin-1-yl)-2-hydroxyethan-1-one;
1-(6-(4-(2-(2,6-Dichlorophenyl)-3-methylimidazo[2,1-f][1,6]naphthyridin-9-yl)-1H-pyrazol-1-yl)-3-azabicyclo[4.1.0]heptan-3-yl)-2-hydroxyethan-1-one;
1-(1-(4-(2-(2,6-Dichlorophenyl)-3-methylimidazo[2,1-f][1,6]naphthyridin-9-yl)-1H-pyrazol-1-yl)-3-azabicyclo[4.1.0]heptan-3-yl)ethan-1-one;
1-(3-(5-(2-(2,6-Dichlorophenyl)-3-methylimidazo[2,1-f][1,6]naphthyridin-9-yl)pyridin-2-yl)azetidin-1-yl)-2-hydroxyethan-1-one;
1-(3-(5-(2-(2,6-Dichlorophenyl)-3-methylimidazo[2,1-f][1,6]naphthyridin-9-yl)pyridin-3-yl)azetidin-1-yl)ethan-1-one;
1-(3-(5-(2-(2,6-Dichlorophenyl)-3-methylimidazo[2,1-f][1,6]naphthyridin-9-yl)phenyl)azetidin-1-yl)ethan-1-one;
(S)-1-(3-(4-(2-(2-Chlorophenyl)-3-(hydroxymethyl)imidazo[2,1-f][1,6]naphthyridin-9-yl)-1H-pyrazol-1-yl)pyrrolidin-1-yl)ethan-1-one;
(S)-1-(3-(4-(2-(2-Chlorophenyl)-3-isopropylimidazo[2,1-f][1,6]naphthyridin-9-yl)-1H-pyrazol-1-yl)pyrrolidin-1-yl)-2-hydroxyethan-1-one;
(S)-1-(3-(4-(3-Bromo-2-(2-chloro-6-fluorophenyl)imidazo[2,1-f][1,6]naphthyridin-9-yl)-1H-pyrazol-1-yl)pyrrolidin-1-yl)-2-hydroxyethan-1-one;
2-Cyclopropyl-3-methyl-9-(1-methyl-1H-pyrazol-4-yl)imidazo[2,1-f][1,6]naphthyridine;
(2-(2,6-Dichlorophenyl)-9-(1-methyl-1H-pyrazol-4-yl)imidazo[2,1-f][1,6]naphthyridin-3-yl)methanol;
(9-(1-Cyclopropyl-1H-pyrazol-4-yl)-2-(2,6-dichlorophenyl)imidazo[2,1-f][1,6]naphthyridin-3-yl)methanol;
(2-(2,6-Dichlorophenyl)-9-(1-(tetrahydrofuran-3-yl)-1H-pyrazol-4-yl)imidazo[2,1-f][1,6]naphthyridin-3-yl)methanol;
(2-(2,6-Dichlorophenyl)-9-(1-(oxetan-3-yl)-1H-pyrazol-4-yl)imidazo[2,1-f][1,6]naphthyridin-3-yl)methanol;
(2-(2,6-Dichlorophenyl)-9-(1-(2-(dimethylamino)ethyl)-1H-pyrazol-4-yl)imidazo[2,1-f][1,6]naphthyridin-3-yl)methanol;
(2-(2,6-Dichlorophenyl)-9-(1-isopropyl-1H-pyrazol-4-yl)imidazo[2,1-f][1,6]naphthyridin-3-yl)methanol;
(2-(2,6-Dichlorophenyl)-9-(1-(difluoromethyl)-1H-pyrazol-4-yl)imidazo[2,1-f][1,6]naphthyridin-3-yl)methanol;
3-(4-(2-(2,6-Dichlorophenyl)-3-(hydroxymethyl)imidazo[2,1-f][1,6]naphthyridin-9-yl)-1H-pyrazol-1-yl)propanenitrile;
1-(4-(2-(2,6-Dichlorophenyl)-3-(hydroxymethyl)imidazo[2,1-f][1,6]naphthyridin-9-yl)-1H-pyrazol-1-yl)-2-methylpropan-2-ol;
2-(4-(2-(2,6-Dichlorophenyl)-3-(hydroxymethyl)imidazo[2,1-f][1,6]naphthyridin-9-yl)-1H-pyrazol-1-yl)acetonitrile;
(2-(2,6-Dichlorophenyl)-9-(1-(1-methylpiperidin-4-yl)-1H-pyrazol-4-yl)imidazo[2,1-f][1,6]naphthyridin-3-yl)methanol;
2-(4-(2-(2,6-Dichlorophenyl)-3-(hydroxymethyl)imidazo[2,1-f][1,6]naphthyridin-9-yl)-1H-pyrazol-1-yl)-2-methylpropan-1-ol;
(2-(2,6-Dichlorophenyl)-9-(1-(2-(methylsulfonyl)ethyl)-1H-pyrazol-4-yl)imidazo[2,1-f][1,6]naphthyridin-3-yl)methanol;
(2-(2,6-Dichlorophenyl)-9-(1-(2,2,2-trifluoroethyl)-1H-pyrazol-4-yl)imidazo[2,1-f][1,6]naphthyridin-3-yl)methanol;
(2-(2,6-Dichlorophenyl)-9-(1-(2-morpholinoethyl)-1H-pyrazol-4-yl)imidazo[2,1-f][1,6]naphthyridin-3-yl)methanol; and
2-(4-(2-(2,6-Dichlorophenyl)-3-(hydroxymethyl)imidazo[2,1-f][1,6]naphthyridin-9-yl)-1H-pyrazol-1-yl)ethan-1-ol,

or a pharmaceutically acceptable of any of the aforementioned.

It is further appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

At various places in the present specification, substituents of compounds of the disclosure are disclosed in groups or in ranges. It is specifically intended that the disclosure include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

For compounds of the disclosure in which a variable appears more than once, each variable can be a different moiety independently selected from the group defining the variable. For example, where a structure is described having two R groups that are simultaneously present on the same compound, the two R groups can represent different moieties independently selected from the group defined for R.

As used herein, the phrase “optionally substituted” means unsubstituted or substituted.

The term “substituted” means that an atom or group of atoms formally replaces hydrogen as a “substituent” attached to another group. The term “substituted”, unless otherwise indicated, refers to any level of substitution, e.g., mono-, di-, tri-, tetra- or penta-substitution, where such substitution is permitted. The substituents are independently selected, and substitution may be at any chemically accessible position. It is to be understood that substitution at a given atom is limited by valency. It is to be understood that substitution at a given atom results in a chemically stable molecule. A single divalent substituent, e.g., oxo, can replace two hydrogen atoms.

As used herein, the term “Cᵢ₋ⱼ,” where i and j are integers, employed in combination with a chemical group, designates a range of the number of carbon atoms in the chemical group with i-j defining the range. For example, C₁₋₆ alkyl refers to an alkyl group having 1, 2, 3, 4, 5, or 6 carbon atoms.

As used herein, the term “alkyl,” employed alone or in combination with other terms, refers to a saturated hydrocarbon group that may be straight-chain or branched. An alkyl group formally corresponds to an alkane with one C—H bond replaced by the point of attachment of the alkyl group to the remainder of the compound. In some embodiments, the alkyl group contains 1 to 6, 1 to 4, or 1 to 3 carbon atoms. Examples of alkyl moieties include, but are not limited to, chemical groups such as methyl, ethyl, n-propyl, isopropyl, n-butyl, isobutyl, sec-butyl, tert-butyl, n-pentyl, 2-methyl-1-butyl, 3-pentyl, n-hexyl, 1,2,2-trimethylpropyl, and the like. In some embodiments, the alkyl group is methyl, ethyl, or propyl.

As used herein, the term “Cᵢ₋ⱼ alkylene,” employed alone or in combination with other terms, means a saturated divalent linking hydrocarbon group that may be straight-chain or branched, having i to j carbons. In some embodiments, the alkylene group contains from 1 to 4 carbon atoms, from 1 to 3 carbon atoms, or from 1 to 2 carbon atoms. Examples of alkylene moieties include, but are not limited to, chemical groups such as methylene, ethylene, 1,1-ethylene, 1,2-ethylene, 1,3-propylene, 1,2-propylene, 1,1-propylene, isopropylene, and the like.

As used herein, “alkenyl,” employed alone or in combination with other terms, refers to a straight-chain or branched hydrocarbon group corresponding to an alkyl group having one or more carbon-carbon double bonds. An alkenyl group formally corresponds to an alkene with one C—H bond replaced by the point of attachment of the alkenyl group to the remainder of the compound. In some embodiments, the alkenyl moiety contains 2 to 6 or 2 to 4 carbon atoms. Example alkenyl groups include, but are not limited to, ethenyl, n-propenyl, isopropenyl, n-butenyl, sec-butenyl, and the like.

As used herein, “alkynyl,” employed alone or in combination with other terms, refers to a straight-chain or branched hydrocarbon group corresponding to an alkyl group having one or more carbon-carbon triple bonds. An alkynyl group formally corresponds to an alkyne with one C—H bond replaced by the point of attachment of the alkyl group to the remainder of the compound. In some embodiments, the alkynyl moiety contains 2 to 6 or 2 to 4 carbon atoms. Example alkynyl groups include, but are not limited to, ethynyl, propyn-1-yl, propyn-2-yl, and the like.

As used herein, the terms “carbamoyl” and “carbamyl” interchangeably refer to a group of formula —C(O)NH₂.

As used herein, the term “carboxy” refers to a group of formula —C(O)OH.

The term “cyano” or “nitrile” refers to a group of formula —C≡N, which also may be written as —CN.

As used herein, the term “C₁₋₃ alkoxy-C₁₋₃ alkyl” refers to a group of formula —(C₁₋₃ alkylene)-(C₁₋₃ alkoxy).

As used herein, the term “C₁₋₃ alkoxy-C₁₋₃ alkoxy” refers to a group of formula —(C₁₋₃ alkoxylene)-(C₁₋₃ alkoxy).

As used herein, the term “HO—C₁₋₃ alkoxy” refers to a group of formula —(C₁₋₃ alkoxylene)-OH.

As used herein, the term “HO—C₁₋₃ alkyl” refers to a group of formula —(C₁₋₃ alkylene)-OH.

As used herein, the term “cyano-C₁₋₃ alkyl” refers to a group of formula —(C₁₋₃ alkylene)-CN.

As used herein, the term “H₂N—C₁₋₃ alkyl” refers to a group of formula —(C₁₋₃ alkylene)-NH₂.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms.

As used herein, the term “Cₙ₋ₘ alkoxycarbonyl” refers to a group of formula —C(O)O-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylcarbonyl” refers to a group of formula —C(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylcarbonylamino” refers to a group of formula —NHC(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylcarbonyloxy” refers to a group of formula —OC(O)-alkyl, wherein the alkyl group has n to m carbon atoms. In some embodiments, the alkyl group has 1 to 6, 1 to 4, or 1 to 3 carbon atoms.

As used herein, the term “aminocarbonyloxy” refers to a group of formula —OC(O)NH₂.

As used herein, the term “Cₙ₋ₘ alkylaminocarbonyloxy” refers to a group of formula —OC(O)NH-alkyl, wherein the alkyl group has n to m carbon atoms. In some embodiments, the alkyl group has 1 to 6, 1 to 4, or 1 to 3 carbon atoms.

As used herein, the term “Cn-m alkylsulfonylamino” refers to a group of formula —NHS(O)₂-alkyl, wherein the alkyl group has n to m carbon atoms. In some embodiments, the alkyl group has 1 to 6, 1 to 4, or 1 to 3 carbon atoms.

As used herein, the term “aminosulfonyl” refers to a group of formula —S(O)₂NH₂.

As used herein, the term “Cₙ₋ₘ alkylaminosulfonyl” refers to a group of formula —S(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonyl” refers to a group of formula —S(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminosulfonylamino” refers to a group of formula —NHS(O)₂NH₂.

As used herein, the term “Cₙ₋ₘ alkylaminosulfonylamino” refers to a group of formula —NHS(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonylamino” refers to a group of formula —NHS(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminocarbonylamino”, employed alone or in combination with other terms, refers to a group of formula —NHC(O)NH₂.

As used herein, the term “Cₙ₋ₘ alkylaminocarbonylamino” refers to a group of formula —NHC(O)NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminocarbonylamino” refers to a group of formula —NHC(O)N(alkyl)₂, wherein each alkyl group independently has n to m carbon atoms. In some embodiments, each alkyl group has, independently, 1 to 6, 1 to 4, or 1 to 3 carbon atoms.

As used herein, the term “Cₙ₋ₘ alkylcarbamyl” refers to a group of formula —C(O)—NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ-alkyl)carbamyl” refers to a group of formula —C(O)N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “thio” refers to a group of formula —SH.

As used herein, the term “Cₙ₋ₘ alkylthio” refers to a group of formula —S-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfinyl” refers to a group of formula —S(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonyl” refers to a group of formula —S(O)₂-alkyl, wherein the alkyl group has n to m carbon atoms. In some embodiments, the alkyl group has 1 to 6, 1 to 4, or 1 to 3 carbon atoms.

As used herein, “halo” or “halogen”, employed alone or in combination with other terms, includes fluoro, chloro, bromo, and iodo. In some embodiments, halo is F or Cl. In some embodiments, halo is F.

As used herein, the term “haloalkyl,” employed alone or in combination with other terms, refers to an alkyl group in which one or more of the hydrogen atoms has been replaced by a halogen atom, having up to the full valency of halogen atom substituents, which may either be the same or different. In some embodiments, the halogen atoms are fluoro atoms. In some embodiments, the alkyl group has 1 to 6, 1 to 4, or 1 to 3 carbon atoms. Example haloalkyl groups include CF₃, C₂F₅, CHF₂, CCl₃, CHCl₂, C₂C₅, and the like.

As used herein, the term “alkoxy,” employed alone or in combination with other terms, refers to a group of formula —O-alkyl. In some embodiments, the alkyl group has 1 to 6, 1 to 4, or 1 to 3 carbon atoms. Example alkoxy groups include methoxy, ethoxy, propoxy (e.g., n-propoxy and isopropoxy), t-butoxy, and the like. In some embodiments, alkoxy is methoxy.

As used herein, “haloalkoxy,” employed alone or in combination with other terms, refers to a group of formula —O-(haloalkyl). In some embodiments, the alkyl group has 1 to 6, 1 to 4, or 1 to 3 carbon atoms. An example haloalkoxy group is —OCF₃.

As used herein, “amino,” employed alone or in combination with other terms, refers to NH₂.

As used herein, the term “alkylamino,” employed alone or in combination with other terms, refers to a group of formula —NH(alkyl). In some embodiments, the alkylamino group has 1 to 6 or 1 to 4 carbon atoms. Example alkylamino groups include methylamino, ethylamino, propylamino (e.g., n-propylamino and isopropylamino), and the like.

As used herein, the term “alkylthio,” employed alone or in combination with other terms, refers to a group of formula —S-alkyl. In some embodiments, the alkyl group has 1 to 6 or 1 to 4 carbon atoms.

As used herein, the term “cycloalkyl,” employed alone or in combination with other terms, refers to a non-aromatic cyclic hydrocarbon including cyclized alkyl and alkenyl groups. The term “Cₙ₋ₘ cycloalkyl” refers to a cycloalkyl that has n to m ring member carbon atoms. Cycloalkyl groups can include mono- or polycyclic (e.g., having 2, 3, or 4 fused, bridged, or spiro rings) ring systems. Also included in the definition of cycloalkyl are moieties that have one or more aromatic rings (e.g., aryl or heteroaryl rings) fused (i.e., having a bond in common with) to the cycloalkyl ring, for example, benzo derivatives of cyclopentane, cyclohexene, cyclohexane, and the like, or pyrido derivatives of cyclopentane or cyclohexane. A cycloalkyl group containing a fused aromatic ring can be attached through any ring-forming atom including a ring-forming atom of the fused aromatic ring. Ring-forming carbon atoms of a cycloalkyl group can be optionally substituted by oxo. Cycloalkyl groups also include cycloalkylidenes. The term “cycloalkyl” also includes bridgehead cycloalkyl groups (e.g., non-aromatic cyclic hydrocarbon moieties containing at least one bridgehead carbon, such as admantan-1-yl) and spirocycloalkyl groups (e.g., non-aromatic hydrocarbon moieties containing at least two rings fused at a single carbon atom, such as spiro[2.5]octane and the like). In some embodiments, the cycloalkyl group has 3 to 10 ring members, or 3 to 7 ring members, or 3 to 6 ring members. In some embodiments, the cycloalkyl group is monocyclic or bicyclic. In some embodiments, the cycloalkyl group is monocyclic. In some embodiments, the cycloalkyl group is a C₇ monocyclic cycloalkyl group. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, tetrahydronaphthalenyl, octahydronaphthalenyl, indanyl, and the like. In some embodiments, the cycloalkyl group is cyclopropyl, cyclobutyl, cyclopentyl, or cyclohexyl.

As used herein, the term “heterocycloalkyl,” employed alone or in combination with other terms, refers to a non-aromatic ring or ring system, which may optionally contain one or more alkenylene or alkynylene groups as part of the ring structure, which has at least one heteroatom ring member independently selected from nitrogen, sulfur, oxygen, and phosphorus, and which has 4-14 ring members, 4-10 ring members, 4-7 ring members, or 4-6 ring members. Included within the term “heterocycloalkyl” are monocyclic 4-, 5-, 6- and 7-membered heterocycloalkyl groups. Heterocycloalkyl groups can include mono- or polycyclic (e.g., having 2, 3 or 4 fused, bridged, or spiro rings) or spirocyclic ring systems. In some embodiments, the heterocycloalkyl group is a monocyclic or bicyclic group having 1, 2, 3, or 4 heteroatoms independently selected from nitrogen, sulfur and oxygen. Also included in the definition of heterocycloalkyl are moieties that have one or more aromatic rings (e.g., aryl or heteroaryl rings) fused (i.e., having a bond in common with) to the non-aromatic heterocycloalkyl ring, for example, 1,2,3,4-tetrahydro-quinoline and the like. Heterocycloalkyl groups can also include bridgehead heterocycloalkyl groups (e.g., a heterocycloalkyl moiety containing at least one bridgehead atom, such as azaadmantan-1-yl and the like) and spiroheterocycloalkyl groups (e.g., a heterocycloalkyl moiety containing at least two rings fused at a single atom, such as [1,4-dioxa-8-aza-spiro[4.5]decan-N-yl] and the like). In some embodiments, the heterocycloalkyl group has 3 to 10 ring-forming atoms, 4 to 10 ring-forming atoms, or 3 to 8 ring forming atoms. In some embodiments, the heterocycloalkyl group has 1 to 5 heteroatoms, 1 to 4 heteroatoms, 1 to 3 heteroatoms, or 1 to 2 heteroatoms. The carbon atoms or heteroatoms in the ring(s) of the heterocycloalkyl group can be oxidized to form a carbonyl, an N-oxide, or a sulfonyl group (or other oxidized linkage) or a nitrogen atom can be quaternized. In some embodiments, the heterocycloalkyl portion is a C₂₇ monocyclic heterocycloalkyl group. In some embodiments, the heterocycloalkyl group is a morpholine ring, pyrrolidine ring, piperazine ring, piperidine ring, dihydropyran ring, tetrahydropyran ring, tetrahyropyridine, azetidine ring, or tetrahydrofuran ring. In some embodiments, the heterocycloalkyl is a 4-7 membered heterocycloalkyl moiety having carbon and 1, 2, or 3 heteroatoms independently selected from N, O and S. In some embodiments, the heterocycloalkyl is 4-10 membered heterocycloalkyl moiety having carbon and 1, 2, or 3 heteroatoms independently selected from N, O and S.

As used herein, the term “aryl,” employed alone or in combination with other terms, refers to a monocyclic or polycyclic (e.g., having 2 fused rings) aromatic hydrocarbon moiety, such as, but not limited to, phenyl, 1-naphthyl, 2-naphthyl, and the like. In some embodiments, aryl groups have from 6 to 10 carbon atoms or 6 carbon atoms. In some embodiments, the aryl group is a monocyclic or bicyclic group. In some embodiments, the aryl group is phenyl.

As used herein, the term “heteroaryl” or “heteroaromatic” employed alone or in combination with other terms, refers to a monocyclic or polycyclic (e.g., having 2 or 3 fused rings) aromatic hydrocarbon moiety, having one or more heteroatom ring members independently selected from nitrogen, sulfur and oxygen. In some embodiments, the heteroaryl group is a monocyclic or bicyclic group having 1, 2, 3, or 4 heteroatoms independently selected from nitrogen, sulfur and oxygen. Example heteroaryl groups include, but are not limited to, pyridyl, pyrimidinyl, pyrazinyl, pyridazinyl, triazinyl, furyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, purinyl, carbazolyl, benzimidazolyl, indolinyl, pyrrolyl, azolyl, quinolinyl, isoquinolinyl, benzisoxazolyl, imidazo[1,2-b]thiazolyl, pyridone, or the like. The carbon atoms or heteroatoms in the ring(s) of the heteroaryl group can be oxidized to form a carbonyl, an N-oxide, or a sulfonyl group (or other oxidized linkage) or a nitrogen atom can be quaternized, provided the aromatic nature of the ring is preserved. In one embodiment the heteroaryl group is a 5 to 10 membered heteroaryl group. In another embodiment the heteroaryl group is a 5 to 6 membered heteroaryl group. In some embodiments, the heteroaryl is a 5-6 membered heteroaryl moiety having carbon and 1, 2, or 3 heteroatoms independently selected from N, O and S. In some embodiments, the heteroaryl is a 5-10 membered heteroaryl moiety having carbon and 1, 2, or 3 heteroatoms independently selected from N, O and S. In some embodiments, the heteroaryl has 5-6 ring atoms and 1 or 2 heteroatom ring members independently selected from nitrogen, sulfur and oxygen. In some embodiments, no more than 2 heteroatoms of a 5-membered heteroaryl moiety are N.

A five-membered heteroaryl ring is a heteroaryl group having five ring atoms wherein one or more (e.g., 1, 2 or 3) ring atoms are independently selected from N, O and S. Exemplary five-membered ring heteroaryls include thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl and 1,3,4-oxadiazolyl.

A six-membered heteroaryl ring is a heteroaryl group having six ring atoms wherein one or more (e.g., 1, 2 or 3) ring atoms are independently selected from N, O and S. Exemplary six-membered ring heteroaryls are pyridyl, pyrazinyl, pyrimidinyl, triazinyl, isoindolyl, and pyridazinyl.

The term “oxo” refers to an oxygen atom as a divalent substituent, forming a carbonyl group when attached to carbon, or attached to a heteroatom forming a sulfoxide or sulfone group, or an N-oxide group. In some embodiments, heterocyclic groups may be optionally substituted by 1 or 2 oxo (═O) substituents.

The term “oxidized” in reference to a ring-forming N atom refers to a ring-forming N-oxide.

The term “oxidized” in reference to a ring-forming S atom refers to a ring-forming sulfonyl or ring-forming sulfinyl.

The term “aromatic” refers to a carbocycle or heterocycle having one or more polyunsaturated rings having aromatic character (i.e., having (4n+2) delocalized π (pi) electrons where n is an integer).

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas an azetidin-3-yl ring is attached at the 3-position.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Resolution of racemic mixtures of compounds can be carried out by methods known in the art. An example method includes fractional recrystallizaion using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids. Other resolving agents suitable for fractional crystallization methods include stereoisomerically pure forms of methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

In some embodiments, the compounds of the disclosure have the (R)-configuration. In other embodiments, the compounds have the (S)-configuration. In compounds with more than one chiral centers, each of the chiral centers in the compound may be independently (R) or (S), unless otherwise indicated.

Compounds of the disclosure also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide-imidic acid pairs, lactam—lactim pairs, enamine —imine pairs, and annular forms where a proton can occupy two or more positions of a heterocyclic system, for example, 1H- and 3H-imidazole, 1H-, 2H- and 4H- 1,2,4-triazole, 1H- and 2H- isoindole, and 1H- and 2H-pyrazole. Tautomeric forms can be in equilibrium or sterically locked into one form by appropriate substitution.

Compounds of the disclosure also include all isotopes of atoms occurring in the intermediates or final compounds. Isotopes include those atoms having the same atomic number but different mass numbers. For example, isotopes of hydrogen include tritium and deuterium. One or more constituent atoms of the compounds of the disclosure can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7, 8, 9, 10, 11 or 12 deuterium atoms. Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can used in various studies such as NMR spectroscopy, metabolism experiments, and/or assays.

Substitution with heavier isotopes such as deuterium, may afford certain therapeutic advantages resulting from greater metabolic stability, for example, increased in vivo half-life or reduced dosage requirements, and hence may be preferred in some circumstances. (A. Kerekes et.al. J. Med. Chem. 2011, 54, 201-210; R. Xu et.al. J. Label Compd. Radiopharm. 2015, 58, 308-312).

The term, “compound,” as used herein is meant to include all stereoisomers, geometric iosomers, tautomers, and isotopes of the structures depicted. The term is also meant to refer to compounds of the disclosure, regardless of how they are prepared, e.g., synthetically, through biological process (e.g., metabolism or enzyme conversion), or a combination thereof.

All compounds, and pharmaceutically acceptable salts thereof, can be found together with other substances such as water and solvents (e.g., in the form of hydrates and solvates) or can be isolated. When in the solid state, the compounds described herein and salts thereof may occur in various forms and may, e.g., take the form of solvates, including hydrates. The compounds may be in any solid state form, such as a polymorph or solvate, so unless clearly indicated otherwise, reference in the specification to compounds and salts thereof should be understood as encompassing any solid state form of the compound.

In some embodiments, the compounds of the disclosure, or salts thereof, are substantially isolated. By “substantially isolated” is meant that the compound is at least partially or substantially separated from the environment in which it was formed or detected. Partial separation can include, for example, a composition enriched in the compounds of the disclosure. Substantial separation can include compositions containing at least about 50%, at least about 60%, at least about 70%, at least about 80%, at least about 90%, at least about 95%, at least about 97%, or at least about 99% by weight of the compounds of the disclosure, or salt thereof. Methods for isolating compounds and their salts are routine in the art.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

The following abbreviations may be used herein: AcOH (acetic acid); Ac₂O (acetic anhydride); aq. (aqueous); atm. (atmosphere(s)); Boc (t-butoxycarbonyl); br (broad); Cbz (carboxybenzyl); calc. (calculated); d (doublet); dd (doublet of doublets); DCM (dichloromethane); DEAD (diethyl azodicarboxylate); DIAD (N,N′-diisopropyl azidodicarboxylate); DIPEA (N,N-diisopropylethylamine); DMF (N,N-dimethylformamide); Et (ethyl); EtOAc (ethyl acetate); g (gram(s)); h (hour(s)); HATU (N,N,N′,N′-tetramethyl-O-(7-azabenzotriazol-1-yl)uronium hexafluorophosphate); HCl (hydrochloric acid); HPLC (high performance liquid chromatography); Hz (hertz); J (coupling constant); LCMS (liquid chromatography—mass spectrometry); m (multiplet); M (molar); mCPBA (3-chloroperoxybenzoic acid); MgSO₄ (magnesium sulfate); MS (Mass spectrometry); Me (methyl); MeCN (acetonitrile); MeOH (methanol); mg (milligram(s)); min. (minutes(s)); mL (milliliter(s)); mmol (millimole(s)); N (normal); NaHCO₃ (sodium bicarbonate); NaOH (sodium hydroxide); Na₂SO₄ (sodium sulfate); NH₄Cl (ammonium chloride); NH₄OH (ammonium hydroxide); NIS (N-iodosuccinimide); nM (nanomolar); NMR (nuclear magnetic resonance spectroscopy); OTf (trifluoromethanesulfonate); Pd (palladium); Ph (phenyl); pM (picomolar); PMB (para-methoxybenzyl), POCl₃ (phosphoryl chloride); RP-HPLC (reverse phase high performance liquid chromatography); s (singlet); SEM (2-trimethylsilylethoxymethyl); t (triplet or tertiary); TBS (tert-butyldimethylsilyl); tert (tertiary); tt (triplet of triplets); t-Bu (tert-butyl); TFA (trifluoroacetic acid); THF (tetrahydrofuran); μg (microgram(s)); μL (microliter(s)); μM (micromolar); wt % (weight percent).

### Synthesis

As will be appreciated by those skilled in the art, the compounds provided herein, including salts and stereoisomers thereof, can be prepared using known organic synthesis techniques and can be synthesized according to any of numerous possible synthetic routes.

The reactions for preparing compounds of the disclosure can be carried out in suitable solvents which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the disclosure can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd. Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatography (HPLC) or thin layer chromatography.

The expressions, “ambient temperature,” “room temperature,” and “r.t.”, as used herein, are understood in the art, and refer generally to a temperature, e.g. a reaction temperature, that is about the temperature of the room in which the reaction is carried out, for example, a temperature from about 20° C. to about 30° C.

Compounds of formula S-11 can be prepared via the synthetic route as outlined in Scheme 1. The commercially available starting material S-1 can undergo a coupling reaction to adduct S-2, in which M is a boronic acid, a boronic ester or an appropriately substituted metal (e.g., M is B(OR)₂, Sn(Alkyl)₃, Zn-Hal, etc.), under standard Suzuki cross-coupling conditions (e.g., in the presence of a palladium catalyst and a suitable base) (Tetrahedron 2002, 58, 9633-9695), or standard Stille cross-coupling conditions (e.g., in the presence of a palladium catalyst) (ACS Catalysis 2015, 5, 3040-3053), or standard Negishi cross-coupling conditions (e.g., in the presence of a palladium catalyst) (ACS Catalysis 2016, 6, 1540-1552), to give derivative S-3. Reduction of the nitro group of compound S-3 under appropriate conditions can deliver compound S-4. Compound S-4 can undergo a halogenation reaction, such as electrophilic aromatic substitution (S_{E}Ar), with an appropriate reagent, such as N-bromosuccinimide (NBS), to afford compound S-5 (Hal is a halide, such as F, Cl, Br, or I). Halide displacement of reagent S-6 with intermediate S-5 via nucleophilic substitution (S_{N}2) generates intermediate S-7.

Compound S-7 can undergo an amination reaction, such as a Buchwald-Hartwig coupling (Chem. Sci. 2011, 2, 27-50) in the presence of a palladium catalyst (e.g., tris(dibenzylideneacetone)dipalladium(0)), appropriate ligand (e.g., 2′-(di-tert-butylphosphino)-N,N-dimethylbiphenyl-2-amine), and a base (e.g., sodium tert-butoxide) to afford diamine S-8. Compound S-8 can be coupled with a carboxylic acid adduct of formula S-9, using an appropriate coupling reagent (e.g. HATU, BOP, or PyBOP), to afford compound S-10. Finally, cyclization of compound S-10 at elevated temperature in the presence of a suitable reagent, such as acetic acid, generates compound S-11.

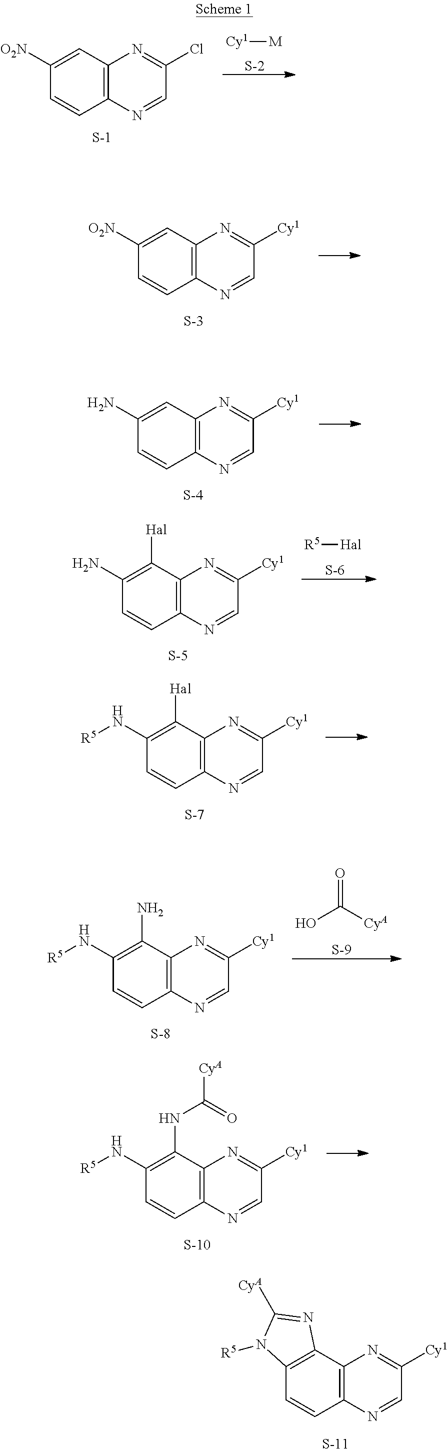

Alternatively, compound S-11 can be prepared via the synthetic route as outlined in Scheme 2. Acylation of compound S-8 with adduct S-12 (Hal is a halide, such as F, Cl, Br, or I) affords intermediate S-10. Cyclization of compound S-10 at elevated temperature in the presence of a suitable reagent, such as acetic acid, generates compound S-11.

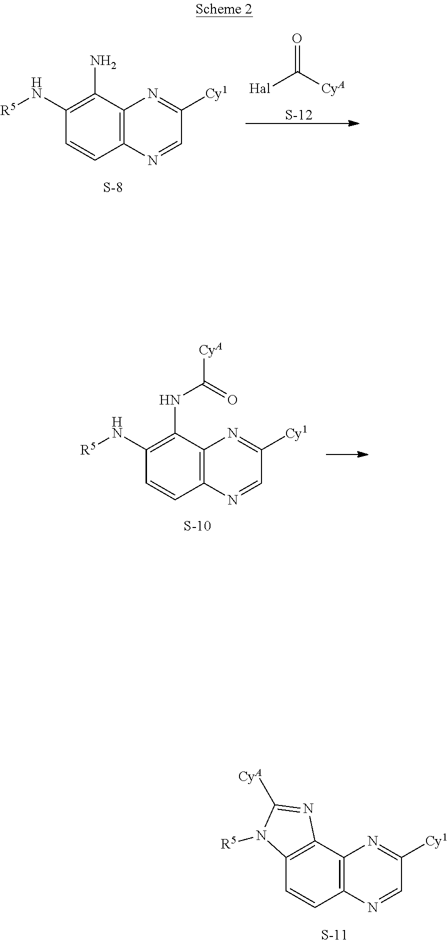

Compounds of formula S-21 can be prepared via the synthetic route as outlined in Scheme 3. Treatment of commercially available compound S-13 with an appropriate reagent, such as phosphoryl chloride (POCl₃), at elevated temperature can afford the chloride adduct

S-14. Chloride displacement of compound S-14 via nucleophilic substitution with aqueous ammonia at elevated temperature can deliver amine S-15. Compound S-15 can then be selectively coupled to an adduct of formula S-2, in which M is a boronic acid, a boronic ester or an appropriately substituted metal (e.g., M is B(OR)₂, Sn(Alkyl)₃, Zn-Hal, etc.), under standard Suzuki cross-coupling conditions (e.g., in the presence of a palladium catalyst and a suitable base), or standard Stille cross-coupling conditions (e.g., in the presence of a palladium catalyst), or standard Negishi cross-coupling conditions (e.g., in the presence of a palladium catalyst), to give derivative S-16. Condensation of compound S-16 with a carbonyl adduct S-17 (Hal is a halide, such as F, Cl, Br, or I) at elevated temperature can generate compound S-18. Tricyclic compound S-18 can undergo a halogenation reaction, such as electrophilic aromatic substitution (S_{E}Ar), with an appropriate reagent, such as N-bromosuccinimide (NBS), to afford compound S-19 (Hal is a halide, such as F, Cl, Br, or I). Introduction of R¹ can then be achieved by the coupling of compound S-19 with an adduct S-20, using similar conditions as described for the preparation of compound S-16 from compound S-15, to afford compound S-21.

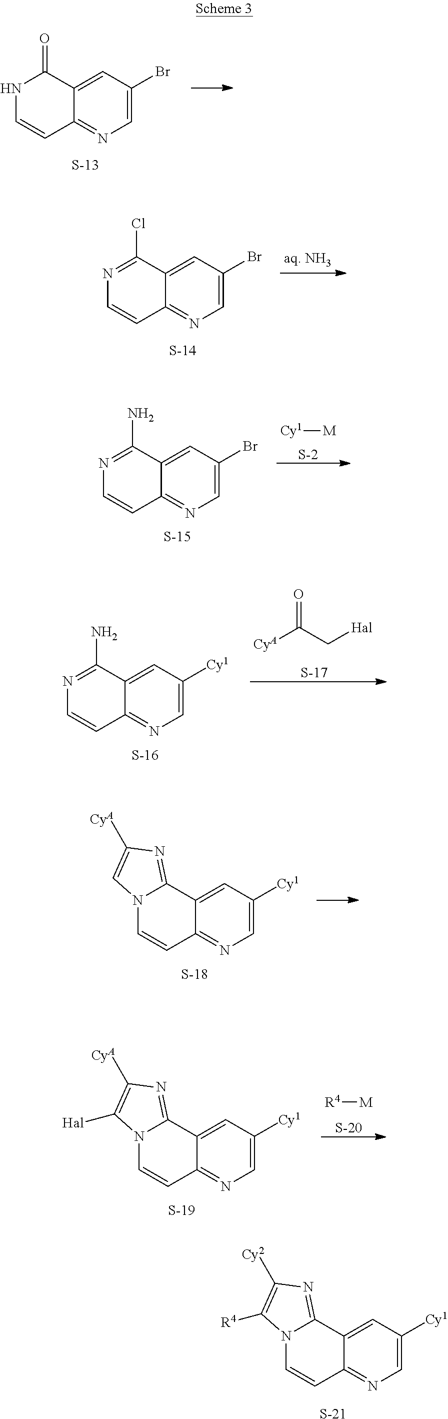

Alternatively, compounds of formula S-21 can be prepared via the synthetic route as outlined in Scheme 4. Condensation of compound S-15 with a carbonyl adduct of formula S-17 (Hal is a halide, such as Cl, Br, or I) at elevated temperature can generate compound S-22. Compound S-22 can then be selectively coupled to an adduct S-2, in which M is a boronic acid, a boronic ester or an appropriately substituted metal [e.g., M is B(OR)₂, Sn(Alkyl)₃, Zn-Hal, etc.], under standard Suzuki cross-coupling conditions (e.g., in the presence of a palladium catalyst and a suitable base), or standard Stille cross-coupling conditions (e.g., in the presence of a palladium catalyst), or standard Negishi cross-coupling conditions (e.g., in the presence of a palladium catalyst), to give a derivative of formula S-18. Compound S-21 can then be prepared from compound S-18 via the synthetic route as outlined in Scheme 3.

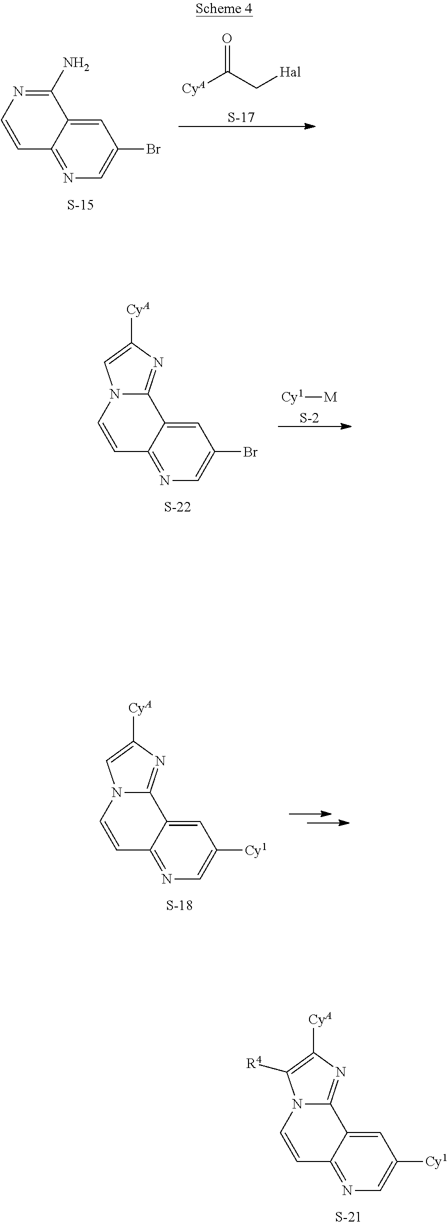

In addition, compound S-21 can be prepared by employing the steps described above in an alternative order. Initial halogenation of compound S-22 using an appropriate reagent, such as N-iodosuccinimide (NIS), can produce a dihalide intermediate which can undergo selective coupling to an adduct S-20 following the synthetic sequence from S-18 to S-21 as shown above in Scheme 3. A similar coupling strategy to an adduct S-2 as shown above in Scheme 4 can be followed to provide access to compound S-21.

Alternatively, compound S-21 can be prepared via the synthetic route as outlined in Scheme 5. Condensation of compound 5-15 with a carbonyl adduct S-23 (Hal is a halide, such as Cl, Br, or I) at elevated temperature can generate compound S-24. Compound S-24 can then be selectively coupled to an adduct S-2, in which M is a boronic acid, a boronic ester or an appropriately substituted metal [e.g., M is B(OR)₂, Sn(Alkyl)₃, Zn-Hal, etc.], under standard Suzuki cross-coupling conditions (e.g., in the presence of a palladium catalyst and a suitable base), or standard Stille cross-coupling conditions (e.g., in the presence of a palladium catalyst), or standard Negishi cross-coupling conditions (e.g., in the presence of a palladium catalyst), to give a derivative S-25. Compound S-25 can undergo a halogenation reaction, such as electrophilic aromatic substitution (S_{E}Ar), with an appropriate reagent, such as N-bromosuccinimide (NBS), to afford compound S-26 (Hal is a halide, such as Cl, Br, or I). Introduction of R¹ can then be achieved by the coupling of compound S-26 with an adduct S-20, using similar conditions as described for the preparation of compound S-25 from compound S-24, to afford compound S-27. Compound S-27 can undergo a second halogenation reaction, such as electrophilic aromatic substitution (S_{E}Ar), with an appropriate reagent, such as N-bromosuccinimide (NBS), to afford compound S-28 (Hal is a halide, such as Cl, Br, or I). Introduction of Cy² can then be achieved by the coupling of compound S-28 with an adduct S-29, using similar conditions as described for the preparation of compound S-25 from compound S-24, to afford compound S-21.

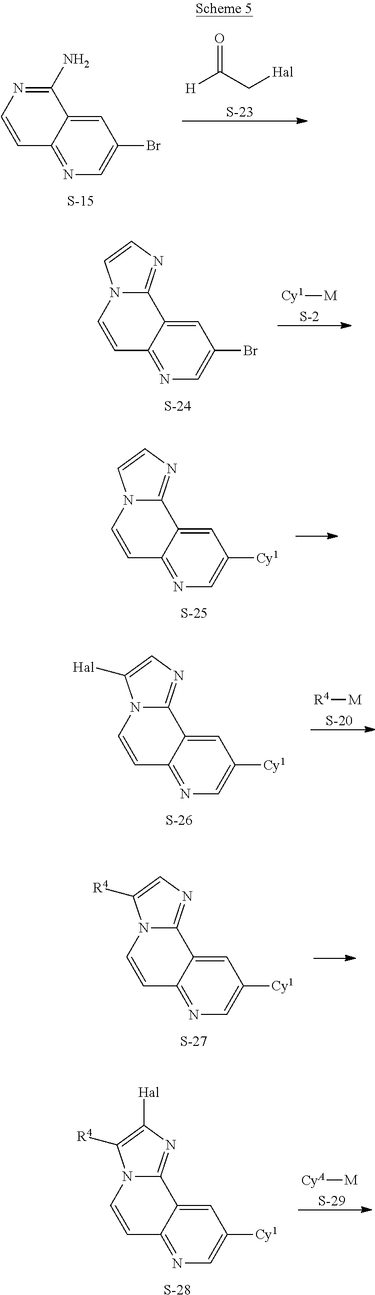

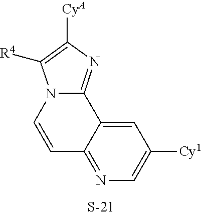

Compounds of formula 5-33 can be prepared via the synthetic route as outlined in Scheme 6. The commercially available starting material S-30 can undergo a selective coupling reaction with an adduct S-2, in which M is a boronic acid, a boronic ester or an appropriately substituted metal [e.g., M is B(OR)₂, Sn(Alkyl)₃, Zn-Hal, etc.], under standard Suzuki cross-coupling conditions (e.g., in the presence of a palladium catalyst and a suitable base), or standard Stille cross-coupling conditions (e.g., in the presence of a palladium catalyst), or standard Negishi cross-coupling conditions (e.g., in the presence of a palladium catalyst), to give a derivative S-31. Compound S-32 can be prepared by the reaction of compound S-31 with aqueous ammonia, using similar conditions as described for the preparation of compound 5-15 from compound S-14 in Scheme 3. Compound S-33 can then be prepared from compound S-32 via the synthetic route as outlined in Scheme 3 starting from compound S-16.

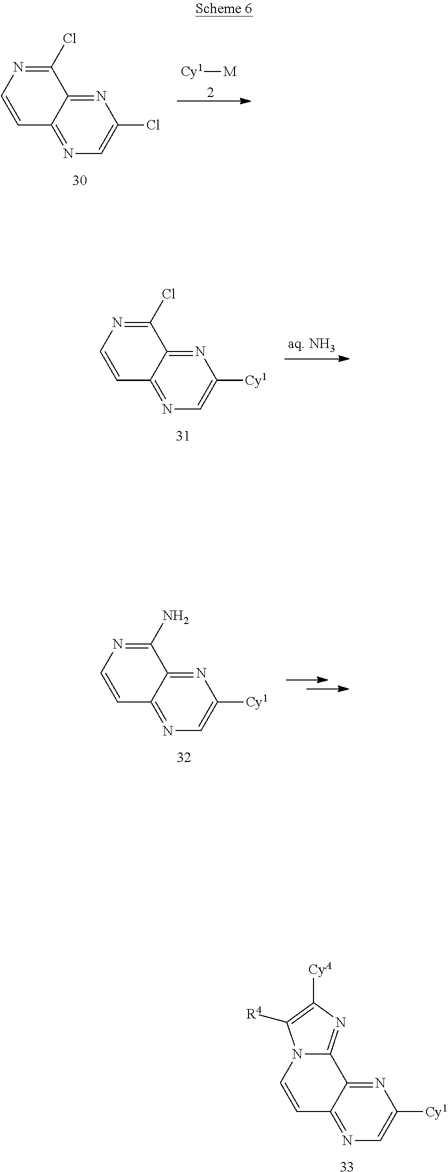

### Methods of Use

Compounds of the present disclosure can inhibit the activity of the FGFR enzyme. For example, compounds of the present disclosure can be used to inhibit activity of an FGFR enzyme in a cell or in an individual or patient in need of inhibition of the enzyme by administering an inhibiting amount of one or more compounds of the present disclosure to the cell, individual, or patient. Compounds of the present disclosure can be used to inhibit activity of the FGFR3 enzyme in a cell or in an individual or patient in need of inhibition of the enzyme by administering an inhibiting amount of one or more compounds of the present disclosure to the cell, individual, or patient.

As FGFR inhibitors, the compounds of the present disclosure are useful in the treatment of various diseases associated with abnormal expression or activity of the FGFR enzyme or FGFR ligands. Compounds which inhibit FGFR will be useful in providing a means of preventing the growth or inducing apoptosis in tumors, particularly by inhibiting angiogenesis.

It is therefore anticipated that compounds of the present disclosure will prove useful in treating or preventing proliferative disorders such as cancers. In particular, tumors with activating mutants of receptor tyrosine kinases or upregulation of receptor tyrosine kinases may be particularly sensitive to the inhibitors.

In certain embodiments, the disclosure provides a method for treating a FGFR-mediated disorder in a patient in need thereof, comprising the step of administering to said patient a compound according to the disclosure, or a pharmaceutically acceptable composition thereof.

In some embodiments, diseases and indications that are treatable using the compounds of the present disclosure include, but are not limited to hematological cancers, sarcomas, lung cancers, gastrointestinal cancers, genitourinary tract cancers, liver cancers, bone cancers, nervous system cancers, gynecological cancers, and skin cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), essential thrombocytosis (ET), 8p11 myeloproliferative syndrome), myelodysplasia syndrome (MDS), T-cell acute lymphoblastic lymphoma (T-ALL), multiple myeloma, cutaneous T-cell lymphoma, adult T-cell leukemia, Waldenstrom's Macroglubulinemia, hairy cell lymphoma, marginal zone lymphoma, chronic myelogenic lymphoma and Burkitt's lymphoma.

Exemplary sarcomas include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, lymphosarcoma, leiomyosarcoma, and teratoma.

Exemplary lung cancers include non-small cell lung cancer (NSCLC), small cell lung cancer, bronchogenic carcinoma (squamous cell, undifferentiated small cell, undifferentiated large cell, adenocarcinoma), alveolar (bronchiolar) carcinoma, bronchial adenoma, chondromatous hamartoma, mesothelioma, pavicellular and non-pavicellular carcinoma, bronchial adenoma and pleuropulmonary blastoma.

Exemplary gastrointestinal cancers include cancers of the esophagus (squamous cell carcinoma, adenocarcinoma, leiomyosarcoma, lymphoma), stomach (carcinoma, lymphoma, leiomyosarcoma), pancreas (exocrine pancreatic carcinoma, ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), colorectal cancer, gall bladder cancer and anal cancer.

Exemplary genitourinary tract cancers include cancers of the kidney (adenocarcinoma, Wilm's tumor [nephroblastoma], renal cell carcinoma), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma) and urothelial carcinoma.

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors Exemplary nervous system cancers include cancers of the skull (osteoma, hemangioma, granuloma, xanthoma, osteitis deformans), meninges (meningioma, meningiosarcoma, gliomatosis), brain (astrocytoma, meduoblastoma, glioma, ependymoma, germinoma (pinealoma), glioblastoma, glioblastoma multiform, oligodendroglioma, schwannoma, retinoblastoma, congenital tumors, neuro-ectodermal tumors), and spinal cord (neurofibroma, meningioma, glioma, sarcoma), neuroblastoma, Lhermitte-Duclos disease and pineal tumors.

Exemplary gynecological cancers include cancers of the breast (ductal carcinoma, lobular carcinoma, breast sarcoma, triple-negative breast cancer, HER2-positive breast cancer, inflammatory breast cancer, papillary carcinoma), uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, squamous cell carcinoma, Kaposi's sarcoma, Merkel cell skin cancer, moles dysplastic nevi, lipoma, angioma, dermatofibroma, and keloids.

Exemplary head and neck cancers include glioblastoma, melanoma, rhabdosarcoma, lymphosarcoma, osteosarcoma, squamous cell carcinomas, adenocarcinomas, oral cancer, laryngeal cancer, nasopharyngeal cancer, nasal and paranasal cancers, thyroid and parathyroid cancers, tumors of the eye, tumors of the lips and mouth and squamous head and neck cancer.

The compounds of the present disclosure can also be useful in the inhibition of tumor metastases.

In addition to oncogenic neoplasms, the compounds of the disclosure are useful in the treatment of skeletal and chondrocyte disorders including, but not limited to, achrondroplasia, hypochondroplasia, dwarfism, thanatophoric dysplasia (TD) (clinical forms TD I and TD II), Apert syndrome, Crouzon syndrome, Jackson-Weiss syndrome, Beare-Stevenson cutis gyrate syndrome, Pfeiffer syndrome, and craniosynostosis syndromes. In some embodiments, the present disclosure provides a method for treating a patient suffering from a skeletal and chondrocyte disorder.

In some embodiments, compounds described herein can be used to treat Alzheimer's disease, HIV, or tuberculosis.

As used herein, the term “8p11 myeloproliferative syndrome” is meant to refer to myeloid/lymphoid neoplasms associated with eosinophilia and abnormalities of FGFR1.

As used herein, the term “cell” is meant to refer to a cell that is in vitro, ex vivo or in vivo.

In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” the FGFR enzyme with a compound described herein includes the administration of a compound described herein to an individual or patient, such as a human, having FGFR, as well as, for example, introducing a compound described herein into a sample containing a cellular or purified preparation containing the FGFR enzyme.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent such as an amount of any of the solid forms or salts thereof as disclosed herein that elicits the biological or medicinal response in a tissue, system, animal, individual or human that is being sought by a researcher, veterinarian, medical doctor or other clinician. An appropriate “effective” amount in any individual case may be determined using techniques known to a person skilled in the art.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit/risk ratio.

As used herein, the phrase “pharmaceutically acceptable carrier or excipient” refers to a pharmaceutically-acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In one embodiment, each component is “pharmaceutically acceptable” as defined herein. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

As used herein, the term “treating” or “treatment” refers to inhibiting the disease; for example, inhibiting a disease, condition or disorder in an individual who is experiencing or displaying the pathology or symptomatology of the disease, condition or disorder (i.e., arresting further development of the pathology and/or symptomatology) or ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

It is appreciated that certain features of the disclosure, which are, for clarity, described in the context of separate embodiments, can also be provided in combination in a single embodiment (while the embodiments are intended to be combined as if written in multiply dependent form). Conversely, various features of the disclosure which are, for brevity, described in the context of a single embodiment, can also be provided separately or in any suitable subcombination.

### Combination Therapy

One or more additional pharmaceutical agents or treatment methods such as, for example, anti-viral agents, chemotherapeutics or other anti-cancer agents, immune enhancers, immunosuppressants, radiation, anti-tumor and anti-viral vaccines, cytokine therapy (e.g., IL2, GM-CSF, etc.), and/or tyrosine kinase inhibitors can be used in combination with compounds described herein for treatment of FGFR-associated diseases, disorders or conditions, or diseases or conditions as described herein. The agents can be combined with the present compounds in a single dosage form, or the agents can be administered simultaneously or sequentially as separate dosage forms.

Compounds described herein can be used in combination with one or more other kinase inhibitors for the treatment of diseases, such as cancer, that are impacted by multiple signaling pathways. For example, a combination can include one or more inhibitors of the following kinases for the treatment of cancer: Akt1, Akt2, Akt3, TGF-βR, Pim, PKA, PKG, PKC, CaM-kinase, phosphorylase kinase, MEKK, ERK, MAPK, mTOR, EGFR, HER2, HER3, HER4, INS-R, IGF-1R, IR-R, PDGFαR, PDGFβR, CSFIR, KIT, FLK-II, KDR/FLK-1, FLK-4, fit-1, FGFR1, FGFR2, FGFR3, FGFR4, c-Met, Ron, Sea, TRKA, TRKB, TRKC, FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. Additionally, the solid forms of the FGFR inhibitor as described herein can be combined with inhibitors of kinases associated with the PIK3/Akt/mTOR signaling pathway, such as PI3K, Akt (including Akt1, Akt2 and Akt3) and mTOR kinases.

In some embodiments, compounds described herein can be used in combination with one or more inhibitors of the enzyme or protein receptors such as HPK1, SBLB, TUT4, A2A/A2B, CD47, CDK2, STING, ALK2, LIN28, ADAR1, MAT2a, RIOK1, HDAC8, WDR5, SMARCA2, and DCLK1 for the treatment of diseases and disorders. Exemplary diseases and disorders include cancer, infection, inflammation and neurodegenerative disorders.

In some embodiments, compounds described herein can be used in combination with a therapeutic agent that targets an epigenetic regulator. Examples of epigenetic regulators include bromodomain inhibitors, the histone lysine methyltransferases, histone arginine methyl transferases, histone demethylases, histone deacetylases, histone acetylases, and DNA methyltransferases. Histone deacetylase inhibitors include, e.g., vorinostat.

For treating cancer and other proliferative diseases, compounds described herein can be used in combination with targeted therapies, including JAK kinase inhibitors (Ruxolitinib, additional JAK1/2 and JAK1-selective, baricitinib or INCB39110), Pim kinase inhibitors (e.g., LGH447, INCB053914 and SGI-1776), PI3 kinase inhibitors including PI3K-delta selective and broad spectrum PI3K inhibitors (e.g., INCB50465 and INCB54707), PI3K-gamma inhibitors such as PI3K-gamma selective inhibitors, MEK inhibitors, CSF1R inhibitors (e.g., PLX3397 and LY3022855), TAM receptor tyrosine kinases inhibitors (Tyro-3, Axl, and Mer; e.g., INCB81776), angiogenesis inhibitors, interleukin receptor inhibitors, Cyclin Dependent kinase inhibitors, BRAF inhibitors, mTOR inhibitors, proteasome inhibitors (Bortezomib, Carfilzomib), HDAC-inhibitors (panobinostat, vorinostat), DNA methyl transferase inhibitors, dexamethasone, bromo and extra terminal family members inhibitors (for example, bromodomain inhibitors or BET inhibitors, such as OTX015, CPI-0610, INCB54329 or INCB57643), LSD1 inhibitors (e.g., GSK2979552, INCB59872 and INCB60003), arginase inhibitors (e.g., INCB1158), indoleamine 2,3-dioxygenase inhibitors (e.g., epacadostat, NLG919 or BMS-986205), PARP inhibiors (e.g., olaparib or rucaparib), and inhibitors of BTK such as ibrutinib. In addition, for treating cancer and other proliferative diseases, compounds described herein can be used in combination with targeted therapies such as, e.g., c-MET inhibitors (e.g., capmatinib), an anti-CD19 antibody (e.g., tafasitamab), an ALK2 inhibitor (e.g., INCB00928); or combinations thereof.

For treating cancer and other proliferative diseases, compounds described herein can be used in combination with chemotherapeutic agents, agonists or antagonists of nuclear receptors, or other anti-proliferative agents. Compounds described herein can also be used in combination with a medical therapy such as surgery or radiotherapy, e.g., gamma-radiation, neutron beam radiotherapy, electron beam radiotherapy, proton therapy, brachytherapy, and systemic radioactive isotopes.

Examples of suitable chemotherapeutic agents include any of: abarelix, abiraterone, afatinib, aflibercept, aldesleukin, alemtuzumab, alitretinoin, allopurinol, altretamine, amidox, amsacrine, anastrozole, aphidicolon, arsenic trioxide, asparaginase, axitinib, azacitidine, bevacizumab, bexarotene, baricitinib, bendamustine, bicalutamide, bleomycin, bortezombi, bortezomib, brivanib, buparlisib, busulfan intravenous, busulfan oral, calusterone, camptosar, capecitabine, carboplatin, carmustine, cediranib, cetuximab, chlorambucil, cisplatin, cladribine, clofarabine, crizotinib, cyclophosphamide, cytarabine, dacarbazine, dacomitinib, dactinomycin, dalteparin sodium, dasatinib, dactinomycin, daunorubicin, decitabine, degarelix, denileukin, denileukin diftitox, deoxycoformycin, dexrazoxane, didox, docetaxel, doxorubicin, droloxafine, dromostanolone propionate, eculizumab, enzalutamide, epidophyllotoxin, epirubicin, epothilones, erlotinib, estramustine, etoposide phosphate, etoposide, exemestane, fentanyl citrate, filgrastim, floxuridine, fludarabine, fluorouracil, flutamide, fulvestrant, gefitinib, gemcitabine, gemtuzumab ozogamicin, goserelin acetate, histrelin acetate, ibritumomab tiuxetan, idarubicin, idelalisib, ifosfamide, imatinib mesylate, interferon alfa 2a, irinotecan, lapatinib ditosylate, lenalidomide, letrozole, leucovorin, leuprolide acetate, levamisole, lonafarnib, lomustine, meclorethamine, megestrol acetate, melphalan, mercaptopurine, methotrexate, methoxsalen, mithramycin, mitomycin C, mitotane, mitoxantrone, nandrolone phenpropionate, navelbene, necitumumab, nelarabine, neratinib, nilotinib, nilutamide, niraparib, nofetumomab, oserelin, oxaliplatin, paclitaxel, pamidronate, panitumumab, panobinostat, pazopanib, pegaspargase, pegfilgrastim, pemetrexed disodium, pentostatin, pilaralisib, pipobroman, plicamycin, ponatinib, porfimer, prednisone, procarbazine, quinacrine, ranibizumab, rasburicase, regorafenib, reloxafine, revlimid, rituximab, rucaparib, ruxolitinib, sorafenib, streptozocin, sunitinib, sunitinib maleate, tamoxifen, tegafur, temozolomide, teniposide, testolactone, tezacitabine, thalidomide, thioguanine, thiotepa, tipifarnib, topotecan, toremifene, tositumomab, trastuzumab, tretinoin, triapine, trimidox, triptorelin, uracil mustard, valrubicin, vandetanib, vinblastine, vincristine, vindesine, vinorelbine, vorinostat, veliparib, talazoparib, and zoledronate.

In some embodiments, compounds described herein can be used in combination with immune checkpoint inhibitors. Exemplary immune checkpoint inhibitors include inhibitors against immune checkpoint molecules such as CD27, CD28, CD40, CD122, CD96, CD73, CD47, OX40, GITR, CSF1R, JAK, PI3K delta, PI3K gamma, TAM, arginase, CD137 (also known as 4-1BB), ICOS, A2AR, B7-H₃, B7-H₄, BTLA, CTLA-4, LAG3 (e.g., INCAGN2385), TIM3 (e.g., INCB2390), VISTA, PD-1, PD-L1 and PD-L2. In some embodiments, the immune checkpoint molecule is a stimulatory checkpoint molecule selected from CD27, CD28, CD40, ICOS, OX40 (e.g., INCAGN1949), GITR (e.g., INCAGN1876) and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the inhibitor of an immune checkpoint molecule is a small molecule PD-L1 inhibitor. In some embodiments, the small molecule PD-L1 inhibitor has an IC50 less than 1 μM, less than 100 nM, less than 10 nM or less than 1 nM in a PD-L1 assay described in US Patent Publication Nos. US 20170107216, US 20170145025, US 20170174671, US 20170174679, US 20170320875, US 20170342060, US 20170362253, and US 20180016260, each of which is incorporated by reference in its entirety for all purposes.

In some embodiments, the inhibitor of an immune checkpoint molecule is an inhibitor of PD-1, e.g., an anti-PD-1 monoclonal antibody. In some embodiments, the anti-PD-1 monoclonal antibody is MGA012 (retifanlimab), nivolumab, pembrolizumab (also known as MK-3475), pidilizumab, SHR-1210, PDR001, ipilumimab or AMP-224. In some embodiments, the anti-PD-1 monoclonal antibody is nivolumab or pembrolizumab. In some embodiments, the anti-PD1 antibody is pembrolizumab. In some embodiments, the anti-PD1 antibody is nivolumab. In some embodiments, the anti-PD-1 monoclonal antibody is MGA012 (retifanlimab). In some embodiments, the anti-PD1 antibody is SHR-1210. Other anti-cancer agent(s) include antibody therapeutics such as 4-1BB (e.g. urelumab, utomilumab.

In some embodiments, the compounds of the disclosure can be used in combination with INCB086550.

In some embodiments, the inhibitor of an immune checkpoint molecule is an inhibitor of PD-L1, e.g., an anti-PD-L1 monoclonal antibody. In some embodiments, the anti-PD-L1 monoclonal antibody is BMS-935559, MEDI4736, MPDL3280A (also known as RG7446), or MSB0010718C. In some embodiments, the anti-PD-L1 monoclonal antibody is MPDL3280A or MEDI4736.

In some embodiments, the inhibitor of an immune checkpoint molecule is an inhibitor of CTLA-4, e.g., an anti-CTLA-4 antibody. In some embodiments, the anti-CTLA-4 antibody is ipilimumab, tremelimumab, AGEN1884, or CP-675,206.

In some embodiments, the inhibitor of an immune checkpoint molecule is an inhibitor of LAG3, e.g., an anti-LAG3 antibody. In some embodiments, the anti-LAG3 antibody is BMS-986016, LAG525, or INCAGN2385.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of GITR, e.g., an anti-GITR antibody. In some embodiments, the anti-GITR antibody is TRX518, MK-4166, INCAGN1876, MK-1248, AMG228, BMS-986156, GWN323, or MEDI1873.

In some embodiments, the inhibitor of an immune checkpoint molecule is an agonist of OX40, e.g., OX40 agonist antibody or OX40L fusion protein. In some embodiments, the anti-OX40 antibody is MEDI0562, MOXR-0916, PF-04518600, GSK3174998, or BMS-986178. In some embodiments, the OX40L fusion protein is MEDI6383.

In some embodiments, the inhibitor of an immune checkpoint molecule is an inhibitor of CD20, e.g., an anti-CD20 antibody. In some embodiments, the anti-CD20 antibody is obinutuzumab or rituximab.

The compounds of the present disclosure can be used in combination with bispecific antibodies. In some embodiments, one of the domains of the bispecific antibody targets PD-1, PD-L1, CTLA-4, GITR, OX40, TIM3, LAG3, CD137, ICOS, CD3 or TGFβ receptor.

In some embodiments, the compounds of the disclosure can be used in combination with one or more metabolic enzyme inhibitors. In some embodiments, the metabolic enzyme inhibitor is an inhibitor of IDO1, TDO, or arginase. Examples of IDO1 inhibitors include epacadostat, NLG919, BMS-986205, PF-06840003, IOM2983, RG-70099 and LY338196.

In some embodiments, the compounds described herein can be used in combination with one or more agents for the treatment of diseases such as cancer. In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM).

Suitable antiviral agents contemplated for use in combination with compounds of the present disclosure can comprise nucleoside and nucleotide reverse transcriptase inhibitors (NRTIs), non-nucleoside reverse transcriptase inhibitors (NNRTIs), protease inhibitors and other antiviral drugs.

Example suitable NRTIs include zidovudine (AZT); didanosine (ddl); zalcitabine (ddC); stavudine (d4T); lamivudine (3TC); abacavir (1592U89); adefovir dipivoxil [bis(POM)-PMEA]; lobucavir (BMS-180194); BCH-10652; emitricitabine [(−)-FTC]; beta-L-FD4 (also called beta-L-D4C and named beta-L-2′, 3′-dicleoxy-5-fluoro-cytidene); DAPD, ((−)-beta-D-2,6,-diamino-purine dioxolane); and lodenosine (FddA). Typical suitable NNRTIs include nevirapine (BI-RG-587); delaviradine (BHAP, U-90152); efavirenz (DMP-266); PNU-142721; AG-1549; MKC-442 (1-(ethoxy-methyl)-5-(1-methylethyl)-6-(phenylmethyl)-(2,4(1H,3H)-pyrimidinedione); and (+)-calanolide A (NSC-675451) and B. Typical suitable protease inhibitors include saquinavir (Ro 31-8959); ritonavir (ABT-538); indinavir (MK-639); nelfnavir (AG-1343); amprenavir (141W94); lasinavir (BMS-234475); DMP-450; BMS-2322623; ABT-378; and AG-1 549. Other antiviral agents include hydroxyurea, ribavirin, IL-2, IL-12, pentafuside and Yissum Project No. 11607.

Suitable agents for use in combination with compounds described herein for the treatment of cancer include chemotherapeutic agents, targeted cancer therapies, immunotherapies or radiation therapy. Compounds described herein may be effective in combination with anti-hormonal agents for treatment of breast cancer and other tumors. Suitable examples are anti-estrogen agents including but not limited to tamoxifen and toremifene, aromatase inhibitors including but not limited to letrozole, anastrozole, and exemestane, adrenocorticosteroids (e.g. prednisone), progestins (e.g. megastrol acetate), and estrogen receptor antagonists (e.g. fulvestrant). Suitable anti-hormone agents used for treatment of prostate and other cancers may also be combined with compounds described herein. These include anti-androgens including but not limited to flutamide, bicalutamide, and nilutamide, luteinizing hormone-releasing hormone (LHRH) analogs including leuprolide, goserelin, triptorelin, and histrelin, LHRH antagonists (e.g. degarelix), androgen receptor blockers (e.g. enzalutamide) and agents that inhibit androgen production (e.g. abiraterone).

The compounds described herein may be combined with or in sequence with other agents against membrane receptor kinases especially for patients who have developed primary or acquired resistance to the targeted therapy. These therapeutic agents include inhibitors or antibodies against EGFR, Her2, VEGFR, c-Met, Ret, IGFR1, or Flt-3 and against cancer-associated fusion protein kinases such as Bcr-Abl and EML4-Alk. Inhibitors against EGFR include gefitinib and erlotinib, and inhibitors against EGFR/Her2 include but are not limited to dacomitinib, afatinib, lapitinib and neratinib. Antibodies against the EGFR include but are not limited to cetuximab, panitumumab and necitumumab. Inhibitors of c-Met may be used in combination with FGFR inhibitors. These include onartumzumab, tivantnib, and INC-280. Agents against Abl (or Bcr-Abl) include imatinib, dasatinib, nilotinib, and ponatinib and those against Alk (or EML4-ALK) include crizotinib.

Angiogenesis inhibitors may be efficacious in some tumors in combination with FGFR inhibitors. These include antibodies against VEGF or VEGFR or kinase inhibitors of VEGFR. Antibodies or other therapeutic proteins against VEGF include bevacizumab and aflibercept. Inhibitors of VEGFR kinases and other anti-angiogenesis inhibitors include but are not limited to sunitinib, sorafenib, axitinib, cediranib, pazopanib, regorafenib, brivanib, and vandetanib

Activation of intracellular signaling pathways is frequent in cancer, and agents targeting components of these pathways have been combined with receptor targeting agents to enhance efficacy and reduce resistance. Examples of agents that may be combined with compounds described herein include inhibitors of the PI3K-AKT-mTOR pathway, inhibitors of the Raf-MAPK pathway, inhibitors of JAK-STAT pathway, and inhibitors of protein chaperones and cell cycle progression.

Agents against the PI3 kinase include but are not limited topilaralisib, idelalisib, buparlisib. Inhibitors of mTOR such as rapamycin, sirolimus, temsirolimus, and everolimus may be combined with FGFR inhibitors. Other suitable examples include but are not limited to vemurafenib and dabrafenib (Raf inhibitors) and trametinib, selumetinib and GDC-0973 (MEK inhibitors). Inhibitors of one or more JAKs (e.g., ruxolitinib, baricitinib, tofacitinib), Hsp90 (e.g., tanespimycin), cyclin dependent kinases (e.g., palbociclib), HDACs (e.g., panobinostat), PARP (e.g., olaparib), and proteasomes (e.g., bortezomib, carfilzomib) can also be combined with compounds described herein. In some embodiments, the JAK inhibitor is selective for JAK1 over JAK2 and JAK3.

Other suitable agents for use in combination with compounds described herein include chemotherapy combinations such as platinum-based doublets used in lung cancer and other solid tumors (cisplatin or carboplatin plus gemcitabine; cisplatin or carboplatin plus docetaxel; cisplatin or carboplatin plus paclitaxel; cisplatin or carboplatin plus pemetrexed) or gemcitabine plus paclitaxel bound particles (Abraxane®).

Suitable chemotherapeutic or other anti-cancer agents include, for example, alkylating agents (including, without limitation, nitrogen mustards, ethylenimine derivatives, alkyl sulfonates, nitrosoureas and triazenes) such as uracil mustard, chlormethine, cyclophosphamide (Cytoxan™), ifosfamide, melphalan, chlorambucil, pipobroman, triethylene-melamine, triethylenethiophosphoramine, busulfan, carmustine, lomustine, streptozocin, dacarbazine, and temozolomide.

Other suitable agents for use in combination with compounds described herein include steroids including 17 alpha-ethinylestradiol, diethylstilbestrol, testosterone, prednisone, fluoxymesterone, methylprednisolone, methyltestosterone, prednisolone, triamcinolone, chlorotrianisene, hydroxyprogesterone, aminoglutethimide, and medroxyprogesteroneacetate.

Other suitable agents for use in combination with compounds described herein include: dacarbazine (DTIC), optionally, along with other chemotherapy drugs such as carmustine (BCNU) and cisplatin; the “Dartmouth regimen,” which consists of DTIC, BCNU, cisplatin and tamoxifen; a combination of cisplatin, vinblastine, and DTIC; or temozolomide. Compounds described herein may also be combined with immunotherapy drugs, including cytokines such as interferon alpha, interleukin 2, and tumor necrosis factor (TNF) in.

Suitable chemotherapeutic or other anti-cancer agents include, for example, antimetabolites (including, without limitation, folic acid antagonists, pyrimidine analogs, purine analogs and adenosine deaminase inhibitors) such as methotrexate, 5-fluorouracil, floxuridine, cytarabine, 6-mercaptopurine, 6-thioguanine, fludarabine phosphate, pentostatine, and gemcitabine.

Suitable chemotherapeutic or other anti-cancer agents further include, for example, certain natural products and their derivatives (for example, vinca alkaloids, antitumor antibiotics, enzymes, lymphokines and epipodophyllotoxins) such as vinblastine, vincristine, vindesine, bleomycin, dactinomycin, daunorubicin, doxorubicin, epirubicin, idarubicin, ara-C, paclitaxel (TAXOL™), mithramycin, deoxycoformycin, mitomycin-C, L-asparaginase, interferons (especially IFN-α), etoposide, and teniposide.

Other cytotoxic agents include navelbene, CPT-11, anastrazole, letrazole, capecitabine, reloxafine, cyclophosphamide, ifosamide, and droloxafine.

Also suitable are cytotoxic agents such as epidophyllotoxin; an antineoplastic enzyme; a topoisomerase inhibitor; procarbazine; mitoxantrone; platinum coordination complexes such as cis-platin and carboplatin; biological response modifiers; growth inhibitors; antihormonal therapeutic agents; leucovorin; tegafur; and haematopoietic growth factors.

Other anti-cancer agent(s) include antibody therapeutics such as trastuzumab (Herceptin), antibodies to costimulatory molecules such as CTLA-4, 4-1BB, PD-L1 and PD-1 antibodies, or antibodies to cytokines (IL-10, TGF-β, etc.).

Other anti-cancer agents also include those that block immune cell migration such as antagonists to chemokine receptors, including CCR2 and CCR4.

Other anti-cancer agents also include those that augment the immune system such as adjuvants or adoptive T cell transfer.

Anti-cancer vaccines include dendritic cells, synthetic peptides, DNA vaccines and recombinant viruses. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV). Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The compounds of the present disclosure can be used in combination with bone marrow transplant for the treatment of a variety of tumors of hematopoietic origin.

Methods for the safe and effective administration of most of these chemotherapeutic agents are known to those skilled in the art. In addition, their administration is described in the standard literature. For example, the administration of many of the chemotherapeutic agents is described in the “Physicians'Desk Reference” (PDR, e.g., 1996 edition, Medical Economics Company, Montvale, NJ), the disclosure of which is incorporated herein by reference as if set forth in its entirety.

As provided throughout, the additional compounds, inhibitors, agents, etc. can be combined with the present compound in a single or continuous dosage form, or they can be administered simultaneously or sequentially as separate dosage forms.

### Pharmaceutical Formulations and Dosage Forms

When employed as pharmaceuticals, compounds described herein can be administered in the form of pharmaceutical compositions which refers to a combination of one or more compounds described herein, and at least one pharmaceutically acceptable carrier or excipient. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal, or intramuscular injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, one or more compounds described herein in combination with one or more pharmaceutically acceptable carriers or excipients. In making the compositions described herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. In some embodiments, the composition is suitable for topical administration.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

The compounds of the disclosure may be milled using known milling procedures such as wet milling to obtain a particle size appropriate for tablet formation and for other formulation types. Finely divided (nanoparticulate) preparations of the compounds of the disclosure can be prepared by processes known in the art see, e.g., WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions described herein can be formulated so as to provide quick, sustained or delayed release of the active ingredient after administration to the patient by employing procedures known in the art.

In some embodiments, the pharmaceutical composition comprises silicified microcrystalline cellulose (SMCC) and at least one compound described herein, or a pharmaceutically acceptable salt thereof. In some embodiments, the silicified microcrystalline cellulose comprises about 98% microcrystalline cellulose and about 2% silicon dioxide w/w.

In some embodiments, the composition is a sustained release composition comprising at least one compound described herein, or a pharmaceutically acceptable salt thereof, and at least one pharmaceutically acceptable carrier or excipient. In some embodiments, the composition comprises at least one compound described herein, or a pharmaceutically acceptable salt thereof, and at least one component selected from microcrystalline cellulose, lactose monohydrate, hydroxypropyl methylcellulose and polyethylene oxide. In some embodiments, the composition comprises at least one compound described herein, or a pharmaceutically acceptable salt thereof, and microcrystalline cellulose, lactose monohydrate and hydroxypropyl methylcellulose. In some embodiments, the composition comprises at least one compound described herein, or a pharmaceutically acceptable salt thereof, and microcrystalline cellulose, lactose monohydrate and polyethylene oxide. In some embodiments, the composition further comprises magnesium stearate or silicon dioxide. In some embodiments, the microcrystalline cellulose is Avicel PH102™. In some embodiments, the lactose monohydrate is Fast-flo 316™. In some embodiments, the hydroxypropyl methylcellulose is hydroxypropyl methylcellulose 2208 K4M (e.g., Methocel K4 M Premier™) and/or hydroxypropyl methylcellulose 2208 K100LV (e.g., Methocel K00LV™). In some embodiments, the polyethylene oxide is polyethylene oxide WSR 1105 (e.g., Polyox WSR 1105™).

In some embodiments, a wet granulation process is used to produce the composition. In some embodiments, a dry granulation process is used to produce the composition.

The compositions can be formulated in a unit dosage form, each dosage containing from, for example, about 5 mg to about 1000 mg, about 5 mg to about 100 mg, about 100 mg to about 500 mgor about 10 to about 30 mg, of the active ingredient. In some embodiments, each dosage contains about 10 mg of the active ingredient. In some embodiments, each dosage contains about 50 mg of the active ingredient. In some embodiments, each dosage contains about 25 mg of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The components used to formulate the pharmaceutical compositions are of high purity and are substantially free of potentially harmful contaminants (e.g., at least National Food grade, generally at least analytical grade, and more typically at least pharmaceutical grade). Particularly for human consumption, the composition is preferably manufactured or formulated under Good Manufacturing Practice standards as defined in the applicable regulations of the U.S. Food and Drug Administration. For example, suitable formulations may be sterile and/or substantially isotonic and/or in full compliance with all Good Manufacturing Practice regulations of the U.S. Food and Drug Administration.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

The therapeutic dosage of a compound of the present disclosure can vary according to, e.g., the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

For preparing solid compositions such as tablets, the principal active ingredient is mixed with a pharmaceutical excipient to form a solid pre-formulation composition containing a homogeneous mixture of one or more compounds described herein. When referring to these pre-formulation compositions as homogeneous, the active ingredient is typically dispersed evenly throughout the composition so that the composition can be readily subdivided into equally effective unit dosage forms such as tablets, pills and capsules. This solid pre-formulation is then subdivided into unit dosage forms of the type described above containing from, for example, 0.1 to about 500 mg of the active ingredient of the present disclosure.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds, or compositions as described herein can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, e.g., liquid paraffin, polyoxyethylene alkyl ether, propylene glycol, white Vaseline, and the like.

Carrier compositions of creams can be based on water in combination with glycerol and one or more other components, e.g., glycerinemonostearate, PEG-glycerinemonostearate and cetylstearyl alcohol. Gels can be formulated using isopropyl alcohol and water, suitably in combination with other components such as, e.g., glycerol, hydroxyethyl cellulose, and the like. In some embodiments, topical formulations contain at least about 0.1, at least about 0.25, at least about 0.5, at least about 1, at least about 2 or at least about 5 wt % of the compound of the disclosure. The topical formulations can be suitably packaged in tubes of, e.g., 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present disclosure can vary according to, for example, the particular use for which the treatment is made, the manner of administration of the compound, the health and condition of the patient, and the judgment of the prescribing physician. The proportion or concentration of the compounds in a pharmaceutical composition can vary depending upon a number of factors including dosage, chemical characteristics (e.g., hydrophobicity), and the route of administration. For example, compounds of the present disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

Compounds described herein can also be formulated in combination with one or more additional active ingredients, which can include any pharmaceutical agent such as anti-viral agents, vaccines, antibodies, immune enhancers, immune suppressants, anti-inflammatory agents and the like.

### Labeled Compounds and Assay Methods

Another aspect of the present disclosure relates to labeled compounds of the disclosure (radio-labeled, fluorescent-labeled, etc.) that would be useful not only in imaging techniques but also in assays, both in vitro and in vivo, for localizing and quantitating FGFR3 protein in tissue samples, including human, and for identifying FGFR3 ligands by inhibition binding of a labeled compound. Substitution of one or more of the atoms of the compounds of the present disclosure can also be useful in generating differentiated ADME (Adsorption, Distribution, Metabolism and Excretion). Accordingly, the present disclosure includes FGFR binding assays that contain such labeled or substituted compounds.

The present disclosure further includes isotopically-labeled compounds of the disclosure. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms (e.g., one or more hydrogen atoms of a C₁₋₆ alkyl group of Formula (I) can be optionally substituted with deuterium atoms, such as —CD₃ being substituted for —CH₃). In some embodiments, alkyl groups in Formula (I) can be perdeuterated.

One or more constituent atoms of the compounds presented herein can be replaced or substituted with isotopes of the atoms in natural or non-natural abundance. In some embodiments, the compound includes at least one deuterium atom. In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms.

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages.

The radionuclide that is incorporated in the instant radio-labeled compounds will depend on the specific application of that radio-labeled compound. For example, for in vitro adenosine receptor labeling and competition assays, compounds that incorporate ³H, ¹⁴C, ⁸²Br, ¹²⁵I, ¹³¹I or ³⁵S can be useful. For radio-imaging applications ¹¹C, ¹⁸F, ¹²⁵I, ¹²³I, ¹²⁴I, ¹³¹I, ⁷⁵Br, ⁷⁶Br or ⁷⁷Br can be useful.

It is understood that a “radio-labeled” or “labeled compound” is a compound that has incorporated at least one radionuclide. In some embodiments, the radionuclide is selected from the group consisting of ³H, ¹⁴C, ¹²⁵I, ³⁵S and ⁸²Br.

The present disclosure can further include synthetic methods for incorporating radio-isotopes into compounds of the disclosure. Synthetic methods for incorporating radio-isotopes into organic compounds are well known in the art, and an ordinary skill in the art will readily recognize the methods applicable for the compounds of disclosure.

A labeled compound of the disclosure can be used in a screening assay to identify and/or evaluate compounds. For example, a newly synthesized or identified compound (i.e., test compound) which is labeled can be evaluated for its ability to bind an FGFR3 protein by monitoring its concentration variation when contacting with the FGFR3, through tracking of the labeling. For example, a test compound (labeled) can be evaluated for its ability to reduce binding of another compound which is known to bind to a FGFR3 protein (i.e., standard compound). Accordingly, the ability of a test compound to compete with the standard compound for binding to the FGFR3 protein directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### Kits

The present disclosure also includes pharmaceutical kits useful, for example, in the treatment or prevention of FGFR-associated diseases or disorders, such as cancer and other diseases referred to herein which include one or more containers containing a pharmaceutical composition comprising a therapeutically effective amount of a compound of the disclosure. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The disclosure will be described in greater detail by way of specific examples. The following examples are offered for illustrative purposes, and are not intended to limit the disclosure in any manner. Those of skill in the art will readily recognize a variety of non-critical parameters which can be changed or modified to yield essentially the same results. The compounds of the Examples were found to be inhibitors of FGFR3 as described below.

### EXAMPLES

Experimental procedures for compounds of the disclosure are provided below. Preparatory LC-MS purifications of some of the compounds prepared were performed on Waters mass directed fractionation systems. The basic equipment setup, protocols, and control software for the operation of these systems have been described in detail in the literature. See e.g. “Two-Pump At Column Dilution Configuration for Preparative LC-MS”, K. Blom, J. Combi. Chem., 4, 295 (2002); “Optimizing Preparative LC-MS Configurations and Methods for Parallel Synthesis Purification”, K. Blom, R. Sparks, J. Doughty, G. Everlof, T. Haque, A. Combs, J. Combi. Chem., 5, 670 (2003); and “Preparative LC-MS Purification: Improved Compound Specific Method Optimization”, K. Blom, B. Glass, R. Sparks, A. Combs, J. Combi. Chem., 6, 874-883 (2004). The compounds separated were typically subjected to analytical liquid chromatography mass spectrometry (LCMS) for purity analysis under the following conditions: Instrument; Agilent 1100 series, LC/MSD, Column: Waters Sunfire™ C₁₈ 5 μm, 2.1×50 mm, Buffers: mobile phase A: 0.025% TFA in water and mobile phase B: acetonitrile; gradient 2% to 80% of B in 3 minutes with flow rate 2.0 mL/minute.

Some of the compounds prepared were also separated on a preparative scale by reverse-phase high performance liquid chromatography (RP-HPLC) with MS detector or flash chromatography (silica gel) as indicated in the Examples. Typical preparative reverse-phase high performance liquid chromatography (RP-HPLC) column conditions are as follows:

pH=2 purifications: Waters Sunfire™ C₁₈ 5 μm, 19×100 mm column, eluting with mobile phase A: 0.1% TFA (trifluoroacetic acid) in water and mobile phase B: acetonitrile; the flow rate was 30 mL/minute, the separating gradient was optimized for each compound using the Compound Specific Method Optimization protocol as described in the literature [see “Preparative LCMS Purification: Improved Compound Specific Method Optimization”, K. Blom, B. Glass, R. Sparks, A. Combs, J. Comb. Chem., 6, 874-883 (2004)]. Typically, the flow rate used with the 30×100 mm column was 60 mL/minute.

pH=10 purifications: Waters XBridge C₁₈ 5 μm, 19×100 mm column, eluting with mobile phase A: 0.15% NH₄OH in water and mobile phase B: acetonitrile; the flow rate was 30 mL/minute, the separating gradient was optimized for each compound using the Compound Specific Method Optimization protocol as described in the literature [See “Preparative LCMS Purification: Improved Compound Specific Method Optimization”, K. Blom, B. Glass, R. Sparks, A. Combs, J. Comb. Chem., 6, 874-883 (2004)]. Typically, the flow rate used with 30×100 mm column was 60 mL/minute.

### Example 1. 2-(2-Chlorophenyl)-3-methyl-9-(1-methyl-1H-pyrazol-4-yl)imidazo[2,1-f][1,6]naphthyridine

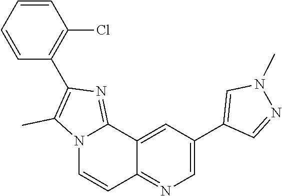 [SMILES: Cc1c(-c2ccccc2Cl)nc2c3cc(-c4cnn(C)c4)cnc3ccn12]

### Step 1. 3-Bromo-5-chloro-1,6-naphthyridine

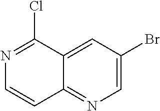 [SMILES: Clc1nccc2ncc(Br)cc12]

A flask containing a mixture of phosphorus(V) oxychloride (41.4 mL, 444 mmol) and 3-bromo-1,6-naphthyridin-5(6H)-one (5.0 g, 22.2 mmol) was stirred at 100° C. for 3 h. The reaction mixture was cooled to room temperature and the reaction mixture was concentrated in vacuo. The resulting residue was treated with saturated aqueous NaHCO₃ and the mixture was extracted with EtOAc. The organic phase was washed with brine, dried over MgSO₄, filtered and the solvent was evaporated in vacuo. The obtained crude product was used in the next step without further purification. LCMS calculated for C₈H₅BrClN₂ (M+H)⁺: m/z=242.9/244.9; Found: 243.0/244.9.

### Step 2. 3-Bromo-1,6-naphthyridin-5-amine

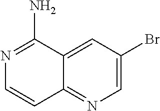 [SMILES: Nc1nccc2ncc(Br)cc12]

A mixture of 3-bromo-5-chloro-1,6-naphthyridine (2.68 g, 11.0 mmol), 1,4-dioxane (9 mL) and ammonium hydroxide solution (9 mL) in a sealed microwave vessel was irradiated at 150° C. for 3 h using a Biotage Initator+ Microwave Synthesizer. The reaction mixture was cooled to room temperature and the solvent was evaporated in vacuo. The obtained crude product was used in the next step without further purification. LCMS calculated for C₈H₇BrN₃ (M+H)⁺: m/z=224.0/226.0; Found: 224.2/226.2.

### Step 3. 3-(1-Methyl-1H-pyrazol-4-yl)-1,6-naphthyridin-5-amine

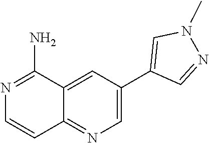 [SMILES: Cn1cc(-c2cnc3ccnc(N)c3c2)cn1]

A vial containing a mixture of 1-methyl-4-(4,4,5,5-tetramethyl-1,3,2-dioxaborolan-2-yl)-1H-pyrazole (1.0 g, 4.91 mmol), 3-bromo-1,6-naphthyridin-5-amine (1.0 g, 4.46 mmol), XPhos Pd G2 (351 mg, 0.45 mmol), and sodium carbonate (1.42 g, 13.4 mmol) was evacuated and backfilled with nitrogen three times, followed by the addition of 1,4-dioxane (20 mL) and water (2.0 mL). The vial was sealed and heated to 100° C. for 1 h. After cooling to room temperature the mixture was filtered directly, washing the filter cake with water, ether, and hexanes, followed by air drying overnight. The obtained crude product was used in the next step without further purification. LCMS calculated for C₁₂H₁₂N₅ (M+H)⁺: m/z=226.1; Found: 226.3.

### Step 4. 2-(2-Chlorophenyl)-9-(1-methyl-1H-pyrazol-4-yl)imidazo[2,1-f][1,6]naphthyridine

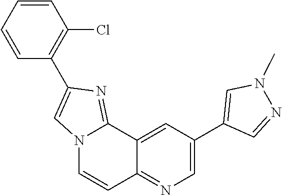 [SMILES: Cn1cc(-c2cnc3ccn4cc(-c5ccccc5Cl)nc4c3c2)cn1]

A vial containing a mixture of 3-(1-methyl-1H-pyrazol-4-yl)-1,6-naphthyridin-5-amine (255 mg, 1.13 mmol), sodium bicarbonate (1.43 g, 1.70 mmol), 2-bromo-1-(2-chlorophenyl)ethan-1-one (214 μL, 1.47 mmol) and absolute ethanol (3 mL) was heated to 100° C. for 3 h. After cooling to room temperature, the solvent was evaporated in vacuo and the remaining residue was filtered. The filter cake was washed with ether followed by air drying overnight. The obtained crude product was used in the next step without further purification. LCMS calculated for C₂₀H₁₅ClN₅ (M+H)⁺: m/z=360.1/362.1; Found: 360.2/362.3.

### Step 5. 3-Bromo-2-(2-chlorophenyl)-9-(1-methyl-1H-pyrazol-4-yl)imidazo[2,1-f][1,6]naphthyridine

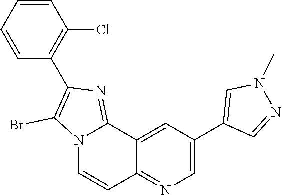 [SMILES: Cn1cc(-c2cnc3ccn4c(Br)c(-c5ccccc5Cl)nc4c3c2)cn1]

To a solution of 2-(2-chlorophenyl)-9-(1-methyl-1H-pyrazol-4-yl)imidazo[2,1-f][1,6]-naphthyridine (407 mg, 1.13 mmol) in CH₂Cl₂ (7.5 mL) at 0° C. was added N-bromosuccinimide (242 mg, 1.36 mmol), followed by stirring at room temperature for 10 min. The solvent was evaporated in vacuo and the resulting residue was purified by Biotage Isolera to give the desired product as a brown solid (411 mg, 83% yield). LCMS calculated for C₂₀H₁₄BrClN₅ (M+H)⁺: m/z=438.0/440.0/442.0; found 438.0/439.9/442.0.

### Step 6. 2-(2-Chlorophenyl)-3-methyl-9-(1-methyl-1H-pyrazol-4-yl)imidazo[2,1-f][1,6]naphthyridine

A vial containing a mixture of 3-bromo-2-(2-chlorophenyl)-9-(1-methyl-1H-pyrazol-4-yl)imidazo[2,1-f][1,6]naphthyridine (10 mg, 0.023 mmol), tetrakis(triphenylphosphine)-palladium(0) (5.3 mg, 0.0046 mmol), and potassium carbonate (6.3 mg, 0.043 mmol) was evacuated and backfilled with nitrogen three times, followed by the addition of 1,4-dioxane (1 mL), water (150 μL), and trimethylboroxine (4.8 μL, 0.034 mmol). The vial was sealed and heated to 100° C. for 1.5 h. After cooling to room temperature, the mixture was filtered through Celite and washed with CH₂Cl₂, followed by concentration of the filtrate in vacuo. The reaction mixture was then diluted with CH₃CN and water and purified with prep-LCMS (XBridge C18 column, eluting with a gradient of acetonitrile/water containing 0.1% TFA, at flow rate of 60 mL/min). The title compound was isolated as the TFA salt. LCMS calculated for C₂₁H₁₇ClN₅ (M+H)⁺: m/z=374.1/376.1; found 374.3/376.3.

### Example 2. 2-(2-Chlorophenyl)-3-(cyclopent-1-en-1-yl)-9-(1-methyl-1H-pyrazol-4-yl)imidazo[2,1-f][1,6]naphthyridine

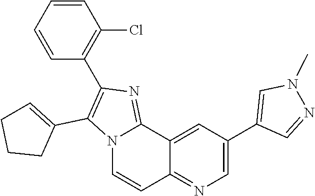 [SMILES: Cn1cc(-c2cnc3ccn4c(C5=CCCC5)c(-c5ccccc5Cl)nc4c3c2)cn1]

This compound was prepared according to the procedures described in Example 1, with 2-(cyclopent-1-en-1-yl)-4,4,5,5-tetramethyl-1,3,2-dioxaborolane replacing trimethylboroxine in Step 6. The title compound was isolated as the TFA salt. LCMS calculated for C₂₅H₂₁ClN₅ (M+H)⁺: m/z=426.1/428.1; Found: 426.3/428.4.

### Example 3. (E)-2-(2-Chlorophenyl)-9-(1-methyl-1H-pyrazol-4-yl)-3-(prop-1-en-1-yl)imidazo[2,1-f][1,6]naphthyridine

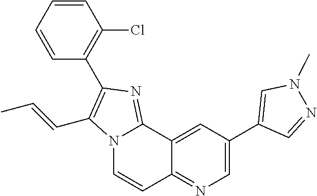 [SMILES: C/C=C/c1c(-c2ccccc2Cl)nc2c3cc(-c4cnn(C)c4)cnc3ccn12]

This compound was prepared according to the procedures described in Example 1, with potassium (E)-trifluoro(prop-1-en-1-yl)borate replacing trimethylboroxine in Step 6. The title compound was isolated as the TFA salt. LCMS calculated for C₂₃H₁₉ClN₅ (M+H)⁺: m/z=400.1/402.1; Found: 400.3/402.4.

### Example 4. 2-(2-Chlorophenyl)-3-(3,5-dimethoxyphenyl)-9-(1-methyl-1H-pyrazol-4-yl)imidazo[2,1-f][1,6]naphthyridine

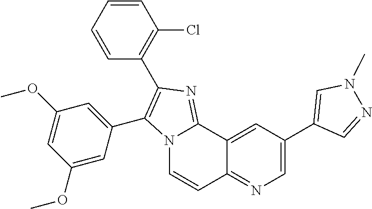 [SMILES: COc1cc(OC)cc(-c2c(-c3ccccc3Cl)nc3c4cc(-c5cnn(C)c5)cnc4ccn23)c1]

This compound was prepared according to the procedures described in Example 1, with 2-(3,5-dimethoxyphenyl)-4,4,5,5-tetramethyl-1,3,2-dioxaborolane replacing trimethylboroxine in Step 6. The title compound was isolated as the TFA salt. LCMS calculated for C₂₈H₂₃ClN₅O₂ (M+H)⁺: m/z=496.2/498.2; Found: 496.1/498.1.

### Example 5. 2-(2-Chlorophenyl)-3,9-bis(1-methyl-1H-pyrazol-4-yl)imidazo[2,1-f][1,6]naphthyridine

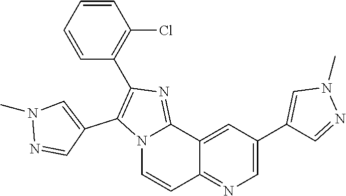 [SMILES: Cn1cc(-c2cnc3ccn4c(-c5cnn(C)c5)c(-c5ccccc5Cl)nc4c3c2)cn1]

This compound was prepared according to the procedures described in Example 1, with 1-methyl-4-(4,4,5,5-tetramethyl-1,3,2-dioxaborolan-2-yl)-1H-pyrazole replacing trimethylboroxine in Step 6. The title compound was isolated as the TFA salt. LCMS calculated for C₂₄H₁₉ClN₇ (M+H)⁺: m/z=440.1/442.1; Found: 440.4/442.3.

### Example 6. 2-(2-Chlorophenyl)-3-(furan-2-yl)-9-(1-methyl-1H-pyrazol-4-yl)imidazo[2,1-f][1,6]naphthyridine

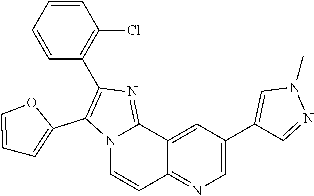 [SMILES: Cn1cc(-c2cnc3ccn4c(-c5ccco5)c(-c5ccccc5Cl)nc4c3c2)cn1]

A vial containing a mixture of 3-bromo-2-(2-chlorophenyl)-9-(1-methyl-1H-pyrazol-4-yl)imidazo[2,1-f][1,6]naphthyridine (10 mg, 0.023 mmol, Example 1, Step 5) and tetrakis(triphenylphosphine)palladium(0) (2.6 mg, 0.0023 mmol) was evacuated and backfilled with nitrogen three times, followed by the addition of 1,4-dioxane (1 mL) and tributyl(furan-2-yl)stannane (7.9 μL, 0.025 mmol). The vial was sealed and heated to 100° C. for 1 h. After cooling to room temperature the mixture was filtered through Celite and washed with CH₂Cl₂, followed by concentration of the filtrate in vacuo. The reaction mixture was then diluted with CH₃CN and water and purified with prep-LCMS (XBridge C18 column, eluting with a gradient of acetonitrile/water containing 0.1% TFA, at flow rate of 60 mL/min). The title compound was isolated as the TFA salt. LCMS calculated for C₂₄H₁₇ClN₅O (M+H)⁺: m/z=426.1/428.1; found 426.2/428.1.

### Example 7. 2-(2-Chlorophenyl)-9-(1-methyl-1H-pyrazol-4-yl)imidazo[2,1-f][1,6]naphthyridine-3-carbonitrile

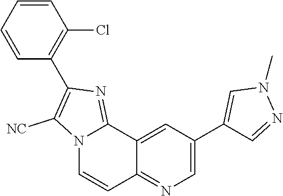 [SMILES: Cn1cc(-c2cnc3ccn4c(C#N)c(-c5ccccc5Cl)nc4c3c2)cn1]

A microwave vessel containing a mixture of 3-bromo-2-(2-chlorophenyl)-9-(1-methyl-1H-pyrazol-4-yl)imidazo[2,1-f][1,6]naphthyridine (Example 1, Step 5) (20 mg, 0.046 mmol), tris(dibenzylideneacetone)dipalladium(0) (2.1 mg, 2.3 μmol), 1,1′-bis(diphenylphosphino)ferrocene (2.5 mg, 4.5 μmol), zinc powder (1.2 mg, 0.02 mmol), and zinc cyanide (5.3 mg, 0.046 mmol) was evacuated and backfilled with nitrogen three times, followed by the addition of DMF (1 mL). The vial was sealed and irradiated at 150° C. for 2 h using a Biotage Initator+ Microwave Synthesizer. After cooling to room temperature, the mixture was filtered through Celite and washed with CH₂Cl₂, followed by concentration of the filtrate in vacuo. The reaction mixture was then diluted with CH₃CN and water and purified with prep-LCMS (XBridge C18 column, eluting with a gradient of acetonitrile/water containing 0.1% TFA, at flow rate of 60 mL/min). The title compound was isolated as the TFA salt. LCMS calculated for C₂₁H₁₄ClN₆ (M+H)⁺: m/z=385.1/387.1; found 385.1/387.1.

### Example 8. 2-(2-Chlorophenyl)-3-cyclopropyl-9-(1-methyl-1H-pyrazol-4-yl)imidazo[2,1-f][1,6]naphthyridine

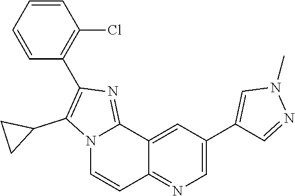 [SMILES: Cn1cc(-c2cnc3ccn4c(C5CC5)c(-c5ccccc5Cl)nc4c3c2)cn1]

A vial containing a mixture of 3-bromo-2-(2-chlorophenyl)-9-(1-methyl-1H-pyrazol-4-yl)imidazo[2,1-f][1,6]naphthyridine (Example 1, Step 5) (10 mg, 0.023 mmol), palladium(II) acetate (1.0 mg, 4.6 μmol), potassium cyclopropyltrifluoroborate (3.4 mg, 0.023 mmol), and di(1-adamantyl)-n-butylphosphine (2.0 mg, 5.7 μmol) was evacuated and backfilled with nitrogen three times, followed by the addition of 1,4-dioxane (1 mL) and water (100 μL). The vial was sealed and heated to 100° C. for 16 h. After cooling to room temperature, the mixture was filtered through Celite and washed with CH₂Cl₂, followed by concentration of the filtrate in vacuo. The reaction mixture was then diluted with CH₃CN and water and purified with prep-LCMS (XBridge C18 column, eluting with a gradient of acetonitrile/water containing 0.1% TFA, at flow rate of 60 mL/min). The title compound was isolated as the TFA salt. LCMS calculated for C₂₃H₁₉ClN₅ (M+H)⁺: m/z=400.1/402.1; found 400.2/402.2.

### Example 9. 2-(2-Chlorophenyl)-3-ethyl-9-(1-methyl-1H-pyrazol-4-yl)imidazo[2,1-f][1,6]naphthyridine

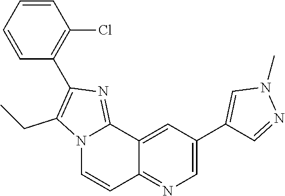 [SMILES: CCc1c(-c2ccccc2Cl)nc2c3cc(-c4cnn(C)c4)cnc3ccn12]

### Step 1. 2-(2-Chlorophenyl)-9-(1-methyl-1H-pyrazol-4-yl)-3-vinylimidazo[2,1-f][1,6]naphthyridine

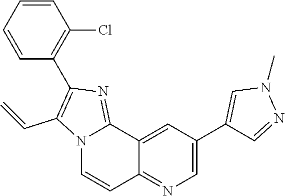 [SMILES: C=Cc1c(-c2ccccc2Cl)nc2c3cc(-c4cnn(C)c4)cnc3ccn12]

This compound was prepared according to the procedures described in Example 1, with 4,4,5,5-tetramethyl-2-vinyl-1,3,2-dioxaborolane replacing trimethylboroxine in Step 6. LCMS calculated for C₂₂H₁₇ClN₅ (M+H)⁺: m/z=386.1/388.1; Found: 386.2/386.1.

### Step 2. 2-(2-Chlorophenyl)-3-ethyl-9-(1-methyl-1H-pyrazol-4-yl)imidazo[2,1-f][1,6]naphthyridine

Palladium hydroxide on carbon (20 wt %, 2.4 mg, 3.4 μmol) was added to a solution of 2-(2-chlorophenyl)-9-(1-methyl-1H-pyrazol-4-yl)-3-vinylimidazo[2,1-f][1,6]naphthyridine (13 mg, 0.034 mmol) in absolute ethanol (2 mL). The reaction flask was connected to a balloon with hydrogen and evacuated and backfilled three times. The reaction mixture was stirred at 60° C. for 2 h. After cooling to room temperature the mixture was filtered through Celite and washed with CH₂Cl₂, followed by concentration of the filtrate in vacuo. The reaction mixture was then diluted with CH₃CN and water and purified with prep-LCMS (XBridge C18 column, eluting with a gradient of acetonitrile/water containing 0.1% TFA, at flow rate of 60 mL/min). The title compound was isolated as the TFA salt. LCMS calculated for C₂₂H₁₉ClN₅ (M+H)⁺: m/z=388.1/390.1; found 388.2/390.2.

### Example 10. 3-Chloro-2-(2-chlorophenyl)-9-(1-methyl-1H-pyrazol-4-yl)imidazo[2,1-f][1,6]naphthyridine

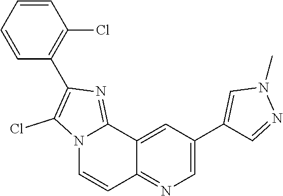 [SMILES: Cn1cc(-c2cnc3ccn4c(Cl)c(-c5ccccc5Cl)nc4c3c2)cn1]

To a solution of 2-(2-chlorophenyl)-9-(1-methyl-1H-pyrazol-4-yl)imidazo[2,1-f][1,6]naphthyridine (Example 1, Step 4) (10 mg, 0.028 mmol) in 1,2-dichloroethane (500 μL) was added N-chlorosuccinimide (4.5 mg, 0.033 mmol), followed by heating to 100° C. for 1 h. The solvent was evaporated in vacuo and the residue was then purified with prep-LCMS (XBridge C18 column, eluting with a gradient of acetonitrile/water containing 0.1% TFA, at flow rate of 60 mL/min). The title compound was isolated as the TFA salt. LCMS calculated for C₂₀H₁₄Cl₂N₅ (M+H)⁺: m/z=394.1/396.1; found 394.3/396.3.

### Example 11. 2-(2,6-Difluorophenyl)-9-(1-methyl-1H-pyrazol-4-yl)imidazo[2,1-f][1,6]naphthyridine

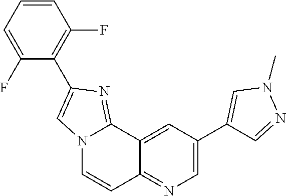 [SMILES: Cn1cc(-c2cnc3ccn4cc(-c5c(F)cccc5F)nc4c3c2)cn1]

This compound was prepared according to the procedures described in Example 1, with 2-bromo-1-(2,6-difluorophenyl)ethan-1-one replacing 2-bromo-1-(2-chlorophenyl)ethan-1-one in Step 4. The title compound was isolated as the TFA salt. LCMS calculated for C₂₀H₁₄F₂N₅ (M+H)⁺: m/z=362.1; Found: 362.2.

### Example 12. 2-(2-Chloro-6-fluorophenyl)-9-(1,3-dimethyl-1H-pyrazol-4-yl)imidazo[2,1-f][1,6]naphthyridine

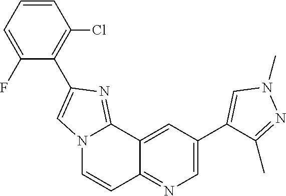 [SMILES: Cc1nn(C)cc1-c1cnc2ccn3cc(-c4c(F)cccc4Cl)nc3c2c1]

### Step 1. 3-(1,3-Dimethyl-1H-pyrazol-4-yl)-1,6-naphthyridin-5-amine

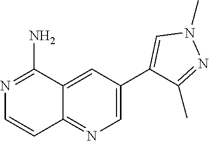 [SMILES: Cc1nn(C)cc1-c1cnc2ccnc(N)c2c1]

This compound was prepared according to the procedures described in Example 1, with 1,3-dimethyl-4-(4,4,5,5-tetramethyl-1,3,2-dioxaborolan-2-yl)-1H-pyrazole replacing 1-methyl-4-(4,4,5,5-tetramethyl-1,3,2-dioxaborolan-2-yl)-1H-pyrazole in Step 3. LCMS calculated for C₁₃H₁₄N₅ (M+H)⁺: m/z=240.1; Found: 240.1.

### Step 2. 2-(2-Chloro-6-fluorophenyl)-9-(1,3-dimethyl-1H-pyrazol-4-yl)imidazo[2,1-f][1,6]naphthyridine

This compound was prepared according to the procedures described in Example 1, with 2-bromo-1-(2-chloro-6-fluorophenyl)ethan-1-one replacing 2-bromo-1-(2-chlorophenyl)ethan-1-one in Step 4. The title compound was isolated as the TFA salt. LCMS calculated for C₂₁H₁₆ClFN₅ (M+H)⁺: m/z=392.1/394.1; Found: 392.2/394.2.

### Intermediate 1. 3-(1-Methyl-1H-pyrazol-4-yl)pyrido[3,4-b]pyrazin-5-amine

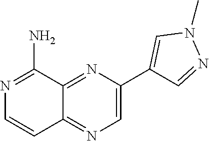 [SMILES: Cn1cc(-c2cnc3ccnc(N)c3n2)cn1]

### Step 1. 5-Chloro-3-(1-methyl-1H-pyrazol-4-yl)pyrido[3,4-b]pyrazine

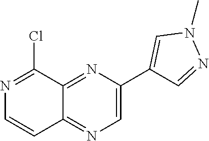 [SMILES: Cn1cc(-c2cnc3ccnc(Cl)c3n2)cn1]

This compound was prepared according to the procedures described in Example 1, with 3,5-dichloropyrido[3,4-b]pyrazine replacing 3-bromo-1,6-naphthyridin-5-amine in Step 3. LCMS calculated for C₁₁H₉ClN₅ (M+H)⁺: m/z=246.1/248.1; Found: 246.2/248.2.

### Step 2. 3-(1-Methyl-1H-pyrazol-4-yl)pyrido[3,4-b]pyrazin-5-amine

This compound was prepared according to the procedures described in Example 1, with 5-chloro-3-(1-methyl-1H-pyrazol-4-yl)pyrido[3,4-b]pyrazine replacing 3-bromo-5-chloro-1,6-naphthyridine in Step 2. LCMS calculated for C₁₁H₁₁N₆ (M+H)⁺: m/z=227.1; Found: 227.2.

### Example 13. 9-(2-Chlorophenyl)-8-cyclopropyl-2-(1-methyl-1H-pyrazol-4-yl)imidazo[1′,2′:1,2]pyrido[3,4-b]pyrazine

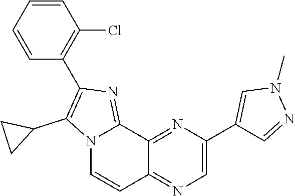 [SMILES: Cn1cc(-c2cnc3ccn4c(C5CC5)c(-c5ccccc5Cl)nc4c3n2)cn1]

This compound was prepared according to the procedures described in Examples 1 and 8, with 3-(1-methyl-1H-pyrazol-4-yl)pyrido[3,4-b]pyrazin-5-amine replacing 3-(1-methyl-1H-pyrazol-4-yl)-1,6-naphthyridin-5-amine in Example 1, Step 4. The title compound was isolated as the TFA salt. LCMS calculated for C₂₂H₁₈ClN₆ (M+H)⁺: m/z=401.1/403.1; Found: 401.2/403.1.

### Example 14. 9-(2-Chloro-6-fluorophenyl)-2-(1-methyl-1H-pyrazol-4-yl)imidazo[1′,2′:1,2]pyrido[3,4-b]pyrazine

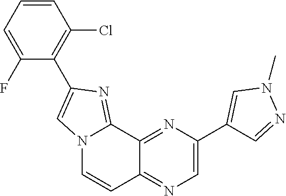 [SMILES: Cn1cc(-c2cnc3ccn4cc(-c5c(F)cccc5Cl)nc4c3n2)cn1]

This compound was prepared according to the procedures described in Example 1, with 3-(1-methyl-1H-pyrazol-4-yl)pyrido[3,4-b]pyrazin-5-amine replacing 3-(1-methyl-1H-pyrazol-4-yl)-1,6-naphthyridin-5-amine in Step 4. The title compound was isolated as the TFA salt. LCMS calculated for C₁₉H₁₃ClFN₆ (M+H)⁺: m/z=379.1/381.1; Found: 379.2/381.1.

### Example 15. 1-(3-(4-(2-(2,6-Dichlorophenyl)imidazo[2,1-f][1,6]naphthyridin-9-yl)-1H-pyrazol-1-yl)pyrrolidin-1-yl)ethan-1-one

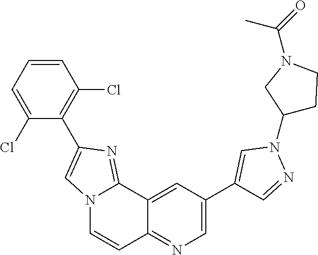 [SMILES: CC(=O)N1CCC(n2cc(-c3cnc4ccn5cc(-c6c(Cl)cccc6Cl)nc5c4c3)cn2)C1]

### Step 1. 9-Bromo-2-(2,6-dichlorophenyl)imidazo[2,1-f][1,6]naphthyridine

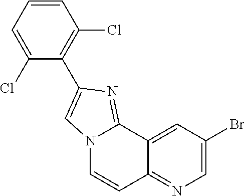 [SMILES: Clc1cccc(Cl)c1-c1cn2ccc3ncc(Br)cc3c2n1]

A microwave vessel containing a mixture of 3-bromo-1,6-naphthyridin-5-amine (Example 1, Step 2) (1.32 g, 5.89 mmol), sodium bicarbonate (742 mg, 8.84 mmol), 2-bromo-1-(2,6-dichlorophenyl)ethan-1-one (1.89 g, 7.07 mmol) and tert-butanol (8 mL) was irradiated at 150° C. for 9 h using a Biotage Initator+ Microwave Synthesizer. After cooling to room temperature the solid was filtered and washed with CH₂Cl₂, followed by concentration of the filtrate in vacuo. The resulting residue was purified by Biotage Isolera to give the desired product as an orange solid. LCMS calculated for C₁₆H₉BrCl₂N₃ (M+H)⁺: m/z=391.9/393.9/395.9; found 392.1/394.1/396.1.

### Step 2. 2-(2,6-Dichlorophenyl)-9-(1-(pyrrolidin-3-yl)-1H-pyrazol-4-yl)imidazo[2,1-f][1,6]naphthyridine

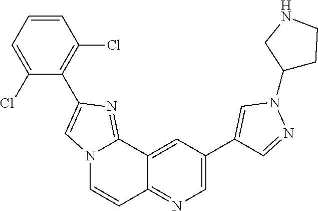 [SMILES: Clc1cccc(Cl)c1-c1cn2ccc3ncc(-c4cnn(C5CCNC5)c4)cc3c2n1]

A vial containing a mixture of tert-butyl 3-(4-(4,4,5,5-tetramethyl-1,3,2-dioxaborolan-2-yl)-1H-pyrazol-1-yl)pyrrolidine-1-carboxylate (102 mg, 0.280 mmol), 9-bromo-2-(2,6-dichlorophenyl)imidazo[2,1-f][1,6]naphthyridine (100 mg, 0.254 mmol), tetrakis(triphenylphosphine)palladium(0) (29 mg, 0.025 mmol), and potassium carbonate (81.0 mg, 0.763 mmol) was evacuated and backfilled with nitrogen three times, followed by the addition of 1,4-dioxane (2 mL) and water (200 μL). The vial was sealed and heated to 100° C. for 2 h. After cooling to room temperature the mixture was filtered through Celite and washed with CH₂Cl₂, followed by concentration of the filtrate in vacuo. The resulting residue was purified by Biotage Isolera to give the desired product as a brown solid. The purified material was then diluted with CH₂Cl₂ (3 mL) and TFA (1 mL) and left to stir at r.t. for 1 h. At completion, volatiles were removed in vacuo. The obtained product was used in the next step without further purification. LCMS calculated for C₂₃H₁₉Cl₂N₆ (M+H)⁺: m/z=449.1/451.1; found 449.3/451.3.

### Step 3. 1-(3-(4-(2-(2,6-Dichlorophenyl)imidazo[2,1-f][1,6]naphthyridin-9-yl)-1H-pyrazol-1-yl)pyrrolidin-1-yl)ethan-1-one

To a vial containing a mixture of 2-(2,6-dichlorophenyl)-9-(1-(pyrrolidin-3-yl)-1H-pyrazol-4-yl)imidazo[2,1-f][1,6]naphthyridine (10 mg, 0.022 mmol) and diisopropylethylamine (19 μL, 0.111 mmol) as a solution in CH₃CN (1 mL) was added acetyl chloride (3 μL, 0.049 mmol). The solution was left to stir at r.t. for 2 h. The reaction mixture was then diluted with CH₃CN and purified with prep-LCMS (XBridge C18 column, eluting with a gradient of acetonitrile/water containing 0.1% TFA, at flow rate of 60 mL/min). The title compound was isolated as the TFA salt. LCMS calculated for C₂₅H₂₁Cl₂N₆O (M+H)⁺: m/z=491.1/493.1; found 491.1/493.1.

### Example 16. (1R,4R)-4-(4-(2-(2-Chlorophenyl)-3-cyclopropylimidazo[2,1-f][1,6]naphthyridin-9-yl)-1H-pyrazol-1-yl)cyclohexane-1-carboxylic acid

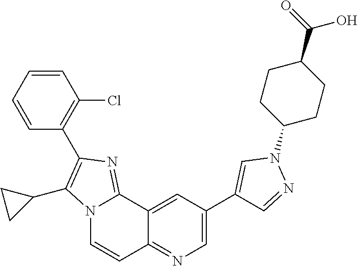 [SMILES: O=C(O)[C@H]1CC[C@H](n2cc(-c3cnc4ccn5c(C6CC6)c(-c6ccccc6Cl)nc5c4c3)cn2)CC1]

### Step 1. 2-(2-Chlorophenyl)-3-cyclopropyl-9-(1-((2-(trimethylsilyl)ethoxy)methyl)-1H-pyrazol-4-yl)imidazo[2,1-f][1,6]naphthyridine

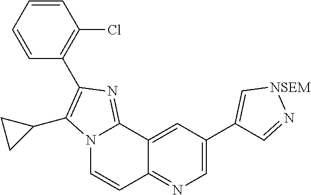 [SMILES: C[Si](C)(C)CCOCn1cc(-c2cnc3ccn4c(C5CC5)c(-c5ccccc5Cl)nc4c3c2)cn1]

This compound was prepared according to the procedures described in Examples 1 and 8, with 4-(4,4,5,5-tetramethyl-1,3,2-dioxaborolan-2-yl)-1-((2-(trimethylsilyl)ethoxy)methyl)-1H-pyrazole replacing 1-methyl-4-(4,4,5,5-tetramethyl-1,3,2-dioxaborolan-2-yl)-1H-pyrazole in Example 1, Step 3. LCMS calculated for C₂₈H₃₁ClN₅OSi (M+H)⁺: m/z=516.2/518.2; Found: 516.4/518.4.

### Step 2. 2-(2-Chlorophenyl)-3-cyclopropyl-9-(1H-pyrazol-4-yl)imidazo[2,1-f][1,6]naphthyridine

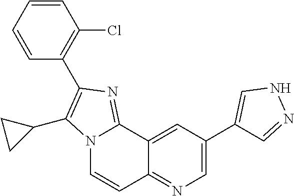 [SMILES: Clc1ccccc1-c1nc2c3cc(-c4cn[nH]c4)cnc3ccn2c1C1CC1]

To a vial containing a mixture of 2-(2-chlorophenyl)-3-cyclopropyl-9-(1-((2-(trimethylsilyl)ethoxy)methyl)-1H-pyrazol-4-yl)imidazo[2,1-f][1,6]naphthyridine (1.39 g, 2.70 mmol) as a solution in CH₂Cl₂ (10 mL) was added 4M HCl in dioxane (10 mL). The solution was left to stir at r.t. overnight. The reaction mixture was concentrated in vacuo. The resulting residue was treated with saturated aqueous NaHCO₃ and the organics were extracted into EtOAc. The organic phase was washed with brine, dried over MgSO₄, filtered and the solvent was evaporated in vacuo. The obtained crude product was used in the next step without further purification. LCMS calculated for C₂₂H₁₇ClN₅ (M+H)⁺: m/z=386.1/388.1; found 386.1/388.1.

### Step 3. (1R,4R)-4-(4-(2-(2-Chlorophenyl)-3-cyclopropylimidazo[2,1-f][1,6]naphthyridin-9-yl)-1H-pyrazol-1-yl)cyclohexane-1-carboxylic acid

To a vial containing a mixture of 2-(2-chlorophenyl)-3-cyclopropyl-9-(1H-pyrazol-4-yl)imidazo[2,1-f][1,6]naphthyridine (20 mg, 0.052 mmol, and cesium carbonate (51 mg, 0.155 mmol) as a solution in CH₃CN (1 mL) was added ethyl (1S,4S)-4-((methylsulfonyl)oxy)cyclohexane-1-carboxylate (19.5 mg, 0.078 mmol). The vial was sealed and heated to 80° C. for 16 h. After cooling to room temperature the mixture was filtered and washed with CH₃CN, followed by concentration of the filtrate in vacuo. The resulting residue was treated with 1,4-dioxane (1 mL) and 2M aq. LiOH (1 mL) and left to stir at r.t. for 2 h. The reaction mixture was then diluted with CH₃CN and purified with prep-LCMS (XBridge C18 column, eluting with a gradient of acetonitrile/water containing 0.1% TFA, at flow rate of 60 mL/min). The title compound was isolated as the TFA salt. LCMS calculated for C₂₉H₂₇ClN₅O₂ (M+H)⁺: m/z=512.2/514.2; found 512.4/514.4.

### Example 17. N-((1R,4R)-4-(4-(2-(2-Chlorophenyl)-3-cyclopropylimidazo[2,1-f][1,6]naphthyridin-9-yl)-1H-pyrazol-1-yl)cyclohexyl)acetamide

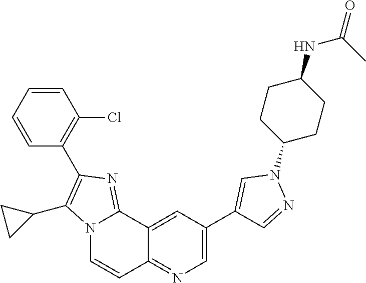 [SMILES: CC(=O)N[C@H]1CC[C@H](n2cc(-c3cnc4ccn5c(C6CC6)c(-c6ccccc6Cl)nc5c4c3)cn2)CC1]

### Step 1. tert-Butyl ((1R,4R)-4-(4-(2-(2-chlorophenyl)-3-cyclopropylimidazo[2,1-f][1,6]naphthyridin-9-yl)-1H-pyrazol-1-yl)cyclohexyl)carbamate

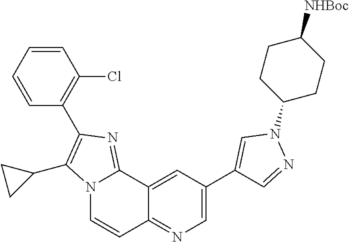 [SMILES: CC(C)(C)OC(=O)N[C@H]1CC[C@H](n2cc(-c3cnc4ccn5c(C6CC6)c(-c6ccccc6Cl)nc5c4c3)cn2)CC1]

To a vial containing a mixture of 2-(2-chlorophenyl)-3-cyclopropyl-9-(1H-pyrazol-4-yl)imidazo[2,1-f][1,6]naphthyridine (20 mg, 0.052 mmol, and cesium carbonate (51 mg, 0.155 mmol) as a solution in CH₃CN (1 mL) was added (1S,4S)-4-((tert-butoxycarbonyl)amino)-cyclohexyl methanesulfonate (22.8 mg, 0.078 mmol). The vial was sealed and heated to 80° C. for 16 h. After cooling to room temperature the mixture was filtered and washed with CH₃CN, followed by concentration of the filtrate in vacuo. The obtained crude product was used in the next step without further purification. LCMS calculated for C₃₃H₃₆ClN₆O₂ (M+H)⁺: m/z=583.3/585.3; Found: 583.4/585.4.

### Step 2. (1R,4R)-4-(4-(2-(2-Chlorophenyl)-3-cyclopropylimidazo[2,1-f][1,6]naphthyridin-9-yl)-1H-pyrazol-1-yl)cyclohexan-1-amine

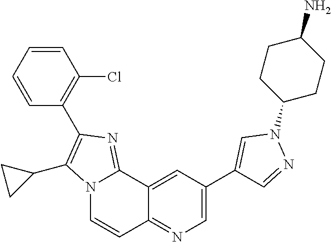 [SMILES: N[C@H]1CC[C@H](n2cc(-c3cnc4ccn5c(C6CC6)c(-c6ccccc6Cl)nc5c4c3)cn2)CC1]

To a vial containing a mixture of tert-butyl ((1R,4R)-4-(4-(2-(2-chlorophenyl)-3-cyclopropylimidazo[2,1-f][1,6]naphthyridin-9-yl)-1H-pyrazol-1-yl)cyclohexyl)carbamate (30 mg, 0.052 mmol) as a solution in CH₂Cl₂ (1 mL) was added TFA (1 mL). The solution was left to stir at r.t. for 1 h. The reaction mixture was concentrated in vacuo. The obtained crude product was used in the next step without further purification. LCMS calculated for C₂₈H₂₈ClN₆ (M+H)⁺: m/z=483.2/485.2; found 483.2/485.2.

### Step 3. N-((1R,4R)-4-(4-(2-(2-Chlorophenyl)-3-cyclopropylimidazo[2,1-f][1,6]naphthyridin-9-yl)-1H-pyrazol-1-yl)cyclohexyl)acetamide

To a vial containing a mixture of (1R,4R)-4-(4-(2-(2-chlorophenyl)-3-cyclopropylimidazo[2,1-f][1,6]naphthyridin-9-yl)-1H-pyrazol-1-yl)cyclohexan-1-amine (23 mg, 0.048 mmol) and diisopropylethylamine (42 μL, 0.238 mmol) as a solution in CH₃CN (1 mL) was added acetyl chloride (5 μL, 0.071 mmol). The solution was left to stir at r.t. for 2 h. The reaction mixture was then diluted with CH₃CN and purified with prep-LCMS (XBridge C18 column, eluting with a gradient of acetonitrile/water containing 0.1% TFA, at flow rate of 60 mL/min). The title compound was isolated as the TFA salt. LCMS calculated for C₃₀H₃₀ClN₆O (M+H)⁺: m/z=525.2/527.2; found 525.3/527.3.

### Example 18. 2-(2-Fluoro-6-methylphenyl)-3-methyl-8-(1-methyl-1H-pyrazol-4-yl)-3H-imidazo[4,5-f]quinoxaline

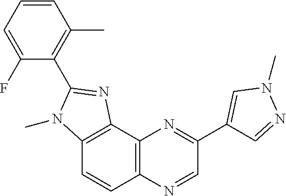 [SMILES: Cc1cccc(F)c1-c1nc2c3nc(-c4cnn(C)c4)cnc3ccc2n1C]

### Step 1. 2-(1-Methyl-1H-pyrazol-4-yl)-7-nitroquinoxaline

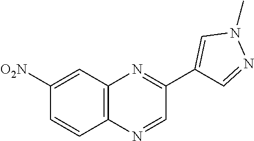 [SMILES: Cn1cc(-c2cnc3ccc([N+](=O)[O-])cc3n2)cn1]

A mixture of 2-chloro-7-nitroquinoxaline (Combi-Blocks Catalog, #QB-1085) (10 g, 48 mmol), XPhos Pd G2 (3.6 g, 4.8 mmol), and 1-methyl-4-(4,4,5,5-tetramethyl-1,3,2-dioxaborolan-2-yl)-1H-pyrazole (12 g, 57 mmol) in 1,4-dioxane (220 mL) and water (22 mL) was purged with nitrogen and stirred at 110° C. for 2 h. The reaction mixture was then cooled to room temperature, and filtered through a Celite plug with EtOAc. The volatiles were removed under reduced pressure and the crude material was taken forward without additional purification. LCMS calculated for C₁₂H₁₀N₅O₂ (M+H)⁺: m/z=256.1; Found: 256.1.

### Step 2. 3-(1-Methyl-1H-pyrazol-4-yl)quinoxalin-6-amine

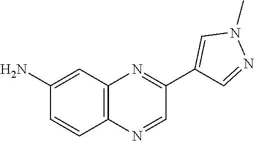 [SMILES: Cn1cc(-c2cnc3ccc(N)cc3n2)cn1]

To a pressure vessel containing 2-(1-methyl-1H-pyrazol-4-yl)-7-nitroquinoxaline (12.2 g, 47.7 mmol) was added iron powder (13.3 g, 239 mmol) and ammonium chloride (15.3 g, 286 mmol), followed by THF (80 mL), MeOH (80 mL), and water (80 mL). The vessel was sealed and heated to 55° C. for 3 h. The reaction mixture was then cooled to room temperature, and filtered through a Celite plug with 10% MeOH/DCM. The volatiles were removed under reduced pressure and the crude material was taken forward without additional purification. LCMS calculated for C₁₂H₁₂N₅ (M+H)⁺: m/z=226.1; Found: 226.3.

### Step 3. 5-Bromo-3-(1-methyl-1H-pyrazol-4-yl)quinoxalin-6-amine

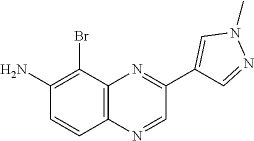 [SMILES: Cn1cc(-c2cnc3ccc(N)c(Br)c3n2)cn1]

To a mixture of 3-(1-methyl-1H-pyrazol-4-yl)quinoxalin-6-amine (8.50 g, 37.7 mmol) in DCM (190 mL) at 0° C. was added N-bromosuccinimide (6.72 g, 37.7 mmol) portion-wise. The reaction mixture was allowed to warm to room temperature, resulting in a homogenous solution. After stirring at room temperature for 30 min, the volatiles were removed under reduced pressure. The desired product was purified by column chromatography eluting with 0-100% EtOAc/hexanes containing 1% triethylamine. LCMS calculated for C₁₂H₁₁BrN₅ (M+H)⁺: m/z=304.0; Found: 304.0.

### Step 4. 5-Bromo-N-methyl-3-(1-methyl-1H-pyrazol-4-yl)quinoxalin-6-amine

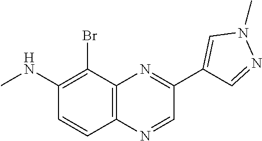 [SMILES: CNc1ccc2ncc(-c3cnn(C)c3)nc2c1Br]

To a solution of 5-bromo-3-(1-methyl-1H-pyrazol-4-yl)quinoxalin-6-amine (3.4 g, 11 mmol) in DMF (56 mL) cooled to 0° C. was added NaH (0.67 g, 17 mmol) as a 60% dispersion in mineral oil. The solution was stirred at 0° C. for 1 h, at which point iodomethane (0.42 mL, 6.8 mmol) was added drop wise. The reaction was stirred at room temperature for 2 h. The reaction was treated with saturated aq. NaHCO₃ and the organics were extracted with 5% isopropyl alcohol/DCM (3×). The combined organics were washed with water (3×) to remove excess DMF, then were dried over anhydrous magnesium sulfate, filtered, and concentrated under reduced pressure. The desired product was isolated by column chromatography eluting with 0-100% EtOAc/hexanes containing 1% triethylamine. LCMS calculated for C₁₃H₁₃BrN₅ (M+H)⁺: m/z=318.0; Found: 318.0.

### Step 5. N⁶-Methyl-3-(1-methyl-1H-pyrazol-4-yl)quinoxaline-5,6-diamine

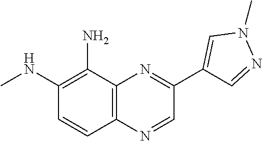 [SMILES: CNc1ccc2ncc(-c3cnn(C)c3)nc2c1N]

To a Schlenk flask was added t-BuDavePhos (0.86 g, 2.5 mmol), Pd₂(dba)₃ (0.68 g, 0.74 mmol), sodium tert-butoxide (1.4 g, 15 mmol), and 5-bromo-N-methyl-3-(1-methyl-1H-pyrazol-4-yl)quinoxalin-6-amine (2.4 g, 7.4 mmol). The flask was evacuated and back-filled with nitrogen, and an ammonia solution in dioxane (93 mL, 37.1 mmol) was added via syringe. The reaction mixture was then heated at 85° C. and stirred for 16 h. The reaction mixture was then cooled to room temperature, and filtered through a Celite plug with EtOAc. The filtrate was concentrated and the desired product was purified by column chromatography eluting with 0-10% MeOH/DCM. LCMS calculated for C₁₃H₁₅N₆ (M+H)⁺: m/z=255.1; Found: 255.1.

### Step 6. 2-Fluoro-6-methyl-N-(3-(1-methyl-1H-pyrazol-4-yl)-6-(methylamino)quinoxalin-5-yl)benzamide

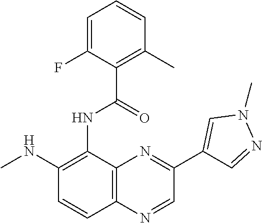 [SMILES: CNc1ccc2ncc(-c3cnn(C)c3)nc2c1NC(=O)c1c(C)cccc1F]

To a prestirred solution of 2-fluoro-6-methylbenzoic acid (18 mg, 0.094 mmol) in DCM (0.80 mL) was added a solution containing HATU (66 mg, 0.17 mmol), diisopropylethylamine (0.030 mL, 0.17 mmol) and DCM (0.80 mL). After the mixture was stirred for 30 min, N⁶-methyl-3-(1-methyl-1H-pyrazol-4-yl)quinoxaline-5,6-diamine (20 mg, 0.079 mmol) was added and the reaction mixture was stirred for 2 h. At completion, water was added and the organics were extracted with DCM, then dried over anhydrous magnesium sulfate, filtered, and concentrated under reduced pressure. The crude material was taken forward without additional purification. LCMS calculated for C₂₁H₂₀FN₆O (M+H)⁺: m/z=391.2; Found: 391.1.

### Step 7. 2-(2-Fluoro-6-methylphenyl)-3-methyl-8-(1-methyl-1H-pyrazol-4-yl)-3H-imidazo[4,5-f]quinoxaline

To a solution of 2-fluoro-6-methyl-N-(3-(1-methyl-1H-pyrazol-4-yl)-6-(methylamino)-quinoxalin-5-yl)benzamide (25 mg, 0.064 mmol) in MeOH (1.0 mL) was added methanesulfonic acid (0.03 mL, 0.39 mmol). The reaction mixture was then heated at 80° C. for 2 h. At completion, the reaction mixture was diluted with CH₃CN and water and purified with prep-LCMS (XBridge C18 column, eluting with a gradient of acetonitrile/water containing 0.1% TFA, at flow rate of 60 mL/min). The title compound was isolated as the TFA salt. LCMS calculated for C₂₁H₁₈FN₆ (M+H)⁺: m/z=373.2; Found: 373.1.

### Example 19. 2-(2-Chloro-4-methoxyphenyl)-3-methyl-8-(1-methyl-1H-pyrazol-4-yl)-3H-imidazo[4,5-f]quinoxaline

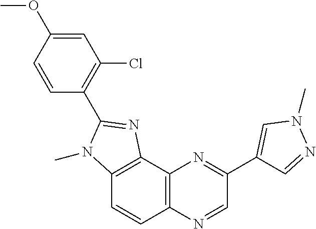 [SMILES: COc1ccc(-c2nc3c4nc(-c5cnn(C)c5)cnc4ccc3n2C)c(Cl)c1]

This compound was prepared using similar procedures as described for Example 18, with 2-chloro-4-methoxybenzoic acid replacing 2-fluoro-6-methylbenzoic acid in Step 6. The title compound was isolated as the TFA salt. LCMS calculated for C₂₁H₁₈ClN₆O (M+H)⁺: m/z=405.1; Found: 405.2.

### Example 20. 3-Methyl-8-(1-methyl-1H-pyrazol-4-yl)-2-(3-methylfuran-2-yl)-3H-imidazo[4,5-f]quinoxaline

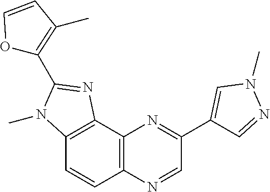 [SMILES: Cc1ccoc1-c1nc2c3nc(-c4cnn(C)c4)cnc3ccc2n1C]

This compound was prepared using similar procedures as described for Example 18, with 3-methylfuran-2-carboxylic acid replacing 2-fluoro-6-methylbenzoic acid in Step 6. The title compound was isolated as the TFA salt. LCMS calculated for C₁₉H₁₇N₆O (M+H)⁺: m/z=345.1; Found: 345.3.

### Example 21. 3-(4-(2-(2-Chloro-6-fluorophenyl)-3-methyl-3H-imidazo[4,5-f]quinoxalin-8-yl)-1H-pyrazol-1-yl)cyclobutan-1-ol

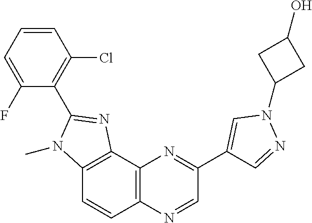 [SMILES: Cn1c(-c2c(F)cccc2Cl)nc2c3nc(-c4cnn(C5CC(O)C5)c4)cnc3ccc21]

### Step 1. N⁶-Methyl-3-(1-((2-(trimethylsilyl)ethoxy)methyl)-1H-pyrazol-4-yl)quinoxaline-5,6-diamine

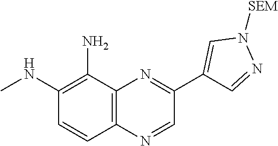 [SMILES: CNc1ccc2ncc(-c3cnn(COCC[Si](C)(C)C)c3)nc2c1N]

This compound was prepared using similar procedures as described for Example 15, Step 1 to Step 5, with 4-(4,4,5,5-tetramethyl-1,3,2-dioxaborolan-2-yl)-1-((2-(trimethylsilyl)ethoxy)methyl)-1H-pyrazole replacing 1-methyl-4-(4,4,5,5-tetramethyl-1,3,2-dioxaborolan-2-yl)-1H-pyrazole in Step 1. LCMS calculated for C₁₈H₂₇N₆OSi (M+H)⁺: m/z=371.2; Found: 371.2.

### Step 2. 2-Chloro-6-fluoro-N-(6-(methylamino)-3-(1-((2-(trimethylsilyl)ethoxy)methyl)-1H-pyrazol-4-yl)quinoxalin-5-yl)benzamide

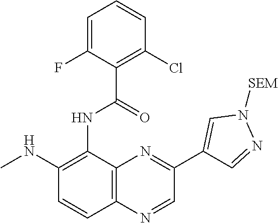 [SMILES: CNc1ccc2ncc(-c3cnn(COCC[Si](C)(C)C)c3)nc2c1NC(=O)c1c(F)cccc1Cl]

To a solution of N⁶-methyl-3-(1-((2-(trimethylsilyl)ethoxy)methyl)-1H-pyrazol-4-yl)quinoxaline-5,6-diamine (0.75 g, 2.01 mmol) and diisopropylethylamine (0.53 mL, 3.02 mmol) in DCM (10 mL) cooled to 0° C. was added 2-chloro-6-fluorobenzoyl chloride (0.29 mL, 2.22 mmol) dropwise. The solution was stirred at 0° C. for 1 h, at which point the reaction was treated with saturated aq. NaHCO₃ and the organics were extracted with DCM. The combined organics were dried over anhydrous magnesium sulfate, filtered, and concentrated under reduced pressure. The crude material was taken forward without additional purification. LCMS calculated for C₂₅H₂₉ClFN₆O₂Si (M+H)⁺: m/z=527.2; Found: 527.1.

### Step 3. 2-(2-Chloro-6-fluorophenyl)-3-methyl-8-(1-((2-(trimethylsilyl)ethoxy)methyl)-1H-pyrazol-4-yl)-3H-imidazo[4,5-f]quinoxaline

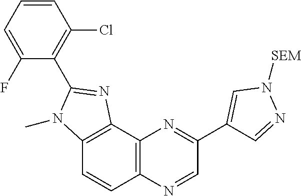 [SMILES: Cn1c(-c2c(F)cccc2Cl)nc2c3nc(-c4cnn(COCC[Si](C)(C)C)c4)cnc3ccc21]

To a vial was added 2-chloro-6-fluoro-N-(6-(methylamino)-3-(1-((2-(trimethylsilyl)ethoxy)-methyl)-1H-pyrazol-4-yl)quinoxalin-5-yl)benzamide (0.850 g, 2.01 mmol) and acetic acid (8.0 mL). The vial was sealed and the reaction mixture was heated at 120° C. for 4 h. The reaction mixture was then cooled to room temperature, and concentrated under reduced pressure. The crude residue was dissolved in DCM and saturated NaHCO₃ was carefully added. The aqueous phase was extracted with DCM (3×) and the combined organics were dried over anhydrous magnesium sulfate, filtered, and concentrated under reduced pressure. The crude material was taken forward without additional purification. LCMS calculated for C₂₅H₂₇ClFN₆OSi (M+H)⁺: m/z=509.2; Found: 508.9.

### Step 4. 2-(2-Chloro-6-fluorophenyl)-3-methyl-8-(1H-pyrazol-4-yl)-3H-imidazo[4,5-f]quinoxaline

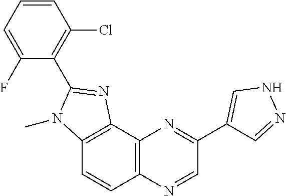 [SMILES: Cn1c(-c2c(F)cccc2Cl)nc2c3nc(-c4cn[nH]c4)cnc3ccc21]

In a flask, 2-(2-chloro-6-fluorophenyl)-3-methyl-8-(1-((2-(trimethylsilyl)ethoxy)methyl)-1H-pyrazol-4-yl)-3H-imidazo[4,5-f]quinoxaline (1.4 g, 2.8 mmol) was dissolved in DCM (28 mL). To the reaction mixture was added 4N HCl in dioxane (10 mL, 41 mmol). After stirring at ambient temperature for 3 h, the reaction mixture was concentrated under reduced pressure. The resultant material was purified by column chromatography eluting with 0-10% MeOH/DCM. LCMS calculated for C₁₉H₁₃ClFN₆ (M+H)⁺: m/z=379.1; Found: 379.1.

### Step 5. 3-(4-(2-(2-Chloro-6-fluorophenyl)-3-methyl-3H-imidazo[4,5-f]quinoxalin-8-yl)-1H-pyrazol-1-yl)cyclobutan-1-ol

To a mixture of 2-(2-chloro-6-fluorophenyl)-3-methyl-8-(1H-pyrazol-4-yl)-3H-imidazo[4,5-f]quinoxaline (40 mg, 0.11 mmol), 3-((tert-butyldimethylsilyl)oxy)cyclobutan-1-ol (70 μL, 0.32 mmol), and triphenylphosphine (55 mg, 0.21 mmol) in THF (1.0 mL) was added DIAD (62 μL, 0.32 mmol). The reaction mixture was purged with nitrogen and stirred at 80° C. for 16 h. At completion, water was added and the organics were extracted with DCM. The combined organics were dried over anhydrous magnesium sulfate, filtered, and concentrated under reduced pressure. To the resulting residue was added CH₃CN (1.0 mL) and 6N HCl (0.20 mL, 1.2 mmol). The reaction mixture was stirred for 30 min at room temperature. The reaction mixture was then diluted with CH₃CN and water and purified with prep-LCMS (XBridge C18 column, eluting with a gradient of acetonitrile/water containing 0.1% TFA, at flow rate of 60 mL/min). The title compound was isolated as the TFA salt. LCMS calculated for C₂₃H₁₉ClFN₆O (M+H)⁺: m/z=449.1; Found: 449.1.

### Example 22. 1-(4-(5-(2-(2-Chloro-6-fluorophenyl)-3-methyl-3H-imidazo[4,5-f]quinoxalin-8-yl)pyridin-2-yl)piperazin-1-yl)ethan-1-one

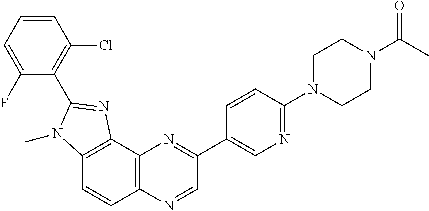 [SMILES: CC(=O)N1CCN(c2ccc(-c3cnc4ccc5c(nc(-c6c(F)cccc6Cl)n5C)c4n3)cn2)CC1]

### Step 1. 5-Bromo-3-(6-fluoropyridin-3-yl)quinoxalin-6-amine

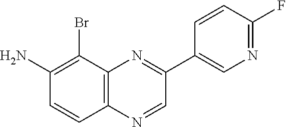 [SMILES: Nc1ccc2ncc(-c3ccc(F)nc3)nc2c1Br]

This compound was prepared using similar procedures as described for Example 18, Step 1 to Step 3, with 2-fluoro-5-(4,4,5,5-tetramethyl-1,3,2-dioxaborolan-2-yl)pyridine replacing 1-methyl-4-(4,4,5,5-tetramethyl-1,3,2-dioxaborolan-2-yl)-1H-pyrazole in Step 1. LCMS calculated for C₁₃H₉BrFN₄ (M+H)⁺: m/z=319.0; Found: 319.0.

### Step 2. N-(5-Bromo-3-(6-fluoropyridin-3-yl)quinoxalin-6-yl)-2-chloro-6-fluorobenzamide

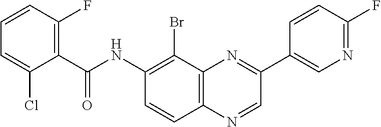 [SMILES: O=C(Nc1ccc2ncc(-c3ccc(F)nc3)nc2c1Br)c1c(F)cccc1Cl]

To a solution of 5-bromo-3-(6-fluoropyridin-3-yl)quinoxalin-6-amine (1.9 g, 6.0 mmol) and diisopropylethylamine (1.6 mL, 9.0 mmol) in DCM (30 mL) cooled to 0° C. was added 2-chloro-6-fluorobenzoyl chloride (0.80 mL, 6.0 mmol) dropwise. The solution was stirred at 0° C. for 1 h, at which point the reaction was treated with saturated NaHCO₃ and the organics were extracted with DCM. The combined organics were dried over anhydrous magnesium sulfate, filtered, and concentrated under reduced pressure. The desired product was isolated by column chromatography eluting with 0-100% EtOAc/hexanes containing 1% triethylamine. LCMS calculated for C₂₀H₁₁BrClF₂N₄O (M+H)⁺: m/z=475.0; Found: 475.0.

### Step 3. N-(5-Bromo-3-(6-fluoropyridin-3-yl)quinoxalin-6-yl)-2-chloro-6-fluoro-N-methylbenzamide

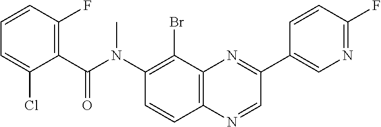 [SMILES: CN(C(=O)c1c(F)cccc1Cl)c1ccc2ncc(-c3ccc(F)nc3)nc2c1Br]

To a solution of N-(5-bromo-3-(6-fluoropyridin-3-yl)quinoxalin-6-yl)-2-chloro-6-fluorobenzamide (1.2 g, 2.5 mmol) in DMF (10 mL) cooled to 0° C. was added NaH (0.20 g, 5.1 mmol) as a 60% dispersion in mineral oil. The solution was stirred at 0° C. for 10 min, at which point iodomethane (0.31 mL, 5.1 mmol) was added dropwise. The reaction was stirred at room temperature for 1 h. The reaction was treated with saturated aqueous NaHCO₃ and the organics were extracted with EtOAc (3×). The combined organics were dried over anhydrous magnesium sulfate, filtered, and concentrated under reduced pressure. The desired product was isolated by column chromatography eluting with 0-100% EtOAc/hexanes containing 1% triethylamine. LCMS calculated for C₂₁H₁₃BrClF₂N₄O (M+H)⁺: m/z=489.0; Found: 489.0.

### Step 4. tert-Butyl 4-(5-(8-bromo-7-(2-chloro-6-fluoro-N-methylbenzamido)quinoxalin-2-yl)pyridin-2-yl)piperazine-1-carboxylate

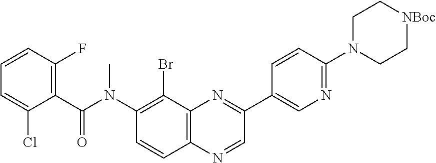 [SMILES: CN(C(=O)c1c(F)cccc1Cl)c1ccc2ncc(-c3ccc(N4CCN(C(=O)OC(C)(C)C)CC4)nc3)nc2c1Br]

To a vial was added N-(5-bromo-3-(6-fluoropyridin-3-yl)quinoxalin-6-yl)-2-chloro-6-fluoro-N-methylbenzamide (0.20 g, 0.41 mmol), tert-butyl piperazine-1-carboxylate (0.11 g, 0.61 mmol), and diisopropylethylamine (0.21 mL, 1.23 mmol) in DMSO (2.0 mL). The vial was sealed and the reaction mixture was heated at 100° C. for 16 h. At completion the reaction mixture was diluted with water and the organics were extracted with EtOAc (3×). The combined organics were dried over anhydrous magnesium sulfate, filtered, and concentrated under reduced pressure. The desired product was isolated by column chromatography eluting with 0-100% EtOAc/hexanes containing 1% triethylamine. LCMS calculated for C₃₀H₃₀BrClFN₆O₃ (M+H)⁺: m/z=655.1; Found: 655.1.

### Step 5. tert-Butyl 4-(5-(8-amino-7-(2-chloro-6-fluoro-N-methylbenzamido)quinoxalin-2-yl)pyridin-2-yl)piperazine-1-carboxylate

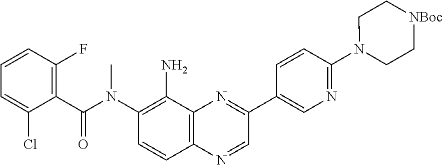 [SMILES: CN(C(=O)c1c(F)cccc1Cl)c1ccc2ncc(-c3ccc(N4CCN(C(=O)OC(C)(C)C)CC4)nc3)nc2c1N]

To a vial was added t-BuDavePhos (33 mg, 0.096 mmol), Pd₂(dba)₃ (26 mg, 0.028 mmol), sodium tert-butoxide (54 mg, 0.56 mmol), and tert-butyl 4-(5-(8-bromo-7-(2-chloro-6-fluoro-N-methylbenzamido)quinoxalin-2-yl)pyridin-2-yl)piperazine-1-carboxylate (190 mg, 0.28 mmol). The vial was evacuated and back-filled with nitrogen, and an ammonia solution in dioxane (3.5 mL, 1.4 mmol) was added via syringe. The reaction mixture was then heated at 85° C. for 16 h. The reaction mixture was then cooled to room temperature, and filtered through a Celite plug with EtOAc. The filtrate was concentrated and the desired product was purified by column chromatography eluting with 0-10% MeOH/DCM. LCMS calculated for C₃₀H₃₂ClFN₇O₃ (M+H)⁺: m/z=592.2; Found: 592.4.

### Step 6. 1-(4-(5-(2-(2-Chloro-6-fluorophenyl)-3-methyl-3H-imidazo[4,5-f]quinoxalin-8-yl)pyridin-2-yl)piperazin-1-yl)ethan-1-one

To a vial was added tert-butyl 4-(5-(8-amino-7-(2-chloro-6-fluoro-N-methylbenzamido)quinoxalin-2-yl)pyridin-2-yl)piperazine-1-carboxylate (30 mg, 0.09 mmol) and acetic acid (1.0 mL). The vial was sealed and the reaction mixture was heated at 120° C. for 16 h. The reaction mixture was then diluted with CH₃CN and water and purified with prep-LCMS (XBridge C18 column, eluting with a gradient of acetonitrile/water containing 0.1% TFA, at flow rate of 60 mL/min). The title compound was isolated as the TFA salt. LCMS calculated for C₂₇H₂₄ClFN₇O (M+H)⁺: m/z=516.2; Found: 516.4.

### Example 23. 3-Methyl-8-(1-methyl-1H-pyrazol-4-yl)-2-(1-methylpiperidin-4-yl)-3H-imidazo[4,5-f]quinoxaline

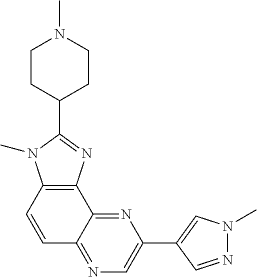

### Step 1. 1-Methyl-N-(3-(1-methyl-1H-pyrazol-4-yl)-6-(methylamino)quinoxalin-5-yl)piperidine-4-carboxamide

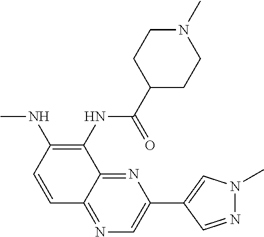

To a vial containing a mixture of N⁶-methyl-3-(1-methyl-1H-pyrazol-4-yl)quinoxaline-5,6-diamine (20 mg, 0.079 mmol, Example 18, Step 5) as a solution in CH₂Cl₂ (0.5 mL) at 0° C. was added diisopropylethylamine (34 μL, 0.197 mmol), followed by 1-methylpiperidine-4-carbonyl chloride hydrochloride (16 mg, 0.079 mmol). The solution was left to stir at 0° C. for 1 h. The reaction mixture was warmed to r.t. and the solvent was removed in vacuo. The residue was then diluted with CH₃CN and purified with prep-LCMS (XBridge C18 column, eluting with a gradient of acetonitrile/water containing 0.1% TFA, at flow rate of 60 mL/min). LCMS calculated for C₂₀H₂₆N₇₀ (M+H)⁺: m/z=380.2; found 380.4.

### Step 2. 3-Methyl-8-(1-methyl-1H-pyrazol-4-yl)-2-(1-methylpiperidin-4-yl)-3H-imidazo[4,5-f]quinoxaline

A vial containing a 1-methyl-N-(3-(1-methyl-1H-pyrazol-4-yl)-6-(methylamino)quinoxalin-5-yl)piperidine-4-carboxamide (5 mg, 0.013 mmol) as a solution in AcOH (0.5 mL) was heated to 120° C. for 16 h. The solution was cooled to r.t., then diluted with CH₃CN and purified with prep-LCMS (XBridge C18 column, eluting with a gradient of acetonitrile/water containing 0.1% TFA, at flow rate of 60 mL/min). The title compound was isolated as the TFA salt. LCMS calculated for C₂₀H₂₄N₇ (M+H)⁺: m/z=362.2; found 362.3.

### Example 24. (1S,5R)-8-(3-Methyl-8-(1-methyl-1H-pyrazol-4-yl)-3H-imidazo[4,5-f]quinoxalin-2-yl)-3,8-diazabicyclo[3.2.1]octan-2-one

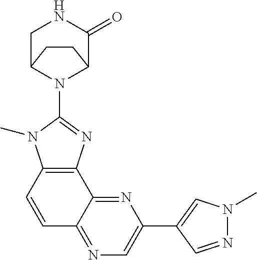

### Step 1. 3-Methyl-8-(1-methyl-1H-pyrazol-4-yl)-1,3-dihydro-2H-imidazo[4,5-f]quinoxalin-2-one

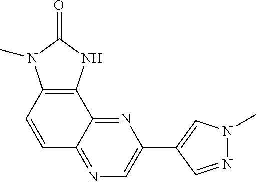

A vial containing a mixture of N⁶-methyl-3-(1-methyl-1H-pyrazol-4-yl)quinoxaline-5,6-diamine (Example 18, Step 5, 100 mg, 0.393 mmol) as a solution in THF (4 mL) was added diisopropylethylamine (103 μL, 0.590 mmol), followed by 1,1′-carbonyldiimidazole (77 mg, 0.472 mmol). The solution was left to stir for 16 h. The volatiles were removed under reduced pressure and the crude material was taken forward without additional purification. LCMS calculated for C₁₄H₁₃N₆O (M+H)⁺: m/z=281.1; Found: 281.1.

### Step 2. 2-Chloro-3-methyl-8-(1-methyl-1H-pyrazol-4-yl)-3H-imidazo[4,5-f]quinoxaline

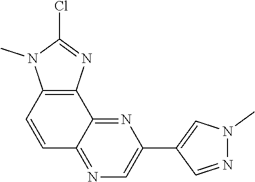

A vial containing a 3-methyl-8-(1-methyl-1H-pyrazol-4-yl)-1,3-dihydro-2H-imidazo[4,5-f]quinoxalin-2-one (110 mg, 0.392 mmol) as a solution in phosphoryl chloride (2 mL, 21.5 mmol) was heated to 100° C. for 1 h. The volatiles were removed in vacuo and the resulting residue was purified by Biotage Isolera to give the desired product as a brown foam (56 mg, 48% yield). LCMS calculated for C₁₄H₁₂ClN₆ (M+H)⁺: m/z=299.1/301.1; found 299.2/301.2.

### Step 3. (1S,5R)-8-(3-Methyl-8-(1-methyl-1H-pyrazol-4-yl)-3H-imidazo[4,5-f]quinoxalin-2-yl)-3,8-diazabicyclo[3.2.1]octan-2-one

A microwave vessel containing 2-chloro-3-methyl-8-(1-methyl-1H-pyrazol-4-yl)-3H-imidazo[4,5-f]quinoxaline (30 mg, 0.100 mmol) as a solution in t-BuOH (1 mL) was added diisopropylethylamine (53 μL, 0.301 mmol), followed by (1S,5R)-3,8-diazabicyclo[3.2.1]octan-2-one (25 mg, 0.201 mmol). The vial was sealed and irradiated at 150° C. for 5 h using a Biotage Initator+ Microwave Synthesizer. After cooling to room temperature, the mixture was diluted with CH₃CN and purified with prep-LCMS (XBridge C18 column, eluting with a gradient of acetonitrile/water containing 0.1% TFA, at flow rate of 60 mL/min). The title compound was isolated as the TFA salt. LCMS calculated for C₂₀H₂₁N₈O (M+H)⁺: m/z=389.2; found 389.3.

### Example 25. (S)-1-(3-(4-(2-(2-Chloro-6-(trifluoromethyl)phenyl)-3-methylimidazo[2,1-f][1,6]naphthyridin-9-yl)-1H-pyrazol-1-yl)pyrrolidin-1-yl)-2-hydroxyethan-1-one

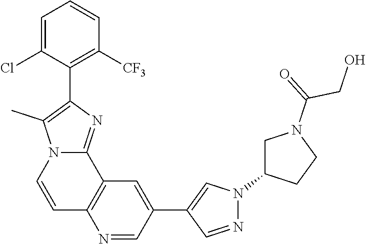 [SMILES: Cc1c(-c2c(Cl)cccc2C(F)(F)F)nc2c3cc(-c4cnn([C@H]5CCN(C(=O)CO)C5)c4)cnc3ccn12]

### Step 1. 9-Bromoimidazo[2,1-f][1,6]naphthyridine

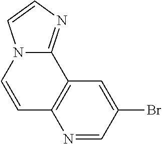 [SMILES: Brc1cnc2ccn3ccnc3c2c1]

To a flask containing a mixture of 3-bromo-1,6-naphthyridin-5-amine (Example 1, Step 2, 4.32 g, 19.3 mmol) as a solution in EtOH (77 mL) was added sodium bicarbonate (3.24 g, 38.6 mmol), followed by chloroacetaldehyde (50% in H₂O, 4.9 mL, 38.6 mmol). The solution was heated to 80° C. and left to stir for 2 h. After cooling to room temperature, the volatiles were removed in vacuo and the crude material was dissolved in DCM and filtered through Celite and washed with CH₂Cl₂. The volatiles were removed under reduced pressure and the crude material was taken forward without additional purification. LCMS calculated for C₁₀H₇BrN₃ (M+H)⁺: m/z=248.0/250.0; Found: 248.1/250.1.

### Step 2. 9-(1-((2-(Trimethylsilyl)ethoxy)methyl)-1H-pyrazol-4-yl)imidazo[2,1-f][1,6]naphthyridine

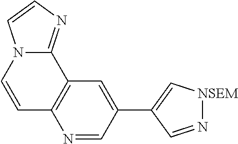 [SMILES: C[Si](C)(C)CCOCn1cc(-c2cnc3ccn4ccnc4c3c2)cn1]

A vial containing a mixture of 9-bromoimidazo[2,1-f][1,6]naphthyridine (1.1 g, 4.43 mmol), 4-(4,4,5,5-tetramethyl-1,3,2-dioxaborolan-2-yl)-1-((2-(trimethylsilyl)ethoxy)methyl)-1H-pyrazole (1.51 g, 4.66 mmol), XPhos Pd G2 (349 mg, 0.443 mmol), and potassium phosphate, tribasic (2.82 g, 13.3 mmol) was evacuated and backfilled with nitrogen three times, followed by the addition of 1,4-dioxane (14 mL) and water (3.5 mL). The vial was sealed and heated to 80° C. for 30 min. After cooling to room temperature, the mixture was filtered through Celite and washed with CH₂Cl₂, followed by concentration of the filtrate in vacuo. The obtained crude product was purified by Biotage Isolera to give the desired product. LCMS calculated for C₁₉H₂₄N₅OSi (M+H)⁺: m/z=366.2; found 366.2.

### Step 3. 3-Bromo-9-(1-((2-(trimethylsilyl)ethoxy)methyl)-1H-pyrazol-4-yl)imidazo[2,1-f][1,6]naphthyridine

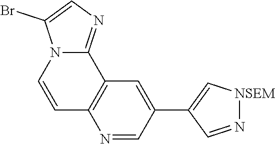 [SMILES: C[Si](C)(C)CCOCn1cc(-c2cnc3ccn4c(Br)cnc4c3c2)cn1]

To a flask containing 9-(1-((2-(trimethylsilyl)ethoxy)methyl)-1H-pyrazol-4-yl)imidazo[2,1-f][1,6]naphthyridine (1.62 g, 4.43 mmol) as a solution in CH₂Cl₂ (44 mL) was added NBS (947 mg, 5.32 mmol) and left to stir at r.t. for 30 min. The volatiles were removed under reduced pressure and the obtained crude product was purified by Biotage Isolera to give the desired product. LCMS calculated for C₁₉H₂₃BrN₅OSi (M+H)⁺: m/z=444.1/446.1; Found: 444.3/446.3.

### Step 4. 3-Methyl-9-(1H-pyrazol-4-yl)imidazo[2,1-f][1,6]naphthyridine

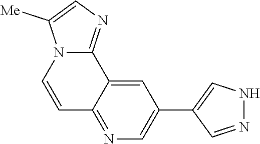 [SMILES: Cc1cnc2c3cc(-c4cn[nH]c4)cnc3ccn12]

A vial containing a mixture of 3-bromo-9-(1-((2-(trimethylsilyl)ethoxy)methyl)-1H-pyrazol-4-yl)imidazo[2,1-f][1,6]naphthyridine (1.97 g, 4.43 mmol), XPhos Pd G2 (349 mg, 0.443 mmol), and potassium phosphate, tribasic (2.82 g, 13.3 mmol) was evacuated and backfilled with nitrogen three times, followed by the addition of 1,4-dioxane (35 mL), water (9 mL), and trimethylboroxine (1.24 mL, 8.87 mmol). The vial was sealed and heated to 100° C. for 5 h. After cooling to room temperature, the mixture was filtered through Celite and washed with CH₂Cl₂, followed by concentration of the filtrate in vacuo. The obtained crude product was purified by Biotage Isolera to give the desired product (1.46 g, 87% yield). The purified material was then dissolved in CH₂Cl₂ (10 mL) and TFA (2 mL) and left to stir at r.t. for 2 h. The reaction mixture was concentrated in vacuo, then dissolved in MeOH (2 mL) and added to a stirring solution of saturated aqueous NaHCO₃ (50 mL). The resulting brown solid precipitate was filtered and collected, followed by drying under vacuum overnight. LCMS calculated for C₁₄H₁₂N₅ (M+H)⁺: m/z=250.1; found 250.2.

### Step 5. tert-Butyl (S)-3-(4-(3-methylimidazo[2,1-f][1,6]naphthyridin-9-yl)-1H-pyrazol-1-yl)pyrrolidine-1-carboxylate

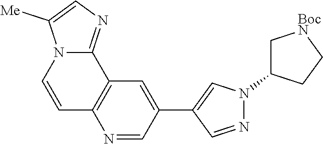 [SMILES: Cc1cnc2c3cc(-c4cnn([C@H]5CCN(C(=O)OC(C)(C)C)C5)c4)cnc3ccn12]

To a vial containing 3-methyl-9-(1H-pyrazol-4-yl)imidazo[2,1-f][1,6]naphthyridine (964 mg, 3.87 mmol) as a solution in MeCN (19 mL) was added cesium carbonate (2.52 g, 7.73 mmol) and tert-butyl (R)-3-((methylsulfonyl)oxy)pyrrolidine-1-carboxylate (1.54 g, 5.80 mmol). The vial was sealed and heated to 80° C. for 3 h. After cooling to room temperature, the mixture was filtered and washed with MeCN, followed by concentration of the filtrate in vacuo. The obtained crude product was purified by Biotage Isolera to give the desired product as a brown solid (1.09 g, 67% yield). LCMS calculated for C₂₃H₂₇N₆O₂ (M+H)⁺: m/z=419.2; found 419.2.

### Step 6. tert-Butyl (S)-3-(4-(2-bromo-3-methylimidazo[2,1-f][1,6]naphthyridin-9-yl)-1H-pyrazol-1-yl)pyrrolidine-1-carboxylate

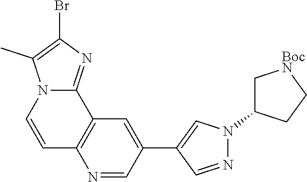 [SMILES: Cc1c(Br)nc2c3cc(-c4cnn([C@H]5CCN(C(=O)OC(C)(C)C)C5)c4)cnc3ccn12]

To a vial containing tert-butyl (S)-3-(4-(3-methylimidazo[2,1-f][1,6]naphthyridin-9-yl)-1H-pyrazol-1-yl)pyrrolidine-1-carboxylate (222 mg, 0.530 mmol) as a solution in CH₂Cl₂ (26 mL) was added NBS (113 mg, 0.637 mmol). The vial was sealed and heated to 80° C. for 2 h. After cooling to room temperature, the solvent was evaporated in vacuo and the resulting residue was purified by Biotage Isolera to give the desired product as a yellow solid (206 mg, 78% yield). LCMS calculated for C₂₃H₂₆BrN₆O₂ (M+H)⁺: m/z=497.1/499.1; found 497.3/499.3.

### Step 7. (S)-2-(2-Chloro-6-(trifluoromethyl)phenyl)-3-methyl-9-(1-(pyrrolidin-3-yl)-1H-pyrazol-4-yl)imidazo[2,1-f][1,6]naphthyridine

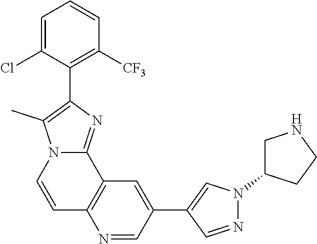 [SMILES: Cc1c(-c2c(Cl)cccc2C(F)(F)F)nc2c3cc(-c4cnn([C@H]5CCNC5)c4)cnc3ccn12]

A vial containing a mixture of tert-butyl (S)-3-(4-(2-bromo-3-methylimidazo[2,1-f][1,6]naphthyridin-9-yl)-1H-pyrazol-1-yl)pyrrolidine-1-carboxylate (20 mg, 0.040 mmol), (2-fluoro-6-(trifluoromethyl)phenyl)boronic acid (17 mg, 0.080 mmol), XPhos Pd G2 (3 mg, 4.00 μmol), and potassium phosphate, tribasic (26 mg, 0.121 mmol) was evacuated and backfilled with nitrogen three times, followed by the addition of 1,4-dioxane (1 mL) and water (250 μL). The vial was sealed and heated to 100° C. for 3 h. After cooling to room temperature, the mixture was filtered through Celite and washed with CH₂Cl₂, followed by concentration of the filtrate in vacuo. The obtained crude product was then dissolved in CH₂Cl₂(2 mL) and TFA (1 mL) and left to stir at r.t. for 1 h. The reaction mixture was concentrated in vacuo, then dissolved in MeOH (1 mL) and added to a stirring solution of saturated aqueous NaHCO₃ (5 mL). The resulting precipitate was filtered and collected, followed by drying under vacuum. LCMS calculated for C₂₅H₂₁ClF₃N₆ (M+H)⁺: m/z=497.1/499.1; found 497.3/499.3.

### Step 8. (S)-1-(3-(4-(2-(2-Chloro-6-(trifluoromethyl)phenyl)-3-methylimidazo[2,1-f][1,6]naphthyridin-9-yl)-1H-pyrazol-1-yl)pyrrolidin-1-yl)-2-hydroxyethan-1-one

To a vial containing (S)-2-(2-chloro-6-(trifluoromethyl)phenyl)-3-methyl-9-(1-(pyrrolidin-3-yl)-1H-pyrazol-4-yl)imidazo[2,1-f][1,6]naphthyridine (20 mg, 0.040 mmol) as a solution in DMF (0.5 mL) was added 2-hydroxyacetic acid (6 mg, 0.080 mmol), diisopropylethylamine (14 μL, 0.080 mmol), and HATU (23 mg, 0.060 mmol). The reaction mixture was left to stir at r.t. for 1 h, upon which time water was added and the resulting solid was collected by filtration and washed with water. The solid was then dissolved with CH₃CN and purified with prep-LCMS (XBridge C18 column, eluting with a gradient of acetonitrile/water containing 0.1% TFA, at flow rate of 60 mL/min). The title compound was isolated as the TFA salt. LCMS calculated for C₂₇H₂₃ClF₃N₆O₂ (M+H)⁺: m/z=555.2; found 555.3.

### Example 26. (S)-1-(3-(4-(2-(2,6-Dimethylphenyl)-3-methylimidazo[2,1-f][1,6]naphthyridin-9-yl)-1H-pyrazol-1-yl)pyrrolidin-1-yl)-2-hydroxyethan-1-one

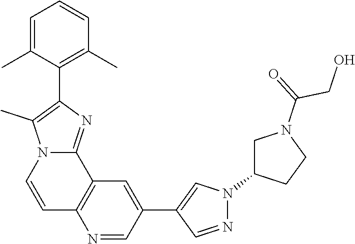 [SMILES: Cc1cccc(C)c1-c1nc2c3cc(-c4cnn([C@H]5CCN(C(=O)CO)C5)c4)cnc3ccn2c1C]

This compound was prepared according to the procedures described in Example 25, with (2,6-dimethylphenyl)boronic acid replacing (2-fluoro-6-(trifluoromethyl)phenyl)boronic acid in Step 7. The title compound was isolated as the TFA salt. LCMS calculated for C₂₈H₂₉N₆O₂ (M+H)⁺: m/z=481.2; Found: 481.4.

### Example 27. (S)-3-Fluoro-2-(9-(1-(1-(2-hydroxyacetyl)pyrrolidin-3-yl)-1H-pyrazol-4-yl)-3-methylimidazo[2,1-f][1,6]naphthyridin-2-yl)benzonitrile

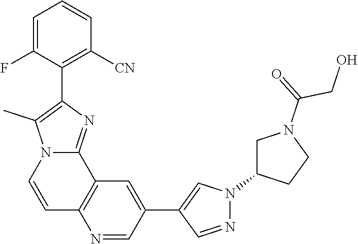 [SMILES: Cc1c(-c2c(F)cccc2C#N)nc2c3cc(-c4cnn([C@H]5CCN(C(=O)CO)C5)c4)cnc3ccn12]

This compound was prepared according to the procedures described in Example 25, with (2-cyano-6-fluorophenyl)boronic acid replacing (2-fluoro-6-(trifluoromethyl)phenyl)boronic acid in Step 7. The title compound was isolated as the TFA salt. LCMS calculated for C₂₇H₂₃FN₇O₂ (M+H)⁺: m/z=496.2; Found: 496.3.

### Example 28. (S)-1-(3-(4-(2-(2-Fluoro-6-methoxyphenyl)-3-methylimidazo[2,1-f][1,6]naphthyridin-9-yl)-1H-pyrazol-1-yl)pyrrolidin-1-yl)-2-hydroxyethan-1-one

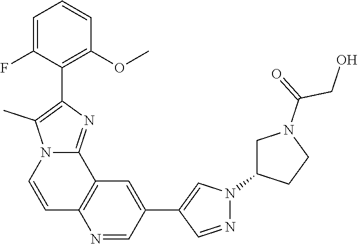 [SMILES: COc1cccc(F)c1-c1nc2c3cc(-c4cnn([C@H]5CCN(C(=O)CO)C5)c4)cnc3ccn2c1C]

This compound was prepared according to the procedures described in Example 25, with (2-fluoro-6-methoxyphenyl)boronic acid replacing (2-fluoro-6-(trifluoromethyl)phenyl)boronic acid in Step 7. The title compound was isolated as the TFA salt. LCMS calculated for C₂₇H₂₆FN₆O₃ (M+H)⁺: m/z=501.2; Found: 501.2.

### Intermediate 2. 2-Bromo-3-methyl-9-(1-methyl-1H-pyrazol-4-yl)imidazo[2,1-f][1,6]naphthyridine

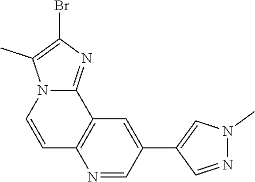

This compound was prepared according to the procedures described in Example 25, Steps 1 through 6, with 1-methyl-4-(4,4,5,5-tetramethyl-1,3,2-dioxaborolan-2-yl)-1H-pyrazole replacing 4-(4,4,5,5-tetramethyl-1,3,2-dioxaborolan-2-yl)-1-((2-(trimethylsilyl)ethoxy)methyl)-1H-pyrazole in Step 2. The title compound was isolated as the TFA salt. LCMS calculated for C₁₅H₁₃BrN₅ (M+H)⁺: m/z=342.0/344.0; Found: 342.0/344.0.

### Example 29. 2-(2,3-Dimethylphenyl)-3-methyl-9-(1-methyl-1H-pyrazol-4-yl)imidazo[2,1-f][1,6]naphthyridine

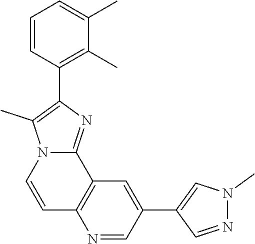

A vial containing a mixture of 2-bromo-3-methyl-9-(1-methyl-1H-pyrazol-4-yl)imidazo[2,1-f][1,6]naphthyridine (Intermediate 2, 10 mg, 0.029 mmol), (2,3-dimethylphenyl)boronic acid (9 mg, 0.058 mmol), XPhos Pd G2 (2 mg, 2.9 μmol), and potassium phosphate, tribasic (19 mg, 0.088 mmol) was evacuated and backfilled with nitrogen three times, followed by the addition of 1,4-dioxane (1 mL) and water (250 μL). The vial was sealed and heated to 80° C. for 2 h. After cooling to room temperature, the mixture was diluted with CH₃CN and purified with prep-LCMS (XBridge C18 column, eluting with a gradient of acetonitrile/water containing 0.1% TFA, at flow rate of 60 mL/min). The title compound was isolated as the TFA salt. LCMS calculated for C₂₃H₂₂N₅ (M+H)⁺: m/z=368.2; Found: 368.2. ¹H NMR (400 MHz, DMSO-d₆) δ 9.34 (d, J=2.2 Hz, 1H), 8.98 (d, J=2.3 Hz, 1H), 8.60 (d, J=7.3 Hz, 1H), 8.48 (s, 1H), 8.12 (s, 1H), 7.64 (d, J=7.5 Hz, 1H), 7.36 (d, J=4.6 Hz, 1H), 7.29 (d, J=4.5 Hz, 2H), 3.94 (s, 3H), 2.48 (s, 3H), 2.37 (s, 3H), 2.21 (s, 3H).

### Example 30. (3-Methyl-4-(3-methyl-9-(1-methyl-1H-pyrazol-4-yl)imidazo[2,1-f][1,6]naphthyridin-2-yl)phenyl)methanol

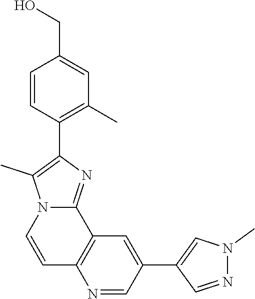

This compound was prepared according to the procedures described in Example 29, with (4-(hydroxymethyl)-2-methylphenyl)boronic acid replacing (2,3-dimethylphenyl)boronic acid. The title compound was isolated as the TFA salt. LCMS calculated for C₂₃H₂₂N₅O (M+H)⁺: m/z=384.2; Found: 384.3.

### Example 31. 2-(2,6-Dichlorophenyl)-3-methyl-9-(1-methyl-1H-pyrazol-4-yl)imidazo[2,1-f][1,6]naphthyridin-8-ol

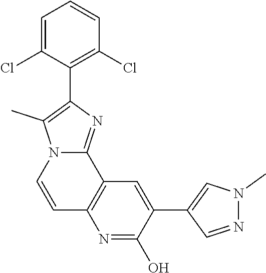

### Step 1. 2-(2,6-Dichlorophenyl)-3-methyl-9-(1-methyl-1H-pyrazol-4-yl)imidazo[2,1-f][1,6]naphthyridine

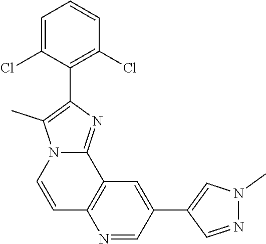

A vial containing a mixture of 2-bromo-3-methyl-9-(1-methyl-1H-pyrazol-4-yl)imidazo[2,1-f][1,6]naphthyridine (Intermediate 2, 600 mg, 1.75 mmol), (2,6-dichlorophenyl)boronic acid (836 mg, 4.38 mmol), potassium fluoride (336 mg, 5.79 mmol) and Pd₂(dba)₃ (321 mg, 0.351 mmol) was evacuated and backfilled with nitrogen three times, followed by the addition of 1,4-dioxane (16 mL), water (1.6 mL), and tri-tert-butylphosphine (1.0 M in toluene, 700 μL, 0.7 mmol). The vial was sealed and heated to 100° C. for 1 h. After cooling to room temperature, the mixture was filtered through Celite and washed with CH₂Cl₂, followed by concentration of the filtrate in vacuo. The obtained crude product was purified by Biotage Isolera to give the desired product as a yellow solid. LCMS calculated for C₂₁H₁₆Cl₂N₅ (M+H)⁺: m/z=408.1/410.1; Found: 408.1/410.1.

### Step 2. 2-(2,6-Dichlorophenyl)-3-methyl-9-(1-methyl-1H-pyrazol-4-yl)imidazo[2,1-f][1,6]naphthyridine 7-oxide

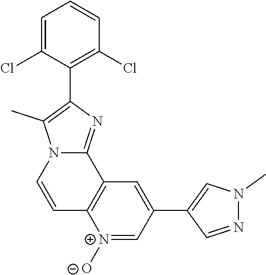

To a vial containing 2-(2,6-dichlorophenyl)-3-methyl-9-(1-methyl-1H-pyrazol-4-yl)imidazo[2,1-f][1,6]naphthyridine (400 mg, 0.980 mmol) as a solution in CH₂Cl₂ (10 mL) was added m-CPBA (220 mg, 0.980 mmol) and left to stir at r.t. After 1 h, added a second portion of m-CPBA (220 mg, 0.980 mmol) and left to stir for 1 h. The volatiles were removed in vacuo and the crude residue was purified by Biotage Isolera to give the desired product as a yellow solid (260 mg, 62.5% yield). LCMS calculated for C₂₁H₁₆Cl₂N₅O (M+H)⁺: m/z=424.1/426.1; Found: 424.0/426.1.

Step 3. 8-Chloro-2-(2,6-dichlorophenyl)-3-methyl-9-(1-methyl-1H-pyrazol-4-yl)imidazo[2,1-f][1,6]naphthyridine & 10-chloro-2-(2,6-dichlorophenyl)-3-methyl-9-(1-methyl-1H-pyrazol-4-yl)imidazo[2,1-f][1,6]naphthyridine

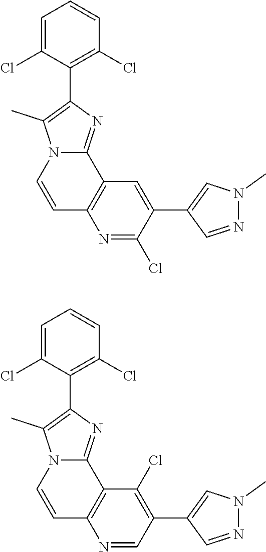

To a vial containing 2-(2,6-dichlorophenyl)-3-methyl-9-(1-methyl-1H-pyrazol-4-yl)imidazo[2,1-f][1,6]naphthyridine 7-oxide (100 mg, 0.236 mmol) as a solution in CH₂Cl₂ (2 mL) was added phosphoryl chloride (879 μL, 9.43 mmol) and left to stir at r.t. for 1 h. The volatiles were removed in vacuo and the resulting residue was treated with saturated aqueous NaHCO₃ and extracted with CH₂Cl₂. The organic phase was washed with brine, dried over MgSO₄, filtered and the solvent was evaporated in vacuo. The resulting residue was purified by Biotage Isolera to give the desired products as yellow solids. LCMS calculated for C₂₁H₁₅Cl₃N₅ (M+H)⁺: m/z=442.0/444.0; Found: 442.2/444.2.

### Step 4. 2-(2,6-Dichlorophenyl)-3-methyl-9-(1-methyl-1H-pyrazol-4-yl)imidazo[2,1-f][1,6]naphthyridin-8-ol

To a vial containing 8-chloro-2-(2,6-dichlorophenyl)-3-methyl-9-(1-methyl-1H-pyrazol-4-yl)imidazo[2,1-f][1,6]naphthyridine (10 mg, 0.023 mmol) as a solution in 1,4-dioxane (0.5 mL) was added lithium hydroxide (2.0 M in H₂O, 500 μL, 1.00 mmol). The vial was sealed and heated to 100° C. for 16 h. After cooling to room temperature, the mixture was diluted with CH₃CN and purified with prep-LCMS (XBridge C18 column, eluting with a gradient of acetonitrile/water containing 0.1% TFA, at flow rate of 60 mL/min). The title compound was isolated as the TFA salt. LCMS calculated for C₂₁H₁₆Cl₂N₅O (M+H)⁺: m/z=424.1/426.1; Found: 424.0/426.1.

### Example 32. (S)-2-(2,6-Dichlorophenyl)-3-methyl-9-(1-methyl-1H-pyrazol-4-yl)-10-((tetrahydrofuran-3-yl)oxy)imidazo[2,1-f][1,6]naphthyridine

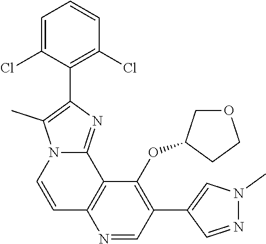

To a vial containing 10-chloro-2-(2,6-dichlorophenyl)-3-methyl-9-(1-methyl-1H-pyrazol-4-yl)imidazo[2,1-f][1,6]naphthyridine (Example 31, Step 3, 15 mg, 0.034 mmol) as a solution in 1,4-dioxane (1 mL) was added cesium carbonate (110 mg, 0.339 mmol) and (S)-tetrahydrofuran-3-ol (27 μL, 0.339 mmol). The vial was sealed and heated to 100° C. for 16 h. After cooling to room temperature, the mixture was diluted with CH₃CN and purified with prep-LCMS (XBridge C18 column, eluting with a gradient of acetonitrile/water containing 0.1% TFA, at flow rate of 60 mL/min). The title compound was isolated as the TFA salt. LCMS calculated for C₂₅H₂₂Cl₂N₅O₂ (M+H)⁺: m/z=494.1/496.1; Found: 494.2/496.2.

### Example 33. 2-(2,6-Dichlorophenyl)-N,N,3-trimethyl-9-(1-methyl-1H-pyrazol-4-yl)imidazo[2,1-f][1,6]naphthyridin-8-amine

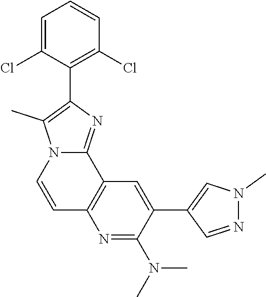

To a vial containing 8-chloro-2-(2,6-dichlorophenyl)-3-methyl-9-(1-methyl-1H-pyrazol-4-yl)imidazo[2,1-f][1,6]naphthyridine (Example 31, Step 3, 25 mg, 0.056 mmol) as a solution in DMSO (565 μL) was added diisopropylethylamine (15 μL, 0.085 mmol) and dimethylamine (2.0 M in THF, 31 μL, 0.062 mmol). The vial was sealed and heated to 100° C. for 16 h. After cooling to room temperature, the mixture was diluted with CH₃CN and purified with prep-LCMS (XBridge C18 column, eluting with a gradient of acetonitrile/water containing 0.1% TFA, at flow rate of 60 mL/min). The title compound was isolated as the TFA salt. LCMS calculated for C₂₃H₂₁Cl₂N₆ (M+H)⁺: m/z=451.1/453.1; Found: 451.1/453.2.

### Example 34. N-(2,4-Dichloro-3-(3-methyl-9-(1-methyl-1H-pyrazol-4-yl)imidazo[2,1-f][1,6]naphthyridin-2-yl)phenyl)acrylamide

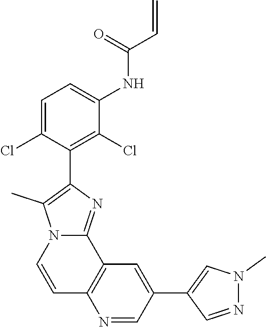

### Step 1. (3-Amino-2,6-dichlorophenyl)boronic acid

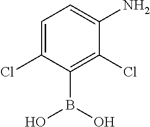 [SMILES: Nc1ccc(Cl)c(B(O)O)c1Cl]

A vial containing (2,6-dichloro-3-nitrophenyl)boronic acid (300 mg, 1.27 mmol) and palladium on carbon (10 wt %, 2.4 mg, 3.4 μmol) as a solution in methanol (10 mL) was connected to a balloon with hydrogen and evacuated and backfilled three times. The reaction mixture was left to stir for 1 h, then filtered through Celite and washed with methanol, followed by concentration of the filtrate in vacuo. The obtained crude product was used in the next step without further purification.

### Step 2. 2,4-Dichloro-3-(3-methyl-9-(1-methyl-1H-pyrazol-4-yl)imidazo[2,1-f][1.6]naphthyridin-2-yl)aniline

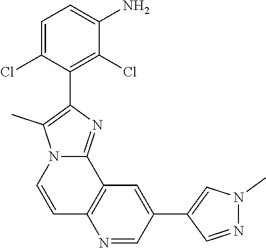

A vial containing a mixture of 2-bromo-3-methyl-9-(1-methyl-1H-pyrazol-4-yl)imidazo[2,1-f][1,6]naphthyridine (Intermediate 2, 20 mg, 0.058 mmol), (3-amino-2,6-dichlorophenyl)boronic acid (12 mg, 0.058 mmol), XPhos Pd G2 (4.2 mg, 5.8 μmol), and potassium phosphate, tribasic (37 mg, 0.175 mmol) was evacuated and backfilled with nitrogen three times, followed by the addition of 1,4-dioxane (1 mL) and water (250 μL). The vial was sealed and heated to 80° C. for 1 h. After cooling to room temperature, the mixture was filtered through Celite and washed with CH₂Cl₂, followed by concentration of the filtrate in vacuo. The obtained crude product was used in the next step without further purification. LCMS calculated for C₂₁H₁₇Cl₂N₆ (M+H)⁺: m/z=423.1/425.1; Found: 423.2/425.2.

### Step 3. N-(2,4-Dichloro-3-(3-methyl-9-(1-methyl-1H-pyrazol-4-yl)imidazo[2,1-f][1,6]naphthyridin-2-yl)phenyl)acrylamide

To a vial containing 2,4-dichloro-3-(3-methyl-9-(1-methyl-1H-pyrazol-4-yl)imidazo[2,1-f][1,6]naphthyridin-2-yl)aniline (25 mg, 0.058 mmol) as a solution in CH₂Cl₂ (1 mL) was added diisopropylethylamine (30 μL, 0.174 mmol) and acryloyl chloride (6 μL, 0.075 mmol). The vial was left to stir at r.t. for 1 h. After the volatiles were removed in vacuo, the mixture was diluted with CH₃CN and purified with prep-LCMS (XBridge C18 column, eluting with a gradient of acetonitrile/water containing 0.1% TFA, at flow rate of 60 mL/min). The title compound was isolated as the TFA salt. LCMS calculated for C₂₄H₁₉Cl₂N₆O (M+H)⁺: m/z=477.1/479.1; Found: 477.1/479.0.

### Example 35. N-(3-(3-Methyl-9-(1-methyl-1H-pyrazol-4-yl)imidazo[2,1-f][1,6]naphthyridin-2-yl)phenyl)acrylamide

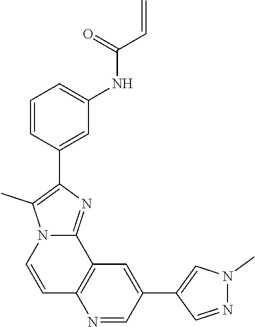

This compound was prepared according to the procedures described in Example 34, with (3-aminophenyl)boronic acid replacing (3-amino-2,6-dichlorophenyl)boronic acid in Step 2. The title compound was isolated as the TFA salt. LCMS calculated for C₂₄H₂₁N₆O (M+H)⁺: m/z=409.2; Found: 409.1. ¹H NMR (400 MHz, DMSO-d₆) δ 10.34 (s, 1H), 9.25 (d, J=2.2 Hz, 1H), 8.95 (d, J=2.2 Hz, 1H), 8.51 (d, J=7.6 Hz, 2H), 8.24 (d, J=1.9 Hz, 1H), 8.17 (s, 1H), 7.73 (dd, J=8.1, 2.0 Hz, 1H), 7.61-7.55 (m, 1H), 7.52 (d, J=7.9 Hz, 1H), 7.50-7.43 (m, 1H), 6.50 (dd, J=17.0, 10.1 Hz, 1H), 6.31 (dd, J=17.0, 2.0 Hz, 1H), 5.81 (dd, J=10.1, 2.0 Hz, 1H), 3.94 (s, 3H), 2.75 (s, 3H).

### Example 36. N-(2-(3-Methyl-9-(1-methyl-1H-pyrazol-4-yl)imidazo[2,1-f][1,6]naphthyridin-2-yl)phenyl)acrylamide

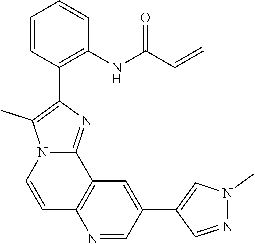

This compound was prepared according to the procedures described in Example 34, with 2-(4,4,5,5-tetramethyl-1,3,2-dioxaborolan-2-yl)aniline replacing (3-amino-2,6-dichlorophenyl)boronic acid in Step 2. The title compound was isolated as the TFA salt. LCMS calculated for C₂₄H₂₁N₆O (M+H)⁺: m/z=409.2; Found: 409.1.

### Example 37. (1-Aminocyclopropyl)(3-(4-(2-(2,6-dichlorophenyl)-3-methylimidazo[2,1-f][1,6]naphthyridin-9-yl)-1H-pyrazol-1-yl)azetidin-1-yl)methanone

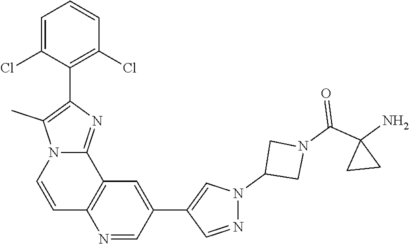 [SMILES: Cc1c(-c2c(Cl)cccc2Cl)nc2c3cc(-c4cnn(C5CN(C(=O)C6(N)CC6)C5)c4)cnc3ccn12]

### Step 1. 2-(2,6-Dichlorophenyl)-9-(1-((2-(trimethylsilyl)ethoxy)methyl)-1H-pyrazol-4-yl)imidazo[2,1-f][1.6]naphthyridine

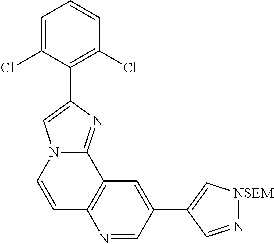 [SMILES: C[Si](C)(C)CCOCn1cc(-c2cnc3ccn4cc(-c5c(Cl)cccc5Cl)nc4c3c2)cn1]

A vial containing a mixture of 9-bromo-2-(2,6-dichlorophenyl)imidazo[2,1-f][1,6]naphthyridine (Example 15, Step 1, 1.0 g, 2.54 mmol), 4-(4,4,5,5-tetramethyl-1,3,2-dioxaborolan-2-yl)-1-((2-(trimethylsilyl)ethoxy)methyl)-1H-pyrazole (866 mg, 2.67 mmol), (1,1′-bis(diphenylphosphino)ferrocene)-dichloropalladium(II) (208 mg, 0.254 mmol), and potassium phosphate, tribasic (1.62 g, 7.63 mmol) was evacuated and backfilled with nitrogen three times, followed by the addition of 1,4-dioxane (13.5 mL) and water (3.5 mL). The vial was sealed and heated to 80° C. for 30 min. After cooling to room temperature, the mixture was filtered through Celite and washed with CH₂Cl₂, followed by concentration of the filtrate in vacuo. The obtained crude residue was purified by Biotage Isolera to give the desired product. LCMS calculated for C₂₅H₂₆Cl₂N₅OSi (M+H)⁺: m/z=510.1/512.1; found 510.1/512.1.

### Step 2. 3-Bromo-2-(2,6-dichlorophenyl)-9-(1-((2-(trimethylsilyl)ethoxy)methyl)-1H-pyrazol-4-yl)imidazo[2,1-f][1.6]naphthyridine

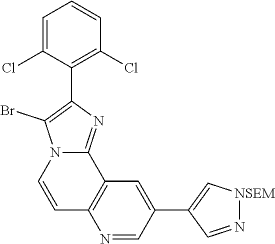 [SMILES: C[Si](C)(C)CCOCn1cc(-c2cnc3ccn4c(Br)c(-c5c(Cl)cccc5Cl)nc4c3c2)cn1]

This compound was prepared according to the procedures described in Example 25, with 2-(2,6-dichlorophenyl)-9-(1-((2-(trimethylsilyl)ethoxy)methyl)-1H-pyrazol-4-yl)imidazo[2,1-f][1,6]naphthyridine replacing 9-(1-((2-(trimethylsilyl)ethoxy)methyl)-1H-pyrazol-4-yl)imidazo[2,1-f][1,6]naphthyridine in Step 3. LCMS calculated for C₂₅H₂₅BrCl₂N₅OSi (M+H)⁺: m/z=588.0/590.0/592.0; Found: 588.2/590.2/592.2.

### Step 3. 2-(2,6-Dichlorophenyl)-3-methyl-9-(1-((2-(trimethylsilyl)ethoxy)methyl)-1H-pyrazol-4-yl)imidazo[2,1-f][1.6]naphthyridine

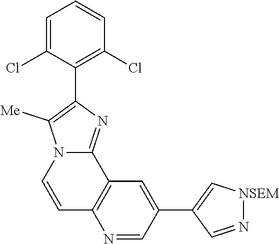 [SMILES: Cc1c(-c2c(Cl)cccc2Cl)nc2c3cc(-c4cnn(COCC[Si](C)(C)C)c4)cnc3ccn12]

This compound was prepared according to the procedures described in Example 1, with 3-bromo-2-(2,6-dichlorophenyl)-9-(1-((2-(trimethylsilyl)ethoxy)methyl)-1H-pyrazol-4-yl)imidazo[2,1-f][1,6]naphthyridine replacing 3-bromo-2-(2-chlorophenyl)-9-(1-methyl-1H-pyrazol-4-yl)imidazo[2,1-f][1,6]naphthyridine in Step 6. LCMS calculated for C₂₆H₂₈Cl₂N₅OSi (M+H)⁺: m/z=524.1/526.1; Found: 524.1/526.1.

### Step 4. 2-(2,6-Dichlorophenyl)-3-methyl-9-(1H-pyrazol-4-yl)imidazo[2,1-f][1,6]naphthyridine

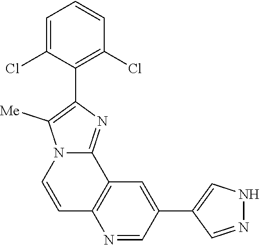 [SMILES: Cc1c(-c2c(Cl)cccc2Cl)nc2c3cc(-c4cn[nH]c4)cnc3ccn12]

To a vial containing a mixture of 2-(2,6-dichlorophenyl)-3-methyl-9-(1-((2-(trimethylsilyl)ethoxy)methyl)-1H-pyrazol-4-yl)imidazo[2,1-f][1,6]naphthyridine (1.33 g, 2.54 mmol) as a solution in CH₂Cl₂ (10 mL) was added TFA (3 mL). The reaction mixture was stirred at r.t. for 3 h, concentrated in vacuo, then dissolved in MeOH (2 mL) and added to a stirring solution of saturated aqueous NaHCO₃ (50 mL). The resulting tan precipitate was filtered and collected, washed with water, followed by drying under vacuum overnight. LCMS calculated for C₂₀H₁₄Cl₂N₅ (M+H)⁺: m/z=394.1/396.1; found 394.0/396.0.

### Step 5. 9-(1-(Azetidin-3-yl)-1H-pyrazol-4-yl)-2-(2,6-dichlorophenyl)-3-methylimidazo[2,1-f][1,6] naphthyridine

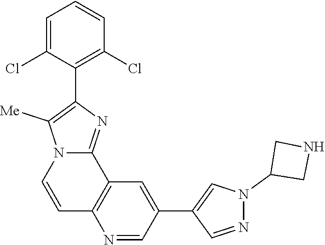 [SMILES: Cc1c(-c2c(Cl)cccc2Cl)nc2c3cc(-c4cnn(C5CNC5)c4)cnc3ccn12]

To a vial containing a mixture of 2-(2,6-dichlorophenyl)-3-methyl-9-(1H-pyrazol-4-yl)imidazo[2,1-f][1,6]naphthyridine (250 mg, 0.634 mmol), tert-butyl 3-((methylsulfonyl)oxy)azetidine-1-carboxylate (319 mg, 1.27 mmol), cesium carbonate (620 mg, 1.90 mmol) was added MeCN (6.3 mL). The vial was sealed and heated to 80° C. for 16 h. After cooling to room temperature, the mixture was filtered through Celite and washed with MeCN, followed by concentration of the filtrate in vacuo. The resulting residue was purified by Biotage Isolera to give the desired product as a tan solid. The purified material was then dissolved in CH₂Cl₂(3 mL) and TFA (1 mL) and left to stir at r.t. for 1 h. The reaction mixture was concentrated in vacuo, then dissolved in MeOH (3 mL) and added to a stirring solution of saturated aqueous NaHCO₃ (15 mL). The resulting solid precipitate was filtered and collected, followed by drying under vacuum overnight. LCMS calculated for C₂₃H₁₉Cl₂N₆ (M+H)⁺: m/z=449.1/451.1; Found: 449.1/451.1.

### Step 6. (1-Aminocyclopropyl)(3-(4-(2-(2,6-dichlorophenyl)-3-methylimidazo[2,1-f][1,6]naphthyridin-9-yl)-1H-pyrazol-1-yl)azetidin-1-yl)methanone

To a vial containing 9-(1-(azetidin-3-yl)-1H-pyrazol-4-yl)-2-(2,6-dichlorophenyl)-3-methylimidazo[2,1-f][1,6]naphthyridine (50 mg, 0.111 mmol) as a solution in DMF (1 mL) was added 1-((tert-butoxycarbonyl)amino)cyclopropane-1-carboxylic acid (34 mg, 0.167 mmol), diisopropylethylamine (39 μL, 0.223 mmol), and HATU (64 mg, 0.167 mmol). The reaction mixture was left to stir at r.t. for 1 h, upon which time water was added and the resulting solid was collected by filtration and washed with water, followed by hexanes and drying under vacuum. The solid was then dissolved with TFA and purified with prep-LCMS (XBridge C18 column, eluting with a gradient of acetonitrile/water containing 0.1% TFA, at flow rate of 60 mL/min). The title compound was isolated as the TFA salt. LCMS calculated for C₂₇H₂₄Cl₂N₇O (M+H)⁺: m/z=532.1/534.1; found 532.2/534.2. ¹H NMR (400 MHz, DMSO-d₆) δ 9.25 (d, J=2.3 Hz, 1H), 8.96 (d, J=2.3 Hz, 1H), 8.81 (s, 1H), 8.53 (s, 2H), 8.46 (d, J=7.5 Hz, 1H), 8.41 (s, 1H), 7.69-7.61 (m, 2H), 7.55 (dd, J=8.9, 7.3 Hz, 1H), 7.41 (d, J=7.5 Hz, 1H), 5.36-5.25 (m, 1H), 4.80-4.25 (m, 4H), 2.39 (s, 3H), 1.56 (m, 2H), 1.29-1.23 (m, 2H).

### Example 38. 1-(3-(4-(2-(2,6-Dichlorophenyl)-3-methylimidazo[2,1-f][1,6]naphthyridin-9-yl)-1H-pyrazol-1-yl)azetidin-1-yl)-2-(1H-imidazol-1-yl)ethan-1-one

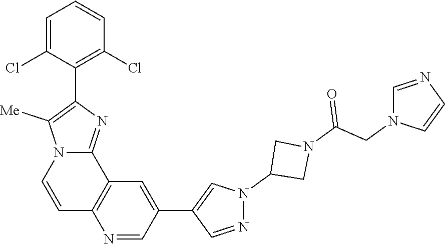 [SMILES: Cc1c(-c2c(Cl)cccc2Cl)nc2c3cc(-c4cnn(C5CN(C(=O)Cn6ccnc6)C5)c4)cnc3ccn12]

This compound was prepared according to the procedures described in Example 37, with 2-(1H-imidazol-1-yl)acetic acid replacing 1-((tert-butoxycarbonyl)amino)cyclopropane-1-carboxylic acid in Step 6. The title compound was isolated as the TFA salt. LCMS calculated for C₂₈H₂₃Cl₂N₈O (M+H)⁺: m/z=557.1/559.1; Found: 557.2/559.3.

### Example 39. (3-(4-(2-(2,6-Dichlorophenyl)-3-methylimidazo[2,1-f][1,6]naphthyridin-9-yl)-1H-pyrazol-1-yl)azetidin-1-yl)(oxazol-4-yl)methanone

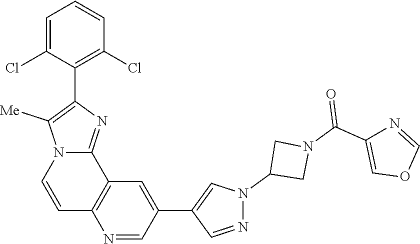 [SMILES: Cc1c(-c2c(Cl)cccc2Cl)nc2c3cc(-c4cnn(C5CN(C(=O)c6cocn6)C5)c4)cnc3ccn12]

This compound was prepared according to the procedures described in Example 37, with oxazole-4-carboxylic acid replacing 1-((tert-butoxycarbonyl)amino)cyclopropane-1-carboxylic acid in Step 6. The title compound was isolated as the TFA salt. LCMS calculated for C₂₇H₂₀Cl₂N₇O₂ (M+H)⁺: m/z=544.1/546.1; Found: 544.1/546.3.

### Example 40. (4-Aminotetrahydro-2H-pyran-4-yl)(3-(4-(2-(2,6-dichlorophenyl)-3-methylimidazo[2,1-f][1,6]naphthyridin-9-yl)-1H-pyrazol-1-yl)azetidin-1-yl)methanone

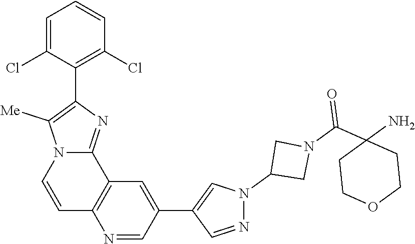 [SMILES: Cc1c(-c2c(Cl)cccc2Cl)nc2c3cc(-c4cnn(C5CN(C(=O)C6(N)CCOCC6)C5)c4)cnc3ccn12]

This compound was prepared according to the procedures described in Example 37, with 4-((tert-butoxycarbonyl)amino)tetrahydro-2H-pyran-4-carboxylic acid replacing 1-((tert-butoxycarbonyl)amino)cyclopropane-1-carboxylic acid in Step 6. The title compound was isolated as the TFA salt. LCMS calculated for C₂₉H₂₈Cl₂N₇O₂ (M+H)⁺: m/z=576.2/578.2; Found: 576.2/578.2.

### Example 41. 3-(3-(4-(2-(2,6-Dichlorophenyl)-3-methylimidazo[2,1-f][1,6]naphthyridin-9-yl)-1H-pyrazol-1-yl)azetidin-1-yl)-3-oxopropanenitrile

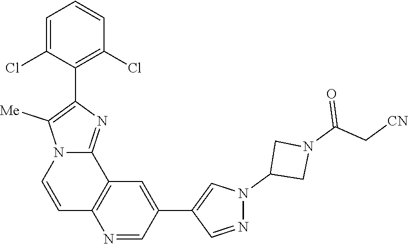 [SMILES: Cc1c(-c2c(Cl)cccc2Cl)nc2c3cc(-c4cnn(C5CN(C(=O)CC#N)C5)c4)cnc3ccn12]

To a vial containing 9-(1-(azetidin-3-yl)-1H-pyrazol-4-yl)-2-(2,6-dichlorophenyl)-3-methylimidazo[2,1-f][1,6]naphthyridinene (Example 37, Step 5, 10 mg, 0.022 mmol) as a solution in ethanol (200 μL) was added DBU (4 μL, 0.022 mmol) and ethyl 2-cyanoacetate (24 μL, 0.223 mmol). The vial was sealed and heated to 100° C. for 4 h. After cooling to room temperature, the mixture was then diluted with CH₃CN and purified with prep-LCMS (XBridge C18 column, eluting with a gradient of acetonitrile/water containing 0.1% TFA, at flow rate of 60 mL/min). The title compound was isolated as the TFA salt. LCMS calculated for C₂₆H₂₀Cl₂N₇O (M+H)⁺: m/z=516.1/518.1; found 516.2/518.1.

### Example 42. 4-((4-(2-(2,6-Dichlorophenyl)imidazo[2,1-f][1,6]naphthyridin-9-yl)-1H-pyrazol-1-yl)methyl)-2-fluorobenzoic acid

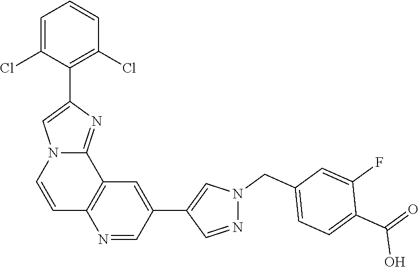 [SMILES: O=C(O)c1ccc(Cn2cc(-c3cnc4ccn5cc(-c6c(Cl)cccc6Cl)nc5c4c3)cn2)cc1F]

To a vial containing a mixture of 2-(2,6-dichlorophenyl)-9-(1H-pyrazol-4-yl)imidazo[2,1-f][1,6]naphthyridine (20 mg, 0.053 mmol, Example 55, Step 2), methyl 4-(bromomethyl)-2-fluorobenzoate (16 mg, 0.063 mmol), and cesium carbonate (34 mg, 0.105 mmol) was added MeCN (1 mL). The vial was sealed and heated to 80° C. for 16 h. After cooling to room temperature, the mixture was filtered through Celite and washed with MeCN, followed by concentration of the filtrate in vacuo. The resulting residue was then dissolved in 1,4-dioxane (1 mL) and lithium hydroxide solution (2 M in H₂O, 0.5 mL) and heated to 50° C. for 1 h. After cooling to room temperature, the volatiles were removed in vacuo and the mixture was diluted with TFA and CH₃CN and purified with prep-LCMS (XBridge C18 column, eluting with a gradient of acetonitrile/water containing 0.1% TFA, at flow rate of 60 mL/min). The title compound was isolated as the TFA salt. LCMS calculated for C₂₇H₁₇Cl₂FN₅O₂ (M+H)⁺: m/z=532.1/534.1; Found: 532.1/534.2.

### Example 43. 4-(4-(2-(2,6-Dichlorophenyl)imidazo[2,1-f][1,6]naphthyridin-9-yl)-1H-pyrazol-1-yl)-N,N-dimethylbenzamide

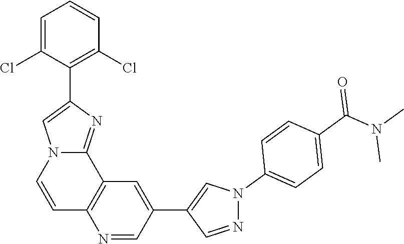 [SMILES: CN(C)C(=O)c1ccc(-n2cc(-c3cnc4ccn5cc(-c6c(Cl)cccc6Cl)nc5c4c3)cn2)cc1]

A vial containing a mixture of 2-(2,6-dichlorophenyl)-9-(1H-pyrazol-4-yl)imidazo[2,1-f][1,6]naphthyridine (15 mg, 0.039 mmol, Example 55, Step 2), 4-bromo-N,N-dimethylbenzamide (16 mg, 0.063 mmol), cesium carbonate (26 mg, 0.079 mmol), and copper(I) iodide (2 mg, 7.9 μmol) was evacuated and backfilled with nitrogen three times, followed by the addition of 1,4-dioxane (1 mL), and trans-N,N′-dimethylcyclohexane-1,2-diamine (5 mg, 0.016 mmol). The vial was sealed and heated to 100° C. for 2 h. After cooling to room temperature, the mixture was diluted with CH₂Cl₂ (1 mL), ammonium hydroxide solution (1 mL) and stirred vigorously for 10 min. The organic layer was then separated and concentrated in vacuo. The resulting residue was then diluted with CH₃CN and water and purified with prep-LCMS (XBridge C18 column, eluting with a gradient of acetonitrile/water containing 0.1% TFA, at flow rate of 60 mL/min). The title compound was isolated as the TFA salt. LCMS calculated for C₂₈H₂₁Cl₂N₆O (M+H)⁺: m/z=527.1/529.1; found 527.3/529.3.

### Example 44. N-((1r,4r)-4-(4-(2-(2,6-Dichlorophenyl)imidazo[2,1-f][1,6]naphthyridin-9-yl)-1H-pyrazol-1-yl)cyclohexyl)-2-methoxyacetamide

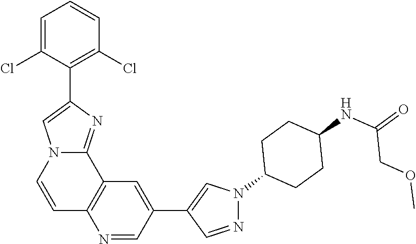 [SMILES: COCC(=O)N[C@H]1CC[C@H](n2cc(-c3cnc4ccn5cc(-c6c(Cl)cccc6Cl)nc5c4c3)cn2)CC1]

### Step 1. (1r,4r)-4-(4-(2-(2,6-Dichlorophenyl)imidazo[2,1-f][1,6]naphthyridin-9-yl)-1H-pyrazol-1-yl)cyclohexan-1-amine

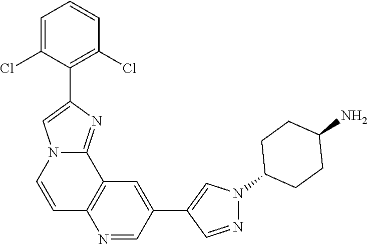 [SMILES: N[C@H]1CC[C@H](n2cc(-c3cnc4ccn5cc(-c6c(Cl)cccc6Cl)nc5c4c3)cn2)CC1]

To a vial containing a mixture of 2-(2,6-dichlorophenyl)-9-(1H-pyrazol-4-yl)imidazo[2,1-f][1,6]naphthyridine (100 mg, 0.263 mmol, Example 55, Step 2), and cesium carbonate (257 mg, 0.789 mmol) as a suspension in CH₃CN (1.3 mL) was added (1s,4s)-4-((tert-butoxycarbonyl)amino)cyclohexyl 4-methylbenzenesulfonate (97 mg, 0.263 mmol). The vial was sealed and heated to 80° C. for 2 h. After cooling to room temperature, the mixture was filtered and washed with CH₃CN, followed by concentration of the filtrate in vacuo. The resulting residue was purified by Biotage Isolera to give a yellow solid. The purified material was then dissolved in CH₂Cl₂(3 mL) and TFA (1 mL) and left to stir at r.t. for 1 h. The reaction mixture was concentrated in vacuo, then dissolved in MeOH (1 mL) and added to a stirring solution of saturated aqueous NaHCO₃ (5 mL). The resulting solid precipitate was filtered and collected, followed by drying under vacuum overnight. LCMS calculated for C₂₅H₂₃Cl₂N₆ (M+H)⁺: m/z=477.1/479.1; Found: 477.2/479.3.

### Step 2. N-((1r,4r)-4-(4-(2-(2,6-Dichlorophenyl)imidazo[2,1-f][1,6]naphthyridin-9-yl)-1H-pyrazol-1-yl)cyclohexyl)-2-methoxyacetamide

To a vial containing (1r,4r)-4-(4-(2-(2,6-dichlorophenyl)imidazo[2,1-f][1,6]naphthyridin-9-yl)-1H-pyrazol-1-yl)cyclohexan-1-amine (15 mg, 0.031 mmol) as a solution in DMF (0.5 mL) was added 2-methoxyacetic acid (3 μL, 0.031 mmol), diisopropylethylamine (17 μL, 0.094 mmol), and HATU (18 mg, 0.047 mmol). The reaction mixture was left to stir at r.t. for 1 h, upon which time water was added and the resulting solid was collected by filtration and washed with water. The solid was then dissolved with CH₃CN and purified with prep-LCMS (XBridge C18 column, eluting with a gradient of acetonitrile/water containing 0.1% TFA, at flow rate of 60 mL/min). The title compound was isolated as the TFA salt. LCMS calculated for C₂₈H₂₇Cl₂N₆O₂ (M+H)⁺: m/z=549.2/551.2; Found: 549.3/551.3. ¹H NMR (400 MHz, DMSO-d₆) δ 9.25 (d, J=2.3 Hz, 1H), 8.93 (d, J=2.3 Hz, 1H), 8.69 (s, 1H), 8.61 (d, J=7.4 Hz, 1H), 8.21 (d, J=6.7 Hz, 2H), 7.67 (d, J=8.2 Hz, 1H), 7.66-7.61 (m, 2H), 7.53 (dd, J=8.8, 7.3 Hz, 1H), 7.38 (d, J=7.4 Hz, 1H), 4.22-4.11 (m, 1H), 3.80 (s, 2H), 3.78-3.70 (m, 1H), 3.31 (s, 3H), 2.18-2.10 (m, 2H), 1.94-1.81 (m, 4H), 1.61-1.47 (m, 2H).

### Example 45. 3-((1r,4r)-4-(4-(2-(2,6-Dichlorophenyl)imidazo[2,1-f][1,6]naphthyridin-9-yl)-1H-pyrazol-1-yl)cyclohexyl)-1,1-dimethylurea

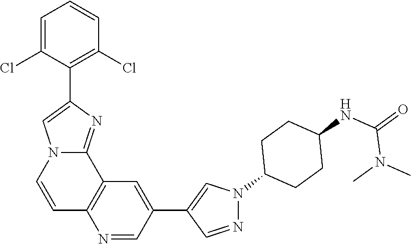 [SMILES: CN(C)C(=O)N[C@H]1CC[C@H](n2cc(-c3cnc4ccn5cc(-c6c(Cl)cccc6Cl)nc5c4c3)cn2)CC1]

To a vial containing a mixture of (1r,4r)-4-(4-(2-(2,6-dichlorophenyl)imidazo[2,1-f][1,6]naphthyridin-9-yl)-1H-pyrazol-1-yl)cyclohexan-1-amine (15 mg, 0.031 mmol, Example 44, Step 1) and diisopropylethylamine (27 μL, 0.157 mmol) as a solution in CH₃CN (1 mL) was added dimethylcarbamyl chloride (6 μL, 0.063 mmol). The solution was left to stir at r.t. for 2 h. The reaction mixture was then diluted with CH₃CN and purified with prep-LCMS (XBridge C18 column, eluting with a gradient of acetonitrile/water containing 0.1% TFA, at flow rate of 60 mL/min). The title compound was isolated as the TFA salt. LCMS calculated for C₂₈H₂₈Cl₂N₇O (M+H)⁺: m/z=548.2/550.2; found 548.3/550.3.

### Example 46. 1-(7-(4-(2-(2,6-Dichlorophenyl)imidazo[2,1-f][1,6]naphthyridin-9-yl)-1H-pyrazol-1-yl)-2-azaspiro[3.5]nonan-2-yl)ethan-1-one

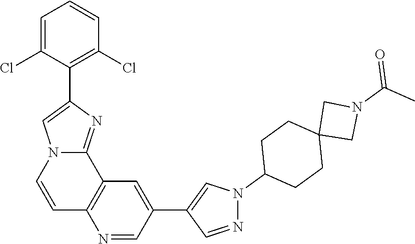 [SMILES: CC(=O)N1CC2(CCC(n3cc(-c4cnc5ccn6cc(-c7c(Cl)cccc7Cl)nc6c5c4)cn3)CC2)C1]

### Step 1. 9-(1-(2-Azaspiro[3.5]nonan-7-yl)-1H-pyrazol-4-yl)-2-(2,6-dichlorophenyl)imidazo[2,1-f][1,6]naphthyridine

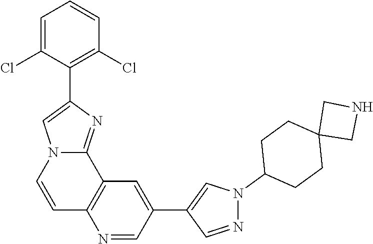 [SMILES: Clc1cccc(Cl)c1-c1cn2ccc3ncc(-c4cnn(C5CCC6(CC5)CNC6)c4)cc3c2n1]

This compound was prepared according to the procedures described in Example 44, with tert-butyl 7-(tosyloxy)-2-azaspiro[3.5]nonane-2-carboxylate replacing (1s,4s)-4-((tert-butoxycarbonyl)amino)cyclohexyl 4-methylbenzenesulfonate in Step 1. LCMS calculated for C₂₇H₂₅Cl₂N₆ (M+H)⁺: m/z=503.2/505.1; Found: 503.2/505.2.

### Step 2. 1-(7-(4-(2-(2,6-Dichlorophenyl)imidazo[2,1-f][1,6]naphthyridin-9-yl)-1H-pyrazol-1-yl)-2-azaspiro[3.5]nonan-2-yl)ethan-1-one

This compound was prepared according to the procedures described in Example 15, with 9-(1-(2-azaspiro[3.5]nonan-7-yl)-1H-pyrazol-4-yl)-2-(2,6-dichlorophenyl)imidazo[2,1-f][1,6]naphthyridine replacing 2-(2,6-dichlorophenyl)-9-(1-(pyrrolidin-3-yl)-1H-pyrazol-4-yl)imidazo[2,1-f][1,6]naphthyridine in Step 3. The title compound was isolated as the TFA salt. LCMS calculated for C₂₉H₂₇Cl₂N₆O (M+H)⁺: m/z=545.2/547.2; Found: 545.2/547.2.

### Example 47. 2-(2,6-Dichlorophenyl)-3-methyl-9-(1-(tetrahydro-2H-pyran-4-yl)-1H-pyrazol-4-yl)imidazo[2,1-f][1,6]naphthyridine

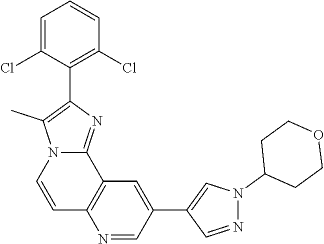 [SMILES: Cc1c(-c2c(Cl)cccc2Cl)nc2c3cc(-c4cnn(C5CCOCC5)c4)cnc3ccn12]

This compound was prepared according to the procedures described in Example 37, Steps 1 through 3, with 1-(tetrahydro-2H-pyran-4-yl)-4-(4,4,5,5-tetramethyl-1,3,2-dioxaborolan-2-yl)-1H-pyrazole replacing 4-(4,4,5,5-tetramethyl-1,3,2-dioxaborolan-2-yl)-1-((2-(trimethylsilyl)ethoxy)methyl)-1H-pyrazole in Step 1. The title compound was isolated as the TFA salt. LCMS calculated for C₂₅H₂₂Cl₂N₅O (M+H)⁺: m/z=478.1/480.1; Found: 478.3/480.4.

### Example 48. (1r,4r)-4-(4-(2-(2,6-Dichlorophenyl)-3-methylimidazo[2,1-f][1,6]naphthyridin-9-yl)-1H-pyrazol-1-yl)cyclohexan-1-ol

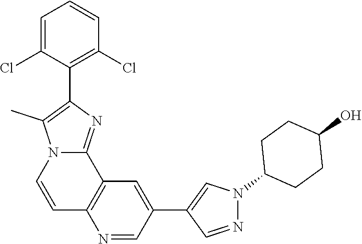 [SMILES: Cc1c(-c2c(Cl)cccc2Cl)nc2c3cc(-c4cnn([C@H]5CC[C@H](O)CC5)c4)cnc3ccn12]

### Step 1. 9-(1-((1 r,4r)-4-((tert-Butyldimethylsilyl)oxy)cyclohexyl)-1H-pyrazol-4-yl)-2-(2,6-dichlorophenyl)-3-methylimidazo[2,1-f][1,6]naphthyridine

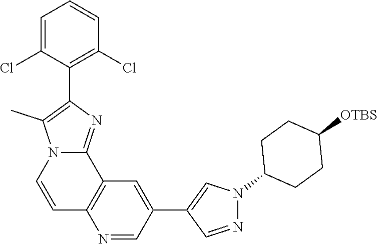 [SMILES: Cc1c(-c2c(Cl)cccc2Cl)nc2c3cc(-c4cnn([C@H]5CC[C@H](O[Si](C)(C)C(C)(C)C)CC5)c4)cnc3ccn12]

This compound was prepared according to the procedures described in Example 37, Steps 1 through 3, with 1-((1r,4r)-4-((tert-butyldimethylsilyl)oxy)cyclohexyl)-4-(4,4,5,5-tetramethyl-1,3,2-dioxaborolan-2-yl)-1H-pyrazole replacing 4-(4,4,5,5-tetramethyl-1,3,2-dioxaborolan-2-yl)-1-((2-(trimethylsilyl)ethoxy)methyl)-1H-pyrazole in Step 1. LCMS calculated for C₃₂H₃₈Cl₂N₅OSi (M+H)⁺: m/z=606.2/608.2; Found: 606.3/608.3.

### Step 2. (1r,4r)-4-(4-(2-(2,6-Dichlorophenyl)-3-methylimidazo[2,1-f][1,6]naphthyridin-9-yl)-1H-pyrazol-1-yl)cyclohexan-1-ol

To a vial containing 9-(1-((1r,4r)-4-((tert-butyldimethylsilyl)oxy)cyclohexyl)-1H-pyrazol-4-yl)-2-(2,6-dichlorophenyl)-3-methylimidazo[2,1-f][1,6]naphthyridine (33 mg, 0.055 mmol) as a solution in 1,4-dioxane (1 mL) was added 5N HCl (0.5 mL). The solution was left to stir at r.t. for 30 min, then diluted with CH₃CN and purified with prep-LCMS (XBridge C18 column, eluting with a gradient of acetonitrile/water containing 0.1% TFA, at flow rate of 60 mL/min). The title compound was isolated as the TFA salt. LCMS calculated for C₂₆H₂₄Cl₂N₅O (M+H)⁺: m/z=492.1/494.1; found 492.1/494.1. ¹H NMR (400 MHz, DMSO-d₆) δ 9.25 (d, J=2.3 Hz, 1H), 8.93 (d, J=2.2 Hz, 1H), 8.65 (s, 1H), 8.46 (d, J=7.5 Hz, 1H), 8.20 (s, 1H), 7.71-7.61 (m, 2H), 7.56 (dd, J=8.9, 7.2 Hz, 1H), 7.43 (d, J=7.5 Hz, 1H), 4.17 (tt, J=11.5, 3.9 Hz, 1H), 3.56-3.44 (m, 1H), 2.40 (s, 3H), 2.08 (d, J=12.6 Hz, 2H), 1.98-1.91 (m, 2H), 1.83 (qd, J=13.0, 3.5 Hz, 2H), 1.44-1.31 (m, 2H).

### Example 49. 4-(4-(2-(2,6-Dichlorophenyl)-3-methylimidazo[2,1-f][1,6]naphthyridin-9-yl)-1H-pyrazol-1-yl)-1-methylcyclohexane-1-carboxylic acid

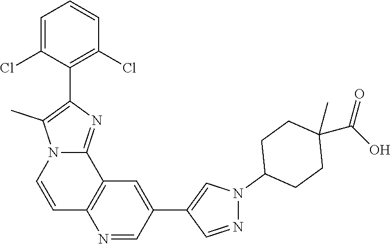 [SMILES: Cc1c(-c2c(Cl)cccc2Cl)nc2c3cc(-c4cnn(C5CCC(C)(C(=O)O)CC5)c4)cnc3ccn12]

### Step 1. Ethyl 1-methyl-4-(tosyloxy)cyclohexane-1-carboxylate

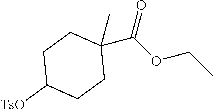 [SMILES: CCOC(=O)C1(C)CCC(OS(=O)(=O)c2ccc(C)cc2)CC1]

To a vial containing ethyl 4-hydroxy-1-methylcyclohexane-1-carboxylate (5.05 g, 27.1 mmol) as a solution in pyridine (27 mL) was added tosyl chloride (6.72 g, 35.2 mmol) and left to stir for 16 h. At completion, 10% aqueous citric acid solution was added and the organics were extracted with EtOAc. The combined organics were then back-extracted with H₂O three times and dried over anhydrous MgSO₄, filtered, and concentrated in vacuo. The crude material was taken forward without additional purification.

### Step 2. 4-(4-(2-(2,6-Dichlorophenyl)-3-methylimidazo[2,1-f][1,6]naphthyridin-9-yl)-1H-pyrazol-1-yl)-1-methylcyclohexane-1-carboxylic acid

To a vial containing a mixture of 2-(2,6-dichlorophenyl)-3-methyl-9-(1H-pyrazol-4-yl)imidazo[2,1-f][1,6]naphthyridine (230 mg, 0.605 mmol, Example 37, Step 4), ethyl 1-methyl-4-(tosyloxy)cyclohexane-1-carboxylate (412 mg, 1.21 mmol), and cesium carbonate (394 mg, 1.21 mmol) was added MeCN (2.4 mL). The vial was sealed and heated to 80° C. for 16 h. After cooling to room temperature, the mixture was filtered through Celite and washed with MeCN, followed by concentration of the filtrate in vacuo. The resulting residue was purified by Biotage Isolera to give a white solid, which was dissolved in 1,4-dioxane (3 mL) and lithium hydroxide solution (2 M in H₂O, 1.5 mL) and heated to 80° C. for 2 h. After cooling to room temperature, the volatiles were removed in vacuo and the mixture was diluted with TFA and CH₃CN and purified with prep-LCMS (XBridge C18 column, eluting with a gradient of acetonitrile/water containing 0.1% TFA, at flow rate of 60 mL/min). The title compound was isolated as the TFA salt. LCMS calculated for C₂₈H₂₆Cl₂N₅O₂ (M+H)⁺: m/z=534.1/536.1; Found: 534.3/536.3.

### Example 50. 3-(4-(2-(2,6-Dichlorophenyl)imidazo[2,1-f][1,6]naphthyridin-9-yl)-1H-pyrazol-1-yl)-N,N-dimethylpyrrolidine-1-carboxamide

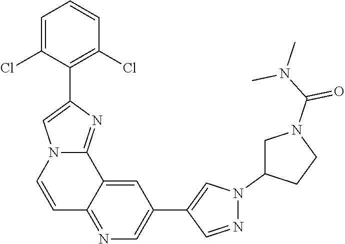 [SMILES: CN(C)C(=O)N1CCC(n2cc(-c3cnc4ccn5cc(-c6c(Cl)cccc6Cl)nc5c4c3)cn2)C1]

This compound was prepared according to the procedures described in Example 15, with dimethylcarbamyl chloride replacing acetyl chloride in Step 3. The title compound was isolated as the TFA salt. LCMS calculated for C₂₆H₂₄Cl₂N₇O (M+H)⁺: m/z=520.1/522.1; Found: 520.1/522.1.

### Example 51. 3-(4-(2-(2,6-Dichlorophenyl)imidazo[2,1-f][1,6]naphthyridin-9-yl)-1H-pyrazol-1-yl)-N,N-dimethylpyrrolidine-1-sulfonamide

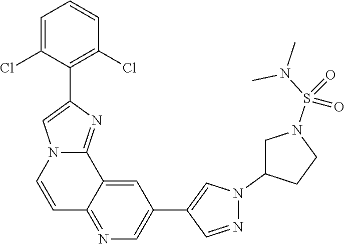 [SMILES: CN(C)S(=O)(=O)N1CCC(n2cc(-c3cnc4ccn5cc(-c6c(Cl)cccc6Cl)nc5c4c3)cn2)C1]

This compound was prepared according to the procedures described in Example 15, with N,N-dimethylsulfamoyl chloride replacing acetyl chloride in Step 3. The title compound was isolated as the TFA salt. LCMS calculated for C₂₅H₂₄Cl₂N₇O₂S (M+H)⁺: m/z=556.1/558.1; Found: 556.1/558.1.

### Example 52. (S)-1-(3-(4-(2-(2,6-Dichlorophenyl)-3-methylimidazo[2,1-f][1,6]naphthyridin-9-yl)-1H-pyrazol-1-yl)pyrrolidin-1-yl)ethan-1-one

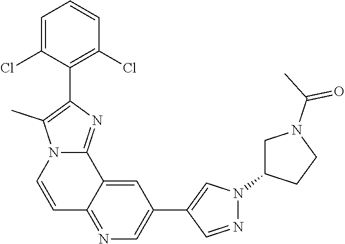 [SMILES: CC(=O)N1CC[C@H](n2cc(-c3cnc4ccn5c(C)c(-c6c(Cl)cccc6Cl)nc5c4c3)cn2)C1]

### Step 1. (S)-2-(2,6-Dichlorophenyl)-3-methyl-9-(1-(pyrrolidin-3-yl)-1H-pyrazol-4-yl)imidazo[2,1-f][1,6]naphthyridine

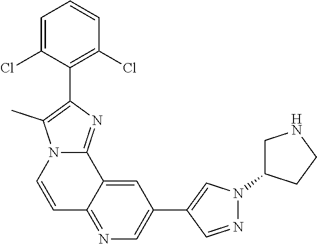 [SMILES: Cc1c(-c2c(Cl)cccc2Cl)nc2c3cc(-c4cnn([C@H]5CCNC5)c4)cnc3ccn12]

This compound was prepared according to the procedures described in Example 37, with tert-butyl (R)-3-((methylsulfonyl)oxy)pyrrolidine-1-carboxylate replacing tert-butyl 3-((methylsulfonyl)oxy)azetidine-1-carboxylate in Step 5. LCMS calculated for C₂₄H₂₁Cl₂N₆ (M+H)⁺: m/z=463.1/465.1; Found: 463.0/465.0.

### Step 2. (S)-1-(3-(4-(2-(2,6-Dichlorophenyl)-3-methylimidazo[2,1-f][1,6]naphthyridin-9-yl)-1H-pyrazol-1-yl)pyrrolidin-1-yl)ethan-1-one

This compound was prepared according to the procedures described in Example 15, with (S)-2-(2,6-dichlorophenyl)-3-methyl-9-(1-(pyrrolidin-3-yl)-1H-pyrazol-4-yl)imidazo[2,1-f][1,6]naphthyridine replacing 2-(2,6-dichlorophenyl)-9-(1-(pyrrolidin-3-yl)-1H-pyrazol-4-yl)imidazo[2,1-f][1,6]naphthyridine in Step 3. The title compound was isolated as the TFA salt. LCMS calculated for C₂₆H₂₃Cl₂N₆O (M+H)⁺: m/z=505.1/507.1; Found: 505.3/507.3.

### Example 53. (S)-1-(3-(4-(2-(2,6-Dichlorophenyl)-3-methylimidazo[2,1-f][1,6]naphthyridin-9-yl)-1H-pyrazol-1-yl)pyrrolidin-1-yl)-2-hydroxyethan-1-one

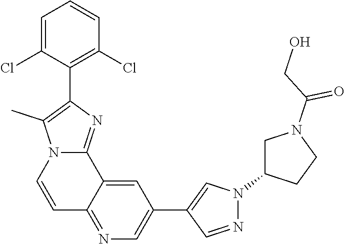 [SMILES: Cc1c(-c2c(Cl)cccc2Cl)nc2c3cc(-c4cnn([C@H]5CCN(C(=O)CO)C5)c4)cnc3ccn12]

This compound was prepared according to the procedures described in Example 25, with (S)-2-(2,6-dichlorophenyl)-3-methyl-9-(1-(pyrrolidin-3-yl)-1H-pyrazol-4-yl)imidazo[2,1-f][1,6]naphthyridine (Example 52, Step 1) replacing (S)-2-(2-chloro-6-(trifluoromethyl)phenyl)-3-methyl-9-(1-(pyrrolidin-3-yl)-1H-pyrazol-4-yl)imidazo[2,1-f][1,6]naphthyridine in Step 8. The title compound was isolated as the TFA salt. LCMS calculated for C₂₆H₂₃Cl₂N₆O₂ (M+H)⁺: m/z=521.1/523.1; Found: 521.1/523.1. ¹H NMR (400 MHz, DMSO-d₆) δ 9.26 (d, J=2.2 Hz, 1H), 8.95 (d, J=2.3 Hz, 1H), 8.72 (d, J=4.6 Hz, 1H), 8.46 (d, J=7.5 Hz, 1H), 8.29 (d, J=5.5 Hz, 1H), 7.69-7.63 (m, 2H), 7.56 (dd, J=8.9, 7.2 Hz, 1H), 7.43 (d, J=7.5 Hz, 1H), 5.05 (dt, J=26.1, 5.2 Hz, 2H), 3.92 (dd, J=11.1, 6.4 Hz, 1H), 3.81 (dt, J=15.6, 4.9 Hz, 2H), 3.67-3.50 (m, 3H), 2.50-2.42 (m, 1H), 2.39 (s, 4H).

### Example 54. 1-(1-(4-(2-(2,6-Dichlorophenyl)-3-methylimidazo[2,1-f][1,6]naphthyridin-9-yl)-1H-pyrazol-1-yl)-3-azabicyclo[3.1.0]hexan-3-yl)-2-hydroxyethan-1-one

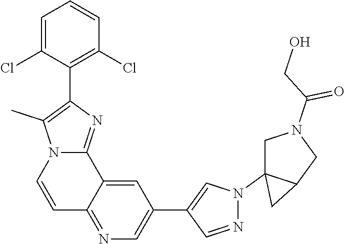 [SMILES: Cc1c(-c2c(Cl)cccc2Cl)nc2c3cc(-c4cnn(C56CC5CN(C(=O)CO)C6)c4)cnc3ccn12]

### Step 1. 9-(1-(3-Azabicyclo[3.1.0]hexan-1-yl)-1H-pyrazol-4-yl)-2-(2,6-dichlorophenyl)-3-methylimidazo[2,1-f][1.6]naphthyridine

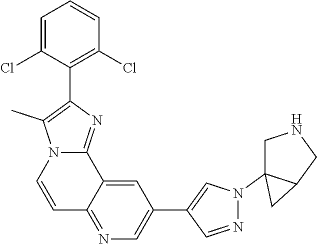 [SMILES: Cc1c(-c2c(Cl)cccc2Cl)nc2c3cc(-c4cnn(C56CNCC5C6)c4)cnc3ccn12]

A vial containing copper(II) acetate (55 mg, 0.304 mmol) and 1,10-phenanthroline monohydrate (60 mg, 0.304 mmol) as a suspension in DCE (2 mL) was sealed and heated to 60° C. for 20 min. A separate vial was charged with a mixture of 2-(2,6-dichlorophenyl)-3-methyl-9-(1H-pyrazol-4-yl)imidazo[2,1-f][1,6]naphthyridine (100 mg, 0.254 mmol, Example 37, Step 4), potassium (3-(tert-butoxycarbonyl)-3-azabicyclo[3.1.0]hexan-1-yl)trifluoroborate (73 mg, 0.254 mmol), and potassium phosphate, tribasic (162 mg, 0.761 mmol). Transferred the copper-ligand solution to the vial containing 2-(2,6-dichlorophenyl)-3-methyl-9-(1H-pyrazol-4-yl)imidazo[2,1-f][1,6]naphthyridine, then added DCE (6 mL) and water (700 μL). The vial was sealed and heated to 80° C. for 16 h. After cooling to room temperature, the mixture was filtered through Celite and washed with CH₂Cl₂, followed by concentration of the filtrate in vacuo. The resulting residue was purified by Biotage Isolera to give the desired product as a yellow solid (98 mg, 67% yield). The purified material was then dissolved in CH₂Cl₂ (3 mL) and TFA (1 mL) and left to stir at r.t. for 1 h. The reaction mixture was concentrated in vacuo, then dissolved in MeOH (1 mL) and added to a stirring solution of saturated aqueous NaHCO₃ (5 mL). The resulting solid precipitate was filtered and collected, followed by drying under vacuum overnight. LCMS calculated for C₂₅H₂₁Cl₂N₆ (M+H)⁺: m/z=475.1/477.1; Found: 475.1/477.1.

### Step 2. 1-(1-(4-(2-(2,6-Dichlorophenyl)-3-methylimidazo[2,1-f][1.6]naphthyridin-9-yl)-1H-pyrazol-1-yl)-3-azabicyclo[3.1.0]hexan-3-yl)-2-hydroxyethan-1-one

This compound was prepared according to the procedures described in Example 25, with 9-(1-(3-azabicyclo[3.1.0]hexan-1-yl)-1H-pyrazol-4-yl)-2-(2,6-dichlorophenyl)-3-methylimidazo[2,1-f][1,6]naphthyridine replacing (S)-2-(2-chloro-6-(trifluoromethyl)phenyl)-3-methyl-9-(1-(pyrrolidin-3-yl)-1H-pyrazol-4-yl)imidazo[2,1-f][1,6]naphthyridine in Step 8. The title compound was isolated as the TFA salt. LCMS calculated for C₂₇H₂₃Cl₂N₆O₂ (M+H)⁺: m/z=533.1/535.1; Found: 533.2/535.2. ¹H NMR (400 MHz, DMSO-d₆) δ 9.27 (d, J=2.3 Hz, 1H), 8.97 (d, J=2.3 Hz, 1H), 8.81 (d, J=6.4 Hz, 1H), 8.47 (d, J=7.5 Hz, 1H), 8.31 (d, J=4.2 Hz, 1H), 7.70-7.63 (m, 2H), 7.56 (dd, J=8.9, 7.3 Hz, 1H), 7.44 (d, J=7.5 Hz, 1H), 4.22 (d, J=11.2 Hz, 1H), 4.13-4.02 (m, 2H), 3.96 (dd, J=15.4, 1.9 Hz, 1H), 3.87-3.55 (m, 3H), 2.40 (s, 3H), 2.37-2.26 (m, 1H), 1.70-1.62 (m, 1H), 1.07 (td, J=5.5, 3.3 Hz, 1H).

### Example 55. (S)-1-(3-(4-(2-(2,6-Dichlorophenyl)-3-(hydroxymethyl)imidazo[2,1-f][1,6]naphthyridin-9-yl)-1H-pyrazol-1-yl)pyrrolidin-1-yl)-2-hydroxyethan-1-one

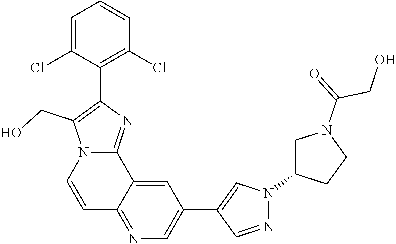 [SMILES: O=C(CO)N1CC[C@H](n2cc(-c3cnc4ccn5c(CO)c(-c6c(Cl)cccc6Cl)nc5c4c3)cn2)C1]

### Step 1. 3-(1-((2-(Trimethylsilyl)ethoxy)methyl)-1H-pyrazol-4-yl)-1,6-naphthyridin-5-amine

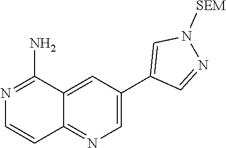 [SMILES: C[Si](C)(C)CCOCn1cc(-c2cnc3ccnc(N)c3c2)cn1]

This compound was prepared according to the procedures described in Example 25, with 3-bromo-1,6-naphthyridin-5-amine (Example 1, Step 2) replacing 9-bromoimidazo[2,1-f][1,6]naphthyridine in Step 2. LCMS calculated for C₁₇H₂₄N₅OSi (M+H)⁺: m/z=342.2; Found: 342.2.

### Step 2. 2-(2,6-Dichlorophenyl)-9-(1H-pyrazol-4-yl)imidazo[2,1-f][1,6]naphthyridine

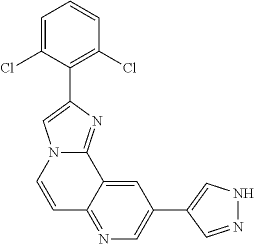 [SMILES: Clc1cccc(Cl)c1-c1cn2ccc3ncc(-c4cn[nH]c4)cc3c2n1]

A microwave vial containing a mixture of 3-(1-((2-(trimethylsilyl)ethoxy)methyl)-1H-pyrazol-4-yl)-1,6-naphthyridin-5-amine (2 g, 5.86 mmol), 2-bromo-1-(2,6-dichlorophenyl)ethan-1-one (1.7 g, 6.44 mmol), sodium bicarbonate (0.738 g, 8.79 mmol), and tert-butanol (10 mL) was irradiated at 150° C. for 14 h using a Biotage Initator+ Microwave Synthesizer. After cooling to room temperature, the solvent was removed in vacuo and the remaining residue was suspended in CH₂Cl₂, filtered, and the filtrate was concentrated in vacuo. The resulting residue was purified by Biotage Isolera to give the desired product as a brown solid. The purified material was then dissolved with CH₂Cl₂ (3 mL) and TFA (2 mL) and left to stir at r.t. for 1 h. At completion, the volatiles were removed in vacuo. The obtained product was used in the next step without further purification. LCMS calculated for C₁₉H₁₂Cl₂N₅ (M+H)⁺: m/z=380.0/382.0; found 380.1/382.0.

### Step 3. tert-Butyl (S)-3-(4-(2-(2,6-dichlorophenyl)imidazo[2,1-f][1,6]naphthyridin-9-yl)-1H-pyrazol-1-yl)pyrrolidine-1-carboxylate

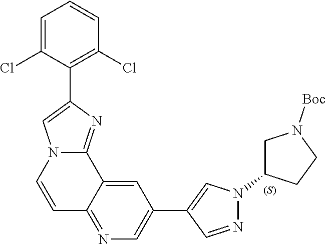

A vial containing 2-(2,6-dichlorophenyl)-9-(1H-pyrazol-4-yl)imidazo[2,1-f][1,6]naphthyridine (2.5 g, 6.57 mmol), tert-butyl (R)-3-((methylsulfonyl)oxy)pyrrolidine-1-carboxylate (2.62 g, 9.86 mmol), cesium carbonate (4.28 g, 13.15 mmol), and acetonitrile (33 mL) was heated to 80° C. and stirred for 16 h. The solution was subsequently cooled to r.t., filtered and the filtrate was concentrated in vacuo then purified by Biotage Isolera to give the desired product as a brown solid. LCMS calculated for C₂₈H₂₇Cl₂N₆O₂ (M+H)⁺: m/z=549.2/551.2; found 549.1/551.1.

### Step 4. tert-Butyl (S)-3-(4-(3-bromo-2-(2,6-dichlorophenyl)imidazo[2,1-f][1,6]naphthyridin-9-yl)-1H-pyrazol-1-yl)pyrrolidine-1-carboxylate

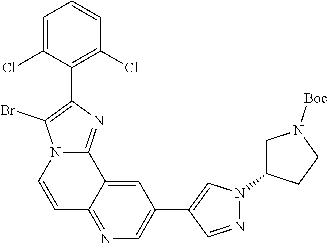 [SMILES: CC(C)(C)OC(=O)N1CC[C@H](n2cc(-c3cnc4ccn5c(Br)c(-c6c(Cl)cccc6Cl)nc5c4c3)cn2)C1]

This compound was prepared according to the procedures described in Example 25, with tert-butyl (S)-3-(4-(2-(2,6-dichlorophenyl)imidazo[2,1-f][1,6]naphthyridin-9-yl)-1H-pyrazol-1-yl)pyrrolidine-1-carboxylate replacing 9-(1-((2-(trimethylsilyl)ethoxy)methyl)-1H-pyrazol-4-yl)imidazo[2,1-f][1,6]naphthyridine in Step 3. LCMS calculated for C₂₈H₂₆BrCl₂N₆O₂ (M+H)⁺: m/z=627.1/629.0/631.0; found 627.2/629.2/631.2.

### Step 5. tert-Butyl (S)-3-(4-(2-(2,6-dichlorophenyl)-3-vinylimidazo[2,1-f][1,6]naphthyridin-9-yl)-1H-pyrazol-1-yl)pyrrolidine-1-carboxylate

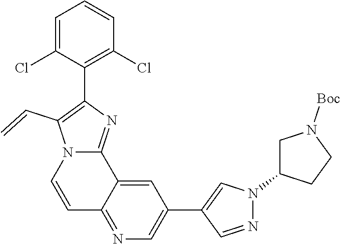 [SMILES: C=Cc1c(-c2c(Cl)cccc2Cl)nc2c3cc(-c4cnn([C@H]5CCN(C(=O)OC(C)(C)C)C5)c4)cnc3ccn12]

A vial containing tert-butyl 3-(4-(3-bromo-2-(2,6-dichlorophenyl)imidazo[2,1-f][1,6]naphthyridin-9-yl)-1H-pyrazol-1-yl)pyrrolidine-1-carboxylate (800 mg, 1.27 mmol), potassium phosphate, tribasic (540 mg, 2.55 mmol), (1,1′-bis(diphenylphosphino)ferrocene)-dichloropalladium(II) (93 mg, 0.127 mmol), and 4,4,5,5-tetramethyl-2-vinyl-1,3,2-dioxaborolane (432 μL, 2.55 mmol) as a solution in 1,4-dioxane (6 mL), and water (600 μL) was purged with nitrogen for 30 sec. and heated to 80° C. for 3 h. Upon cooling to r.t. the solution was diluted with CH₂Cl₂, filtered through Celite and concentrated the filtrate in vacuo. The resulting residue was purified by Biotage Isolera to give the desired product as a brown solid. LCMS calculated for C₃₀H₂₉Cl₂N₆O₂ (M+H)⁺: m/z=575.2/577.2; found 575.1/577.2.

### Step 6. (S)-(2-(2,6-Dichlorophenyl)-9-(1-(pyrrolidin-3-yl)-1H-pyrazol-4-yl)imidazo[2,1-f][1,6]naphthyridin-3-yl)methanol

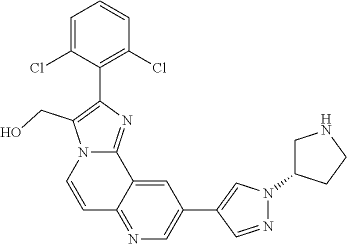 [SMILES: OCc1c(-c2c(Cl)cccc2Cl)nc2c3cc(-c4cnn([C@H]5CCNC5)c4)cnc3ccn12]

To a vial was added tert-butyl (S)-3-(4-(2-(2,6-dichlorophenyl)-3-vinylimidazo[2,1-f][1,6]naphthyridin-9-yl)-1H-pyrazol-1-yl)pyrrolidine-1-carboxylate (600 mg, 1.04 mmol), THF (16 mL), water (5 mL) and osmium tetroxide (4 wt. % in H₂O, 409 μL, 0.052 mmol), followed by sodium periodate (1.11 g, 5.21 mmol). The reaction mixture was stirred at r.t. for 3 h, and upon completion the reaction was quenched with saturated aqueous Na₂S₂O₃ and extracted into EtOAc. The combined organic layers were concentrated in vacuo and the residue was dissolved in isopropanol (10 mL), cooled to 0° C., and NaBH₄ (39 mg, 1.04 mmol) was added with stirring while allowing the reaction to slowly warm to r.t. The reaction was then cooled to 0° C. and quenched by addition of saturated aqueous NH₄Cl. The volatiles were removed in vacuo and the resulting residue was purified by Biotage Isolera to give a yellow solid. This product was taken up in CH₂Cl₂(3 mL) and TFA (1 mL) and stirred at r.t. for 1 h. The volatiles were removed in vacuo and the obtained product was used in the next step without further purification. LCMS calculated for C₂₄H₂₁Cl₂N₆O (M+H)⁺: m/z=479.1/481.1; found 479.1/481.1.

### Step 7. (S)-1-(3-(4-(2-(2,6-Dichlorophenyl)-3-(hydroxymethyl)imidazo[2,1-f][1,6]naphthyridin-9-yl)-1H-pyrazol-1-yl)pyrrolidin-1-yl)-2-hydroxyethan-1-one

To a vial containing (S)-(2-(2,6-dichlorophenyl)-9-(1-(pyrrolidin-3-yl)-1H-pyrazol-4-yl)imidazo[2,1-f][1,6]naphthyridin-3-yl)methanol (15 mg, 0.031 mmol) was added 2-hydroxyacetic acid (3 mg, 0.031 mmol), DMF (0.5 mL) and DIPEA (11 μL, 0.063 mmol). This solution was stirred for 1 min before HATU (18 mg, 0.047 mmol) was added and left to stir for 1 h. Upon completion the reaction mixture was then diluted with acetonitrile/methanol and purified with prep-LCMS (XBridge C18 column, eluting with a gradient of acetonitrile/water containing 0.1% TFA, at flow rate of 60 mL/min). The title compound was isolated as the TFA salt. LCMS calculated for C₂₆H₂₃Cl₂N₆O₃ (M+H)⁺: m/z=537.1/539.1; found 537.1/539.1. ¹H NMR (400 MHz, DMSO-d₆) δ 9.28 (d, J=2.3 Hz, 1H), 8.98 (d, J=2.3 Hz, 1H), 8.73 (d, J=4.4 Hz, 1H), 8.54 (d, J=7.5 Hz, 1H), 8.30 (d, J=5.4 Hz, 1H), 7.68-7.62 (m, 2H), 7.55 (dd, J=9.0, 7.2 Hz, 1H), 7.45 (d, J=7.5 Hz, 1H), 5.05 (dt, J=26.1, 5.5 Hz, 1H), 4.68 (s, 2H), 4.04 (d, J=9.4 Hz, 2H), 3.96-3.77 (m, 2H), 3.67-3.52 (m, 2H), 2.47 (d, J=6.3 Hz, 1H), 2.38 (d, J=6.6 Hz, 1H).

### Example 56. (S)-1-(3-(4-(2-(2,6-Dichlorophenyl)-3-(hydroxymethyl)imidazo[2,1-f][1,6]naphthyridin-9-yl)-1H-pyrazol-1-yl)pyrrolidin-1-yl)ethan-1-one

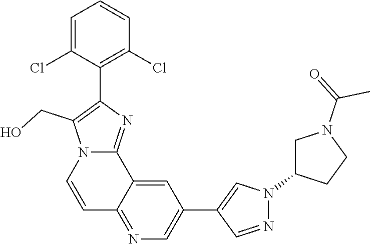 [SMILES: CC(=O)N1CC[C@H](n2cc(-c3cnc4ccn5c(CO)c(-c6c(Cl)cccc6Cl)nc5c4c3)cn2)C1]

This compound was prepared according to the procedures described in Example 55, with acetic acid replacing 2-hydroxyacetic acid in Step 7. The title compound was isolated as the TFA salt. LCMS calculated for C₂₆H₂₃Cl₂N₆O₂ (M+H)⁺: m/z=521.1/523.1; Found: 521.1/523.1.

### Example 57. (S)-(3-(4-(2-(2,6-Dichlorophenyl)-3-(hydroxymethyl)imidazo[2,1-f][1,6]naphthyridin-9-yl)-1H-pyrazol-1-yl)pyrrolidin-1-yl)(morpholino)methanone

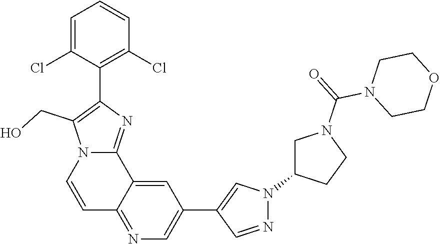 [SMILES: O=C(N1CCOCC1)N1CC[C@H](n2cc(-c3cnc4ccn5c(CO)c(-c6c(Cl)cccc6Cl)nc5c4c3)cn2)C1]

To a vial containing (S)-(2-(2,6-dichlorophenyl)-9-(1-(pyrrolidin-3-yl)-1H-pyrazol-4-yl)imidazo[2,1-f][1,6]naphthyridin-3-yl)methanol (10 mg, 0.021 mmol, Example 55, Step 6) was added morpholine-4-carbonyl chloride (3 μL, 0.025 mmol), DMF (0.5 mL) and diisopropylethylamine (5 μL, 0.025 mmol) and left to stir for 1 h. Upon completion the reaction mixture was then diluted with acetonitrile and methanol and purified with prep-LCMS (XBridge C18 column, eluting with a gradient of acetonitrile/water containing 0.1% TFA, at flow rate of 60 mL/min). The title compound was isolated as the TFA salt. LCMS calculated for C₂₉H₂₈Cl₂N₇O₃ (M+H)⁺: m/z=592.2/594.2; found 592.2/594.1.

### Example 58. 1-((3S)-3-(4-(2-(2,6-dichlorophenyl)-3-(1-hydroxyethyl)imidazo[2,1-f][1,6]naphthyridin-9-yl)-1H-pyrazol-1-yl)pyrrolidin-1-yl)-2-hydroxyethan-1-one

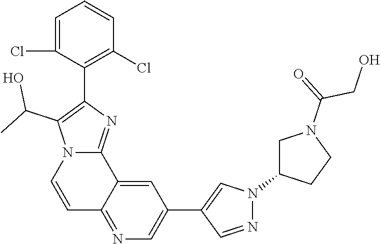 [SMILES: CC(O)c1c(-c2c(Cl)cccc2Cl)nc2c3cc(-c4cnn([C@H]5CCN(C(=O)CO)C5)c4)cnc3ccn12]

### Step 1. tert-Butyl (S)-3-(4-(2-(2,6-dichlorophenyl)-3-(prop-1-en-2-yl)imidazo[2,1-f][1,6]naphthyridin-9-yl)-1H-pyrazol-1-yl)pyrrolidine-1-carboxylate

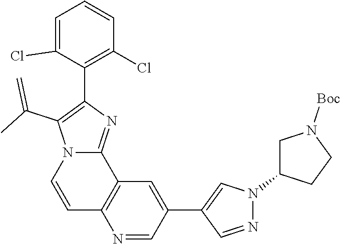 [SMILES: C=C(C)c1c(-c2c(Cl)cccc2Cl)nc2c3cc(-c4cnn([C@H]5CCN(C(=O)OC(C)(C)C)C5)c4)cnc3ccn12]

A mixture of tert-butyl (S)-3-(4-(3-bromo-2-(2,6-dichlorophenyl)imidazo[2,1-f][1,6]naphthyridin-9-yl)-1H-pyrazol-1-yl)pyrrolidine-1-carboxylate (200 mg, 0.318 mmol, Example 55, Step 6), potassium phosphate, tribasic (135 mg, 0.637 mmol), (1,1′-bis(diphenylphosphino)ferrocene)-dichloropalladium(II) (23 mg, 0.032 mmol), and isopropenylboronic acid pinacol ester (120 μL, 0.637 mmol) was suspended in 1,4-dioxane (1.4 mL), and water (0.145 mL). The reaction vial was purged with nitrogen for 30 sec., sealed and heated to 80° C. for 3 h. Upon cooling to r.t. the reaction mixture was diluted with CH₂Cl₂, filtered through Celite and concentrated in vacuo. The resulting residue was purified by Biotage Isolera to give the desired product as a brown solid, LCMS calculated for C₃₁H₃₁Cl₂N₆O₂ (M+H)⁺: m/z=589.2/591.2; found 589.2/591.2.

### Step 2. 1-(2-(2,6-Dichlorophenyl)-9-(1-((S)-pyrrolidin-3-yl)-1H-pyrazol-4-yl)imidazo[2,1-f][1,6]naphthyridin-3-yl)ethan-1-ol

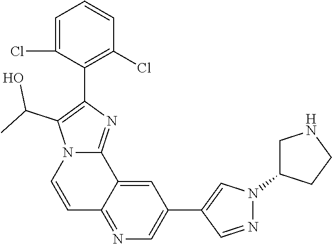 [SMILES: CC(O)c1c(-c2c(Cl)cccc2Cl)nc2c3cc(-c4cnn([C@H]5CCNC5)c4)cnc3ccn12]

This compound was prepared according to the procedures described in Example 55, with tert-butyl (S)-3-(4-(2-(2,6-dichlorophenyl)-3-(prop-1-en-2-yl)imidazo[2,1-f][1,6]naphthyridin-9-yl)-1H-pyrazol-1-yl)pyrrolidine-1-carboxylate replacing tert-butyl (S)-3-(4-(2-(2,6-dichlorophenyl)-3-vinylimidazo[2,1-f][1,6]naphthyridin-9-yl)-1H-pyrazol-1-yl)pyrrolidine-1-carboxylate in Step 6. LCMS calculated for C₂₅H₂₃Cl₂N₆O (M+H)⁺: m/z=493.1/495.1; found 493.3/495.3.

### Step 3. 1-((3S)-3-(4-(2-(2,6-Dichlorophenyl)-3-(1-hydroxyethyl)imidazo[2,1-f][1,6]naphthyridin-9-yl)-1H-pyrazol-1-yl)pyrrolidin-1-yl)-2-hydroxyethan-1-one

This compound was prepared according to the procedures described in Example 25, with 1-(2-(2,6-dichlorophenyl)-9-(1-((S)-pyrrolidin-3-yl)-1H-pyrazol-4-yl)imidazo[2,1-f][1,6]naphthyridin-3-yl)ethan-1-ol replacing (S)-2-(2-chloro-6-(trifluoromethyl)phenyl)-3-methyl-9-(1-(pyrrolidin-3-yl)-1H-pyrazol-4-yl)imidazo[2,1-f][1,6]naphthyridine in Step 8. The title compound was isolated as the TFA salt. LCMS calculated C₂₇H₂₅Cl₂N₆O₃ (M+H)⁺: m/z=551.1/553.1; found 551.2/553.2.

### Example 59. 1-(6-(4-(2-(2,6-Dichlorophenyl)-3-methylimidazo[2,1-f][1,6]naphthyridin-9-yl)-1H-pyrazol-1-yl)-3-azabicyclo[4.1.0]heptan-3-yl)-2-hydroxyethan-1-one

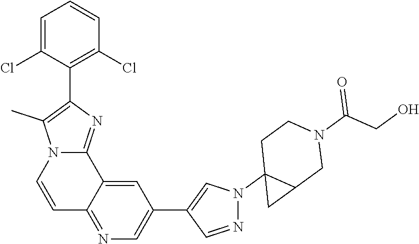 [SMILES: Cc1c(-c2c(Cl)cccc2Cl)nc2c3cc(-c4cnn(C56CCN(C(=O)CO)CC5C6)c4)cnc3ccn12]

This compound was prepared according to the procedures described in Example 54, with potassium (3-(tert-butoxycarbonyl)-3-azabicyclo[4.1.0]heptan-6-yl)trifluoroborate replacing potassium (3-(tert-butoxycarbonyl)-3-azabicyclo[3.1.0]hexan-1-yl)trifluoroborate in Step 1. The title compound was isolated as the TFA salt. LCMS calculated for C₂₈H₂₅Cl₂N₆O₂ (M+H)⁺: m/z=547.1/549.1; found 547.1/549.1. ¹H NMR (400 MHz, DMSO-d₆) δ 9.28 (d, J=2.2 Hz, 1H), 8.97 (d, J=2.2 Hz, 1H), 8.75 (s, 1H), 8.49 (d, J=7.5 Hz, 1H), 8.24 (s, 1H), 7.71-7.64 (m, 2H), 7.57 (dd, J=8.9, 7.2 Hz, 1H), 7.46 (d, J=7.5 Hz, 1H), 4.11 (t, J=4.6 Hz, 2H), 3.89 (dd, J=13.7, 5.4 Hz, 1H), 3.83-3.55 (m, 1H), 3.54-3.19 (m, 2H), 2.41 (s, 3H), 2.33 (s, 1H), 2.26 (s, 1H), 1.89 (s, 1H), 1.55-1.45 (m, 1H), 1.06 (d, J=9.7 Hz, 1H).

### Example 60. 1-(1-(4-(2-(2,6-Dichlorophenyl)-3-methylimidazo[2,1-f][1,6]naphthyridin-9-yl)-1H-pyrazol-1-yl)-3-azabicyclo[4.1.0]heptan-3-yl)ethan-1-one

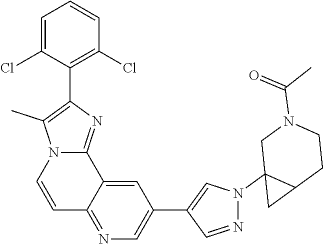 [SMILES: CC(=O)N1CCC2CC2(n2cc(-c3cnc4ccn5c(C)c(-c6c(Cl)cccc6Cl)nc5c4c3)cn2)C1]

### Step 1. 9-(1-(3-Azabicyclo[4.1.0]heptan-1-yl)-1H-pyrazol-4-yl)-2-(2,6-dichlorophenyl)-3-methylimidazo[2,1-f][1.6]naphthyridine

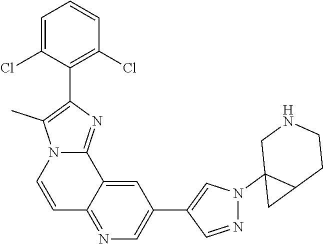 [SMILES: Cc1c(-c2c(Cl)cccc2Cl)nc2c3cc(-c4cnn(C56CNCCC5C6)c4)cnc3ccn12]

This compound was prepared according to the procedures described in Example 54, with potassium (3-(tert-butoxycarbonyl)-3-azabicyclo[4.1.0]heptan-1-yl)trifluoroborate replacing potassium (3-(tert-butoxycarbonyl)-3-azabicyclo[3.1.0]hexan-1-yl)trifluoroborate in Step 1. LCMS calculated for C₂₆H₂₃Cl₂N₆ (M+H)⁺: m/z=489.1/491.1; found 489.1/491.

### Step 2. 1-(1-(4-(2-(2,6-Dichlorophenyl)-3-methylimidazo[2,1-f][1.6]naphthyridin-9-yl)-1H-pyrazol-1-yl)-3-azabicyclo[4.1.0]heptan-3-yl)ethan-1-one

To a vial containing 9-(1-(3-azabicyclo[4.1.0]heptan-1-yl)-1H-pyrazol-4-yl)-2-(2,6-dichlorophenyl)-3-methylimidazo[2,1-f][1,6]naphthyridine (15 mg, 0.031 mmol) was added acetic acid (2 μL, 0.031 mmol), DMF (0.5 mL) and DIPEA (11 μL, 0.061 mmol). This was stirred for 1 min before HATU (18 mg, 0.046 mmol) was added and left to stir for 1 h. Upon completion the reaction mixture was then diluted with acetonitrile/methanol and purified with prep-LCMS (XBridge C18 column, eluting with a gradient of acetonitrile/water containing 0.1% TFA, at flow rate of 60 mL/min). The title compound was isolated as the TFA salt. LCMS calculated for C₂₈H₂₅Cl₂N₆O₂ (M+H)+: m/z=531.1/533.1; found 531.2/533.1.

### Example 61. 1-(3-(5-(2-(2,6-Dichlorophenyl)-3-methylimidazo[2,1-f][1,6]naphthyridin-9-yl)pyridin-2-yl)azetidin-1-yl)-2-hydroxyethan-1-one

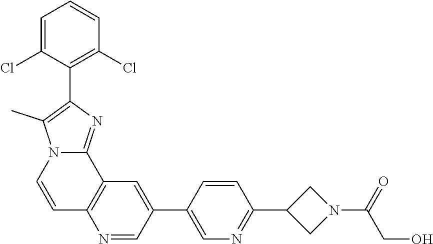 [SMILES: Cc1c(-c2c(Cl)cccc2Cl)nc2c3cc(-c4ccc(C5CN(C(=O)CO)C5)nc4)cnc3ccn12]

### Step 1. tert-Butyl 3-(5-(4,4,5,5-tetramethyl-1,3,2-dioxaborolan-2-yl)pyridin-2-yl)azetidine-1-carboxylate

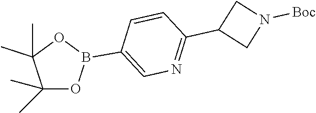 [SMILES: CC(C)(C)OC(=O)N1CC(c2ccc(B3OC(C)(C)C(C)(C)O3)cn2)C1]

A vial containing zinc(0) dust (173 mg, 2.65 mmol), 1,2-dibromoethane (15 μL, 0.177 mmol), TMSCl (22 μL, 0.177 mmol) in THF (3.53 mL) was purged with nitrogen for 30 sec., sealed and heated to 60° C. for 15 min. After cooling to room temperature, a solution of tert-butyl 3-iodoazetidine-1-carboxylate (500 mg, 1.76 mmol) in DMA (3.5 mL) was added. The vial was sealed and heated to 60° C. for another 15 min and, upon subsequent cooling to room temperature, 2,5-dibromopyridine (439 mg, 1.85 mmol), [1,1′-bis(diphenylphosphino)ferrocene]dichloropalladium(II) (72 mg, 0.088 mmol) and copper(I) iodide (17 mg, 0.088 mmol) were added. The vial was sealed and heated to 80° C. for 4 h. After cooling to r.t., the reaction mixture was diluted with CH₂Cl₂, filtered through Celite and the filtrate was concentrated in vacuo. The residue was purified by Biotage Isolera to give tert-butyl 3-(5-bromopyridin-2-yl)azetidine-1-carboxylate (400 mg, 1.277 mmol), which was added to a vial with [1,1′-bis(diphenylphosphino)ferrocene]dichloropalladium(II) (104 mg, 0.128 mmol), bis(pinacolato)diboron (0.649 g, 2.55 mmol), and potassium acetate (307 mg, 3.19 mmol), followed by 1,4-dioxane (4 mL). The solution was purged with nitrogen for 30 sec., then sealed and heated to 80° C. for 2 h. At completion, the volatiles were removed in vacuo to give the desired product which was used directly for the next step. LCMS calculated for the corresponding boronic acid, 3-(5-boronopyridin-2-yl)azetidine-1-carboxylic acid, observed C₉H₁₂BN₂O₄ (M+H−tBu)+: m/z=223.1; found=223.1.

### Step 2. tert-Butyl 3-(5-(2-(2,6-dichlorophenyl)imidazo[2,1-f][1,6]naphthyridin-9-yl)pyridin-2-yl)azetidine-1-carboxylate

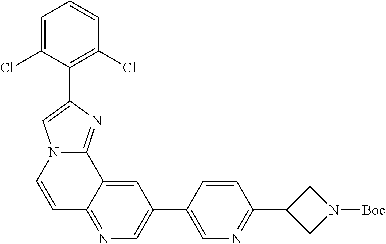 [SMILES: CC(C)(C)OC(=O)N1CC(c2ccc(-c3cnc4ccn5cc(-c6c(Cl)cccc6Cl)nc5c4c3)cn2)C1]

This compound was prepared according to the procedures described in Example 37, with tert-butyl 3-(5-(4,4,5,5-tetramethyl-1,3,2-dioxaborolan-2-yl)pyridin-2-yl)azetidine-1-carboxylate replacing 4-(4,4,5,5-tetramethyl-1,3,2-dioxaborolan-2-yl)-1-((2-(trimethylsilyl)ethoxy)methyl)-1H-pyrazole in Step 1. LCMS calculated for C₂₉H₂₆Cl₂N₅O₂ (M+H)+: m/z=546.1/548.1; found 546.2/548.2.

### Step 3. tert-Butyl 3-(5-(3-bromo-2-(2,6-dichlorophenyl)imidazo[2,1-f][1.6]naphthyridin-9-yl)pyridin-2-yl)azetidine-1-carboxylate

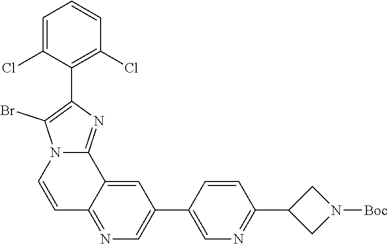 [SMILES: CC(C)(C)OC(=O)N1CC(c2ccc(-c3cnc4ccn5c(Br)c(-c6c(Cl)cccc6Cl)nc5c4c3)cn2)C1]

This compound was prepared according to the procedures described in Example 25, with tert-butyl 3-(5-(2-(2,6-dichlorophenyl)imidazo[2,1-f][1,6]naphthyridin-9-yl)pyridin-2-yl)azetidine-1-carboxylate replacing 9-(1-((2-(trimethylsilyl)ethoxy)methyl)-1H-pyrazol-4-yl)imidazo[2,1-f][1,6]naphthyridine in Step 3. LCMS calculated for C₂₉H₂₅BrCl₂N₅O₂ (M+H)⁺: m/z=624.1/626.1/628.1; Found: 624.2/626.2/628.2.

### Step 4. 9-(6-(Azetidin-3-yl)pyridin-3-yl)-2-(2,6-dichlorophenyl)-3-methylimidazo[2,1-f][1,6]naphthyridine

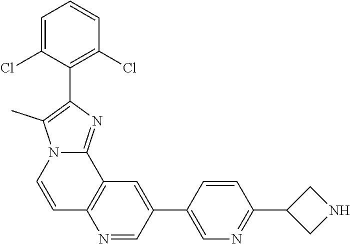 [SMILES: Cc1c(-c2c(Cl)cccc2Cl)nc2c3cc(-c4ccc(C5CNC5)nc4)cnc3ccn12]

A mixture of tert-butyl 3-(5-(3-bromo-2-(2,6-dichlorophenyl)imidazo[2,1-f][1,6]naphthyridin-9-yl)pyridin-2-yl)azetidine-1-carboxylate (500 mg, 0.800 mmol), potassium phosphate, tribasic (339 mg, 1.60 mmol), and RuPhos Pd G2 (62 mg, 0.080 mmol) was suspended in 1,4-dioxane (3.6 mL), and water (0.360 mL). The vial was purged with nitrogen for 30 sec., followed by the addition of trimethylboroxine (447 μL, 3.20 mmol). The vial was sealed and heated to 80° C. for 2 h. After cooling to room temperature, the reaction mixture was diluted with CH₂Cl₂, filtered through Celite and the filtrate was concentrated in vacuo. The resulting residue was purified by Biotage Isolera. The purified material was then dissolved in CH₂Cl₂ (4 mL) and TFA (1 mL) and stirred at r.t. for 1 h. At completion, the volatiles were removed in vacuo to give the desired product. LCMS calculated for C₂₅H₂₀Cl₂N₅ (M+H)⁺: m/z=460.1/462.1; 460.1/462.1.

### Step 5. 1-(3-(5-(2-(2,6-Dichlorophenyl)-3-methylimidazo[2,1-f][1.6]naphthyridin-9-yl)pyridin-2-yl)azetidin-1-yl)-2-hydroxyethan-1-one

To a vial containing 9-(6-(azetidin-3-yl)pyridin-3-yl)-2-(2,6-dichlorophenyl)-3-methylimidazo[2,1-f][1,6]naphthyridine (10 mg, 0.022 mmol) was added 2-hydroxyacetic acid (2 mg, 0.022 mmol), DMF (0.5 mL) and diisopropylethylamine (8 μL, 0.043 mmol). This was stirred for 1 min before HATU (13 mg, 0.033 mmol) was added and left to stir for 1 h. Upon completion, the reaction mixture was then diluted with acetonitrile and methanol and purified with prep-LCMS (XBridge C18 column, eluting with a gradient of acetonitrile/water containing 0.1% NH₄OH, at flow rate of 60 mL/min). The title compound was isolated as the TFA salt. LCMS calculated for C₂₇H₂₂Cl₂N₅O₂ (M+H)⁺: m/z=518.1/520.1; found 518.2/520.1. ¹H NMR (400 MHz, DMSO-d₆) δ 9.32 (d, J=2.4 Hz, 1H), 9.18 (d, J=2.4 Hz, 1H), 9.05 (d, J=2.4 Hz, 1H), 8.55 (d, J=7.5 Hz, 1H), 8.36 (dd, J=8.1, 2.4 Hz, 1H), 7.69-7.62 (m, 2H), 7.59-7.50 (m, 2H), 7.47 (d, J=7.2 Hz, 1H), 4.94 (t, J=6.0 Hz, 1H), 4.58 (t, J=8.3 Hz, 1H), 4.40 (dd, J=8.8, 5.3 Hz, 1H), 4.33-4.25 (m, 1H), 4.15-4.09 (m, 2H), 3.96 (d, J=6.0 Hz, 2H), 2.41 (s, 3H).

### Example 62. 1-(3-(5-(2-(2,6-Dichlorophenyl)-3-methylimidazo[2,1-f][1,6]naphthyridin-9-yl)pyridin-3-yl)azetidin-1-yl)ethan-1-one

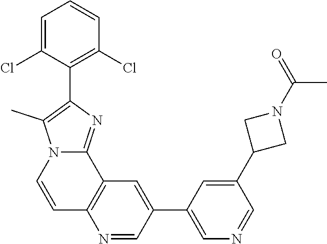 [SMILES: CC(=O)N1CC(c2cncc(-c3cnc4ccn5c(C)c(-c6c(Cl)cccc6Cl)nc5c4c3)c2)C1]

### Step 1. 9-(5-(Azetidin-3-yl)pyridin-3-yl)-2-(2,6-dichlorophenyl)-3-methylimidazo[2,1-f][1,6]naphthyridine

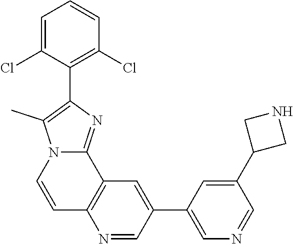 [SMILES: Cc1c(-c2c(Cl)cccc2Cl)nc2c3cc(-c4cncc(C5CNC5)c4)cnc3ccn12]

This compound was prepared according to the procedures described in Example 61, with 3,5-dibromopyridine replacing 2,5-dibromopyridine in Step 1. LCMS calculated for C₂₅H₂₀Cl₂N₅ (M+H)⁺: m/z=460.1/462.1; 460.1/462.0.

### Step 2. 1-(3-(5-(2-(2,6-Dichlorophenyl)-3-methylimidazo[2,1-f][1.6]naphthyridin-9-yl)pyridin-3-yl)azetidin-1-yl)ethan-1-one

To a vial containing 9-(5-(azetidin-3-yl)pyridin-3-yl)-2-(2,6-dichlorophenyl)-3-methylimidazo[2,1-f][1,6]naphthyridine (10 mg, 0.022 mmol) was added acetic acid (2 μL, 0.022 mmol), DMF (0.5 mL) and diisopropylethylamine (8 μL, 0.043 mmol). This was stirred for 1 min before HATU (13 mg, 0.033 mmol) was added and left to stir for 1 h. Upon completion, the reaction mixture was then diluted with acetonitrile and methanol and purified with prep-LCMS (XBridge C18 column, eluting with a gradient of acetonitrile/water containing 0.1% NH₄OH, at flow rate of 60 mL/min). The title compound was isolated as the TFA salt. LCMS calculated for C₂₇H₂₂Cl₂N₅O (M+H)⁺: m/z=502.1/504.1; Found: 502.2/504.2.

### Example 63. 1-(3-(5-(2-(2,6-Dichlorophenyl)-3-methylimidazo[2,1-f][1,6]naphthyridin-9-yl)phenyl)azetidin-1-yl)ethan-1-one

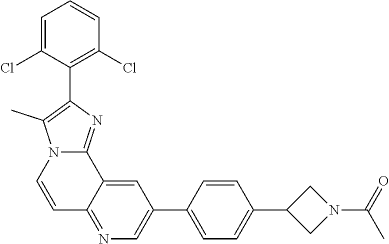 [SMILES: CC(=O)N1CC(c2ccc(-c3cnc4ccn5c(C)c(-c6c(Cl)cccc6Cl)nc5c4c3)cc2)C1]

### Step 1. tert-Butyl 3-(4-chlorophenyl)azetidine-1-carboxylate

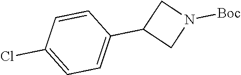 [SMILES: CC(C)(C)OC(=O)N1CC(c2ccc(Cl)cc2)C1]

A vial containing zinc(0) dust (0.346 g, 5.30 mmol), 1,2-dibromoethane (30 μL, 0.353 mmol), TMSCl (45 μL, 0.353 mmol) in THF (7.06 mL) was purged with N₂ for 30 sec., sealed and heated to 60° C. for 15 min. After which a solution of tert-butyl 3-iodoazetidine-1-carboxylate (1 g, 3.53 mmol) in DMA (3.5 mL) was added. This mixture was heated at 60° C. for another 15 min and, upon subsequent cooling to room temperature, 1-chloro-4-iodobenzene (0.842 g, 13.5 mmol), [11,11′-bis(diphenylphosphino)ferrocene]dichloropalladium(II) (0.144 g, 0.177 mmol) and CuI (34 mg, 0.177 mmol) were added. Following addition the reaction was heated to 80° C. and stirred for 4 h. Upon completion this was diluted with DCM, filtered through celite and concentrated in vacuo. The residue was purified by Biotage Isolera and concentrated in vacuo. LCMS calculated for C₁₀H₁₁ClNO₂ (M+H−t−Bu)+: m/z=212.0/214.0; found=212.1/214.1.

### Step 2. tert-Butyl 3-(4-(4,4,5,5-tetramethyl-1,3,2-dioxaborolan-2-yl)phenyl)azetidine-1-carboxylate

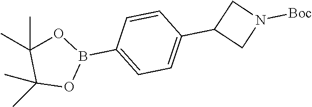 [SMILES: CC(C)(C)OC(=O)N1CC(c2ccc(B3OC(C)(C)C(C)(C)O3)cc2)C1]

To a vial was added tert-butyl 3-(4-chlorophenyl)azetidine-1-carboxylate (0.5 g, 1.867 mmol), XPhos Pd G2 (0.147 g, 0.187 mmol), bis(pinacolato)diboron (0.711 g, 2.80 mmol), potassium acetate (0.448 g, 4.67 mmol) in 1,4-dioxane (6 ml) The solution was purged with N₂ for 30 sec., sealed and heated to 80° C. for 2 h. At completion, volatiles were removed in vacuo to give the desired product, which was used directly for the next step. LCMS calculated for C₁₆H₂₃BNO₄ (M+H−t−Bu)+: m/z=304.2 found=304.1.

### Step 3. 9-(4-(Azetidin-3-yl)phenyl)-2-(2,6-dichlorophenyl)-3-methylimidazo[2,1-f][1,6]naphthyridine

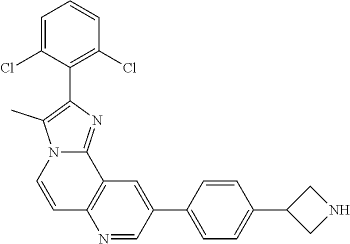 [SMILES: Cc1c(-c2c(Cl)cccc2Cl)nc2c3cc(-c4ccc(C5CNC5)cc4)cnc3ccn12]

This compound was prepared according to the procedures described in Example 61, with tert-butyl 3-(4-(4,4,5,5-tetramethyl-1,3,2-dioxaborolan-2-yl)phenyl)azetidine-1-carboxylate replacing tert-butyl 3-(5-(4,4,5,5-tetramethyl-1,3,2-dioxaborolan-2-yl)pyridin-2-yl)azetidine-1-carboxylate in Step 2. LCMS calculated for C₂₆H₂₁Cl₂N₄ (M+H)⁺: m/z=459.1/461.1; 459.1/461.1.

### Step 4. 1-(3-(4-(2-(2,6-Dichlorophenyl)-3-methylimidazo[2,1-f][1.6]naphthyridin-9-yl)phenyl)azetidin-1-yl)ethan-1-one

This compound was prepared according to the procedures described in Example 62 step 2, with 9-(4-(azetidin-3-yl)phenyl)-2-(2,6-dichlorophenyl)-3-methylimidazo[2,1-f][1,6]naphthyridine replacing 9-(5-(azetidin-3-yl)pyridin-3-yl)-2-(2,6-dichlorophenyl)-3-methylimidazo[2,1-f][1,6]naphthyridine. The title compound was isolated as the TFA salt. LCMS calculated for C₂₈H₂₃Cl₂N₄O (M+H)⁺: m/z=501.1/503.1; Found: 501.2/503.2

### Example 64. (S)-1-(3-(4-(2-(2-Chlorophenyl)-3-(hydroxymethyl)imidazo[2,1-f][1,6]naphthyridin-9-yl)-1H-pyrazol-1-yl)pyrrolidin-1-yl)ethan-1-one

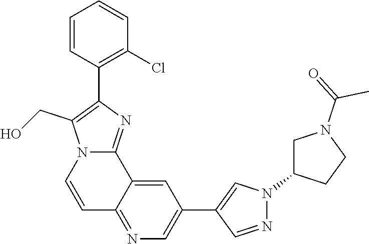 [SMILES: CC(=O)N1CC[C@H](n2cc(-c3cnc4ccn5c(CO)c(-c6ccccc6Cl)nc5c4c3)cn2)C1]

### Step 1. (S)-(2-(2-Chlorophenyl)-9-(1-(pyrrolidin-3-yl)-1H-pyrazol-4-yl)imidazo[2,1-f][1,6]naphthyridin-3-yl)methanol

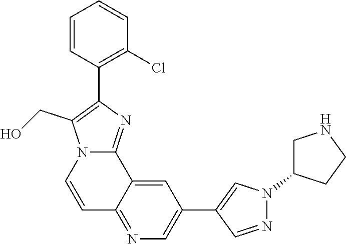 [SMILES: OCc1c(-c2ccccc2Cl)nc2c3cc(-c4cnn([C@H]5CCNC5)c4)cnc3ccn12]

This compound was prepared according to the procedures described in Example 55, with 2-bromo-1-(2-chlorophenyl)ethan-1-one replacing 2-bromo-1-(2,6-dichlorophenyl)ethan-1-one in Step 2. LCMS calculated for C₂₄H₂₂Cl N₆O (M+H)⁺: m/z=445.2/447.2; Found: 445.1/447.1.

### Step 2. (S)-1-(3-(4-(2-(2-Chlorophenyl)-3-(hydroxymethyl)imidazo[2,1-f][1,6]naphthyridin-9-yl)-1H-pyrazol-1-yl)pyrrolidin-1-yl)ethan-1-one

To a vial containing (S)-(2-(2-chlorophenyl)-9-(1-(pyrrolidin-3-yl)-1H-pyrazol-4-yl)imidazo[2,1-f][1,6]naphthyridin-3-yl)methanol (10 mg, 0.022 mmol) was added acetic acid (2 μL, 0.022 mmol), DMF (0.5 mL) and DIPEA (8 μL, 0.045 mmol). This was stirred for 1 min before HATU (13 mg, 0.034 mmol) was added and left to stir for 1 h. Upon completion the reaction mixture was then diluted with acetonitrile/methanol and purified with prep-LCMS (XBridge C18 column, eluting with a gradient of acetonitrile/water containing 0.1% TFA, at flow rate of 60 mL/min). The title compound was isolated as the TFA salt. LCMS calculated for C₂₆H₂₄Cl N₆O₂ (M+H)⁺: m/z=487.2/489.2; Found: 487.1/489.2.

### Example 65. (S)-1-(3-(4-(2-(2-Chlorophenyl)-3-isopropylimidazo[2,1-f][1,6]naphthyridin-9-yl)-1H-pyrazol-1-yl)pyrrolidin-1-yl)-2-hydroxyethan-1-one

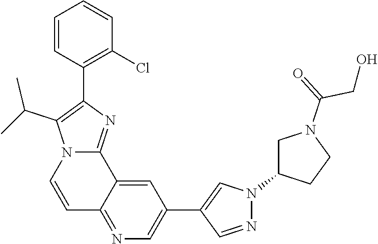 [SMILES: CC(C)c1c(-c2ccccc2Cl)nc2c3cc(-c4cnn([C@H]5CCN(C(=O)CO)C5)c4)cnc3ccn12]

### Step 1. tert-Butyl (S)-3-(4-(2-(2-chlorophenyl)-3-(prop-1-en-2-yl)imidazo[2,1-f][1,6]naphthyridin-9-yl)-1H-pyrazol-1-yl)pyrrolidine-1-carboxylate

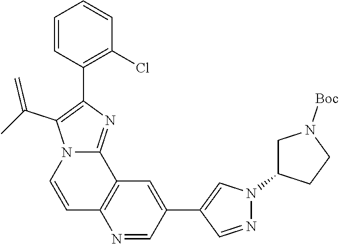 [SMILES: C=C(C)c1c(-c2ccccc2Cl)nc2c3cc(-c4cnn([C@H]5CCN(C(=O)OC(C)(C)C)C5)c4)cnc3ccn12]

This compound was prepared according to the procedures described in Examples 55 and 58, with 2-bromo-1-(2-chlorophenyl)ethan-1-one replacing 2-bromo-1-(2,6-dichlorophenyl)ethan-1-one in Example 55, Step 2. LCMS calculated for C₃₁H₃₂Cl N₆O₂ (M+H)⁺: m/z=555.2/557.2; Found: 555.2/557.2.

### Step 2. (S)-2-(2-Chlorophenyl)-3-isopropyl-9-(1-(pyrrolidin-3-yl)-1H-pyrazol-4-yl)imidazo[2,1-f][1,6]naphthyridine

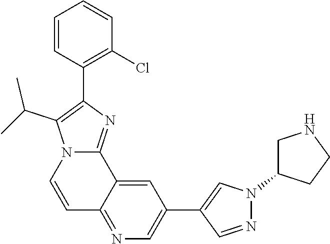 [SMILES: CC(C)c1c(-c2ccccc2Cl)nc2c3cc(-c4cnn([C@H]5CCNC5)c4)cnc3ccn12]

A vial containing tert-butyl (S)-3-(4-(2-(2-chlorophenyl)-3-(prop-1-en-2-yl)imidazo[2,1-f][1,6]naphthyridin-9-yl)-1H-pyrazol-1-yl)pyrrolidine-1-carboxylate (60 mg, 0.108 mmol) and palladium hydroxide (20 wt %, 12 mg, 0.017 mmol) as a solution in ethanol (540 μL) was purged with hydrogen gas for 1 min and left to stir under an atmosphere of hydrogen gas (balloon pressure) for 18 h. Upon completion the reaction mixture was diluted with CH₂Cl₂, filtered through celite and the filtrate was concentrated in vacuo. The resulting residue was dissolved in CH₂Cl₂ (1 mL) and TFA (0.5 mL) and stirred at r.t. for 1 h. At completion, the volatiles were removed in vacuo to give the desired product. LCMS calculated for C₂₆H₂₆Cl N₆ (M+H)⁺: m/z=457.2/459.2; Found: 457.4/459.4.

### Step 3. (S)-1-(3-(4-(2-(2-Chlorophenyl)-3-isopropylimidazo[2,1-f][1,6]naphthyridin-9-yl)-1H-pyrazol-1-yl)pyrrolidin-1-yl)-2-hydroxyethan-1-one

This compound was prepared according to the procedures described in Example 25, with (S)-2-(2-chlorophenyl)-3-isopropyl-9-(1-(pyrrolidin-3-yl)-1H-pyrazol-4-yl)imidazo[2,1-f][1,6]naphthyridine replacing (S)-2-(2-chloro-6-(trifluoromethyl)phenyl)-3-methyl-9-(1-(pyrrolidin-3-yl)-1H-pyrazol-4-yl)imidazo[2,1-f][1,6]naphthyridine in Step 8. The title compound was isolated as the TFA salt. LCMS calculated for C₂₈H₂₈Cl N₆O₂ (M+H)+: m/z=515.2/517.2; found 515.3/517.3.

### Example 66. (S)-1-(3-(4-(3-Bromo-2-(2-chloro-6-fluorophenyl)imidazo[2,1-f][1,6]naphthyridin-9-yl)-1H-pyrazol-1-yl)pyrrolidin-1-yl)-2-hydroxyethan-1-one

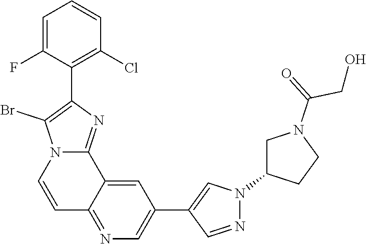 [SMILES: O=C(CO)N1CC[C@H](n2cc(-c3cnc4ccn5c(Br)c(-c6c(F)cccc6Cl)nc5c4c3)cn2)C1]

### Step 1. tert-Butyl (S)-3-(4-(3-bromo-2-(2-chloro-6-fluorophenyl)imidazo[2,1-f][1,6]naphthyridin-9-yl)-1H-pyrazol-1-yl)pyrrolidine-1-carboxylate

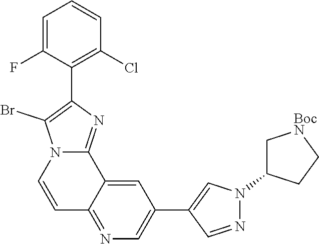 [SMILES: CC(C)(C)OC(=O)N1CC[C@H](n2cc(-c3cnc4ccn5c(Br)c(-c6c(F)cccc6Cl)nc5c4c3)cn2)C1]

This compound was prepared according to the procedures described in Example 56, Steps 1 through 4, with 2-bromo-1-(2-chloro-6-fluorophenyl)ethan-1-one replacing 2-bromo-1-(2,6-dichlorophenyl)ethan-1-one in Step 2. LCMS calculated for C₂₈H₂₆BrClFN₆O₂ (M+H)⁺: m/z=611.1/613; found 611.2/613.3.

### Step 2. (S)-1-(3-(4-(3-Bromo-2-(2-chloro-6-fluorophenyl)imidazo[2,1-f][1,6]naphthyridin-9-yl)-1H-pyrazol-1-yl)pyrrolidin-1-yl)-2-hydroxyethan-1-one

tert-Butyl (S)-3-(4-(3-bromo-2-(2-chloro-6-fluorophenyl)imidazo[2,1-f][1,6]naphthyridin-9-yl)-1H-pyrazol-1-yl)pyrrolidine-1-carboxylate (15 mg, 0.024 mmol) was dissolved in CH₂Cl₂ (1 mL) and TFA (0.5 mL) and stirred at r.t. for 1 hr. At completion, the volatiles were removed in vacuo to give (S)-3-bromo-2-(2-chloro-6-fluorophenyl)-9-(1-(pyrrolidin-3-yl)-1H-pyrazol-4-yl)imidazo[2,1-f][1,6]naphthyridine (10 mg, 0.022 mmol), to which was added 2-hydroxyacetic acid (1.7 mg, 0.022 mmol), DMF (0.5 mL) and diisopropylethylamine (8 μL, 0.043 mmol). The reaction mixture was stirred for 1 min before HATU (12.4 mg, 0.147 mmol) was added and left to stir at r.t. for 1 h. Upon completion the reaction mixture was then diluted with acetonitrile and methanol and purified with prep-LCMS (XBridge C18 column, eluting with a gradient of acetonitrile/water containing 0.1% TFA, at flow rate of 60 mL/min). The title compound was isolated as the TFA salt. LCMS calculated for C₂₅H₂₀BrClFN₆O₂ (M+H)⁺: m/z=569.1/571.0; found 569.2/571.2.

### Example 67. 2-Cyclopropyl-3-methyl-9-(1-methyl-1H-pyrazol-4-yl)imidazo[2,1-f][1,6]naphthyridine

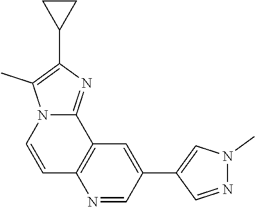

### Step 1. 2-Cyclopropyl-3-methyl-9-(1H-pyrazol-4-yl)imidazo[2,1-f][1,6]naphthyridine

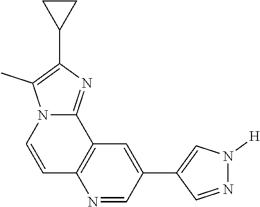 [SMILES: Cc1c(C2CC2)nc2c3cc(-c4cn[nH]c4)cnc3ccn12]

This compound was prepared according to the procedures described in Examples 37 and 55, with 2-bromo-1-cyclopropylethan-1-one replacing 2-bromo-1-(2,6-dichlorophenyl)ethan-1-one in Example 55, Step 2. LCMS calculated for C₁₇H₁₆N₅ (M+H)⁺: m/z=290.1; found 290.1.

### Step 2. 2-Cyclopropyl-3-methyl-9-(1-methyl-1H-pyrazol-4-yl)imidazo[2,1-f][1,6]naphthyridine

A vial containing a mixture of 2-cyclopropyl-3-methyl-9-(1H-pyrazol-4-yl)imidazo[2,1-f][1,6]naphthyridine (20 mg, 0.069 mmol), methyl iodide (2M in tert-butyl methyl ether, 38 μL, 0.076 mmol), cesium carbonate (34 mg, 0.104 mmol), in acetonitrile (0.346 mL) was heated to 40° C. Upon completion, the reaction mixture was diluted with CH₂Cl₂ and filtered through Celite, followed by concentration of the filtrate in vacuo. The residue was diluted with acetonitrile and methanol and purified with prep-LCMS (XBridge C18 column, eluting with a gradient of acetonitrile/water containing 0.1% TFA, at flow rate of 60 mL/min). The title compound was isolated as the TFA salt. LCMS calculated for C₁₈H₁₈N₅ (M+H)+: m/z=304.2; found 304.1.

### Example 68. (2-(2,6-Dichlorophenyl)-9-(1-methyl-1H-pyrazol-4-yl)imidazo[2,1-f][1,6]naphthyridin-3-yl)methanol

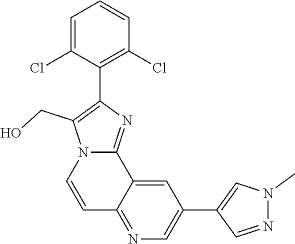 [SMILES: Cn1cc(-c2cnc3ccn4c(CO)c(-c5c(Cl)cccc5Cl)nc4c3c2)cn1]

### Step 1. 9-Bromo-2-(2,6-dichlorophenyl)-3-iodoimidazo[2,1-f][1,6]naphthyridine

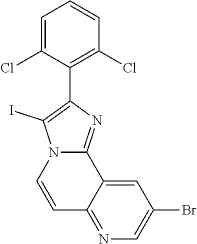 [SMILES: Clc1cccc(Cl)c1-c1nc2c3cc(Br)cnc3ccn2c1I]

A vial containing 9-bromo-2-(2,6-dichlorophenyl)imidazo[2,1-f][1,6]naphthyridine (Example 15, Step 1, 200 mg, 0.509 mmol), NIS (114 mg, 0.509 mmol) in acetonitrile (2 mL) was stirred at 60° C. for 4 h. The solution was subsequently cooled to room temperature, concentrated in vacuo and purified by Biotage Isolera to give the desired product as a brown solid. LCMS calculated for C₁₆H₈BrCl₂IN₃ (M+H)⁺: m/z=517.8/519.8; found 517.9/519.7.

### Step 2. 9-Bromo-2-(2,6-dichlorophenyl)-3-vinylimidazo[2,1-f][1,6]naphthyridine

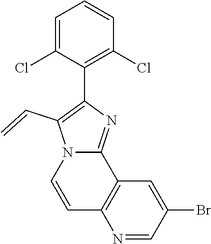 [SMILES: C=Cc1c(-c2c(Cl)cccc2Cl)nc2c3cc(Br)cnc3ccn12]

A vial containing 9-bromo-2-(2,6-dichlorophenyl)-3-iodoimidazo[2,1-f][1,6]naphthyridine (150 mg, 0.289 mmol), potassium phosphate, tribasic (123 mg, 0.578 mmol), tetrakis(triphenylphosphine)palladium(0) (33 mg, 0.029 mmol) and 4,4,5,5-tetramethyl-2-vinyl-1,3,2-dioxaborolane (49 μL, 0.289 mmol) was suspended in 1,4-dioxane (2 mL), and water (200 μL). This was purged with nitrogen for 30 sec. and heated to 70° C. for 2 h. Upon cooling to room temperature, the solution was diluted with CH₂Cl₂, filtered through Celite and the filtrate was concentrated in vacuo. The resulting residue was purified by Biotage Isolera to give the desired product as a brown solid. LCMS calculated for C₁₈H₁₁BrCl₂N₃ (M+H)⁺: m/z=418.0/419.9; found 418.1/420.1.

### Step 3. (9-Bromo-2-(2,6-dichlorophenyl)imidazo[2,1-f][1,6]naphthyridin-3-yl)methanol

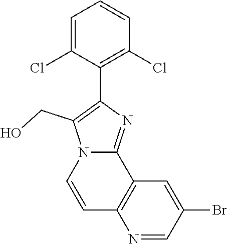 [SMILES: OCc1c(-c2c(Cl)cccc2Cl)nc2c3cc(Br)cnc3ccn12]

To a vial was added 9-bromo-2-(2,6-dichlorophenyl)-3-vinylimidazo[2,1-f][1,6]naphthyridine (80 mg, 0.191 mmol), THF (3 mL), water (1 mL) and osmium tetroxide (4 wt. % in H₂O, 75 μL, 9.54 μmol), followed by sodium periodate (204 mg, 0.954 mmol). The reaction mixture was stirred at room temperature for 3 h, and upon completion the reaction was quenched with saturated aqueous Na₂S₂O₃ and extracted into EtOAc. The combined organic layers were concentrated in vacuo and the residue was dissolved in isopropanol (4 mL), cooled to 0° C., and NaBH₄ (7.22 mg, 0.191 mmol) was added with stirring while allowing the reaction to slowly warm to room temperature. The reaction was then cooled to 0° C. and quenched by addition of saturated aqueous NH₄Cl. The volatiles were removed in vacuo and the residue was extracted into 20:1 CH₂Cl₂/MeOH. The resulting organic layers were combined and concentrated in vacuo. The obtained product was used in the next step without further purification. LCMS calculated for C₁₇H₁₁BrCl₂N₃O (M+H)⁺: m/z=421.9/423.9; found 421.9/424.0.

### Step 4. (2-(2,6-Dichlorophenyl)-9-(1-methyl-1H-pyrazol-4-yl)imidazo[2,1-f][1,6]naphthyridin-3-yl)methanol

To a vial was added (9-bromo-2-(2,6-dichlorophenyl)imidazo[2,1-f][1,6]naphthyridin-3-yl)methanol (20 mg, 0.047 mmol), 1-methyl-4-(4,4,5,5-tetramethyl-1,3,2-dioxaborolan-2-yl)-1H-pyrazole (10 mg, 0.047 mmol), potassium phosphate, tribasic (30 mg, 0.142 mmol), and (1,1′-bis(diphenylphosphino)ferrocene)dichloropalladium(II) (3 mg, 4.73 μmol) in 1,4-dioxane (1 mL) and water (100 μL). This was purged with nitrogen for 1 min, sealed and heated to 80° C. for 30 min. Upon completion the reaction mixture was cooled to room temperature and flushed through a SiliaPrep SPE thiol cartridge (SPE-R51030B-06P), diluted with acetonitrile/methanol and purified with prep-LCMS (XBridge C18 column, eluting with a gradient of acetonitrile/water containing 0.1% TFA, at flow rate of 60 mL/min). The title compound was isolated as the TFA salt. LCMS calculated for C₂₁H₁₆Cl₂N₅O (M+H)⁺: m/z=424.1/426.1; found 424.0/426.1.

### Example 69. (9-(1-Cyclopropyl-1H-pyrazol-4-yl)-2-(2,6-dichlorophenyl)imidazo[2,1-f][1,6]naphthyridin-3-yl)methanol

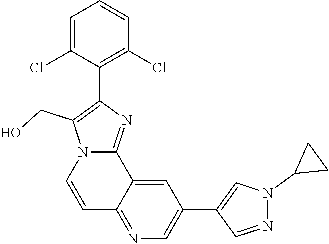 [SMILES: OCc1c(-c2c(Cl)cccc2Cl)nc2c3cc(-c4cnn(C5CC5)c4)cnc3ccn12]

This compound was prepared according to the procedures described in Example 68, with 1-cyclopropyl-4-(4,4,5,5-tetramethyl-1,3,2-dioxaborolan-2-yl)-1H-pyrazole replacing 1-methyl-4-(4,4,5,5-tetramethyl-1,3,2-dioxaborolan-2-yl)-1H-pyrazole in Step 4. The title compound was isolated as the TFA salt. LCMS calculated for C₂₃H₁₈Cl₂N₅O (M+H)⁺: m/z=450.1/452.1; Found: 450.1/452.0.

### Example 70. (2-(2,6-Dichlorophenyl)-9-(1-(tetrahydrofuran-3-yl)-1H-pyrazol-4-yl)imidazo[2,1-f][1,6]naphthyridin-3-yl)methanol

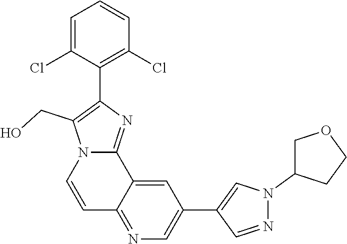 [SMILES: OCc1c(-c2c(Cl)cccc2Cl)nc2c3cc(-c4cnn(C5CCOC5)c4)cnc3ccn12]

This compound was prepared according to the procedures described in Example 68, with 1-(tetrahydrofuran-3-yl)-4-(4,4,5,5-tetramethyl-1,3,2-dioxaborolan-2-yl)-1H-pyrazole replacing 1-methyl-4-(4,4,5,5-tetramethyl-1,3,2-dioxaborolan-2-yl)-1H-pyrazole in Step 4. The title compound was isolated as the TFA salt. LCMS calculated for C₂₄H₂₀Cl₂N₅O₂ (M+H)⁺: m/z=480.1/482.1; Found: 480.1/482.0.

### Example 71. (2-(2,6-Dichlorophenyl)-9-(1-(oxetan-3-yl)-1H-pyrazol-4-yl)imidazo[2,1-f][1,6]naphthyridin-3-yl)methanol

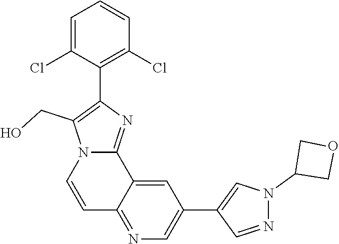 [SMILES: OCc1c(-c2c(Cl)cccc2Cl)nc2c3cc(-c4cnn(C5COC5)c4)cnc3ccn12]

This compound was prepared according to the procedures described in Example 68, with 1-(oxetan-3-yl)-4-(4,4,5,5-tetramethyl-1,3,2-dioxaborolan-2-yl)-1H-pyrazole replacing 1-methyl-4-(4,4,5,5-tetramethyl-1,3,2-dioxaborolan-2-yl)-1H-pyrazole in Step 4. The title compound was isolated as the TFA salt. LCMS calculated for C₂₃H₁₈Cl₂N₅O₂ (M+H)⁺: m/z=466.1/468.1; Found: 466.0/468.0.

### Example 72. (2-(2,6-Dichlorophenyl)-9-(1-(2-(dimethylamino)ethyl)-1H-pyrazol-4-yl)imidazo[2,1-f][1,6]naphthyridin-3-yl)methanol

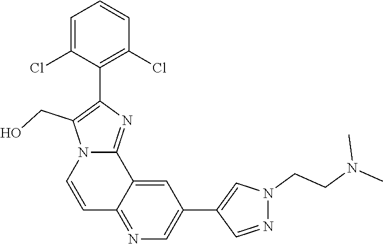 [SMILES: CN(C)CCn1cc(-c2cnc3ccn4c(CO)c(-c5c(Cl)cccc5Cl)nc4c3c2)cn1]

This compound was prepared according to the procedures described in Example 68, with N,N-dimethyl-2-(4-(4,4,5,5-tetramethyl-1,3,2-dioxaborolan-2-yl)-1H-pyrazol-1-yl)ethan-1-amine replacing 1-methyl-4-(4,4,5,5-tetramethyl-1,3,2-dioxaborolan-2-yl)-1H-pyrazole in Step 4. The title compound was isolated as the TFA salt. LCMS calculated for C₂₄H₂₃Cl₂N₆O (M+H)⁺: m/z=481.1/483.1; Found: 481.1/483.1.

### Example 73. (2-(2,6-Dichlorophenyl)-9-(1-isopropyl-1H-pyrazol-4-yl)imidazo[2,1-f][1,6]naphthyridin-3-yl)methanol

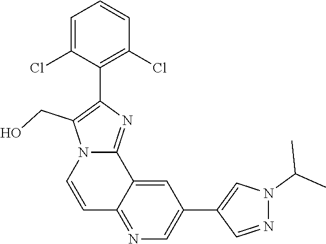 [SMILES: CC(C)n1cc(-c2cnc3ccn4c(CO)c(-c5c(Cl)cccc5Cl)nc4c3c2)cn1]

This compound was prepared according to the procedures described in Example 68, with 1-isopropyl-4-(4,4,5,5-tetramethyl-1,3,2-dioxaborolan-2-yl)-1H-pyrazole replacing 1-methyl-4-(4,4,5,5-tetramethyl-1,3,2-dioxaborolan-2-yl)-1H-pyrazole in Step 4. The title compound was isolated as the TFA salt. LCMS calculated for C₂₃H₂₀Cl₂N₅O (M+H)⁺: m/z=452.1/454.1; Found: 452.1/454.0.

### Example 74. (2-(2,6-Dichlorophenyl)-9-(1-(difluoromethyl)-1H-pyrazol-4-yl)imidazo[2,1-f][1,6]naphthyridin-3-yl)methanol

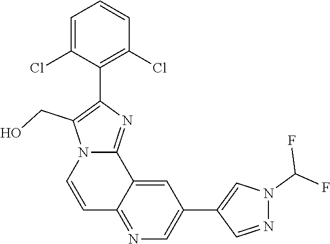 [SMILES: OCc1c(-c2c(Cl)cccc2Cl)nc2c3cc(-c4cnn(C(F)F)c4)cnc3ccn12]

This compound was prepared according to the procedures described in Example 68, with 1-(difluoromethyl)-4-(4,4,5,5-tetramethyl-1,3,2-dioxaborolan-2-yl)-1H-pyrazole replacing 1-methyl-4-(4,4,5,5-tetramethyl-1,3,2-dioxaborolan-2-yl)-1H-pyrazole in Step 4. The title compound was isolated as the TFA salt. LCMS calculated for C₂₁H₁₄Cl₂F₂N₅O (M+H)⁺: m/z=460.1/462.1; Found: 460.0/462.0.

### Example 75. 3-(4-(2-(2,6-Dichlorophenyl)-3-(hydroxymethyl)imidazo[2,1-f][1,6]naphthyridin-9-yl)-1H-pyrazol-1-yl)propanenitrile

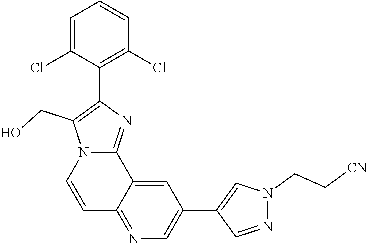 [SMILES: N#CCCn1cc(-c2cnc3ccn4c(CO)c(-c5c(Cl)cccc5Cl)nc4c3c2)cn1]

This compound was prepared according to the procedures described in Example 68, with 3-(4-(4,4,5,5-tetramethyl-1,3,2-dioxaborolan-2-yl)-1H-pyrazol-1-yl)propanenitrile replacing 1-methyl-4-(4,4,5,5-tetramethyl-1,3,2-dioxaborolan-2-yl)-1H-pyrazole in Step 4. The title compound was isolated as the TFA salt. LCMS calculated for C₂₃H₁₇Cl₂N₆O (M+H)⁺: m/z=463.1/465.1; Found: 463.1/465.0.

### Example 76. 1-(4-(2-(2,6-Dichlorophenyl)-3-(hydroxymethyl)imidazo[2,1-f][1,6]naphthyridin-9-yl)-1H-pyrazol-1-yl)-2-methylpropan-2-ol

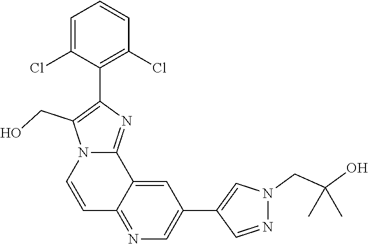 [SMILES: CC(C)(O)Cn1cc(-c2cnc3ccn4c(CO)c(-c5c(Cl)cccc5Cl)nc4c3c2)cn1]

This compound was prepared according to the procedures described in Example 68, with 2-methyl-1-(4-(4,4,5,5-tetramethyl-1,3,2-dioxaborolan-2-yl)-1H-pyrazol-1-yl)propan-2-ol replacing 1-methyl-4-(4,4,5,5-tetramethyl-1,3,2-dioxaborolan-2-yl)-1H-pyrazole in Step 4. The title compound was isolated as the TFA salt. LCMS calculated for C₂₄H₂₂Cl₂N₅O₂ (M+H)⁺: m/z=482.1/484.1; Found: 482.1/484.0.

### Example 77. 2-(4-(2-(2,6-Dichlorophenyl)-3-(hydroxymethyl)imidazo[2,1-f][1,6]naphthyridin-9-yl)-1H-pyrazol-1-yl)acetonitrile

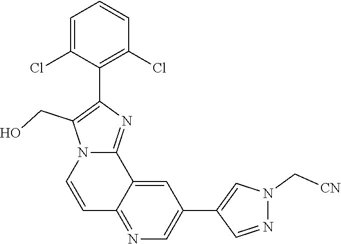 [SMILES: N#CCn1cc(-c2cnc3ccn4c(CO)c(-c5c(Cl)cccc5Cl)nc4c3c2)cn1]

This compound was prepared according to the procedures described in Example 68, with 2-(4-(4,4,5,5-tetramethyl-1,3,2-dioxaborolan-2-yl)-1H-pyrazol-1-yl)acetonitrile replacing 1-methyl-4-(4,4,5,5-tetramethyl-1,3,2-dioxaborolan-2-yl)-1H-pyrazole in Step 4. The title compound was isolated as the TFA salt. LCMS calculated for C₂₂H₁₅Cl₂N₆O (M+H)⁺: m/z=449.1/451.1; Found: 449.2/451.2.

### Example 78. (2-(2,6-Dichlorophenyl)-9-(1-(1-methylpiperidin-4-yl)-1H-pyrazol-4-yl)imidazo[2,1-f][1,6]naphthyridin-3-yl)methanol

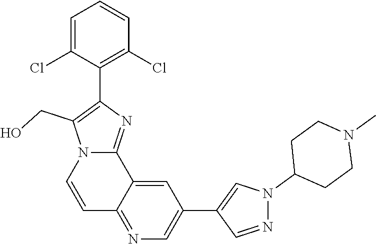 [SMILES: CN1CCC(n2cc(-c3cnc4ccn5c(CO)c(-c6c(Cl)cccc6Cl)nc5c4c3)cn2)CC1]

This compound was prepared according to the procedures described in Example 68, with 1-methyl-4-(4-(4,4,5,5-tetramethyl-1,3,2-dioxaborolan-2-yl)-1H-pyrazol-1-yl)piperidine replacing 1-methyl-4-(4,4,5,5-tetramethyl-1,3,2-dioxaborolan-2-yl)-1H-pyrazole in Step 4. The title compound was isolated as the TFA salt. LCMS calculated for C₂₆H₂₅Cl₂N₆O (M+H)⁺: m/z=507.1/509.1; Found: 507.3/509.2.

### Example 79. 2-(4-(2-(2,6-Dichlorophenyl)-3-(hydroxymethyl)imidazo[2,1-f][1,6]naphthyridin-9-yl)-1H-pyrazol-1-yl)-2-methylpropan-1-ol

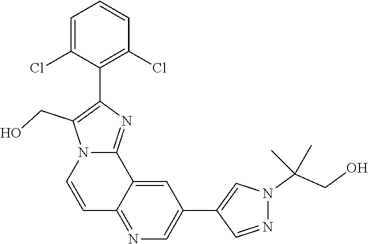 [SMILES: CC(C)(CO)n1cc(-c2cnc3ccn4c(CO)c(-c5c(Cl)cccc5Cl)nc4c3c2)cn1]

This compound was prepared according to the procedures described in Example 68, with 2-methyl-2-(4-(4,4,5,5-tetramethyl-1,3,2-dioxaborolan-2-yl)-1H-pyrazol-1-yl)propan-1-ol replacing 1-methyl-4-(4,4,5,5-tetramethyl-1,3,2-dioxaborolan-2-yl)-1H-pyrazole in Step 4. The title compound was isolated as the TFA salt. LCMS calculated for C₂₄H₂₂Cl₂N₅O₂ (M+H)⁺: m/z=482.1/484.1; Found: 482.3/484.3.

### Example 80. (2-(2,6-Dichlorophenyl)-9-(1-(2-(methylsulfonyl)ethyl)-1H-pyrazol-4-yl)imidazo[2,1-f][1,6]naphthyridin-3-yl)methanol

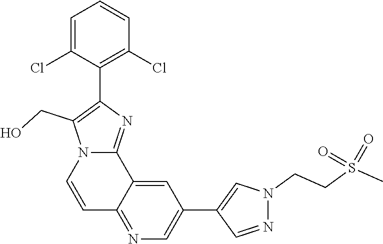 [SMILES: CS(=O)(=O)CCn1cc(-c2cnc3ccn4c(CO)c(-c5c(Cl)cccc5Cl)nc4c3c2)cn1]

This compound was prepared according to the procedures described in Example 68, with 1-(2-(methylsulfonyl)ethyl)-4-(4,4,5,5-tetramethyl-1,3,2-dioxaborolan-2-yl)-1H-pyrazole replacing 1-methyl-4-(4,4,5,5-tetramethyl-1,3,2-dioxaborolan-2-yl)-1H-pyrazole in Step 4. The title compound was isolated as the TFA salt. LCMS calculated for C₂₃H₂₀Cl₂N₅O₃S (M+H)⁺: m/z=516.1/518.1; Found: 516.1/518.1.

### Example 81. (2-(2,6-Dichlorophenyl)-9-(1-(2,2,2-trifluoroethyl)-1H-pyrazol-4-yl)imidazo[2,1-f][1,6]naphthyridin-3-yl)methanol

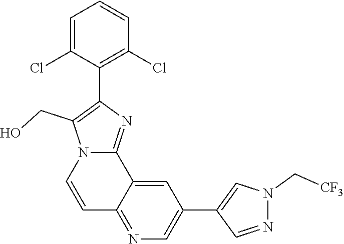 [SMILES: OCc1c(-c2c(Cl)cccc2Cl)nc2c3cc(-c4cnn(CC(F)(F)F)c4)cnc3ccn12]

This compound was prepared according to the procedures described in Example 68, with 4-(4,4,5,5-tetramethyl-1,3,2-dioxaborolan-2-yl)-1-(2,2,2-trifluoroethyl)-1H-pyrazole replacing 1-methyl-4-(4,4,5,5-tetramethyl-1,3,2-dioxaborolan-2-yl)-1H-pyrazole in Step 4. The title compound was isolated as the TFA salt. LCMS calculated for C₂₂H₁₅Cl₂F₃N₅O (M+H)⁺: m/z=492.1/494.1; Found: 492.2/494.2.

### Example 82. (2-(2,6-Dichlorophenyl)-9-(1-(2-morpholinoethyl)-1H-pyrazol-4-yl)imidazo[2,1-f][1,6]naphthyridin-3-yl)methanol

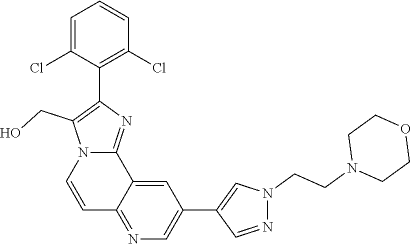 [SMILES: OCc1c(-c2c(Cl)cccc2Cl)nc2c3cc(-c4cnn(CCN5CCOCC5)c4)cnc3ccn12]

This compound was prepared according to the procedures described in Example 68, with 4-(2-(4-(4,4,5,5-tetramethyl-1,3,2-dioxaborolan-2-yl)-1H-pyrazol-1-yl)ethyl)morpholine replacing 1-methyl-4-(4,4,5,5-tetramethyl-1,3,2-dioxaborolan-2-yl)-1H-pyrazole in Step 4. The title compound was isolated as the TFA salt. LCMS calculated for C₂₆H₂₅Cl₂N₆O₂ (M+H)⁺: m/z=523.1/525.1; Found: 523.2/525.2.

### Example 83. 2-(4-(2-(2,6-Dichlorophenyl)-3-(hydroxymethyl)imidazo[2,1-f][1,6]naphthyridin-9-yl)-1H-pyrazol-1-yl)ethan-1-ol

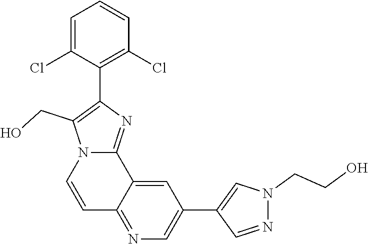 [SMILES: OCCn1cc(-c2cnc3ccn4c(CO)c(-c5c(Cl)cccc5Cl)nc4c3c2)cn1]

This compound was prepared according to the procedures described in Example 68, with 2-(4-(4,4,5,5-tetramethyl-1,3,2-dioxaborolan-2-yl)-1H-pyrazol-1-yl)ethan-1-ol replacing 1-methyl-4-(4,4,5,5-tetramethyl-1,3,2-dioxaborolan-2-yl)-1H-pyrazole in Step 4. The title compound was isolated as the TFA salt. LCMS calculated for C₂₂H₁₈Cl₂N₅O₂ (M+H)⁺: m/z=454.1/456.1; Found: 454.2/456.2.

### Example A: FGFR Enzymatic Assay

The inhibitor potency of the exemplified compounds was determined in an enzyme discontinuous assay that measures peptide phosphorylation using FRET measurements to detect product formation. Inhibitors were serially diluted in DMSO and a volume of 0.2 μL was transferred to the wells of a 384-well plate. A 5 μL/well volume of enzyme isoforms of FGFR (−1, −2, −3 wild-type and mutant isoforms, −4) including phosphorylated and un-phosphorylated proteins diluted in assay buffer (50 mM HEPES, 10 mM MgCl₂, 1 mM EGTA, 0.01% Tween-20, 5 mM DTT, pH 7.5) was added to the plate and pre-incubated with inhibitor for 5 to 15 minutes at ambient temperature. Appropriate controls (enzyme blank and enzyme with no inhibitor) were included on the plate. The reaction was initiated by the addition of a 5 μL/well volume containing both biotinylated EQEDEPEGDYFEWLE peptide substrate (SEQ ID NO: 1) and ATP in assay buffer. The 10 μL/well reaction concentration of the peptide substrate was 500 nM whereas the ATP concentration was maintained near or below the ATP Km. The ATP Km values were pre-determined in a separate series of experiments. The reaction plate was incubated at 25° C. for 1 hr and the reactions were ended with the addition of 5 μL/well of quench solution (50 mM Tris, 150 mM NaCl, 0.5 mg/mL BSA, pH 7.8; 45 mM EDTA, 600 nM staurosporin, with Perkin Elmer Lance Reagents at 3.75 nM Eu-antibody PY20 and 180 nM APC-Streptavidin). The plate was allowed to equilibrate for ˜10 minutes at ambient temperature before scanning on a PheraStar plate reader (BMG Labtech) instrument.

Either GraphPad prism or XLfit was used to analyze the data. The IC₅₀ values were derived by fitting the data to a four parameter logistic equation producing a sigmoidal dose-response curve with a variable Hill coefficient. Prism equation: Y=Bottom+(Top−Bottom)/(1+10\hat{ }((LogIC₅₀−X)*Hill slope)); XLfit equation: Y=(A+((B−A)/(1+((X/C)\hat{ }D)))) where X is the logarithm of inhibitor concentration and Y is the response. Compounds having an IC₅₀ of 1 μM or less are considered active.

Table 1 provides IC₅₀ data for compounds of the disclosure assayed in the FGFR Enzymatic Assay after dilution in assay buffer, added to the plate and pre-incubated for 4 hours. The symbol: “+” indicates an IC₅₀ less than 10 nM; “++” indicates an IC₅₀ greater than or equal to 10 nM but less than 100 nM; “+++” indicates an IC₅₀ greater than or equal to 100 nM but less than 400 nM; and “++++” indicates an IC₅₀ greater than or equal to 400 nM.

The data in Table 1 was measured in wild-type un-phosphorylated FGFR3 protein.

TABLE 1

| Example | FGFR3_Ki, nM |
|---|---|
| 1 | ++ |
| 2 | +++ |
| 3 | ++ |
| 4 | + |
| 5 | +++ |
| 6 | ++ |
| 7 | +++ |
| 8 | + |
| 9 | ++ |
| 10 | ++ |
| 11 | ++ |
| 12 | ++ |
| 13 | + |
| 14 | ++ |
| 15 | + |
| 16 | + |
| 17 | + |
| 18 | ++ |
| 19 | +++ |
| 20 | +++ |
| 21 | + |
| 22 | + |
| 23 | ++++ |
| 24 | ++++ |
| 25 | + |
| 26 | + |
| 27 | ++ |
| 28 | ++ |
| 29 | ++ |
| 30 | ++ |
| 31 | ++ |
| 32 | +++ |
| 33 | ++ |
| 34 | + |
| 35 | ++ |
| 36 | +++ |
| 37 | + |
| 38 | + |
| 39 | + |
| 40 | + |
| 41 | + |
| 42 | + |
| 43 | ++ |
| 44 | + |
| 45 | + |
| 46 | + |
| 47 | + |
| 48 | + |
| 49 | + |
| 50 | + |
| 51 | + |
| 52 | + |
| 53 | + |
| 54 | + |
| 55 | + |
| 56 | + |
| 57 | + |
| 58 | + |
| 59 | + |
| 60 | + |
| 61 | ++ |
| 62 | ++ |
| 63 | + |
| 64 | + |
| 65 | ++ |
| 66 | + |
| 67 | +++ |
| 68 | + |
| 69 | + |
| 70 | + |
| 71 | + |
| 72 | + |
| 73 | + |
| 74 | + |
| 75 | + |
| 76 | + |
| 77 | + |
| 78 | + |
| 79 | + |
| 80 | + |
| 81 | + |
| 82 | + |
| 83 | + |

### Example B: Luminescent Viability Assay

RT112 cells are purchased from ATCC (Manassas, VA) and maintained in RPMI, 10% FBS (Gibco/Life Technologies). To measure the effect of test compounds on the viability of cells, the cells are plated with RPMI 10% FBS (5×10³ cells/well/in 50 μL) into black 96-well Greiner polystyrene in the presence or absence of 50 ul of a concentration range of test compounds. After 3 days, 100 ul of CellTiter-Glo Reagent (Promega) is added. Luminescence is read with a TopCount (PerkinElmer). IC₅₀ determination is performed by fitting the curve of percent inhibition versus the log of the inhibitor concentration using the GraphPad Prism 5.0 software.

### Example C: pFGFR2 and pFGFR1,3 Functional Cell HTRF Assay

To measure phosphorylated Fibroblast Growth Factor Receptor 2 (FGFR2), KATOIII cells (Human Gastric Carcinoma) are purchased from ATCC and maintained in Iscove's with 20% FBS (Gibco/Life Technologies). For the pFGFR2 assay, KATOIII cells are plated overnight in 5% FBS and Iscove's medium at 5×10⁴ cells/well into Corning 96-well flat-bottom tissue culture treated plates. The next morning, 50 μl of fresh media with 0.5% FBS is incubated in the presence or absence of a concentration range of test compounds also at 50 ul, for 1 hour at 37° C., 5% CO2. Cell are washed with PBS, lysed with Cell Signaling Lysis Buffer with standard Protease inhibitors for 45 min at room temperature. 4 μl total of Cis Bio Anti Phospho-YAP d2 and Cis Bio Anti Phospho-YAP Cryptate together are added to the lysate and mixed well (following directions of the kit). 16 μl is then transferred to 384 well Greiner white plates and stored at 4° C. overnight in the dark. Plates are read on the Pherastar plate reader at 665 nm and 620 nm wavelengths. IC₅₀ determination is performed by fitting the curve of inhibitor percent inhibition versus the log of the inhibitor concentration using the GraphPad Prism 5.0 software.

To measure phosphorylated Fibroblast Growth Factor Receptor 3 (FGFR3), in house stable cell lines BAF3-TEL-FGFR1 or BAF3-TEL-FGFR3 are maintained in RPMI with 10% FBS and 1 ug/ml puromycin (Gibco/Life Technologies). For the assay, 12 nl of BAF3-TEL-FGFR1 or BAF3-TEL-FGFR3 cells in serum free and puromycin free RPMI media at 1×10⁶ cell/ml are added to 384 Greiner white plate already containing 20nl dots of compounds at a concentration range. The plates are gently shaken (100 rpm) for 2 minutes at room temperature to mix well and incubate for 2 hours in a single layer at 37° C., 5% CO2. 4 μl/well of 1/25 dilution of lysis buffer #3 (Cis Bio) is added with standard Protease inhibitors and shaken at 200 rpm at room temperature for 20 minutes. 4 μl total of the Cis Bio Tb-pFGFR Ab (10 ng) and d2-FGFR3 (1 ng) together are added to the lysate and mixed well. The plates are sealed and incubated at room temperature overnight in the dark. The plates are read on the Pherastar plate reader at 665 nm and 620 nm wavelengths. IC₅₀ determination is performed by fitting the curve of inhibitor percent inhibition versus the log of the inhibitor concentration using the GraphPad Prism 5.0 software.

### Example D: pFGFR3 Functional Whole Blood HTRF Assay

To measure phosphorylated Fibroblast Growth Factor Receptor 3 (FGFR3) in a whole blood assay, in house stable cell lines BAF3-TEL-FGFR3 are maintained in RPMI with 10% FBS and 1 μg/ml puromycin (Gibco/Life Technologies). For the assay, 100 ul BAF3-TEL-FGFR3 cells in 10% FBS and puromycin free RPMI media at 5×10⁴ cell/well are added to fibronectin coated 96 well tissue culture plate (5ug/ml) overnight at 37° C., 5% CO2. The next day, serum is separated from the top of the blood by a low speed spin, 1200, RPM, and heat inactivated by incubating at 56° C. for 15 minutes. 30 μl of the cooled serum is added to a 96 well plate pre dotted with 70 nM dots of compounds at a concentration range. Cell plates are washed gently with media, all the blood/compound mixture is added to the plates, and the plates are incubated for 2 hours at 37° C., 5% CO2. Blood from the plate is gently washed twice by adding media to the side of the wells and then dumping media from the plate, and allowing the plate to briefly sit on a paper towel to drain. 70 μl/well of 1× of lysis buffer #1 (Cis Bio) are added with standard Protease inhibitors, and are shaken at 400 rpm at room temperature for 30 minutes. Following lysis, the plate is spun down for 5 minutes and 16 uL of lysate is transferred into a 384-well small volume plate. 4 μl total of the Cis Bio Tb-pFGFR Ab (10 ng) and d2-FGFR3 (1 ng) together are added to the lysate and mixed well. The plates are sealed and incubated at room temperature overnight in the dark. Plates are read on the Pherastar plate reader at 665 nm and 620 nm wavelengths. IC₅₀ determination is performed by fitting the curve of inhibitor percent inhibition versus the log of the inhibitor concentration using the GraphPad Prism 5.0 software.

### Example E: KATOIII Whole Blood pFGFR2a ELISA Assay

To measure tyrosine-phosphorylated Fibroblast Growth Factor Receptor 2 alpha (FGFR2α) in KATO III spiked whole blood assay, KATO III cells are purchased from ATCC and maintained in Iscove's medium with 20% FBS (Gibco/Life Technologies). To measure the inhibition of FGFR2α activity of test compounds, the cells are resuspended with Iscove's, 0.2% FBS at 5×10⁶ cells/ml. 50 μL of the cells are then spiked into a 96-deep well 2 ml polypropylene assay block (Costar,) in the presence or absence of a concentration range of test compounds and 300 ul human heparinized whole blood (Biological Specialty Corp, Colmar PA). After 4 hours incubation in 37° C., the red cells are lysed using Qiagen EL buffer and the cell lysates are resuspended in lysis buffer (Cell Signaling) containing standard protease inhibitor cocktail (Calbiochem/EMD,) and PMSF (Sigma) for 30 minutes ice. The lysates are transferred to a standard V bottom propylene tissue culture plate and frozen overnight at −80° C. Samples are tested an in an R & D Systems DuoSet IC Human Phospho-FGF R2α ELISA and the plate is measured using a SpectraMax M5 microplate set to 450 nm with a wavelength correction of 540. IC₅₀ determination is performed by fitting the curve of inhibitor percent inhibition versus the log of the inhibitor concentration using the GraphPad Prism 5.0 software.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A compound having Formula (I): 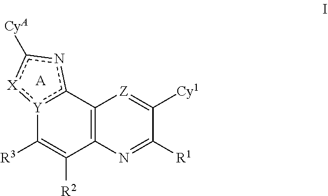
or a pharmaceutically acceptable salt thereof, wherein:
X is NR⁵;
Y is C; [symbol]
represents a single bond or a double bond;
Z is N or CR⁶;
Cy¹ is selected from C₆₋₁₀ aryl and 5-10 membered heteroaryl; wherein each 5-10 membered heteroaryl has at least one ring-forming carbon atom and 1, 2, 3, or 4 ring-forming heteroatoms independently selected from N, O, and S; wherein the N and S are optionally oxidized; wherein a ring-forming carbon atom of 5-10 membered heteroaryl is optionally substituted by oxo to form a carbonyl group; and wherein the C₆₋₁₀ aryl and 5-10 membered heteroaryl are each optionally substituted with 1, 2, 3 or 4 substituents independently selected from R¹⁰;
Cy^{A} is selected from C₃₋₁₀ cycloalkyl, 4-10 membered heterocycloalkyl, C₆₋₁₀ aryl and 5-10 membered heteroaryl; wherein the 4-10 membered heterocycloalkyl and 5-10 membered heteroaryl each has at least one ring-forming carbon atom and 1, 2, 3, or 4 ring-forming heteroatoms independently selected from N, O, and S; wherein the N and S are optionally oxidized; wherein a ring-forming carbon atom of 5-10 membered heteroaryl and 4-10 membered heterocycloalkyl is optionally substituted by oxo to form a carbonyl group; and wherein the C₃₋₁₀ cycloalkyl, 4-10 membered heterocycloalkyl, C₆₋₁₀ aryl and 5-10 membered heteroaryl are each optionally substituted with 1, 2, 3 or 4 substituents independently selected from R²⁰;
R¹, R², R³, and R⁶ are each independently selected from H, D, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, halo, CN, OR^{a9}, SR^{a9}, C(O)R^{b9}, C(O)NR^{c9}R^{d9}, C(O)OR^{a9}, NR^{c9}R^{d9}, NR^{c9}C(O)R^{b9}, NR^{c9}C(O)OR^{a9}, NR^{c9}C(O)NR^{c9}R^{d9}, NR^{c9}S(O)R^{b9}, NR^{c9}S(O)₂R^{b9}, NR^{c9}S(O)₂NR^{c9}R^{d9}, S(O)R^{b9}, S(O)NR^{c9}R^{d9}, S(O)₂R^{b9}, and S(O)₂NR^{c9}R^{d9}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, and 5-10 membered heteroaryl, are each optionally substituted with 1, 2, 3, or 4 substituents independently selected from R³⁰;
R⁵ is selected from H, D, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, and 5-10 membered heteroaryl, are each optionally substituted with 1, 2, 3, or 4 substituents independently selected from R⁵⁰;
each R¹⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{a1})R^{b1}, C(═NOR^{a1})R^{b1}, C(═NR^{c1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R¹¹;
each R¹¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a3}, SR^{a3}, C(O)R^{b3}, C(O)NR^{c3}R^{d3}, C(O)OR^{a3}, NR^{c3}R^{d3}, NR^{c3}C(O)R^{b3}, NR^{c3}(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R¹²;
each R¹² is independently selected from C₁₋₆ alkyl, C₂₋₆ alkenyl, C₂₋₆ alkynyl, C₁₋₆ haloalkyl, C₃₋₆ cycloalkyl, C₆₋₁₀ aryl, 5-6 membered heteroaryl, 4-7 membered heterocycloalkyl, halo, D, CN, OR^{a5}, SR^{a5}, C(O)R^{b5}, C(O)NR^{c5}R^{d5}, C(O)OR^{a5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5}; wherein said C₁₋₆ alkyl, C₂₋₆ alkenyl, C₂₋₆ alkynyl, C₃₋₆ cycloalkyl, C₆₋₁₀ aryl, 5-6 membered heteroaryl and 4-7 membered heterocycloalkyl, are each optionally substituted with 1, 2, 3, or 4 substituents independently selected from R^{g};
each R²⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NOR^{a2})R^{b2}, C(═NR^{e2})NR^{c2}R^{d2} NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R²¹;
each R²¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a4}, SR^{a4}, C(O)R^{b4}, C(O)NR^{c4}R^{d4}, C(O)OR^{a4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R²²;
each R²² is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a6}, SR^{a6}, C(O)R^{b6}, C(O)NR^{c6}R^{d6}, C(O)OR^{a6}, NR^{c6}R^{d6}, NR^{c6}C(O)R^{b6}, NR^{c6}C(O)OR^{a6}, NR^{c6}C(O)NR^{c6}R^{d6}, NR^{c6}S(O)R^{b6}, NR^{c6}S(O)₂R^{b6}, NR^{c6}S(O)₂NR^{c6}R^{d6}, S(O)R^{b6}, S(O)NR^{c6}R^{d6}, S(O)₂R^{b6}, and S(O)₂NR^{c6}R^{d6}; wherein said C₁₋₆ alkyl, C₂₋₆ alkenyl, C₂₋₆ alkynyl, C₃₋₆ cycloalkyl, phenyl, 5-6 membered heteroaryl and 4-7 membered heterocycloalkyl, are each optionally substituted with 1, 2, 3, or 4 substituents independently selected from R^{g};
each R³⁰ is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a10}, SR^{a10}, C(O)R^{b10}, C(O)NR^{c10}R^{d10}, C(O)OR^{a10}, NR^{c10}R^{d10}, NR^{c10}C(O)R^{b10}, NR^{c10}C(O)OR^{a10}, NR^{c10}C(O)NR^{c10}R^{d10}, NR^{c10}S(O)R^{b10}, NR^{c10}S(O)₂R^{b10}, NR^{c10}S(O)₂NR^{c10}R^{d10}, S(O)R^{b10}, S(O)NR^{c10}R^{d10}, S(O)₂R^{b10}, and S(O)₂NR^{c10}R^{d10}; wherein said C₁₋₆ alkyl, C₂₋₆ alkenyl, C₂₋₆ alkynyl, C₃₋₆ cycloalkyl, phenyl, 5-6 membered heteroaryl and 4-7 membered heterocycloalkyl, are each optionally substituted with 1, 2, 3, or 4 substituents independently selected from R^{g};
each R⁵⁰ is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a11}, SR^{a11}, C(O)R^{b11}, C(O)NR^{c11}R^{d11}, C(O)OR^{a11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)R^{b11}, NR^{c11}S(O)₂R^{b11}, NR^{c11}S(O)₂NR^{c11}R^{d11}, S(O)R^{b11}, S(O)NR^{c11}R^{d11}, S(O)₂R^{b11}, and S(O)₂NR^{c11}R^{d11}; wherein said C₁₋₆ alkyl, C₂₋₆ alkenyl, C₂₋₆ alkynyl, C₃₋₆ cycloalkyl, phenyl, 5-6 membered heteroaryl and 4-7 membered heterocycloalkyl, are each optionally substituted with 1, 2, 3, or 4 substituents independently selected from R^{g};
each R^{a1}, R^{c1} and R^{d1} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R¹¹;
or any R^{c1} and R^{d1} attached to the same N atom, together with the N atom to which they are attached, form a 4-, 5-, 6- or 7-membered heterocycloalkyl group optionally substituted with 1, 2, 3, or 4 substituents independently selected from R¹¹;
each R^{b1} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R¹¹;
each R^{e1} is independently selected from H, CN, C₁₋₆ alkyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{a2}, R^{c2} and R^{d2}, is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl are each optionally substituted with 1, 2, 3, or 4 substituents independently selected from R²¹;
or any R^{c2} and R^{d2} attached to the same N atom, together with the N atom to which they are attached, form a 4-, 5-, 6- or 7-membered heterocycloalkyl group optionally substituted with 1, 2 or 3 substituents independently selected from R²¹;
each R^{b2} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl are each optionally substituted with 1, 2, 3, or 4 substituents independently selected from R²¹;
each R^{e2} is independently selected from H, CN, C₁₋₆ alkyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{a3}, R^{c3} and R^{d3}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R¹²;
or any R^{c3} and R^{d3} attached to the same N atom, together with the N atom to which they are attached, form a 4-, 5-, 6- or 7-membered heterocycloalkyl group optionally substituted with 1, 2 or 3 substituents independently selected from R¹²;
each R^{b3} is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl, are each optionally substituted with 1, 2, 3, or 4 substituents independently selected from R¹²;
each R^{a4}, R^{c4} and R^{d4}, is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R²²;
or any R^{c4} and R^{d4} attached to the same N atom, together with the N atom to which they are attached, form a 4-, 5-, 6- or 7-membered heterocycloalkyl group optionally substituted with 1, 2 or 3 substituents independently selected from R²²;
each R^{b4} is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R²²;
each R^{a5}, R^{c5} and R^{d5}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
or any R^{c5} and R^{d5} attached to the same N atom, together with the N atom to which they are attached, form a 4-, 5-, 6- or 7-membered heterocycloalkyl group optionally substituted with 1, 2, 3, or 4 substituents independently selected from R^{g};
each R^{b5} is independently selected from C₁₋₆ alkyl, C₂₋₆ alkenyl, C₂₋₆ alkynyl and C₁₋₆ haloalkyl; wherein said C₁₋₆ alkyl C₂₋₆ alkenyl and C₂₋₆ alkynyl are each optionally substituted with 1, 2, 3, or 4 substituents independently selected from R^{g};
each R^{a6}, R^{c6} and R^{d6}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{b6} is independently selected from C₁₋₆ alkyl, C₂₋₆ alkenyl, C₂₋₆ alkynyl, and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{a9}, R^{c9}, and R^{d9}, is independently selected from H, C₁₋₆ alkyl, C₁₋₆alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³⁰;
or any R^{c9} and R^{d9} attached to the same N atom, together with the N atom to which they are attached, form a 4-, 5-, 6- or 7-membered heterocycloalkyl group optionally substituted with 1, 2 or 3 substituents independently selected from R³⁰;
each R^{b9} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³⁰;
each Ra¹⁰, R^{c10} and R^{d10}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{b10} is independently selected from C₁₋₆ alkyl, C₂₋₆ alkenyl, C₂₋₆ alkynyl, and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{a11}, R^{c11} and R^{d11}, is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{b11} is independently selected from C₁₋₆ alkyl, C₂₋₆ alkenyl, C₂₋₆ alkynyl, and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g}; and
each R^{g} is independently selected from D, OH, NO₂, CN, halo, C₁₋₆ alkyl, C₂₋₆ alkenyl, C₂₋₆ alkynyl, C₁₋₆ haloalkyl, C₃₋₆ cycloalkyl, C₃₋₆ cycloalkyl-C₁₋₂ alkylene, C₁₋₆ alkoxy, C₁₋₆ haloalkoxy, C₁₋₃ alkoxy-C₁₋₃ alkyl, C₁₋₃ alkoxy-C₁₋₃ alkoxy, HO—C₁₋₃ alkoxy, HO—C₁₋₃ alkyl, cyano-C₁₋₃ alkyl, H₂N—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkoxycarbonylamino, C₁₋₆ alkylcarbonyloxy, aminocarbonyloxy, C₁₋₆ alkylaminocarbonyloxy, di(C₁₋₆ alkyl)aminocarbonyloxy, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

2. The compound of claim 1, or a pharmaceutically acceptable salt thereof, wherein:
ring A is aromatic;
Y is C;
X is NR⁵; [symbol]
represents a single bond or a double bond;
Z is N or CR⁶;
Cy¹ is selected from C₆₋₁₀ aryl and 5-10 membered heteroaryl; wherein each 5-10 membered heteroaryl has at least one ring-forming carbon atom and 1, 2, 3, or 4 ring-forming heteroatoms independently selected from N, O, and S; wherein the N and S are optionally oxidized; wherein a ring-forming carbon atom of 5-10 membered heteroaryl is optionally substituted by oxo to form a carbonyl group; and wherein the C₆₋₁₀ aryl and 5-10 membered heteroaryl are each optionally substituted with 1, 2, 3 or 4 substituents independently selected from R¹⁰;
Cy^{A} is selected from C₃₋₁₀ cycloalkyl, 4-10 membered heterocycloalkyl, C₆₋₁₀ aryl and 5-10 membered heteroaryl; wherein the 4-10 membered heterocycloalkyl and 5-10 membered heteroaryl each has at least one ring-forming carbon atom and 1, 2, 3, or 4 ring-forming heteroatoms independently selected from N, O, and S; wherein the N and S are optionally oxidized; wherein a ring-forming carbon atom of 5-10 membered heteroaryl and 4-10 membered heterocycloalkyl is optionally substituted by oxo to form a carbonyl group; and wherein the C₃₋₁₀ cycloalkyl, 4-10 membered heterocycloalkyl, C₆₋₁₀ aryl and 5-10 membered heteroaryl are each optionally substituted with 1, 2, 3 or 4 substituents independently selected from R²⁰;
R¹, R², R³, and R⁶ are each independently selected from H, D, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, halo, CN, OR^{a9}, SR^{a9}, C(O)R^{b9}, C(O)NR^{c9}R^{d9}, C(O)OR^{a9}, NR^{c9}R^{d9}, NR^{c9}C(O)R^{b9}, NR^{c9}C(O)OR^{a9}, NR^{c9}S(O)R^{b9}, NR^{c9}S(O)₂R^{b9}, NR^{c9}S(O)₂NR^{c9}R^{d9}, S(O)R^{b9}, S(O)NR^{c9}R^{d9}, S(O)₂R^{b9}, and S(O)₂NR^{b9}R^{d9}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, and 5-10 membered heteroaryl, are each optionally substituted with 1, 2, 3, or 4 substituents independently selected from R³⁰;
R⁵ is selected from C₁₋₆ alkyl, C₂₋₆ alkenyl, C₂₋₆ alkynyl, C₁₋₆ haloalkyl, C₃₋₄ cycloalkyl, 4-10 membered heterocycloalkyl, C₆₋₁₀ aryl, and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, and 5-10 membered heteroaryl, are each optionally substituted with 1, 2, 3, or 4 substituents independently selected from R⁵⁰;
each R¹⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NOR^{a1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R¹¹;
each R¹¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a3}, SR^{a3}, C(O)R^{b3}, C(O)NR^{c3}R^{d3}, C(O)OR^{a3}, NR^{c3}R^{d3}, NR^{c3}C(O)R^{b3}, NR^{c3}C(O)OR^{a3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R¹²;
each R¹² is independently selected from C₁₋₆ alkyl, C₂₋₆ alkenyl, C₂₋₆ alkynyl, C₁₋₆ haloalkyl, C₃₋₆ cycloalkyl, C₆₋₁₀ aryl, 5-6 membered heteroaryl, 4-7 membered heterocycloalkyl, halo, D, CN, OR^{a5}, SR^{a5}, C(O)R^{b5}, C(O)NR^{c5}R^{d5}, C(O)OR^{a5}, NR^{c5}R^{d5}, NR^{c5}C(O)R^{b5}, NR^{c5}C(O)OR^{a5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5}; wherein said C₁₋₆ alkyl, C₂₋₆ alkenyl, C₂₋₆ alkynyl, C₃₋₆ cycloalkyl, C₆₋₁₀ aryl, 5-6 membered heteroaryl and 4-7 membered heterocycloalkyl, are each optionally substituted with 1, 2, 3, or 4 substituents independently selected from R^{g};
each R²⁰ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2} NR^{c2}C(O)R^{b2}, NR^{c2}C(O)OR^{a2}, NR^{c2}C(O)NR^{c2}R^{d2}, C(═NR^{e2})R^{b2}, C(═NOR^{a2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R²¹;
each R²¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, D, CN, OR^{a4}, SR^{a4}, C(O)R^{b4}, C(O)NR^{c4}R^{d4}, C(O)OR^{a4}, NR^{c4}R^{d4}, NR^{c4}C(O)R^{b4}, NR^{c4}C(O)OR^{a4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene are each optionally substituted with 1, 2, 3, or 4 substituents independently selected from R²²;
each R²² is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a6}, SR^{a6}, C(O)R^{b6}, C(O)NR^{c6}R^{d6}, C(O)OR^{a6}, NR^{c6}R^{d6}, NR^{c6}C(O)R^{b6}, NR^{c6}C(O)OR^{a6}, NR^{c6}S(O)R^{b6}, NR^{c6}S(O)₂R^{b6}, NR^{c6}S(O)₂NR^{c6}R^{d6}, S(O)R^{b6}, S(O)NR^{c6}R^{d6}, S(O)₂R^{b6}, and S(O)₂NR^{c6}R^{d6}; wherein said C₁₋₆ alkyl, C₂₋₆ alkenyl, C₂₋₆ alkynyl, C₃₋₆ cycloalkyl, phenyl, 5-6 membered heteroaryl and 4-7 membered heterocycloalkyl, are each optionally substituted with 1, 2, 3, or 4 substituents independently selected from R^{g};
each R³⁰ is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a10}, SR^{a10}, C(O)R^{b10}, C(O)NR^{c10}R^{d10}, C(O)OR^{a10}, NR^{c10}R^{d10}, NR^{c10}C(O)R^{b10}, NR^{c10}(O)OR^{a10}, NR^{c10}S(O)R^{b10}, NR^{c10}S(O)₂R^{b10}, NR^{c10}S(O)₂NR^{c10}R^{d10}, S(O)R^{b10}, S(O)NR^{c10}R^{d10}, S(O)₂R^{b10}, and S(O)₂NR^{c10}R^{d10}; wherein said C₁₋₆ alkyl, C₂₋₆ alkenyl, C₂₋₆ alkynyl, C₃₋₆ cycloalkyl, phenyl, 5-6 membered heteroaryl and 4-7 membered heterocycloalkyl, are each optionally substituted with 1, 2, 3, or 4 substituents independently selected from R^{g};
each R⁵⁰ is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl, 4-7 membered heterocycloalkyl, halo, D, CN, OR^{a11}, SR^{a11}, C(O)R^{b11}, C(O)NR^{c11}R^{d11}, C(O)OR^{a11}, NR^{c11}R^{d11}, NR^{c11}C(O)R^{b11}, NR^{c11}C(O)OR^{a11}, NR^{c11}S(O)R^{b11}, NR^{c11}S(O)₂R^{b11}, NR^{c11}S(O)₂NR^{c11}R^{d11}, S(O)R^{b11}, S(O)NR^{c11}R^{d11}, S(O)₂R^{b11}, and S(O)₂NR^{c11}R^{d11}; wherein said C₁₋₆ alkyl, C₂₋₆ alkenyl, C₂₋₆ alkynyl, C₃₋₆ cycloalkyl, phenyl, 5-6 membered heteroaryl and 4-7 membered heterocycloalkyl, are each optionally substituted with 1, 2, 3, or 4 substituents independently selected from R^{g};
each R^{a1}, R^{c1} and R^{d1} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R¹¹;
or any R^{c1} and R^{d1} attached to the same N atom, together with the N atom to which they are attached, form a 4-, 5-, 6- or 7-membered heterocycloalkyl group optionally substituted with 1, 2, 3, or 4 substituents independently selected from R¹¹;
each R^{b1} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R¹¹;
each R^{e1} is independently selected from H, CN, C₁₋₆ alkyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{a2}, R^{c2} and R^{d2} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl are each optionally substituted with 1, 2, 3, or 4 substituents independently selected from R²¹;
or any R^{c2} and R^{d2} attached to the same N atom, together with the N atom to which they are attached, form a 4-, 5-, 6- or 7-membered heterocycloalkyl group optionally substituted with 1, 2 or 3 substituents independently selected from R²¹;
each R^{b2} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl are each optionally substituted with 1, 2, 3, or 4 substituents independently selected from R²¹;
each R^{e2} is independently selected from H, CN, C₁₋₆ alkyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{a3}, R^{c3} and R^{d3} is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R¹²;
or any R^{c3} and R^{d3} attached to the same N atom, together with the N atom to which they are attached, form a 4-, 5-, 6- or 7-membered heterocycloalkyl group optionally substituted with 1, 2 or 3 substituents independently selected from R¹²;
each R^{b3} is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl, C₂₋₆ alkenyl, C₂₋₆ alkynyl, C₃₋₆ cycloalkyl, phenyl, 5-6 membered heteroaryl and 4-7 membered heterocycloalkyl, are each optionally substituted with 1, 2, 3, or 4 substituents independently selected from R¹²;
each R^{a4}, R^{c4} and R^{d4} is independently selected from H, C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl; wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R²²;
or any R^{c4} and R^{d4} attached to the same N atom, together with the N atom to which they are attached, form a 4-, 5-, 6- or 7-membered heterocycloalkyl group optionally substituted with 1, 2 or 3 substituents independently selected from R²²;
each R^{b4} is independently selected from C₁₋₆ alkyl, C₂₋₆ alkenyl, C₂₋₆ alkynyl, C₁₋₆ haloalkyl, C₃₋₆ cycloalkyl, phenyl, 5-6 membered heteroaryl and 4-7 membered heterocycloalkyl;
wherein said C₁₋₆ alkyl C₂₋₆ alkenyl, C₂₋₆ alkynyl, C₃₋₆ cycloalkyl, phenyl, 5-6 membered heteroaryl and 4-7 membered heterocycloalkyl are each optionally substituted with 1, 2, 3, or 4 substituents independently selected from R²²;
each R^{a5}, R^{c5} and R^{d5} is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
or any R^{c5} and R^{d5} attached to the same N atom, together with the N atom to which they are attached, form a 4-, 5-, 6- or 7-membered heterocycloalkyl group optionally substituted with 1, 2, 3, or 4 substituents independently selected from R^{g};
each R^{b5} is independently selected from C₁₋₆ alkyl, C₂₋₆ alkenyl, C₂₋₆ alkynyl and C₁₋₆ haloalkyl; wherein said C₁₋₆ alkyl C₂₋₆ alkenyl and C₂₋₆ alkynyl are each optionally substituted with 1, 2, 3, or 4 substituents independently selected from R^{g};
each R^{a6}, R^{c6} and R^{d6} is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{b6} is independently selected from C₁₋₆ alkyl, C₂₋₆ alkenyl, C₂₋₆ alkynyl, and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{a9}, R^{c9}, and R^{d9} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³⁰;
or any R^{c9} and R^{d9} attached to the same N atom, together with the N atom to which they are attached, form a 4-, 5-, 6- or 7-membered heterocycloalkyl group optionally substituted with 1, 2 or 3 substituents independently selected from R³⁰;
each R^{b9} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein said C₁₋₆ alkyl, C₂₋₆ alkenyl, C₂₋₆ alkynyl, C₃₋₁₀ cycloalkyl, 4-10 membered heterocycloalkyl, C₆₋₁₀ aryl and 5-10 membered heteroaryl, are each optionally substituted with 1, 2, 3, or 4 substituents independently selected from R³⁰;
each R^{a10}, R^{c10} and R^{d10} is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{b10} is independently selected from C₁₋₆ alkyl, C₂₋₆ alkenyl, C₂₋₆ alkynyl, and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{a11}, R^{c11} and R^{d11} is independently selected from H, C₁₋₆ alkyl, C₂₋₆ alkenyl, C₂₋₆ alkynyl and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g};
each R^{b11} is independently selected from C₁₋₆ alkyl, C₂₋₆ alkenyl, C₂₋₆ alkynyl, and C₁₋₆ haloalkyl; wherein said C₁₋₆ alkyl, C₂₋₆ alkenyl and C₂₋₆ alkynyl are each optionally substituted with 1, 2, 3, or 4 substituents independently selected from R^{g}; and
each R^{g} is independently selected from D, OH, NO₂, CN, halo, C₁₋₆ alkyl, C₂₋₆ alkenyl, C₂₋₆ alkynyl, C₁₋₆ haloalkyl, C₃₋₆ cycloalkyl, C₃₋₆ cycloalkyl-C₁₋₂ alkylene, C₁₋₆ alkoxy, C₁₋₆ haloalkoxy, C₁₋₃ alkoxy-C₁₋₃ alkyl, C₁₋₃ alkoxy-C₁₋₃ alkoxy, HO—C₁₋₃ alkoxy, HO—C₁₋₃ alkyl, cyano-C₁₋₃ alkyl, H₂N—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkoxycarbonylamino, C₁₋₆ alkylcarbonyloxy, aminocarbonyloxy, C₁₋₆ alkylaminocarbonyloxy, di(C₁₋₆ alkyl)aminocarbonyloxy, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆alkyl)aminosulfonylamino, aminocarbonylamino, C₁₋₆ alkylaminocarbonylamino, and di(C₁₋₆ alkyl)aminocarbonylamino.

3-6. (canceled)

7. The compound of claim 1, or a pharmaceutically acceptable salt thereof, wherein Cy¹ is 5-10 membered heteroaryl optionally substituted with 1, 2, 3 or 4 substituents independently selected from R¹⁰.

8. The compound of claim 1, or a pharmaceutically acceptable salt thereof, wherein Cy¹ is pyrazolyl or pyridinyl, each optionally substituted with 1 or 2 substituents independently selected from R¹⁰.

9. The compound of claim 1, or a pharmaceutically acceptable salt thereof, wherein Cy¹ is pyrazolyl, pyridinyl, or phenyl, each optionally substituted with 1 or 2 substituents independently selected from R¹⁰.

10. The compound of claim 1, or a pharmaceutically acceptable salt thereof, wherein Cy¹ is selected from 1-methyl-1H-pyrazol-4-yl, 1,3-dimethyl-1H-pyrazol-4-yl, 1-(1-acetylpyrrolidin-3-yl)-1H-pyrazol-4-yl, 1-(4-carboxycyclohexyl)-1H-pyrazol-4-yl, 1-(4-acetamidocyclohexyl)-1H-pyrazol-4-yl, 1-(3-hydroxycyclobutyl)-1H-pyrazol-4-yl, and 6-(4-acetylpiperazin-1-yl)pyridin-3-yl.

11. The compound of claim 1, or a pharmaceutically acceptable salt thereof, wherein Cy¹ is selected from 1-methyl-1H-pyrazol-4-yl, 1,3-dimethyl-1H-pyrazol-4-yl, 1-(1-acetylpyrrolidin-3-yl)-1H-pyrazol-4-yl, 1-(4-carboxycyclohexyl)-1H-pyrazol-4-yl, 1-(4-acetamidocyclohexyl)-1H-pyrazol-4-yl, 1-(3-hydroxycyclobutyl)-1H-pyrazol-4-yl, 6-(4-acetylpiperazin-1-yl)pyridin-3-yl, 1-(1-(2-hydroxyacetyl)pyrrolidin-3-yl)-1H-pyrazol-4-yl, 1-(1-(1-aminocyclopropane-1-carbonyl)azetidin-3-yl)-1H-pyrazol-4-yl, 1-(1-(2-(1H-imidazol-1-yl)acetyl)azetidin-3-yl)-1H-pyrazol-4-yl, 1-(1-(oxazole-4-carbonyl)azetidin-3-yl)-1H-pyrazol-4-yl, 1-(1-(4-aminotetrahydro-2H-pyran-4-carbonyl)azetidin-3-yl)-1H-pyrazol-4-yl, 1-(1-(2-cyanoacetyl)azetidin-3-yl)-1H-pyrazol-4-yl, 1-(4-carboxy-3-fluorobenzyl)-1H-pyrazol-4-yl, 1-(4-(dimethylcarbamoyl)phenyl)-1H-pyrazol-4-yl, 1-((1r,4r)-4-(2-methoxyacetamido)cyclohexyl)-1H-pyrazol-4-yl, 1-((1r,4r)-4-(3,3-dimethylureido)cyclohexyl)-1H-pyrazol-4-yl, 1-(2-acetyl-2-azaspiro[3.5]nonan-7-yl)-1H-pyrazol-4-yl, 1-(tetrahydro-2H-pyran-4-yl)-1H-pyrazol-4-yl, 1-((1r,4r)-4-hydroxycyclohexyl)-1H-pyrazol-4-yl, 1-(4-carboxy-4-methylcyclohexyl)-1H-pyrazol-4-yl, 1-(1-(dimethylcarbamoyl)pyrrolidin-3-yl)-1H-pyrazol-4-yl, 1-(1-(N,N-dimethylsulfamoyl)pyrrolidin-3-yl)-1H-pyrazol-4-yl, 1-(3-(2-hydroxyacetyl)-3-azabicyclo[3.1.0]hexan-1-yl)-1H-pyrazol-4-yl, 1-(1-(morpholine-4-carbonyl)pyrrolidin-3-yl)-1H-pyrazol-4-yl, 1-(3-(2-hydroxyacetyl)-3-azabicyclo[4.1.0]heptan-6-yl)-1H-pyrazol-4-yl, 1-(3-acetyl-3-azabicyclo[4.1.0]heptan-1-yl)-1H-pyrazol-4-yl, 6-(1-(2-hydroxyacetyl)azetidin-3-yl)pyridin-3-yl, 5-(1-acetylazetidin-3-yl)pyridin-3-yl, 4-(1-acetylazetidin-3-yl)phenyl), 1-cyclopropyl-1H-pyrazol-4-yl, 1-(tetrahydrofuran-3-yl)-1H-pyrazol-4-yl, 1-(oxetan-3-yl)-1H-pyrazol-4-yl, 1-(2-(dimethylamino)ethyl)-1H-pyrazol-4-yl, 1-isopropyl-1H-pyrazol-4-yl, 1-(difluoromethyl)-1H-pyrazol-4-yl, 1-(2-cyanoethyl)-1H-pyrazol-4-yl, 1-(2-hydroxy-2-methylpropyl)-1H-pyrazol-4-yl, 1-(cyanomethyl)-1H-pyrazol-4-yl, 1-(1-methylpiperidin-4-yl)-1H-pyrazol-4-yl, 1-(1-hydroxy-2-methylpropan-2-yl)-1H-pyrazol-4-yl, 1-(2-(methylsulfonyl)ethyl)-1H-pyrazol-4-yl, 1-(2,2,2-trifluoroethyl)-1H-pyrazol-4-yl, 1-(2-morpholinoethyl)-1H-pyrazol-4-yl, and 1-(2-hydroxyethyl)-1H-pyrazol-4-yl.

12. The compound of claim 1, or a pharmaceutically acceptable salt thereof, wherein each R¹⁰ is independently selected from C₁₋₆ alkyl, 4-10 membered heterocycloalkyl, and C₃₋₁₀ cycloalkyl, each of which is optionally substituted with 1 or 2 substituents independently selected from R¹¹.

13. The compound of claim 1, or a pharmaceutically acceptable salt thereof, wherein each R¹⁰ is independently selected from C₁₋₆ alkyl, C₁₋₆ haloalkyl, phenyl, 4-10 membered heterocycloalkyl, and C₃₋₁₀ cycloalkyl, each of which is optionally substituted with 1 or 2 substituents independently selected from R¹¹.

14. The compound of claim 1, or a pharmaceutically acceptable salt thereof, wherein each R¹⁰ is independently selected from methyl, ethyl, isopropyl, 2-methylpropyl, difluoromethyl, cyclopropyl, cyclobutyl, cyclohexyl, phenyl, piperzinyl, azetidinyl, pyrrolidinyl, azaspiro[3.5]nonanyl, tetrahydro-2H-pyranyl, azabicyclo[3.1.0]hexanyl, azabicyclo[4.1.0]heptanyl, tetrahydrofuranyl, oxetanyl, and piperidinyl, each of which is optionally substituted with 1 or 2 substituents independently selected from R¹¹.

15. The compound of claim 1, or a pharmaceutically acceptable salt thereof, wherein each R¹¹ is independently selected from C(O)R^{b3}, C(O)OR^{a3}, NR^{c3}C(O)R^{b3}, and OR^{a3}.

16. The compound of claim 1, or a pharmaceutically acceptable salt thereof, wherein each R¹¹ is independently selected from C₁₋₆ alkyl, CN, C(O)R^{b3}, C(O)OR^{a3}, NR^{c3}C(O)R^{b3}, OR^{a3}, C(O)NR^{c3}R^{d3}, NR^{c3}R^{d3}, NR^{c3}C(O)NR^{c3}R^{d3}, S(O)₂NR^{c3}R^{d3}, S(O)₂R^{b3}, phenyl, and 4-6 membered heterocycloalkyl, wherein said C₁₋₆ alkyl, phenyl, and 4-6 membered heterocycloalkyl are each optionally substituted with 1 or 2 substituents independently selected from R¹².

17. The compound of claim 1, or a pharmaceutically acceptable salt thereof, wherein each R¹¹ is independently selected from C(O)CH₃, C(O)OH, NHC(O)CH₃, and OH.

18. The compound of claim 1, or a pharmaceutically acceptable salt thereof, wherein each R¹¹ is independently selected from methyl, CN, OH, C(O)CH₃, C(O)OH, NHC(O)CH₃, C(O)CH₂OH, C(O)N(CH₃)₂, NHC(O)CH₂OCH₃, NHC(O)N(CH₃)₂, S(O)₂N(CH₃)₂, N(CH₃)₂, S(O)₂CH₃, 2-cyanoacetyl, morpholine-4-carbonyl, morpholinyl, 1-aminocyclopropane-1-carbonyl, (1H-imidazol-1-yl)acetyl, oxazole-4-carbonyl, 4-aminotetrahydro-2H-pyran-4-carbonyl, and 4-carboxy-3-fluorophenyl.

19. The compound of claim 1, or a pharmaceutically acceptable salt thereof, wherein Cy^{A} is selected from phenyl and 5-6 membered heteroaryl, wherein the phenyl and 5-6 membered heteroaryl are each optionally substituted with 1, 2, 3 or 4 substituents independently selected from R²⁰.

20. The compound of claim 1, or a pharmaceutically acceptable salt thereof, wherein Cy^{A} is phenyl or furanyl, each optionally substituted with 1, 2, or 3 substituents independently selected from R²⁰.

21. The compound of claim 1, or a pharmaceutically acceptable salt thereof, wherein Cy^{A} is selected from 2-chlorophenyl, 2,6-difluorophenyl, 2-chloro-6-fluorophenyl, 2,6-dichlorophenyl, 2-fluoro-6-methylphenyl, 2-chloro-4-methoxyphenyl, and 3-methylfuran-2-yl.

22. The compound of claim 1, or a pharmaceutically acceptable salt thereof, wherein Cy^{A} is selected from 2-chlorophenyl, 2,6-difluorophenyl, 2-chloro-6-fluorophenyl, 2,6-dichlorophenyl, 2-fluoro-6-methylphenyl, 2-chloro-4-methoxyphenyl, 3-methylfuran-2-yl, 1-methylpiperidin-4-yl, (1S,5R)-2-oxo-3,8-diazabicyclo[3.2.1]octan-8-yl, 2-chloro-6-(trifluoromethyl)phenyl, 2,6-dimethylphenyl, 2-cyano-6-fluorophenyl, 2-fluoro-6-methoxyphenyl, 2,3-dimethylphenyl, 4-(hydroxymethyl)-2-methylphenyl, 3-acrylamido-2,6-dichlorophenyl, 3-acrylamidophenyl, 2-acrylamidophenyl, and cyclopropyl.

23. The compound of claim 1, or a pharmaceutically acceptable salt thereof, wherein each R²⁰ is independently selected from halo, C₁₋₆ alkyl, and OR^{a2}.

24. The compound of claim 1, or a pharmaceutically acceptable salt thereof, wherein each R²⁰ is independently selected from halo, CN, C₁₋₆ alkyl, C₁₋₆ haloalkyl, and NR^{c2}C(O)R^{b2}, wherein the C₁₋₆ alkyl is optionally substituted with OR^{a4}.

25. The compound of claim 1, or a pharmaceutically acceptable salt thereof, wherein each R²⁰ is independently selected from chloro, fluoro, methyl, and methoxy.

26. The compound of claim 1, or a pharmaceutically acceptable salt thereof, wherein each R²⁰ is independently selected from CN, chloro, fluoro, methyl, methoxy, trifluoromethyl, hydroxymethyl, and acrylamido.

27. The compound of claim 1, or a pharmaceutically acceptable salt thereof, wherein R¹ is H.

28. The compound of claim 1, or a pharmaceutically acceptable salt thereof, wherein R¹ is H, OR^{a9}, or NR^{c9}R^{d9}.

29. The compound of claim 1, or a pharmaceutically acceptable salt thereof, wherein R² is H.

30. The compound of claim 1, or a pharmaceutically acceptable salt thereof, wherein R³ is H.

31-35. (canceled)

36. The compound of claim 1, or a pharmaceutically acceptable salt thereof, wherein R⁵ is C₁₋₆ alkyl.

37. The compound of claim 1, or a pharmaceutically acceptable salt thereof, wherein R⁵ is methyl.

38. The compound of claim 1, or a pharmaceutically acceptable salt thereof, wherein R⁶ is H.

39. The compound of claim 1, or a pharmaceutically acceptable salt thereof, wherein R⁶ is H or OR^{a9}.

40. The compound of claim 1, or a pharmaceutically acceptable salt thereof, wherein the compound has Formula IIIa:

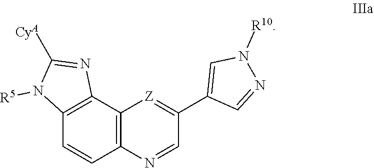

41. The compound of claim 1, or a pharmaceutically acceptable salt thereof, wherein the compound has Formula IIIb:

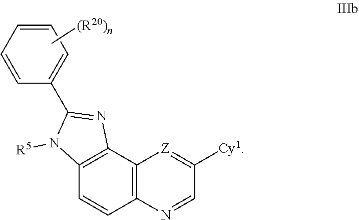

42. The compound of claim 1, or a pharmaceutically acceptable salt thereof, wherein the compound has Formula IIIc:

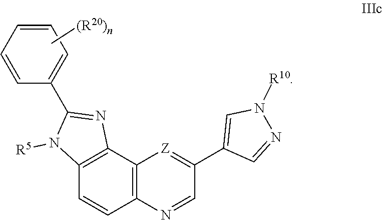

43. The compound of claim 1, having Formula (IA): 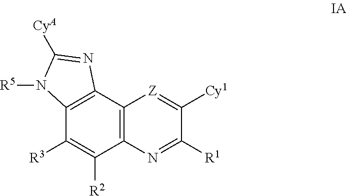
or a pharmaceutically acceptable salt thereof, wherein:
Z is N;
Cy¹ is 5-10 membered heteroaryl; wherein each 5-10 membered heteroaryl has at least one ring-forming carbon atom and 1, 2, 3, or 4 ring-forming heteroatoms independently selected from N, O, and S; wherein the N and S are optionally oxidized; wherein a ring-forming carbon atom of 5-10 membered heteroaryl is optionally substituted by oxo to form a carbonyl group; and wherein the C₆₋₁₀ aryl and 5-10 membered heteroaryl are each optionally substituted with 1, 2, 3 or 4 substituents independently selected from R¹⁰;
Cy^{A} is selected from C₃₋₁₀ cycloalkyl, 4-10 membered heterocycloalkyl, C₆₋₁₀ aryl and 5-10 membered heteroaryl; wherein the 4-10 membered heterocycloalkyl and 5-10 membered heteroaryl each has at least one ring-forming carbon atom and 1, 2, 3, or 4 ring-forming heteroatoms independently selected from N, O, and S; wherein the N and S are optionally oxidized; wherein a ring-forming carbon atom of 5-10 membered heteroaryl and 4-10 membered heterocycloalkyl is optionally substituted by oxo to form a carbonyl group; and wherein the C₃₋₁₀ cycloalkyl, 4-10 membered heterocycloalkyl, C₆₋₁₀ aryl and 5-10 membered heteroaryl are each optionally substituted with 1, 2, 3 or 4 substituents independently selected from R²⁰;
R¹, R², and R³ are each independently selected from H, D, C₁₋₆ alkyl, C₁₋₆ haloalkyl, halo, CN, OR^{a9}, and NR^{c9}R^{d9};
R⁵ is selected from C₁₋₆ alkyl, C₁₋₆ haloalkyl, C₃₋₁₀ cycloalkyl, and C₆₋₁₀ aryl; wherein said C₁₋₆ alkyl, C₃₋₁₀ cycloalkyl, and C₆₋₁₀ aryl, are each optionally substituted with 1, 2, 3, or 4 substituents independently selected from R⁵⁰;
each R¹⁰ is independently selected from C₁₋₆ alkyl, C₁₋₆ haloalkyl, C₃₋₁₀ cycloalkyl, 4-10 membered heterocycloalkyl, halo, D, CN, OR^{a1}, and NR^{c1}R^{d1}; wherein said C₁₋₆ alkyl, C₃₋₁₀ cycloalkyl, and 4-10 membered heterocycloalkyl, are each optionally substituted with 1, 2, 3, or 4 substituents independently selected from R¹¹;
each R¹¹ is independently selected from C₁₋₆ alkyl, C₁₋₆ haloalkyl, C₃₋₁₀ cycloalkyl, halo, D, CN, OR^{a3}, C(O)R^{b3}, C(O)NR^{c3}R^{d3}, C(O)OR^{a3}, NR^{c3}R^{d3}, and S(O)₂R^{b3}; wherein said C₁₋₆ alkyl, and C₃₋₁₀ cycloalkyl, are each optionally substituted with 1, 2, 3, or 4 substituents independently selected from R¹²;
each R¹² is independently selected from C₁₋₆ alkyl, halo, D, CN, OR^{a5} and NR^{c5}R^{d5};
each R²⁰ is independently selected from C₁₋₆ alkyl, C₁₋₆ haloalkyl, C₃₋₁₀ cycloalkyl, halo, D, CN, OR^{a2}, C(O)R^{b2}, C(O)NR^{c2}R^{d2}, C(O)OR^{a2}, S(O)NR^{c2}R^{d2}, and S(O)₂R^{b2}; wherein said C₁₋₆ alkyl, and C₃₋₁₀ cycloalkyl are each optionally substituted with 1, 2, 3, or 4 substituents independently selected from R²¹;
each R²¹ is independently selected from C₁₋₆ alkyl, C₁₋₆ haloalkyl, halo, D, CN, OR^{a4}, and NR^{c4}R^{d4};
each R⁵⁰ is independently selected from C₁₋₆ alkyl, C₁₋₆ haloalkyl, halo, D, CN, OR^{a11}, and NR^{c11}R^{d11};
each R^{a1}, R^{c1} and R^{d1} is independently selected from H, C₁₋₆ alkyl, C₁₋₆ haloalkyl, C₃₋₁₀ cycloalkyl, and 4-10 membered heterocycloalkyl; wherein said C₁₋₆ alkyl, C₃₋₁₀ cycloalkyl, and 4-10 membered heterocycloalkyl, are each optionally substituted with 1, 2, 3, or 4 substituents independently selected from R¹¹;
or any R^{c1} and R^{d1} attached to the same N atom, together with the N atom to which they are attached, form a 4-, 5-, or 6-membered heterocycloalkyl group optionally substituted with 1, 2, 3, or 4 substituents independently selected from R¹¹;
each R^{a2}, R^{c2} and R^{d2} is independently selected from H, C₁₋₆ alkyl, C₁₋₆ haloalkyl, and C₃₋₁₀ cycloalkyl; wherein said C₁₋₆ alkyl and C₃₋₁₀ cycloalkyl, are each optionally substituted with 1, 2, 3, or 4 substituents independently selected from R²¹;
each R^{b2} is independently selected from C₁₋₆ alkyl, C₁₋₆ haloalkyl, and C₃₋₁₀ cycloalkyl;
wherein said C₁₋₆ alkyl and C₃₋₁₀ cycloalkyl, are each optionally substituted with 1, 2, 3, or 4 substituents independently selected from R²¹;
each R^{a3}, R^{c3} and R^{d3} is independently selected from H, C₁₋₆ alkyl, C₁₋₆ haloalkyl, and C₃₋₆ cycloalkyl; wherein said C₁₋₆ alkyl and C₃₋₆ cycloalkyl, are each optionally substituted with 1, 2, 3, or 4 substituents independently selected from R¹²;
each R^{b3} is independently selected from C₁₋₆ alkyl, C₁₋₆ haloalkyl, and C₃₋₆ cycloalkyl; wherein said C₁₋₆ alkyl and C₃₋₆ cycloalkyl, are each optionally substituted with 1, 2, 3, or 4 substituents independently selected from R¹²;
each R^{a4}, R^{c4} and R^{d4} is independently selected from H, C₁₋₆ alkyl, and C₁₋₆ haloalkyl;
each R^{a5}, R^{c5} and R^{d5} is independently selected from H, C₁₋₆ alkyl, and C₁₋₆ haloalkyl;
each R^{a9}, R^{c9}, and R^{d9} is independently selected from H, C₁₋₆ alkyl, and C₁₋₆ haloalkyl; and
each R^{a11}, R^{c11}, and R^{d11} is independently selected from H, C₁₋₆ alkyl, and C₁₋₆ haloalkyl.

44. The compound of claim 1, having Formula (IA): 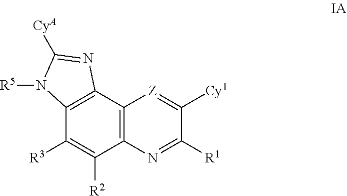
or a pharmaceutically acceptable salt thereof, wherein:
Z is N;
Cy¹ is selected from 5-10 membered heteroaryl; wherein each 5-10 membered heteroaryl has at least one ring-forming carbon atom and 1, 2, 3, or 4 ring-forming heteroatoms independently selected from N, O, and S; wherein a ring-forming carbon atom of 5-10 membered heteroaryl is optionally substituted by oxo to form a carbonyl group; and wherein the 5-10 membered heteroaryl are each optionally substituted with 1 or 2 substituents independently selected from R¹⁰;
Cy^{A} is selected from 4-10 membered heterocycloalkyl, C₆₋₁₀ aryl and 5-10 membered heteroaryl; wherein the 4-10 membered heterocycloalkyl and 5-10 membered heteroaryl each has at least one ring-forming carbon atom and 1, 2, 3, or 4 ring-forming heteroatoms independently selected from N, O, and S; wherein a ring-forming carbon atom of 5-10 membered heteroaryl and 4-10 membered heterocycloalkyl is optionally substituted by oxo to form a carbonyl group; and wherein the 4-10 membered heterocycloalkyl, C₆₋₁₀ aryl and 5-10 membered heteroaryl are each optionally substituted with 1, 2, or 3 substituents independently selected from R²⁰;
R¹, R², and R³ are each independently selected from H, D, C₁₋₆ alkyl, C₁₋₆ haloalkyl halo, and CN;
R⁵ is selected from H, D, C₁₋₆ alkyl, and C₁₋₆ haloalkyl;
each R¹⁰ is independently selected from C₁₋₆ alkyl, C₁₋₆ haloalkyl, C₃₋₁₀ cycloalkyl, 4-10 membered heterocycloalkyl, halo, D, CN, OR^{a1}, and NR^{c1}R^{d1}; wherein said C₁₋₆ alkyl, C₃₋₁₀ cycloalkyl, and 4-10 membered heterocycloalkyl, are each optionally substituted with 1, 2, or 3 substituents independently selected from R¹¹;
each R¹¹ is independently selected from C₁₋₆ alkyl, C₁₋₆ haloalkyl, halo, D, CN, OR^{a3} C(O)R^{b3}, C(O)NR^{c3}R^{d3}, and NR^{c3}R^{d3};
each R²⁰ is independently selected from C₁₋₆ alkyl, C₁₋₆ haloalkyl, halo, D, CN, OR^{a2}, and NR^{c2}R^{d2};
each R^{a1}, R^{c1} and R^{d1} is independently selected from H, C₁₋₆ alkyl, C₁₋₆ haloalkyl, C₃₋₁₀ cycloalkyl, and 4-10 membered heterocycloalkyl; wherein said C₁₋₆ alkyl, C₃₋₁₀ cycloalkyl, and 4-10 membered heterocycloalkyl, are each optionally substituted with 1 or 2 substituents independently selected from R¹¹;
or any R^{c1} and R^{d1} attached to the same N atom, together with the N atom to which they are attached, form a 4-, 5-, or 6-membered heterocycloalkyl group optionally substituted with 1 or 2 substituents independently selected from R¹¹;
each R^{a2}, R^{c2} and R^{d2}, is independently selected from H, C₁₋₆ alkyl, and C₁₋₆ haloalkyl;
each R^{a3}, R^{c3} and R^{d3}, is independently selected from H, C₁₋₆ alkyl, and C₁₋₆ haloalkyl; and
each R^{b3} is independently selected from C₁₋₆ alkyl, and C₁₋₆ haloalkyl.

45-46. (canceled)

47. The compound of claim 1, wherein the compound is selected from:
2-(2-Fluoro-6-methyl) 1)-3-methyl-8-(1-methyl-1H-pyrazol-4-yl)-3H-imidazo[4,5-f]quinoxaline;
2-(2-Chloro-4-methoxyphenyl)-3-methyl-8-(1-methyl-H-pyrazol-4-yl)-3H-imidazo[4,5-f]quinoxaline;
3-Methyl-8-(1-methyl-1H-pyrazol-4-yl)-2-(3-methylfuran-2-yl)-3H-imidazo[4,5-f]quinoxaline;
3-(4-(2-(2-Chloro-6-fluorophenyl)-3-methyl-3H-imidazo[4,5-f]quinoxalin-8-yl)-1H-pyrazol-1-yl)cyclobutan-1-ol; and
1-(4-(5-(2-(2-Chloro-6-fluorophenyl)-3-methyl-3H-imidazo[4,5-f]quinoxalin-8-yl)pyridin-2-yl)piperazin-1-yl)ethan-1-one,
or a pharmaceutically acceptable of any of the aforementioned.

48. The compound of claim 1, wherein the compound is selected from
3-Methyl-8-(1-methyl-1H-pyrazol-4-yl)-2-(1-methylpiperidin-4-yl)-3H-imidazo[4,5-f]quinoxaline; and
(1S,5R)-8-(3-Methyl-8-(1-methyl-1H-pyrazol-4-yl)-3H-imidazo[4,5-f]quinoxalin-2-yl)-3,8-diazabicyclo[3.2.1]octan-2-one
or a pharmaceutically acceptable salt ofany of the aforementioned.

49. A pharmaceutical composition comprising a compound of claim 1 or a pharmaceutically acceptable salt thereof, and a pharmaceutically acceptable carrier or excipient.

50. A method of inhibiting an FGFR3 enzyme comprising contacting said enzyme with a compound of claim 1 or a pharmaceutically acceptable salt thereof.

51. A method of treating cancer in a patient comprising administering to said patient a therapeutically effective amount of a compound of claim 1 or a pharmaceutically acceptable salt thereof.

52. A method of treating cancer in a patient comprising administering to said patient a therapeutically effective amount of a compound of claim 1 or a pharmaceutically acceptable salt thereof, in combination with another therapy or therapeutic agent.

53. The method of claim 51, wherein said cancer is selected from hepatocellular cancer, bladder cancer, breast cancer, cervical cancer, colorectal cancer, endometrial cancer, gastric cancer, head and neck cancer, kidney cancer, liver cancer, lung cancer, ovarian cancer, prostate cancer, esophageal cancer, gall bladder cancer, pancreatic cancer, thyroid cancer, skin cancer, leukemia, multiple myeloma, chronic lymphocytic lymphoma, adult T cell leukemia, B-cell lymphoma, acute myelogenous leukemia, Hodgkin's or non-Hodgkin's lymphoma, Waldenstrom's Macroglubulinemia, hairy cell lymphoma, Burkett's lymphoma, glioblastoma, melanoma, and rhabdosarcoma.

54. The method of claim 51, wherein said cancer is selected from hepatocellular cancer, breast cancer, bladder cancer, colorectal cancer, melanoma, mesothelioma, lung cancer, prostate cancer, pancreatic cancer, testicular cancer, thyroid cancer, squamous cell carcinoma, glioblastoma, neuroblastoma, uterine cancer, and rhabdosarcoma.

55. A method for treating a skeletal or chondrocyte disorder in a patient comprising administering to said patient a therapeutically effective amount of a compound of claim 1 or a pharmaceutically acceptable salt thereof.

56. The method of claim 55 wherein said skeletal or chondrocyte disorder is selected from achrondroplasia, hypochondroplasia, dwarfism, thanatophoric dysplasia (TD), Apert syndrome, Crouzon syndrome, Jackson-Weiss syndrome, Beare-Stevenson cutis gyrate syndrome, Pfeiffer syndrome, and craniosynostosis syndrome.
